(19) United States
(12) Patent Application Publication
(10) Pub. No.: US 2022/0415476 A1
(43) Pub. Date: Dec. 29, 2022

(54) Wearable Device and System for Nutritional Intake Monitoring and Management

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 17/903,746

(22) Filed: Sep. 6, 2022

Related U.S. Application Data
(63) Continuation-in-part of application No. 17/239,960, filed on Apr. 26, 2021.
Application No. 17/239,960 is a continuation-in-part of application No. 16/737,052, filed on Jan. 8, 2020.
Application No. 16/737,052 is a continuation-in-part of application No. 16/568,580, filed on Sep. 12, 2019, now Pat. No. 11,478,158.
Application No. 16/568,580 is a continuation-in-part of application No. 16/737,052, filed on Jan. 8, 2020.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/294,746, filed on Oct. 16, 2016, now Pat. No. 10,627,861.
Application No. 15/294,746 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/418,620, filed on Jan. 27, 2017, now abandoned.
Application No. 15/418,620 is a continuation-in-part of application No. 15/294,746, filed on Oct. 16, 2016, now Pat. No. 10,627,861.
Application No. 15/963,061 is a continuation-in-part of application No. 14/992,073, filed on Jan. 11, 2016, now abandoned.
Application No. 14/992,073 is a continuation-in-part of application No. 14/550,953, filed on Nov. 22, 2014, now abandoned.
Application No. 14/550,953 is a continuation-in-part of application No. 15/431,769, filed on Feb. 14, 2017, now abandoned.
Application No. 15/431,769 is a continuation-in-part of application No. 14/951,475, filed on Nov. 24, 2015, now Pat. No. 10,314,492.
Application No. 14/951,475 is a continuation-in-part of application No. 15/294,746, filed on Oct. 16, 2016, now Pat. No. 10,627,861.
Application No. 15/294,746 is a continuation-in-part of application No. 15/206,215, filed on Jul. 8, 2016, now abandoned.
Application No. 15/206,215 is a continuation-in-part of application No. 14/992,073, filed on Jan. 11, 2016, now abandoned.
Application No. 14/992,073 is a continuation-in-part of application No. 14/330,649, filed on Jul. 14, 2014, now abandoned.
Application No. 14/330,649 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/948,308, filed on Nov. 21, 2015, now abandoned.
Application No. 14/992,073 is a continuation-in-part of application No. 14/562,719, filed on Dec. 7, 2014, now Pat. No. 10,130,277.
Application No. 14/562,719 is a continuation-in-part of application No. 13/616,238, filed on Sep. 14, 2012, now abandoned.
Application No. 14/951,475 is a continuation-in-part of application No. 14/071,112, filed on Nov. 4, 2013, now abandoned.
Application No. 14/071,112 is a continuation-in-part of application No. 13/901,131, filed on May 23, 2013, now Pat. No. 9,536,449.
Application No. 14/948,308 is a continuation-in-part of application No. 14/550,953, filed on Nov. 22, 2014, now abandoned.
Application No. 14/550,953 is a continuation-in-part of application No. 14/449,387, filed on Aug. 1, 2014, now abandoned.
Application No. 14/449,387 is a continuation-in-part of application No. 14/132,292, filed on Dec. 18, 2013, now Pat. No. 9,442,100.
Application No. 14/132,292 is a continuation-in-part of application No. 13/901,099, filed on May 23, 2013, now Pat. No. 9,254,099.
Application No. 14/330,649 is a continuation-in-part of application No. 13/523,739, filed on Jun. 14, 2012, now Pat. No. 9,042,596.
(60) Provisional application No. 63/279,773, filed on Nov. 16, 2021.
(60) Provisional application No. 63/171,838, filed on Apr. 7, 2021.
(60) Provisional application No. 62/930,013, filed on Nov. 4, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/800,478, filed on Feb. 2, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/549,587, filed on Aug. 24, 2017.
(60) Provisional application No. 62/439,147, filed on Dec. 26, 2016.
(60) Provisional application No. 62/439,147, filed on Dec. 26, 2016.
(60) Provisional application No. 62/349,277, filed on Jun. 13, 2016.
(60) Provisional application No. 62/311,462, filed on Mar. 22, 2016.
(60) Provisional application No. 62/297,827, filed on Feb. 20, 2016.
(60) Provisional application No. 62/349,277, filed on Jun. 13, 2016.
(60) Provisional application No. 62/245,311, filed on Oct. 23, 2015.
(60) Provisional application No. 62/349,277, filed on Jun. 13, 2016.
(60) Provisional application No. 61/932,517, filed on Jan. 28, 2014.

(51) Int. Cl. G16H 20/60; A61B 5/00; G06F 1/16

(52) U.S. Cl. CPC G16H 20/60; A61B 5/681; G06F 1/163; A61B 2562/0219

(57) ABSTRACT

This invention is a wearable device which helps a person to track their food intake as part of a system for nutritional intake monitoring and management. This invention can be embodied in a smart watch and/or wrist band with a camera, a display and/or camera viewfinder, a spectroscopic sensor, and an eating detector. The eating detector can be an accelerometer, a gyroscope, a magnetometer, a microphone, or an EMG sensor. The camera and/or the spectroscopic sensor can be automatically activated when the person eats food, but otherwise remain off to help maintain privacy.

### CROSS-REFERENCE TO RELATED APPLICATIONS

This application is a continuation-in-part of U.S. patent application Ser. No. 16/568,580 filed on 2019 Sep. 12. This application is a continuation-in-part of U.S. patent application Ser. No. 16/737,052 filed on 2020 Jan. 8. This application is a continuation-in-part of U.S. patent application Ser. No. 17/239,960 filed on 2021 Apr. 26. This application claims the priority benefit of U.S. provisional application 63/279,773 filed on 2021 Nov. 16.

U.S. patent application Ser. No. 17/239,960 claimed the priority benefit of U.S. provisional application 63/171,838 filed on 2021 Apr. 7. U.S. patent application Ser. No. 17/239,960 was a continuation-in-part of U.S. patent application Ser. No. 16/737,052 filed on 2020 Jan. 8.

U.S. patent application Ser. No. 16/737,052 claimed the priority benefit of U.S. provisional application 62/930,013 filed on 2019 Nov. 4. U.S. patent application Ser. No. 16/737,052 claimed the priority benefit of U.S. provisional application 62/857,942 filed on 2019 Jun. 6. U.S. patent application Ser. No. 16/737,052 claimed the priority benefit of U.S. provisional application 62/814,713 filed on 2019 Mar. 6. U.S. patent application Ser. No. 16/737,052 claimed the priority benefit of U.S. provisional application 62/814,692 filed on 2019 Mar. 6. U.S. patent application Ser. No. 16/737,052 claimed the priority benefit of U.S. provisional application 62/800,478 filed on 2019 Feb. 2. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25 which issued as U.S. patent Ser. No. 10/772,559 on 2020 Sep. 15. U.S. patent application Ser. No. 16/737,052 was a continuation-in-part of U.S. patent application Ser. No. 15/725,330 filed on 2017 Oct. 5 which issued as U.S. patent Ser. No. 10/607,507 on 2020 Mar. 31. U.S. patent application Ser. No. 16/737,052 was a continuation-in-part of U.S. patent application Ser. No. 15/431,769 filed on 2017 Feb. 14. U.S. patent application Ser. No. 16/737,052 was a continuation-in-part of U.S. patent application Ser. No. 15/294,746 filed on 2016 Oct. 16 which issued as U.S. patent Ser. No. 10/627,861 on 2020 Apr. 21.

U.S. patent application Ser. No. 16/568,580 claimed the priority benefit of U.S. provisional application 62/857,942 filed on 2019 Jun. 6. U.S. patent application Ser. No. 16/568,580 claimed the priority benefit of U.S. provisional application 62/814,713 filed on 2019 Mar. 6. U.S. patent application Ser. No. 16/568,580 claimed the priority benefit of U.S. provisional application 62/814,692 filed on 2019 Mar. 6. U.S. patent application Ser. No. 16/568,580 was a continuation-in-part of U.S. patent application Ser. No. 15/963,061 filed on 2018 Apr. 25 which issued as U.S. patent Ser. No. 10/772,559 on 2020 Sep. 15. U.S. patent application Ser. No. 16/568,580 was a continuation-in-part of U.S. patent application Ser. No. 15/725,330 filed on 2017 Oct. 5 which issued as U.S. patent Ser. No. 10/607,507 on 2020 Mar. 31. U.S. patent application Ser. No. 16/568,580 was a continuation-in-part of U.S. patent application Ser. No. 15/431,769 filed on 2017 Feb. 14. U.S. patent application Ser. No. 16/568,580 was a continuation-in-part of U.S. patent application Ser. No. 15/418,620 filed on 2017 Jan. 27. U.S. patent application Ser. No. 16/568,580 was a continuation-in-part of U.S. patent application Ser. No. 15/294,746 filed on 2016 Oct. 16 which issued as U.S. patent Ser. No. 10/627,861 on 2020 Apr. 21.

U.S. patent application Ser. No. 15/963,061 was a continuation-in-part of U.S. patent application Ser. No. 14/992,073 filed on 2016 Jan. 11. U.S. patent application Ser. No. 15/963,061 was a continuation-in-part of U.S. patent application Ser. No. 14/550,953 filed on 2014 Nov. 22.

U.S. patent application Ser. No. 15/725,330 claimed the priority benefit of U.S. provisional application 62/549,587 filed on 2017 Aug. 24. U.S. patent application Ser. No. 15/725,330 claimed the priority benefit of U.S. provisional application 62/439,147 filed on 2016 Dec. 26. U.S. patent application Ser. No. 15/725,330 was a continuation-in-part of U.S. patent application Ser. No. 15/431,769 filed on 2017 Feb. 14. U.S. patent application Ser. No. 15/725,330 was a continuation-in-part of U.S. patent application Ser. No. 14/951,475 filed on 2015 Nov. 24 which issued as U.S. patent Ser. No. 10/314,492 on 2019 Jun. 11.

U.S. patent application Ser. No. 15/431,769 claimed the priority benefit of U.S. provisional application 62/439,147 filed on 2016 Dec. 26. U.S. patent application Ser. No. 15/431,769 claimed the priority benefit of U.S. provisional application 62/349,277 filed on 2016 Jun. 13. U.S. patent application Ser. No. 15/431,769 claimed the priority benefit of U.S. provisional application 62/311,462 filed on 2016 Mar. 22. U.S. patent application Ser. No. 15/431,769 was a continuation-in-part of U.S. patent application Ser. No. 15/294,746 filed on 2016 Oct. 16 which issued as U.S. patent Ser. No. 10/627,861 on 2020 Apr. 21. U.S. patent application Ser. No. 15/431,769 was a continuation-in-part of U.S. patent application Ser. No. 15/206,215 filed on 2016 Jul. 8. U.S. patent application Ser. No. 15/431,769 was a continuation-in-part of U.S. patent application Ser. No. 14/992,073 filed on 2016 Jan. 11. U.S. patent application Ser. No. 15/431,769 was a continuation-in-part of U.S. patent application Ser. No. 14/330,649 filed on 2014 Jul. 14.

U.S. patent application Ser. No. 15/418,620 claimed the priority benefit of U.S. provisional application 62/297,827 filed on 2016 Feb. 20. U.S. patent application Ser. No. 15/418,620 was a continuation-in-part of U.S. patent application Ser. No. 14/951,475 filed on 2015 Nov. 24 which issued as U.S. patent Ser. No. 10/314,492 on 2019 Jun. 11.

U.S. patent application Ser. No. 15/294,746 claimed the priority benefit of U.S. provisional application 62/349,277 filed on 2016 Jun. 13. U.S. patent application Ser. No. 15/294,746 claimed the priority benefit of U.S. provisional application 62/245,311 filed on 2015 Oct. 23. U.S. patent application Ser. No. 15/294,746 was a continuation-in-part of U.S. patent application Ser. No. 14/951,475 filed on 2015 Nov. 24 which issued as U.S. patent Ser. No. 10/314,492 on 2019 Jun. 11.

U.S. patent application Ser. No. 15/206,215 claimed the priority benefit of U.S. provisional application 62/349,277 filed on 2016 Jun. 13. U.S. patent application Ser. No. 15/206,215 was a continuation-in-part of U.S. patent application Ser. No. 14/951,475 filed on 2015 Nov. 24 which issued as U.S. patent Ser. No. 10/314,492 on 2019 Jun. 11. U.S. patent application Ser. No. 15/206,215 was a continuation-in-part of U.S. patent application Ser. No. 14/948,308 filed on 2015 Nov. 21.

U.S. patent application Ser. No. 14/992,073 was a continuation-in-part of U.S. patent application Ser. No. 14/562,719 filed on 2014 Dec. 7 which issued as U.S. patent Ser. No. 10/130,277 on 2018 Nov. 20. U.S. patent application Ser. No. 14/992,073 was a continuation-in-part of U.S. patent application Ser. No. 13/616,238 filed on 2012 Sep. 14.

U.S. patent application Ser. No. 14/951,475 was a continuation-in-part of U.S. patent application Ser. No. 14/071,112 filed on 2013 Nov. 4. U.S. patent application Ser. No. 14/951,475 was a continuation-in-part of U.S. patent application Ser. No. 13/901,131 filed on 2013 May 23 which issued as U.S. Pat. No. 9,536,449 on 2017 Jan. 3.

U.S. patent application Ser. No. 14/948,308 was a continuation-in-part of U.S. patent application Ser. No. 14/550,953 filed on 2014 Nov. 22. U.S. patent application Ser. No. 14/948,308 was a continuation-in-part of U.S. patent application Ser. No. 14/449,387 filed on 2014 Aug. 1. U.S. patent application Ser. No. 14/948,308 was a continuation-in-part of U.S. patent application Ser. No. 14/132,292 filed on 2013 Dec. 18 which issued as U.S. Pat. No. 9,442,100 on 2016 Sep. 13. U.S. patent application Ser. No. 14/948,308 was a continuation-in-part of U.S. patent application Ser. No. 13/901,099 filed on 2013 May 23 which issued as U.S. Pat. No. 9,254,099 on 2016 Feb. 9.

U.S. patent application Ser. No. 14/562,719 claimed the priority benefit of U.S. provisional application 61/932,517 filed on 2014 Jan. 28.

U.S. patent application Ser. No. 14/330,649 was a continuation-in-part of U.S. patent application Ser. No. 13/523,739 filed on 2012 Jun. 14 which issued as U.S. Pat. No. 9,042,596 on 2015 May 26.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable devices for measuring food consumption.

### Introduction

Many health problems are caused by poor nutrition. Many people consume too much unhealthy food or not enough healthy food. Although there are complex behavioral reasons for poor dietary habits, better nutritional monitoring and awareness concerning the types and quantities of food consumed can help people to improve their dietary habits and health. Information concerning the types and quantities of food consumed can be part of a system that provides constructive feedback and/or incentives to help people improve their nutritional intake. People can try to track the types and quantities of food consumed without technical assistance. Their unassisted estimates of the types and quantities of consumed food can be translated into types and quantities of nutrients consumed. However, such unassisted tracking can be subjective. Also, such unassisted tracking can be particularly challenging for non-standardized food items such as food prepared in an ad hoc manner at restaurants or in homes. It would be useful to have a relatively-unobtrusive wearable device which can help people to accurately track the types and quantities of food which they consume.

### Review of the Relevant Art

In the patent literature, U.S. patent application publications 20090012433 (Fernstrom et al., Jan. 8, 2009, “Method, Apparatus and System for Food Intake and Physical Activity Assessment”), 20130267794 (Fernstrom et al., Oct. 10, 2013, “Method, Apparatus and System for Food Intake and Physical Activity Assessment”), and 20180348187 (Fernstrom et al., Dec. 6, 2018, “Method, Apparatus and System for Food Intake and Physical Activity Assessment”), as well as U.S. Pat. No. 9,198,621 (Fernstrom et al., Dec. 1, 2015, “Method, Apparatus and System for Food Intake and Physical Activity Assessment”) and 10006896 (Fernstrom et al., Jun. 26, 2018, “Method, Apparatus and System for Food Intake and Physical Activity Assessment”), disclose wearable buttons and necklaces for monitoring eating with cameras. U.S. patent Ser. No. 10/900,943 (Fernstrom et al, Jan. 26, 2021, “Method, Apparatus and System for Food Intake and Physical Activity Assessment”) discloses monitoring food consumption using a wearable device with two video cameras and an infrared sensor.

U.S. patent application publication 20160073953 (Sazonov et al., Mar. 17, 2016, “Food Intake Monitor”) discloses monitoring food consumption using a wearable device with a jaw motion sensor and a hand gesture sensor. U.S. patent application publication 20180242908 (Sazonov et al., Aug. 30, 2018, “Food Intake Monitor”) and U.S. patent Ser. No. 10/736,566 (Sazonov, Aug. 11, 2020, “Food Intake Monitor”) disclose monitoring food consumption using an ear-worn device or eyeglasses with a pressure sensor and accelerometer.

U.S. patent application publication 20190333634 (Vleugels et al., Oct. 31, 2019, “Method and Apparatus for Tracking of Food Intake and Other Behaviors and Providing Relevant Feedback”), 20170220772 (Vleugels et al., Aug. 3, 2017, “Method and Apparatus for Tracking of Food Intake and Other Behaviors and Providing Relevant Feedback”), and 20180300458 (Vleugels et al., Oct. 18, 2018, “Method and Apparatus for Tracking of Food Intake and Other Behaviors and Providing Relevant Feedback”), as well as U.S. patent Ser. No. 10/102,342 (Vleugels et al., Oct. 16, 2018, “Method and Apparatus for Tracking of Food Intake and Other Behaviors and Providing Relevant Feedback”) and 10373716 (Vleugels et al., Aug. 6, 2019, “Method and Apparatus for Tracking of Food Intake and Other Behaviors and Providing Relevant Feedback”), disclose a method for detecting, identifying, analyzing, quantifying, tracking, processing and/or influencing food consumption. U.S. patent application publication 20190236465 (Vleugels, Aug. 1, 2019, “Activation of Ancillary Sensor Systems Based on Triggers from a Wearable Gesture Sensing Device”) discloses an eating monitor with gesture recognition.

U.S. patent application publication 20200294645 (Vleugels, Sep. 17, 2020, “Gesture-Based Detection of a Physical Behavior Event Based on Gesture Sensor Data and Supplemental Information from at Least One External Source”) discloses an automated medication dispensing system which recognizes gestures. U.S. patent Ser. No. 10/790,054 (Vleugels et al., Sep. 29, 2020, “Method and Apparatus for Tracking of Food Intake and Other Behaviors and Providing Relevant Feedback”) discloses a computer-based method of detecting gestures. U.S. patent application publication 20200381101 (Vleugels, Dec. 3, 2020, “Method and Apparatus for Tracking of Food Intake and Other Behaviors and Providing Relevant Feedback”) discloses methods for detecting, identifying, analyzing, quantifying, tracking, processing and/or influencing, related to the intake of food, eating habits, eating patterns, and/or triggers for food intake events, eating habits, or eating patterns.

U.S. patent application publications 20160299061 (Goldring et al., Oct. 13, 2016, “Spectrometry Systems, Methods, and Applications”), 20170160131 (Goldring et al., Jun. 8, 2017, “Spectrometry Systems, Methods, and Applications”), 20180085003 (Goldring et al., Mar. 29, 2018, “Spectrometry Systems, Methods, and Applications”), 20180120155 (Rosen et al., May 3, 2018, “Spectrometry Systems, Methods, and Applications”), and 20180180478 (Goldring et al., Jun. 28, 2018, “Spectrometry Systems, Methods, and Applications”) disclose a handheld spectrometer to measure the spectra of objects. U.S. patent application publication 20180136042 (Goldring et al., May 17, 2018, “Spectrometry System with Visible Aiming Beam”) discloses a handheld spectrometer with a visible aiming beam. U.S. patent application publication 20180252580 (Goldring et al., Sep. 6, 2018, “Low-Cost Spectrometry System for End-User Food Analysis”) discloses a compact spectrometer that can be used in mobile devices such as smart phones. U.S. patent application publication 20190033130 (Goldring et al., Jan. 31, 2019, “Spectrometry Systems, Methods, and Applications”) discloses a hand held spectrometer with wavelength multiplexing. U.S. patent application publication 20190033132 (Goldring et al., Jan. 31, 2019, “Spectrometry System with Decreased Light Path”) discloses a spectrometer with a plurality of isolated optical channels. U.S. patent application publication 20190041265 (Rosen et al., Feb. 7, 2019, “Spatially Variable Filter Systems and Methods”) discloses a compact spectrometer system with a spatially variable filter.

U.S. patent application publication 20150302160 (Muthukumar et al., Oct. 22, 2015, “Method and Apparatus for Monitoring Diet and Activity”) discloses a method and device for analyzing food with a camera and a spectroscopic sensor. U.S. patent Ser. No. 10/143,420 (Contant, Dec. 4, 2018, “Eating Utensil to Monitor and Regulate Dietary Intake”) discloses a dietary intake regulating device that also monitors physical activity.

U.S. patent application publication 20160148535 (Ashby, May 26, 2016, “Tracking Nutritional Information about Consumed Food”) discloses an eating monitor which monitors swallowing and/or chewing. U.S. patent application publication 20160148536 (Ashby, May 26, 2016, “Tracking Nutritional Information about Consumed Food with a Wearable Device”) discloses an eating monitor with a camera. U.S. patent application publication 20190213416 (Cho et al., Jul. 11, 2019, “Electronic Device and Method for Processing Information Associated with Food”) discloses a food tracking device with a camera. U.S. patent application publication 20170061821 (Choi et al., Mar. 2, 2017, “Systems and Methods for Performing a Food Tracking Service for Tracking Consumption of Food Items”) discloses a food tracking service. U.S. patent application publication 20190167190 (Choi et al., Jun. 6, 2019, “Healthcare Apparatus and Operating Method Thereof”) discloses a dietary monitoring device which emits light of different wavelengths.

U.S. patent application publication 20160163037 (Dehais et al., Jun. 9, 2016, “Estimation of Food Volume and Carbs”) discloses an image-based food identification system including a projected light pattern. U.S. patent application publication 20170249445 (Devries et al., Aug. 31, 2017, “Portable Devices and Methods for Measuring Nutritional Intake”) discloses a nutritional intake monitoring system with biosensors. U.S. patent application publication 20150294450 (Eyring, Oct. 15, 2015, “Systems and Methods for Measuring Calorie Intake”) discloses an image-based system for measuring caloric input. U.S. patent application publication 20150325142 (Ghalavand, Nov. 12, 2015, “Calorie Balance System”) discloses a calorie balance system with smart utensils and/or food scales.

U.S. patent application publication 20190295440 (Hadad, Sep. 26, 2019, “Systems and Methods for Food Analysis, Personalized Recommendations and Health Management”) discloses a method for developing a food ontology. U.S. patent application publications 20190244541 (Hadad et al., Aug. 8, 2019, “Systems and Methods for Generating Personalized Nutritional Recommendations”), 20140255882 (Hadad et al., Sep. 11, 2014, “Interactive Engine to Provide Personal Recommendations for Nutrition, to Help the General Public to Live a Balanced Healthier Lifestyle”), and 20190290172 (Hadad et al., Sep. 26, 2019, “Systems and Methods for Food Analysis, Personalized Recommendations, and Health Management”) disclose methods to provide nutrition recommendations based on a person's preferences, habits, medical and activity. U.S. patent application publication 20160103910 (Kim et al., Apr. 14, 2016, “System and Method for Food Categorization”) discloses a food categorization engine. U.S. patent application publication 20190244704 (Kim et al., Aug. 8, 2019, “Dietary Habit Management Apparatus and Method”) discloses a dietary habit management apparatus using biometric measurements.

U.S. patent application publication 20160140869 (Kuwahara et al., May 19, 2016, “Food Intake Controlling Devices and Methods”) discloses image-based technologies for controlling food intake. U.S. patent application publication 20170156634 (Li et al., Jun. 8, 2017, “Wearable Device and Method for Monitoring Eating”) and patent Ser. No. 10/499,833 (Li et al., Dec. 10, 2019, “Wearable Device and Method for Monitoring Eating”) disclose a wearable device with an acceleration sensor to monitor eating. U.S. patent application publication 20160313241 (Ochi et al., Nov. 27, 2016, “Calorie Measurement Device”) disclose, Mar. 17, 2016, “Food Intake Monitor”) discloses a jaw motion sensor to measure food intake. U.S. patent application publication 20180005545 (Pathak et al., Jan. 4, 2018, “Assessment of Nutrition Intake Using a Handheld Tool”) discloses a smart food utensil for measuring food mass.

U.S. patent application publication 20160091419 (Watson et al., Mar. 31, 2016, “Analyzing and Correlating Spectra, Identifying Samples and Their Ingredients, and Displaying Related Personalized Information”) discloses a spectral analysis method for food analysis. U.S. patent application publications 20170292908 (Wilk et al., Oct. 12, 2017, “Spectrometry System Applications”) and 20180143073 (Goldring et al., May 24, 2018, “Spectrometry System Applications”) disclose a spectrometer system to determine spectra of an object. U.S. patent application publication 20170193854 (Yuan et al., 2016 Jan. 5, “Smart Wearable Device and Health Monitoring Method”) discloses a wearable device with a camera to monitor eating. U.S. Pat. No. 9,146,147 (Bakhsh, Sep. 29, 2015, “Dynamic Nutrition Tracking Utensils”) discloses nutritional intake tracking with a smart utensil. U.S. patent Ser. No. 10/058,283 (Zerick et al., 2016 Apr. 6, “Determining Food Identities with Intra-Oral Spectrometer Devices”) discloses an intra-oral device for food analysis. U.S. Pat. No. 9,349,297 (Ortiz et al., May 24, 2016, “System and Method for Nutrition Analysis Using Food Image Recognition”) discloses a system and method for determining the nutritional value of a food item. U.S. Pat. No. 9,364,106 (Ortiz, Jun. 14, 2016, “Apparatus and Method for Identifying, Measuring and Analyzing Food Nutritional Values and Consumer Eating Behaviors”) discloses a food container for determining the nutritional value of a food item.

U.S. patent Ser. No. 10/249,214 (Novotny et al., Apr. 2, 2019, “Personal Wellness Monitoring System”) discloses a personal nutrition, health, wellness and fitness monitor which analyzes food images. U.S. patent Ser. No. 10/359,381 (Lewis et al., Jul. 23, 2019, “Methods and Systems for Determining an Internal Property of a Food Product”) discloses a system and method for measuring an internal property of a food item. U.S. patent Ser. No. 10/423,045 (Roberts et al., Sep. 24, 2019, “Electro-Optical Diffractive Waveplate Beam Shaping System”) discloses optical beam shaping systems with a diffractive waveplate diffuser. U.S. patent Ser. No. 10/901,509 (Aimone et al., Jan. 26, 2021, “Wearable Computing Apparatus and Method”) discloses a wearable computing device comprising at least one brainwave sensor.

In the non-patent literature, Amft et al., 2005 (“Detection of Eating and Drinking Arm Gestures Using Inertial Body-Worn Sensors”) discloses eating detection by analyzing arm gestures. Bedri et al., 2015 (“Detecting Mastication: A Wearable Approach”; access to abstract only) discloses eating detection using an ear-worn devices with a gyroscope and proximity sensors. Bedri et al., 2017 (“EarBit: Using Wearable Sensors to Detect Eating Episodes in Unconstrained Environments”) discloses eating detection using an ear-worn device with inertial, optical, and acoustic sensors. Bedri et al., 2020a (“FitByte: Automatic Diet Monitoring in Unconstrained Situations Using Multimodal Sensing on Eyeglasses”) discloses food consumption monitoring using a device with a motion sensor, an infrared sensor, and a camera which is attached to eyeglasses. Bell et al., 2020 (“Automatic, Wearable-Based, In-Field Eating Detection Approaches for Public Health Research: A Scoping Review”) reviews wearable sensors for eating detection.

Bi et al., 2016 (“AutoDietary: A Wearable Acoustic Sensor System for Food Intake Recognition in Daily Life”) discloses eating detection using a neck-worn device with sound sensors. Bi et al., 2017 (“Toward a Wearable Sensor for Eating Detection”) discloses eating detection using ear-worn and neck-worn devices with sound sensors and EMG sensors. Bi et al., 2018 (“Auracle: Detecting Eating Episodes with an Ear-Mounted Sensor”) discloses eating detection using an ear-worn device with a microphone. Borrell, 2011 (“Every Bite You Take”) discloses food consumption monitoring using a neck-worn device with GPS, a microphone, an accelerometer, and a camera. Brenna et al., 2019 (“A Survey of Automatic Methods for Nutritional Assessment) reviews automatic methods for nutritional assessment. Chun et al., 2018 (“Detecting Eating Episodes by Tracking Jawbone Movements with a Non-Contact Wearable Sensor”) discloses eating detection using a necklace with an accelerometer and range sensor.

Chung et al., 2017 (“A Glasses-Type Wearable Device for Monitoring the Patterns of Food Intake and Facial Activity”) discloses eating detection using a force-based chewing sensor on eyeglasses. Dimitratos et al., 2020 (“Wearable Technology to Quantify the Nutritional Intake of Adults: Validation Study”) discloses high variability in food consumption monitoring using only a wristband with a motion sensor. Dong et al., 2009 (“A Device for Detecting and Counting Bites of Food Taken by a Person During Eating”) discloses bite counting using a wrist-worn orientation sensor. Dong et al., 2011 (“Detecting Eating Using a Wrist Mounted Device During Normal Daily Activities”) discloses eating detection using a watch with a motion sensor. Dong et al., 2012b (“A New Method for Measuring Meal Intake in Humans via Automated Wrist Motion Tracking”) discloses bite counting using a wrist-worn gyroscope. Dong et al., 2014 (“Detecting Periods of Eating During Free-Living by Tracking Wrist Motion”) discloses eating detection using a wrist-worn device with motion sensors.

Farooq et al., 2016 (“A Novel Wearable Device for Food Intake and Physical Activity Recognition”) discloses eating detection using eyeglasses with a piezoelectric strain sensor and an accelerometer. Farooq et al., 2017 (“Segmentation and Characterization of Chewing Bouts by Monitoring Temporalis Muscle Using Smart Glasses With Piezoelectric Sensor”) discloses chew counting using eyeglasses with a piezoelectric strain sensor. Fontana et al., 2014 (“Automatic Ingestion Monitor: A Novel Wearable Device for Monitoring of Ingestive Behavior”) discloses food consumption monitoring using a device with a jaw motion sensor, a hand gesture sensor, and an accelerometer. Fontana et al., 2015 (“Energy Intake Estimation from Counts of Chews and Swallows”) discloses counting chews and swallows using wearable sensors and video analysis. Jasper et al., 2016 (“Effects of Bite Count Feedback from a Wearable Device and Goal-Setting on Consumption in Young Adults”) discloses the effect of feedback based on bite counting.

Liu et al., 2012 (“An Intelligent Food-Intake Monitoring System Using Wearable Sensors”) discloses food consumption monitoring using an ear-worn device with a microphone and camera. Magrini et al., 2017 (“Wearable Devices for Caloric Intake Assessment: State of Art and Future Developments”) reviews wearable devices for automatic recording of food consumption. Makeyev et al., 2012 (“Automatic Food Intake Detection Based on Swallowing Sounds”) discloses swallowing detection using wearable sound sensors. Merck et al., 2016 (“Multimodality Sensing for Eating Recognition”; access to abstract only) discloses eating detection using eyeglasses and smart watches on each wrist, combining motion and sound sensors.

Mirtchouk et al., 2016 (“Automated Estimation of Food Type and Amount Consumed from Body-Worn Audio and Motion Sensors”; access to abstract only) discloses food consumption monitoring using in-ear audio plus head and wrist motion. Mirtchouk et al., 2017 (“Recognizing Eating from Body-Worn Sensors: Combining Free-Living and Laboratory Data”) discloses eating detection using head-worn and wrist-worn motion sensors and sound sensors. O'Loughlin et al., 2013 (“Using a Wearable Camera to Increase the Accuracy of Dietary Analysis”) discloses food consumption monitoring using a combination of a wearable camera and self-reported logging. Prioleau et al., 2017 (“Unobtrusive and Wearable Systems for Automatic Dietary Monitoring”) reviews wearable and hand-held approaches to dietary monitoring. Rahman et al., 2015 (“Unintrusive Eating Recognition Using Google Glass”) discloses eating detection using eyeglasses with an inertial motion sensor.

Sazonov et al., 2008 (“Non-Invasive Monitoring of Chewing and Swallowing for Objective Quantification of Ingestive Behavior”) discloses counting chews and swallows using ear-worn and/or neck-worn strain and sound sensors. Sazonov et al., 2009 (“Toward Objective Monitoring of Ingestive Behavior in Free-Living Population”) discloses counting chews and swallows using strain sensors. Sazonov et al., 2010a (“The Energetics of Obesity: A Review: Monitoring Energy Intake and Energy Expenditure in Humans”) reviews devices for monitoring food consumption. Sazonov et al., 2010b (“Automatic Detection of Swallowing Events by Acoustical Means for Applications of Monitoring of Ingestive Behavior”) discloses swallowing detection using wearable sound sensors. Sazonov et al., 2012 (“A Sensor System for Automatic Detection of Food Intake Through Non-Invasive Monitoring of Chewing”) discloses eating detection using a wearable piezoelectric strain gauge.

Schiboni et al., 2018 (“Automatic Dietary Monitoring Using Wearable Accessories”) reviews wearable devices for dietary monitoring. Sen et al., 2018 (“Annapurna: Building a Real-World Smartwatch-Based Automated Food Journal”; access to abstract only) discloses food consumption monitoring using a smart watch with a motion sensor and a camera. Sun et al., 2010 (“A Wearable Electronic System for Objective Dietary Assessment”) discloses food consumption monitoring using a wearable circular device with earphones, microphones, accelerometers, or skin-surface electrodes. Tamura et al., 2016 (“Review of Monitoring Devices for Food Intake”) reviews wearable devices for eating detection and food consumption monitoring. Thomaz et al., 2013 (“Feasibility of Identifying Eating Moments from First-Person Images Leveraging Human Computation”) discloses eating detection through analysis of first-person images. Thomaz et al., 2015 (“A Practical Approach for Recognizing Eating Moments with Wrist-Mounted Inertial Sensing”) discloses eating detection using a smart watch with an accelerometer.

Vu et al., 2017 (“Wearable Food Intake Monitoring Technologies: A Comprehensive Review”) reviews sensing platforms and data analytic approaches to solve the challenges of food-intake monitoring, including ear-based chewing and swallowing detection systems and wearable cameras. Young, 2020 (“FitByte Uses Sensors on Eyeglasses to Automatically Monitor Diet: CMU Researchers Propose a Multimodal System to Track Foods, Liquid Intake”) discloses food consumption monitoring using a device with a motion sensor, an infrared sensor, and a camera which is attached to eyeglasses. Zhang et al., 2016 (“Diet Eyeglasses: Recognising Food Chewing Using EMG and Smart Eyeglasses”; access to abstract only) discloses eating detection using eyeglasses with EMG sensors. Zhang et al., 2018a (“Free-Living Eating Event Spotting Using EMG-Monitoring Eyeglasses”; access to abstract only) discloses eating detection using eyeglasses with EMG sensors. Zhang et al., 2018b (“Monitoring Chewing and Eating in Free-Living Using Smart Eyeglasses”) discloses eating detection using eyeglasses with EMG sensors.

### SUMMARY OF THE INVENTION

This invention is a wearable device or system which helps a person to track their food intake, including the quantities and types of food which they eat. Quantities and types of food can be further broken down into (e.g. correlated with) quantities and types of nutrients as part of an overall system for nutritional intake monitoring and management. This invention can be embodied in a smart watch and/or wrist band with a camera which records food images. Food images are analyzed as part of the identification of food types and quantities.

Such a smart watch and/or wrist band can also include a display and/or camera viewfinder, a spectroscopic sensor, and an eating detector. The spectroscopic sensor has a light emitter and a light receiver. The light emitter emits light rays toward food. The light receiver receives the light rays after the rays have been reflected by food. Light rays reflected by food are analyzed for spectroscopic identification of food types and/or composition. The eating detector can be an accelerometer, a gyroscope, a magnetometer, a microphone, or an EMG sensor. The camera and/or the spectroscopic sensor can be automatically activated when the person eats food, but otherwise remain off to help maintain privacy.

### BRIEF DESCRIPTION OF THE FIGURES

FIG. 1 shows a system for nutritional monitoring and management which includes a camera, a spectroscopic sensor, a fiducial component, a biometric sensor, a smart utensil, a passive feedback mechanism, an active stimulus mechanism, and a data processor.

FIG. 2 shows smart eyewear for measuring food consumption with a camera.

FIG. 3 shows smart eyewear for measuring food consumption with a camera activated by chewing.

FIG. 4 shows smart eyewear for measuring food consumption with a camera activated by chewing and hand-to-mouth proximity.

FIG. 5 shows a smart watch or wrist band for measuring food consumption with an eating-related motion sensor.

FIG. 6 shows a smart watch or wrist band for measuring food consumption with a camera activated by eating-related motion.

FIG. 7 shows a smart watch or wrist band for measuring food consumption with an eating-related motion sensor and a spectroscopic sensor.

FIG. 8 shows a smart watch or wrist band for measuring food consumption with a camera activated by eating-related motion, and also a spectroscopic sensor.

FIG. 9 shows a wearable system for measuring food consumption with an eyewear camera activated by eating-related wrist motion.

FIG. 10 shows a wearable system for measuring food consumption with an eyewear camera and a wrist-based camera activated by eating-related wrist motion.

FIG. 11 shows a wearable system for measuring food consumption with an eyewear camera activated by eating-related wrist motion, and also a spectroscopic sensor.

FIG. 12 shows a wearable system for measuring food consumption with an eyewear camera and a wrist-based camera activated by eating-related wrist motion, and also a spectroscopic sensor.

FIG. 13 shows a wearable system for measuring food consumption with an eyewear camera activated by eating-related wrist motion and chewing.

FIG. 14 shows a wearable system for measuring food consumption with an eyewear camera and a wrist-based camera activated by eating-related wrist motion and chewing.

FIG. 15 shows a wearable system for measuring food consumption with an eyewear camera activated by eating-related wrist motion and chewing, and also a spectroscopic sensor.

FIG. 16 shows a wearable system for measuring food consumption with an eyewear camera and a wrist-based camera activated by eating-related wrist motion and chewing, and also a spectroscopic sensor.

FIG. 17 shows a wearable system for measuring food consumption with an eyewear camera activated by eating-related wrist motion, chewing, and hand-to-mouth proximity.

FIG. 18 shows a wearable system for measuring food consumption with an eyewear camera and a wrist-worn camera activated by eating-related wrist motion, chewing, and hand-to-mouth proximity.

FIG. 19 shows a wearable system for measuring food consumption with an eyewear camera activated by eating-related wrist motion, chewing, and hand-to-mouth proximity, and also a spectroscopic sensor.

FIG. 20 shows a wearable system for measuring food consumption with an eyewear camera and a wrist-worn camera activated by eating-related wrist motion, chewing, and hand-to-mouth proximity, and also a spectroscopic sensor.

FIG. 21 shows a basic type of smart watch in the prior art for comparison purposes.

FIG. 22 shows a wrist-worn device for tracking food intake with a camera, a primary display, a viewfinder, a spectroscopic sensor, and an eating detector.

FIG. 23 shows the device of FIG. 22 in operation tracking food intake.

FIG. 24 shows a wrist-worn device for tracking food intake with a camera, a primary display which serves as a viewfinder, a spectroscopic sensor, and an eating detector.

FIG. 25 shows a wrist-worn device for tracking food intake with a band, a primary housing, a flip-up display, a camera on the band, a spectroscopic sensor, and an eating detector.

FIG. 26 shows a wrist-worn device for tracking food intake with a primary housing, a flip-up display, a camera on the flip-up display, a spectroscopic sensor, and an eating detector.

### DETAILED DESCRIPTION OF THE FIGURES

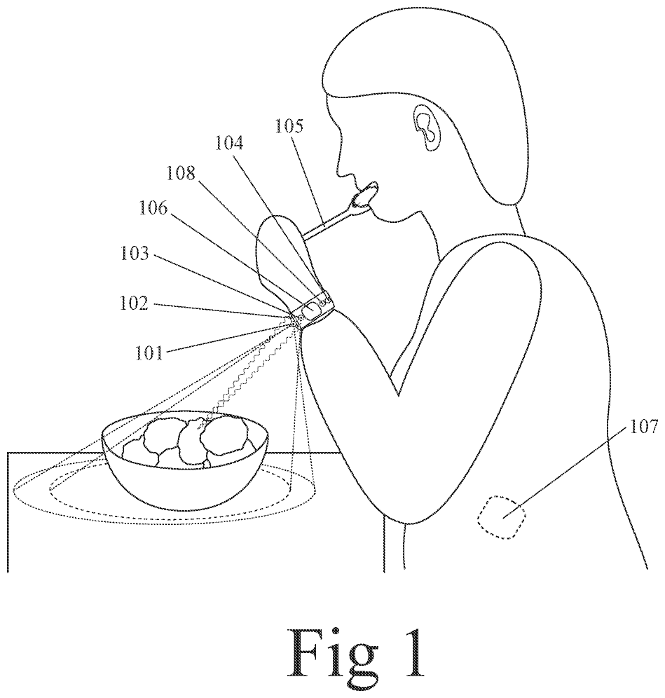

FIG. 1 shows an example of a system for nutritional monitoring and management comprising: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (e) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (f) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); (g) an active stimulus mechanism which automatically responds to food consumption by the person, wherein the active stimulus mechanism automatically modifies a person's physiological processes (e.g. by delivering a therapeutic agent, such as insulin, into the person's body; by delivering a therapeutic pattern of electromagnetic energy to a selected portion of the person's body, such as the vagus nerve; or by delivering a taste-modifying substance into a person's mouth); and (h) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

Specifically, FIG. 1 shows a system for nutritional monitoring and management comprising: (a) camera 101; (b) spectroscopic sensor 102; (c) fiducial component 103; (d) wearable biometric sensor 104; (e) smart utensil 105; (f) passive feedback mechanism 106; (g) active stimulus mechanism 107; and (h) data processor 108. In the example shown in FIG. 1, camera 101 is worn on a person's wrist and records images of food items, spectroscopic sensor 102 is worn on the person's wrist and scans nearby food; fiducial component 103 is a light projector worn on the person's wrist and projects a laser pattern on or near food to help calibrate the distance, size, shape, color, and/or brightness of the food; wearable biometric sensor 104 is a motion sensor worn on the person's wrist which tracks arm, wrist, and/or hand motion; smart utensil 105 is a smart spoon held by the person which measures the weight of each spoonfull of food; passive feedback mechanism 106 is a display screen which is worn on the person's wrist and provides visual information concerning the person's food consumption; active stimulus mechanism 107 delivers insulin to the person's body based on food consumption; and data processor 108 processes data from the camera, spectroscopic sensor, and/or biometric sensor. We now discuss example variations on systems for nutritional monitoring and management. The example variations which follow and also the example variations which are disclosed in priority-linked applications can be applied where relevant to the system which is shown in FIG. 1. Also, simplified versions of this system without all of the system components shown in FIG. 1 can be adequate for some nutritional monitoring and management applications and are also within the scope of this invention.

In an example, a system for nutritional monitoring and management can include a general purpose handheld device (such as a smart phone or electronic tablet). In an example, a system can incorporate information from a camera, a touch screen, a microphone, and/or a motion sensor on a general purpose handheld device. In an example, a system can include a software application for nutritional monitoring and management which runs on a general purpose handheld device (such as a smart phone or electronic tablet).

In an example, a system for nutritional monitoring and management can include a handheld camera. In an example, a system can include a handheld electronic tablet. In an example, a system can include a handheld food imaging device. In an example, a system can include a handheld food probe. In an example, a system can include a handheld food scanner. In an example, a system can include a handheld invasive food probe. In an example, a system can include a handheld non-invasive spectroscopic food scanner. In an example, a system can include a handheld removable accessory for a cell phone. In an example, a system can include a handheld removable attachment for a conventional food utensil. In an example, a system can include a removable component of a smart watch or wrist band. In an example, a system can include a smart phone component. In an example, a system can include a smart phone, cell phone, and/or mobile phone. In an example, a system can include a smart utensil.

In an example, a system for nutritional monitoring and management can include a specialized handheld device, such as a specialized handheld device with a camera, spectroscopic sensor, and motion sensor. In an example, a system can include a specialized handheld device with a spectroscopic sensor, a camera, and a laser beam projector. In an example, a laser can form a light pattern near food which serves as a fiducial marker for analyzing food size and/or color. In an example, a system can include a specialized handheld device with a spectroscopic sensor, a camera, and a food interior probe. In an example, a handheld spectroscopic sensor can be placed in juxtaposition with a food item for spectroscopic analysis of the food item. In an example, a handheld spectroscopic sensor can be placed over different locations on a meal to perform spectroscopic analyses of different food items in the meal and/or different locations within a non-homogenous food item.

In an example, a system for nutritional monitoring and management can include a smart food utensil (e.g. smart spoon, fork, or chop sticks) or beverage holder (e.g. smart cup, glass, or mug). In an example, a smart food utensil or beverage holder can have a camera which takes pictures of nearby food and/or food being transported by the utensil or beverage holder. In an example, a smart food utensil or beverage holder can have a spectroscopic sensor which scans nearby food and/or food being transported by the utensil or beverage holder to measure the reflection or absorption spectrum of the food and thereby identify the molecular composition of the food. In an example, a spoon can have a transparent cup (distal concave) portion which contains a spectroscopic sensor. In an example, data on the molecular composition of food in this cup portion can be collected by the spectroscopic sensor.

In an example, a system for nutritional monitoring and management can include a smart spoon with a scale which tracks the individual weights (and cumulative weight) of mouthfuls of food carried and/or consumed during an eating event. In an example, a smart spoon can approximate the weights of mouthfuls of food carried by the spoon by measuring the effect of those mouthfuls on the motion of the spoon as a whole or the relative motion of one part of the spoon relative to another. In an example, a smart spoon can include a motion sensor and/or inertial sensor. In an example, a smart spoon can include one or more accelerometers in different, motion-variable locations along the length of the spoon. In an example, a smart spoon can include a spring and/or strain gauge between the food-carrying scoop of the spoon and the handle of the spoon. In an example, food weight can estimated by measuring distension of the spring and/or strain gauge as food is brought up to a person's mouth.

In an example, a system for nutritional monitoring and management can include a food utensil rest that functions as a bite counter and/or food scale. In an example, it can track the number of times that a utensil is put down or weigh each bite or mouthful. In an example, a food scale can be incorporated into a smart utensil which tracks the cumulative weight of cumulative mouthfuls of food during an eating event. In an example, a smart utensil can approximate the weight of mouthfuls of food by measuring the effect of food carried by the utensil on an accelerometer or other inertial sensor. In an example, a smart utensil can incorporate a spring between the food-carrying portion and the handheld portion of a utensil and food weight can be estimated by measuring distension of the spring as food is brought up to a person's mouth.

In an example, a system for nutritional monitoring and management can include a smart food utensil with a motion sensor to detect when a person is eating. A food utensil with a motion sensor can be less prone to false alarms than a motion sensor worn on a person's wrist, hand, arm, or finger because the utensil is only used when the person eats food. Since the utensil is only used for food consumption, analysis of complex motion and differentiation of food consumption actions vs. other hand gestures is less important with a utensil than it is with a device that is worn on the person's body. In an example, a smart utensil can estimate the amount of food consumed by the number of hand-to-mouth motions (combined with information concerning how much food is conveyed by the utensil with each movement). In an example, a smart utensil can encourage a person to eat slower. The idea is that if the person eats more slowly, then they will tend to not overeat past the point of internal identification of satiety.

In an example, a system for nutritional monitoring and management can include a smart utensil (e.g. smart spoon or smart fork) which uses a motion sensor to estimate the weight of the distal (food-carrying) end of the utensil at a first point in time (such as during an upswing motion as the utensil carries a mouthful of food up to the person's mouth) and also at a second point in time (such as during a downswing motion as the person lowers the utensil from their mouth). In an example, a smart utensil can estimate the weight of food actually consumed by calculating the difference in food weights between the first and second points in time. In an example, a system can track cumulative food consumption by tracking the cumulative weights of multiple mouthfuls of (different types of) food during an eating event or during a defined period of time (such as a day or week). In an example, a smart utensil can use an inertial sensor, accelerometer, or strain gauge to estimate the weight of the distal (food-carrying) end of the utensil at a first time (during an upswing motion as the utensil carries a mouthful of food up to the person's mouth), can use this sensor to estimate the weight of the food-carrying end of the utensil at a second time (during a downswing motion as the person lowers the utensil from their mouth), and can estimate the weight of the mouthful of food by calculating the difference in weight between the first and second times.

In an example, a system for nutritional monitoring and management can include a smart utensil which identifies types and quantities of consumed foods, ingredients, or nutrients by being in optical communication with food. In an example, a smart utensil can identify food item types and quantities by recording images of food. In an example, a smart utensil can record images of food that is within a reachable distance of a person. In an example, a smart utensil can record images of food on a plate. In an example, a smart utensil can record images of a portion of food as that food is conveyed to a person's mouth via the utensil.

In an example, a system for nutritional monitoring and management can include a smart utensil (e.g. smart fork, smart spoon, or smart chop sticks). In an example, a smart utensil can identifies food type, nutritional composition, and/or molecular composition by optically analyzing food. In an example, a system can include a smart utensil with a spectroscopic sensor which identifies food type, nutritional composition, and/or molecular composition by spectroscopic analysis. In an example, a smart utensil can identify the type, nutritional composition, and/or molecular composition of a food item by projecting light beams toward the food item and then receiving those light beams after they have been reflected by (or passed through) the food item. In an example, the effects of interaction with food on the spectral distribution of light beams can provide information on food type and/or nutritional composition. In an example, a smart utensil can spectroscopically analyze food as that food is being brought up to a person's mouth using the utensil. In an example, a smart utensil can spectroscopically analyze a nearby food item before a portion of the food item is brought onto the utensil. In an example, a smart utensil can spectroscopically analyze a nearby food item while the food item is still on a plate or in a bowl.

In an example, a system for nutritional monitoring and management can include a smart utensil (e.g. smart fork, smart spoon, or smart chop sticks) which measures the weight of a piece and/or portion of food which is carried by the utensil. In an example, a smart utensil can have a moveable portion such as a flexible joint or bend sensor between the distal (food carrying) end of the utensil and the handle of the utensil. In an example, the weight and/or momentum of a piece of food being carried by the distal end of a utensil can cause this moveable portion to bend or flex. In an example, bending or flexing of this moveable portion can be measured by a force sensor, strain sensor, bend sensor, goniometer, or pressure sensor in order to estimate the weight of a piece or portion of food being carried by the utensil. In an example, a smart fork can estimate the weight of solid food on the tines of the fork using a force sensor, strain sensor, bend sensor, or pressure sensor. In an example, a smart spoon can estimate the weight of a liquid in the concavity of the spoon using a force sensor, strain sensor, bend sensor, or pressure sensor.

In an example, a system for nutritional monitoring and management can include a smart utensil (e.g. smart fork, smart spoon, or smart chop sticks) with two motion sensors, a first motion sensor in the distal (e.g. food carrying) end of the utensil and a second motion sensor in the handle of the utensil, wherein the first and second motion sensors are separated by a moveable portion such as a flexible joint. In an example, differences in motion patterns between the first and second motion sensors can be analyzed in order to estimate the weight of a piece of food carried by the utensil. In an example, the greater the weight of a piece or portion of food being carried by the distal end of a smart utensil, the greater the bending and/or flexing of a joint between the distal end of the utensil and the proximal handle of the utensil. In an example, the faster a piece or portion of food is conveyed up to a person's mouth, the greater the bending and/or flexing of a joint between the distal end of the utensil and the proximal handle of the utensil.

In an example, a system for nutritional monitoring and management can include a smart utensil (e.g. smart fork, smart spoon, or smart chop sticks) which performs a function selected from the group consisting of: communicating information concerning food type and/or quantity to other system components; detecting use of the utensil for eating; estimating the nutritional and/or molecular composition of food via spectroscopic analysis; identifying a type of food via image analysis; identifying a type of food via spectroscopic analysis; influencing and/or changing the amount of food consumed by a person via visual, audio, or haptic stimuli; influencing and/or changing the speed of a person's food consumption via visual, audio, or haptic stimuli; measuring the amount of food consumed via a bend sensor, force sensor, or pressure sensor; measuring the amount of food consumed via a motion sensor; measuring the amount of food consumed via image analysis; measuring the speed, rate, or pace of food consumption via a bend sensor, force sensor, or pressure sensor; measuring the speed, rate, or pace of food consumption via a motion sensor; measuring the speed, rate, or pace of food consumption via image analysis; providing a user with feedback concerning the speed, rate, or pace of food consumption via light, sound, or vibration; signaling the amount of food consumed to a user via light, sound, or vibration; and signaling the speed, rate, or pace of food consumption to a user via light signals, sound signals, or haptic signals.

In an example, a system for nutritional monitoring and management can include a smart utensil (e.g. smart fork, smart spoon, or smart chop sticks) with a camera which records images of nearby food (e.g. food within a person's reach). In an example, a system can include a smart utensil with a camera which records images of a piece or portion of food being carried to a person's mouth by the utensil. In an example, a smart utensil can have a nutritional, molecular, and/or chemical composition sensor. In an example, a smart utensil can have a spectroscopic sensor which emits light beams toward food and receives these light beams after they have been reflected by (or passed through) food. In this manner, a spectroscopic sensor can scan nearby food and/or food being carried to a person's mouth by a utensil in order to estimate the nutritional composition of the food. In an example, a system can include a smart spoon with a spectroscopic sensor which scans food being carried in the concavity of the spoon. In an example, a system can include a smart fork with a spectroscopic sensor which scans food being carried on the tines of the fork.

In an example, a system for nutritional monitoring and management can include a smart utensil (e.g. a smart fork, smart spoon, or smart chop sticks). In an example, a smart utensil can have a motion sensor (e.g. an accelerometer and/or gyroscope) that tracks how many times and/or how quickly a person brings the utensil up to their mouth. In an example, analysis of the roll, pitch, and yaw of smart utensil motion can be analyzed to help identify the types and quantities of food items consumed by a person. In an example, the speed, acceleration, or distance of smart utensil motion can be analyzed to help identify the types and quantities of food items consumed by a person.

In an example, a system for nutritional monitoring and management can include both a smart utensil (e.g. smart fork, smart spoon, or smart chop sticks) and a wearable device (e.g. smart watch, smart ring, or augmented reality eyewear). In an example, a wearable component of such a system can continually monitor whether a person is eating, but the smart utensil component of the system may only be triggered (e.g. activated) when a person starts eating. In an example, a system can prompt a person to use a smart utensil when the system detects that a person has started to eat but is not using the smart utensil. In an example, a system can monitor the proximity of a smart utensil to a wrist-worn device. In an example, a system can compare the motion of a smart utensil to the motion of a wrist-worn device. In an example, this comparison can determine whether the smart utensil is being used when a person eats. In an example, differences in motion between the motion of a smart utensil and the motion of a wearable device (such as a smart watch or finger ring) can be analyzed to help identify types and quantities of food being consumed.

In an example, a smart food utensil or beverage holder can have a motion sensor (e.g. accelerometer and/or gyroscope) which measures the number of times and/or the frequency with which a person brings the utensil or beverage holder up to their mouth. The number and frequency with which a utensil or beverage holder is brought up to a person's mouth can help to estimate the amount of food that a person actually consumes. In an example, details concerning the movement and acceleration of the utensil or beverage holder can help to identify the weight and type of food, as well as the quantity of food, actually consumed by a person. In an example, specific sequential patterns of roll, pitch, and yaw can be associated with specific types and/or weights of food. In an example, a smart food utensil can include a force, bend, and/or strain sensor. In an example, a force, bend, and/or strain sensor can be on a moveable joint between a food holding portion of a smart utensil and the handle of the utensil. In an example, such a force, bend, and/or strain sensor can measure the force and/or inertia of food relative to the utensil handle, thereby helping to measure food weight.

In an example, a system for nutritional monitoring and management can include a food probe with actuators which move a spectroscopic sensor up and down a longitudinal axis of the probe, thereby scanning different depths of the interior of a food item. In an example, a food probe can have a spectroscopic sensor which slides within the probe in a longitudinal manner, scanning different depths of the interior of a food item. In an example, a food probe can have moving mirrors or lenses which change the location, distance, and/or depth from which the food probe spectroscopically scans the interior of food. In an example, a food probe can have a spectroscopic sensor which rotates within the probe, thereby scanning different radial sections of the interior of a food item. In an example, a food probe can have a spectroscopic sensor which rotates around a longitudinal axis of the food probe, thereby scanning different radial sections of the interior of a food item.

In an example, a system for nutritional monitoring and management can include a food probe which is inserted into the interior of a food item. In an example, a system can include a handheld food probe. In an example, a food probe can have a spectroscopic sensor which takes spectroscopic scans of the interior of a food item. This is particularly useful for food which is not homogenous, such as food with different interior layers or structures. In an example, a food probe can have a longitudinal protrusion (like a fork tine) which is inserted into the interior of a food item. In an example, a food probe can have a transparent exterior surface and a spectroscopic sensor located inside this transparent exterior surface. For example, a food probe can be light a transparent fork tine with a spectroscopic sensor inside the tine. In an example, a food probe can be a removable component of a wearable device. In an example, a food probe can be removed from a wearable device, inserted into the interior of food, cleaned off, and then inserted back into the wearable device.

In an example, a system for nutritional monitoring and management can include a handheld food probe which is inserted into food to analyze the molecular composition of the food interior. In an example, this food probe can measure impedance inside a food item. In an example, this food probe can perform spectroscopic analysis of the interior of a food item. In an example, this food probe can use sound (e.g. low frequency or high frequency) to scan the interior of a food item. In an example, a food probe can scan different layers or depths of the interior of a food item using spectroscopic analysis, ultrasound scanning, and/or electromagnetic impedance analysis. In an example, a spectroscopic sensor inside a food probe can rotate within the food probe and/or move in a proximal-to-distal manner within the food probe to scan different areas of the interior of a food item.

In an example, a system for nutritional monitoring and management can include a wearable device. In an example, a system can include smart eyewear (such as smart eyeglasses, augmented reality eyeglasses, goggles, a smart visor, or a smart contact lens). In an example, smart eyewear can have a camera. In an example, smart eyewear can have two stereoscopic cameras for 3D imaging. In an example, smart eyewear can have augmented reality (AR) functionality. In an example, smart eyewear with AR functionality can serve as a computer-to-human interface, displaying information about food in a person's field of view. In an example, smart eyewear with AR functionality can serve as a human-to-computer interface, enabling a person to input user information about food in the person's field of view. In an example, a person can input user information about food via voice (e.g. speech recognition), gesture (e.g. gesture recognition), touch (e.g. touch screen), text (e.g. via a keypad), or thought (e.g. via a mobile EEG sensor device).

In an example, a system for nutritional monitoring and management can include a wrist-worn device (e.g. smart watch, smart watch band, wrist band, fitness band, smart bracelet, smart sleeve, or smart cuff) with a camera for recording images of nearby food items, wherein the camera is located on the anterior/palmar/lower side or a lateral/narrow side of a person's wrist. In an example, a system can include a wrist-worn device (e.g. smart watch, smart watch band, wrist band, fitness band, smart bracelet, smart sleeve, or smart cuff) with a spectroscopic sensor for scanning nearby food items, wherein the spectroscopic sensor is located on the anterior/palmar/lower side or a lateral/narrow side of a person's wrist. In an example, a system can include a watch band with two cameras facing in different directions, wherein the two cameras collectively record images of interaction between a person's hand and nearby food; and interaction between the person's mouth and food.

In an example, a system for nutritional monitoring and management can include a device which is worn on a person's wrist and/or arm (such as a smart watch, smart watch band, smart wrist band, fitness band, smart glove, or smart bracelet). In an example, a system can include a watch that is not so smart, but has potential and should be given a chance. In an example, a wearable device on a person's wrist and/or arm can include a motion sensor which detects when a person is eating based on a series of distinctive upward/downward, pausing, and roll/tilt motions. In an example, a wearable device on a person's wrist and/or arm can include a camera which takes pictures of food at different and/or selected times during upward/downward and roll/tilt motions when a person is eating. In an example, a wearable device on a person's wrist and/or arm can include (a circumferential array of) one or more biometric sensors which measure biometric parameters associated with food consumption. In an example, a wearable device on a person's wrist and/or arm can include (a circumferential array of) one or more spectroscopic sensors which measure biometric parameters associated with food consumption. In an example, a wearable device on a person's wrist and/or arm can include (a circumferential array of) one or more electromagnetic energy sensors which measure biometric parameters associated with food consumption.

In an example, a system for nutritional monitoring and management can include a finger-worn device (e.g. finger ring) with a camera for recording images of nearby food items. In an example, a system can include a finger-worn device (e.g. finger ring) with a spectroscopic sensor for scanning nearby food items. In an example, a system can include a finger ring with two cameras facing in different directions, wherein the two cameras collectively record images of interaction between a person's hand and nearby food; and interaction between the person's mouth and food.

In an example, a system for nutritional monitoring and management can include a device (such as a smart finger ring or finger nail attachment) which is worn on a person's finger. In an example, a finger ring can include a motion sensor which detects when a person is eating based on a series of distinctive upward/downward and roll/tilt motions. In an example, a finger ring can include a camera which takes pictures of food at different and/or selected times during upward/downward and roll/tilt motions. In an example, a finger ring can include (a circumferential array of) one or more biometric sensors which measure biometric parameters associated with food consumption. In an example, a finger ring can include (a circumferential array of) one or more spectroscopic sensors which measure biometric parameters associated with food consumption. In an example, a finger ring can include (a circumferential array of) one or more electromagnetic energy sensors which measure biometric parameters associated with food consumption.

In an example, a system for nutritional monitoring and management can include a device (e.g. earware or “hearable”) which is worn in (or on) a person's ear. In an example, this device can include a smart ear ring. In an example, a smart ear ring can include a camera, a pulse oximeter, and/or a glucose sensor. In an example, this device can include an ear bud. In an example, a smart ear-worn device can encircle at least two-thirds of the perimeter of a person's outer ear. In an example, a smart ear-worn device can encircle at least two-thirds of the perimeter of a person's outer ear and have an extension (e.g. arm or prong) which extends from the perimeter of the ear onto a portion of a person's temple and/or forehead. In an example, this extension can include an electromagnetic energy sensor (such as an EEG sensor) whose data is also used by the system to: detect food consumption by a person; and/or evaluate the types and quantities of food consumed by the person.

In an example, a system for nutritional monitoring and management can include a device which is worn on (or around) a person's neck. In an example, a system can include a smart necklace and/or pendant with a camera which records images of food in front of a person and/or food near the person's mouth. In an example, a smart necklace and/or pendant can monitor movement of a person's hand up to their mouth as part of nutritional intake tracing. In an example, a system can include a smart collar, scarf, or tie which is worn around a person's neck. In an example, a smart collar, scarf, or tie can have a microphone which monitors sounds associated with eating such as chewing, swallowing, and teeth grinding sounds. In an example, a smart collar, scarf, or tie can have an electromagnetic energy sensor (such as an EMG sensor) which monitors muscle movements associated with eating. In an example, a smart collar, scarf, or tie can have a camera which records images of food in front of a person.

In an example, a system for nutritional monitoring and management can include a handheld device which is selected from the group consisting of: handheld camera; handheld electronic tablet; handheld food imaging device; handheld food probe; handheld food scanner; handheld invasive food probe; handheld non-invasive spectroscopic food scanner; handheld removable accessory for a cell phone; handheld removable attachment for a conventional food utensil; removable component of a smart watch or wrist band; smart phone component; smart phone, cell phone, and/or mobile phone; and smart utensil. In an example, a system can include a wearable device which is selected from the group consisting of: arm band, augmented reality (AR) eyewear, smart belt, bluetooth device, bracelet, brooch, smart button, collar, cuff link, dog tags, ear bud or insert, ear plug, ear ring, ear-mounted bluetooth device, smart eyeglasses, finger ring, fitness band, headband, hearing aid, intra-oral device, mobile EEG device, smart necklace, pendant, smart pants, smart shirt, smart sleeve or cuff, wearable mouth microphone, watch phone, wrist band, and wrist watch. In an example, a system can include both a wearable device component and a handheld device component.

In an example, a system for nutritional monitoring and management can include one or more wearable devices selected from the group consisting of: an adhesive sensor patch or strip which is worn directly on a person's skin; an article of smart clothing (such as clothing with embedded or integrated biometric sensors); a face-worn device other than eyewear (such as a nose ring); a head-worn circumferential device (such as a head band, hat, or cap); a head-worn half-circumferential device (such as headphones); a leg-worn device (such as an ankle band, garter, or sock); a smart pin-type button; a sweat sensor; and a torso-worn device (such as a smart belt or chest strap).

In an example, a system for nutritional monitoring and management can include an implanted device. In an example, such a system can include a pacemaker or implanted neurological sensor. In an example, such a system can include an intra-oral device, such as a smart dental fixture, retainer, device attached to palate, tongue ring, or device attached below tongue. In an example, such a system can include an implanted drug delivery device. In an example, such a system can include an implanted neurostimulation device. In an example, an implanted device can have an electromagnetic energy sensor. In an example, an implanted device can have a spectroscopic sensor.

In an example, a system for nutritional monitoring and management can include a camera which records images (e.g. takes pictures) of food. In an example, a system can include a camera which records images of food at different times (e.g. at different times during a meal). In an example, a system can include a camera which moves to record multiple still images of food from different angles and/or distances (e.g. from different locations above a meal). In an example, a camera can record videos (e.g. moving pictures) of food. In an example, recorded food images can be automatically analyzed to identify food item types and estimate food item quantities. In an example, a system can include a food-imaging camera on a handheld device. In an example, a system can include a food-imaging camera on a wearable device. In an example, a system can include a food-imaging camera on a wrist-worn device (such as a smart watch and/or smart watch band). In an example, a camera can be located on the side of a person's wrist, where the main housing of a conventional wrist watch is generally located. In an example, a camera can be located on the opposite side of a person's wrist, opposite where the main housing of a conventional wrist watch is generally located.

In an example, a system for nutritional monitoring and management can comprise a plurality of cameras which simultaneously record images of food items from different locations, angles, and/or distances. In an example, images of food items from different angles and/or distances can be integrated to create a 3D (three-dimensional, volumetric) model of the food items which is useful for identification of food item types and estimating food item quantities. In an example, a system can include a device with two cameras (e.g. stereoscopic cameras) which simultaneously record images of food items from different locations to create stereoscopic images of food items. In an example, smart eyewear can have two cameras, one on the right side of the eyewear and one on the left side of the eyewear. In an example, a smart watch can have two cameras on different sides of the watch housing or on different sides of the watch band. In an example, a system can comprise one camera which faces away from a person (e.g. toward nearby food on a table) and one camera which faces toward the person (e.g. toward the person's face and mouth).

In an example, a system for nutritional monitoring and management can have two (or more) cameras which are worn on the narrow sides of a person's wrist (between the posterior and anterior surfaces of the wrist) such that the moving field of vision of a first camera automatically encompasses the person's mouth (as the person moves their arm when they eat) and the moving field of vision of a second camera automatically encompasses nearby food items (as the person moves their arm when they eat). This design is comparable to a wrist-watch that has been rotated 90 degrees around a person's wrist, with a first camera located where the watch face would normally be and a second camera located on the opposite side of the wrist. In an example, a system can have two (or more) cameras which record images of food at different times, from different directions, and/or with different focal lengths.

In an example, a system for nutritional monitoring and management can have two cameras for recording images of food. In an example, these two cameras can point in generally the same direction. In an example, these two cameras can be stereoscopic. In an example, these two cameras can point in different (e.g. opposite) directions. In an example, fields of vision from two cameras can collectively and automatically encompass both nearby food items and a person's mouth as the person eats. In an example, fields of vision from two wrist-worn cameras can encompass both nearby food items and a person's mouth as the person moves their arm (and wrist) while eating. In an example, a system can have two cameras which are both on the same wearable device. Alternatively, a system can have two cameras which are worn on two different wearable devices. In an example, a system can include a first camera which is worn on a first body member (e.g. wrist, hand, lower arm, or finger) wherein the field of vision from the first camera automatically encompasses the person's mouth as the person eats and a second camera is worn on a second body member (e.g. neck, head, torso, or upper arm) wherein the field of vision from the second camera automatically encompasses nearby food items as the person eats. In an example, a system can include a first camera in a wearable device and a second camera in a non-wearable (e.g. handheld) device.

In an example, a system for nutritional monitoring and management can include a wide-angle camera. A wide-angle camera can automatically record images of a person's mouth, nearby food items, or both as the person moves their arm (and hand) while eating. In an example, a wide-angle camera can be worn on the anterior surface of a person's wrist (or upper arm) in a manner similar to a conventional watch or bracelet that has been rotated approximately 180 degrees. In an example, a camera can be worn on a person's finger in a manner similar to a finger ring, such that the camera automatically records images of the person's mouth, nearby food items, or both as the person moves their arm and hand while eating.

In an example, a system for nutritional monitoring and management can include two (or more) cameras on two (or more) different locations, respectively, around the circumference of a person's wrist. In an example, a system can comprise two cameras which are located on opposite sides of a person's wrist. In an example, these two cameras can be directed radially outward from the person's wrist. In an example, having cameras mounted on opposite sides of a person's wrist can increase the probability of encompassing both a person's mouth and nearby food items as the person moves their arm (and hand) to get a food item and then moves the food item up to their mouth. In an example, two cameras in different locations can generally track different things. For example, a first camera can generally track a person's hand and fingers (including interaction between the person's hand and nearby food) while a second camera can generally track the person's mouth (including interaction between the person's mouth and handheld or utensil-carried food). Tracking both types of interactions can provide more accurate estimates of actual food consumption by the person than tracking either interaction alone.

In an example, a system for nutritional monitoring and management can have two cameras (on one or more wearable devices) which move when a person eats. In an example, a system can include a wearable device with a first camera which records images along an imaging vector which generally points toward a person's mouth (when the person eats) and a second camera which records images along an imaging vector which generally points toward nearby food items (when the person eats). In an example, a system can comprise a first camera that is worn on a person's wrist, hand, arm, or finger (such that the field of vision from this camera automatically encompasses the person's mouth as the person eats) and a second camera that is worn on the person's neck, head, or torso (such that the field of vision from this camera automatically encompasses nearby food items as the person eats).

In an example, a system for nutritional monitoring and management can include two separate devices, each of which has at least one camera, wherein the separate devices simultaneously record images of nearby food items from different locations, angles, and/or distances. In an example, a system can include smart eyewear with a camera to record images of food items from a first perspective and a smart watch with a camera to record images of food items from a second perspective. In an example, a system can include smart eyewear with a camera to record images of food items from a first perspective and a smart phone with a camera to record images of food items from a second perspective. In an example, a system can include smart earware with a camera to record images of food items from a first perspective and a smart watch with a camera to record images of food items from a second perspective.

In an example, a system for nutritional monitoring and management can include a camera which automatically scans in selected directions or tracks selected objects in order to detect eating behavior and/or food items. In an example, a camera can track the location of a person's hand and/or mouth in order to detect eating behavior and/or foot items. In an example, a camera can continuously track the a person's hand and/or mouth. In an example, a camera can only be activated to track a person's hand and/or mouth when some less intrusive sensor (e.g. a motion sensor) indicates that the person is eating. In an example, a camera can track a person's hand scan near the person's hand to detect food items. In an example, a camera can track a person's hand scan near the person's hand to detect interaction between the person's hand and food items. In an example, a camera can track a person's mouth and scan near the person's mouth to detect food items. In an example, a camera can track a person's mouth and scan near the person's mouth to detect interaction between the person's mouth and food items.

In an example, a system for nutritional monitoring and management can include a camera which scans nearby space for a person's hand in order to detect and identify food items. In an example, a system can include a camera with a focal direction which points away from a person's body in order to capture interaction between the person's hand and food. In an example, a system can include a camera which records images along an imaging vector which points toward a person's mouth and/or face when the person eats. In an example, a system can use face recognition methods to adjust the direction and/or focal length of a camera in order to stay focused on a person's mouth and/or face. Face recognition methods and/or gesture recognition methods can be used to detect and measure hand-to-mouth proximity and interaction.

In an example, a system for nutritional monitoring and management can include a camera whose focal direction and/or depth is moved automatically to track a person's hand, a person's mouth, and/or nearby food items (which have been detected near a person's hand and/or mouth). In an example, the focal direction and/or depth of a camera can be changed independently of movement of a body member to which a camera is attached. In an example, a camera on a wearable device can be moved automatically to maintain a line of sight to a person's hand, person's mouth, or nearby food item despite movement of a body member to which the camera is attached. In an example, a camera lens can be moved automatically so that the camera tracks a person's hand, the person's mouth, and/or a food item. In an example, a reflective member (e.g. mirror) can be moved so that a camera tracks a person's hand, the person's mouth, and/or a food item. In an example, a system can use face recognition to track the location of a person's mouth and automatically move a camera lens and/or mirror so that the person's mouth remains in the camera's field of view. In an example, a system can use pattern recognition to track the location of nearby food and automatically move a camera lens and/or mirror so that the nearby food remains in the camera's field of view. In an example, a system can include a camera which scans nearby space in a spiral, radial, or back-and-forth pattern in order to track a person's hand, the person's mouth, and/or nearby food items. In an example, this scanning and/or tracking activity may be done only eating activity is detected by a less-intrusive sensor modality (such as a wearable motion sensor).

In an example, a system for nutritional monitoring and management can integrate video or sequential still images from a single moving camera (which is moves relative to food items) in order to create a 3D and/or volumetric model of the food items for analyzing food item types and/or quantities. In an example, a single moving camera can sequentially record images of food items from different angles and/or distances. In an example, a system can automatically move a camera relative to food items in order to capture sequential images of the food items from different angles and/or distances. In an example, a system can include a wrist-worn device (such as a “Willpower Watch”) with multiple cameras which is worn on a person's arm. In an example, such a wrist-worn device can record sequential images from different locations as a person moves their arm while eating, thereby sequentially recording images of nearby food from different angles and distances as the arm moves. In an example, a first camera in such a wrist-worn device can tend to capture images of a food source (e.g. on a plate on a table) while a second camera in the device can tend to capture images of a person's mouth eating the food. A combination of images of both a food item and a person's mouth eating the food item can better determine types and quantities of food consumed than either images of a food item alone or images of the person's mouth alone.

In an example, a system for nutritional monitoring and management can prompt a person and/or guide the person concerning how to move a camera (in a selected pattern) relative to food items in order to capture images of the food items from different angles and/or distances. In an example, a system can prompt and/or guide a person how to move a mobile device (such as a smart phone) in a selected pattern relative to food items in order to record images of the food items from selected different angles and/or distances to create a 3D (three-dimensional) model of the food items. In an example, a system can prompt and/or guide a person how to move a smart watch in a selected pattern relative to food items in order to record images of the food items from different angles and/or distances. In an example, a system can prompt and/or guide a person to continue moving a device relative to food items until a sufficient variety of food images from different angles and/or distances has been collected to determine food item types and quantities with a desired level of accuracy.

In an example, a system for nutritional monitoring and management can prompt and/or guide a person how to move a device with a camera in a selected pattern relative to nearby food items. In an example, this prompting and/or guidance can be visual (e.g. through augmented reality or via a light beam projected from a device). In an example, this prompting and/or guidance can be auditory (e.g. through verbal commands or sequential changes in sounds associated with sequential movement of a device). In an example, this prompting and/or guidance can be haptic (e.g. through a sequence of vibrations indicating a sequence of movement directions). In an example, a person can be prompted and/or guided to move a device with a camera in a selected pattern relative to nearby food items, wherein this selected pattern is selected from the group consisting of: movement in circles around (or above) the food items; movement in a spiral around (or above) the food items; movement back and forth (e.g. in a zigzag or sinusoidal manner) over the food items; movement toward and away from the food items; and movement along an arcuate light path which is displayed virtually in augmented reality in the person's field of view.

In an example, a system for nutritional monitoring and management can estimate the distance from a handheld or wearable device to a food item using: an infrared light emitter and receiver, a visible light projector and image analysis, a spectroscopic sensor, a radio wave emitter and receiver, or a sound (e.g. ultrasonic) energy emitter and receiver. In an example, the distance from a handheld or wearable device to a food item can be estimated via the timing and/or angle of light reflected by the food item. In an example, the distance from a handheld or wearable device to a food item can be estimated via the timing and/or angle of radio waves reflected by the food item. In an example, the distance from a handheld or wearable device to a food item can be estimated by analyzing the shape and size of a light pattern projected onto (or near) the food item.

In an example, a mobile device can project one or more visible beams of (coherent) light toward food. In an example, a mobile device can have one or more lasers which project one or more visible beams of light toward food. In an example, beams of light projected from a device can form a pattern on (or near) a food item which helps to calibrate food images and determine food item distance, angle, size, shape, orientation, and/or quantity. In an example, a mobile device can project an oscillating (or otherwise moving) beam of light on (or near) food items, wherein the size, shape, and/or orientation of a (geometric) figure formed by this oscillating (or otherwise moving) beam of light helps to calibrate food images and determine food distance, angle, size, shape, orientation, and/or quantity. In an example, a (geometric) figure projected onto (or near) food items can be selected from the group consisting of: line, cross, triangle, circle, square, rectangle, sine wave, spiral, checkerboard, dot array, hexagonal mesh, and matrix. In an example, a mobile device can further comprise an infrared distance finder to estimate the distance from the mobile device to food items. In an example, a mobile device can further comprise a radio wave distance finder to estimate the distance from the mobile device to food items.

In an example, a system for nutritional monitoring and management can include an ambient light sensor. In an example, if there is insufficient ambient light to record a good picture of nearby food, then the system can activate a light (e.g. flash) toward food to illuminate the food so that a good picture of the food can be recorded. In an example, a system can determine whether a camera is directed toward nearby food so that the food is within the field of view of the camera. If the nearby food is not within the field of view of the camera, then a person can be notified and/or guided by the system concerning how to move the camera and/or the food so that the food is brought within the field of view of the camera. In an example, a system can determine whether a nearby food is in focus by a camera. If the food is not in focus, then a person can be notified and/or guided by the system concerning how to move the camera and/or the food so that the food is brought into focus. In an example, a device can project a light beam and/or pattern toward nearby food to help a person to move a camera and/or to move the food so that the food is brought within the field of view of the camera and brought within focus by the camera.

In an example, a system for nutritional monitoring and management can include a wearable device with one or more cameras. In an example, this wearable device with one or more cameras can be selected from the group consisting of: augmented reality (AR) eyewear, bracelet, brooch, button, collar, contact lens, cuff link, dog tag, ear ring, ear-mounted bluetooth device, eyeglasses, finger ring, fitness band, headband, mobile EEG device, necklace, pendant, shirt, sleeve or cuff, visor, watch phone, wrist band, and wrist watch.

In an example, a system for nutritional monitoring and management can include one (or more) cameras which are worn on one (or more) locations on a person from which the one (or more) cameras have a line of sight to the person's mouth and a line of sight to a nearby food item. In an example, these one (or more) cameras can simultaneously or sequentially record images along at least two different vectors, one of which points toward a person's mouth and one of which points toward a food item. In an example, a system can comprise multiple cameras that are worn on a person's wrist, hand, arm, or finger, wherein some cameras point toward the person's mouth (when the person eats) and some cameras point toward nearby food items (when the person eats). In an example, a system can comprise one (or more) cameras that record images of interaction (e.g. biting, chewing, or swallowing) between a person's mouth and food. In an example, a system can comprise one (or more) cameras which collectively and automatically record images of a person's mouth when the person eats and record images of nearby food items when the person eats. In an example, these images can be automatically analyzed to estimate types and quantities of food consumed by the person.

In an example, a commonly-available object (e.g. a coin, dollar bill, credit card, die, paper clip, or ruler) of known size (and color) can be placed near food to serve as a fiducial marker in a food image for calibration of food size (and color) in image analysis. In an example, a (second) mobile device (such as a second smart phone) displaying an image of known size and colors can be placed near food to serve as a fiducial marker in the image for calibration of food size (and color) in image analysis. In an example, technical details of the display hardware of a particular type and/or brand of mobile device can also be considered in the calibration of food images. In an example, a mobile or wearable device can project one or more (coherent) light beams toward food and the resulting light beam pattern can serve as a fiducial marker in the image for calibration of food size (and color) in image analysis. In an example, one or more projected light beams can form a projected geometric shape on (or near) food. In an example, the size, shape, and/or orientation of this projected geometric shape on (or near) food can be used to help determine (e.g. calibrate) the distance, size, shape, orientation, and volume of the food.

In an example, a system for nutritional monitoring and management can include a light projector which light beams toward food. In an example, the light beams can be coherent. In an example, the light projector can be a laser. In an example, projected beams of light can form a geometric pattern on (or near) food items. In an example, a projected pattern of light can serve as a fiducial marker to estimate and/or calibrate food item distance, food item size, food item orientation, and/or food item color. In an example, a projected pattern of light can be selected from the group consisting of: a single line; a plurality of parallel lines; two intersecting lines; a grid of intersecting lines; a checkerboard pattern; a square; a hexagon; a circle; an array of concentric circles; and a (different type of) conic section.

In an example, a system for nutritional monitoring and management can include a light projector which projects a pattern of light onto food (or a surface within 12 inches of the food). In an example, the light pattern can serve as fiducial marker to calibrate and/or determine the size and/or quantity of the food. In an example, this light pattern can serve as fiducial marker to calibrate and/or determine the color of the food. In an example, a light projector can include one or more LEDs. In an example, a light projector can include one or more lasers. In an example, a light projector can project a pattern of coherent light onto food. In an example, a system can comprise a laser which projects coherent light beams onto nearby food (or on a surface near the food), wherein these light beams comprise a fiducial marker which helps to calibrate and/or measure the food scale, size, shape, volume, quantity, and/or color. In an example, a light projector can emit ultraviolet light or infrared light. In an example, a light projector can project collimated light. In an example, a projected light pattern can be used to link different locations on a food image with the results of spectroscopic scans at those different locations.

In an example, a system can project a circular pattern or ring of light onto food and/or a surface near food. In an example, a circle or ring of light can be a circle or ring of points (or dots) of light. In an example, a circle or ring of light can be a continuous circle or ring of light, such as is produced when a projecting member is rotated. In an example, a circle or ring of light can be a continuous circle or ring of light, such as is produced by a rotating micro-mirror onto which a beam of light is directed. In an example, the angle of the food or the surface on which the food is resting can be estimated by the degree of distortion of the circle or ring. If the food is imaged from directly above the food (or surface), then the projected light pattern is a circle, but if the food is imaged from an angle then it will be an ellipse. The angle of imaging can be determined by the compression of the observed ellipse. In an example, the light pattern projector can project a convex light pattern onto food or surfaces near the food.

In an example, a system can project a linear pattern of light onto food and/or a surface near food. In an example, a light pattern projector can project a polygonal light pattern onto food and/or a surface near food. In an example, a light pattern projector can project an array of three points of light onto food or a surface near the food. In an example, a light pattern projector can project a triangular light pattern onto food or a surface near food. In an example, a light pattern projector can project a matrix or grid of light onto food or a surface near food. In an example, a light pattern projector can project a matrix or grid of points (or dots) of light onto food or a surface near food. In an example, a light pattern projector can project an orthogonal light grid onto food. In an example, a light pattern projector can project a two-dimensional array of points of light onto or near food.

In an example, a light pattern which is projected from a projector can be moved across the surface of food by one or more moving micro-mirrors and/or lenses. In an example, an array of moving micromirrors or lenses can move a beam of light across food (or a surface near food) in order to create a pattern or configuration of light. In an example, an array of moving micromirrors or lenses can move a beam of light across food (or a surface near food) in order to create a line of light on the food. In an example, an array of moving micromirrors or lenses can move a beam of light across food (or a surface near food) in order to create a ring or other arcuate configuration of light on the food. In an example, an array of moving micromirrors or lenses can move a beam of light across food (or a surface near food) in order to create a grid or matrix of light on the food.

In an example, a system for nutritional monitoring and management can identify types of food items and/or their nutritional composition via spectroscopy. In an example, types of food, ingredients, and/or nutrients can be identified by the spectral patterns of light which has been reflected from (absorbed by) food at different wavelengths. In an example, an optical sensor can emit and/or detect white light, infrared light, or ultraviolet light. In an example, a system can include a spectroscopic sensor which is selected from the group consisting of: ambient light spectroscopic sensor, backscattering spectrometry sensor, coherent light spectroscopic sensor, infrared spectroscopic sensor, ion mobility spectroscopic sensor, mass spectrometry sensor, near-infrared spectroscopic sensor, Raman spectroscopic sensor, spectral measurement sensor, spectrometry sensor, spectrophotometer, ultraviolet spectroscopic sensor, visible light spectroscopic sensor, and white light spectroscopic sensor.

In an example, a system for nutritional monitoring and management can include a spectroscopic sensor (or, using the noun form as a modifier, a “spectroscopy sensor”). In an example, a spectroscopic sensor can collect data to identify a food item type by projecting light beams toward the food item and receiving those light beams after they have interacted with (e.g. passed through or been reflected by) the food item. In an example, changes in the spectral distribution of the light beams caused by interaction with a food item can be analyzed in order to identify food item type. In an example, a spectroscopic sensor can collect data concerning the nutritional composition and/or molecular composition of a food item by projecting light beams toward the food item and receiving those light beams after they have interacted with (e.g. passed through or been reflected by) the food item. In an example, changes in the spectral distribution of the light beams caused by interaction with a food item can be analyzed in order to estimate the nutritional and/or molecular composition of the food item.

In an example, a system for nutritional monitoring and management can include a wearable device with a spectroscopic sensor which collects data concerning a person's biometric parameters by projecting light beams toward the person's body and receiving those light beams after they have interacted with (e.g. passed through or been reflected by) body tissue. In example, changes in the spectral distribution of the light beams caused by interaction with body tissue can be analyzed in order to estimate biometric parameters. In an example, a wearable device can have a spectroscopic sensor which collects data concerning the molecular composition of body tissue by projecting light beams toward a person's body and receiving those light beams after they have interacted with (e.g. passed through or been reflected by) body tissue. In example, changes in the spectral distribution of the light beams caused by interaction with body tissue can be analyzed in order to estimate the molecular composition of the body tissue.

In an example, a system for nutritional monitoring and management can have a light receiver which collects data concerning light reflected from food items at two different times, wherein a light emitter which directs light beams toward food items is turned on at a first point in time but is turned off at a second point in time. In an example, during the first point in time, the light receiver receives a combination of light from the light emitter and ambient light which has been reflected by (or passed through) the food items. However, during the second point in time, the light receiver only receives ambient light which has been reflected by (or passed through) the food items. In an example, analyzing differences in light received by the receiver at these two different points in time can help to control for the effects of variation in ambient light on spectroscopic analysis of food. In an example, analyzing light reflected by (or passed through) the food items at these two different times can control for the effects of variation in ambient light on spectroscopic analysis of food. In an example, analyzing light received by the light receiver at these two different times can isolate interaction between food items and light beams from the light emitter vs. interaction between food items and ambient light.

In an example, a system for nutritional monitoring and management can have a light receiver which collects data concerning light reflected from body tissue at two different times, wherein a light emitter which directs light beams toward body tissue is turned on at a first point in time but is turned off at a second point in time. In an example, during the first point in time, the light receiver receives a combination of light from the light emitter and ambient light which has been reflected by (or passed through) the body tissue. However, during the second point in time, the light receiver only receives ambient light which has been reflected by (or passed through) the body tissue. In an example, analyzing differences in light received by the receiver at these two different points in time can help to control for the effects of variation in ambient light on spectroscopic analysis of biometric parameters. In an example, analyzing light reflected by (or passed through) the body tissue at these two different times can control for the effects of variation in ambient light on spectroscopic analysis of biometric parameters. In an example, analyzing light received by the light receiver at these two different times can isolate interaction between body tissue and light beams from the light emitter vs. interaction between body tissue and ambient light.

In an example, a system for nutritional monitoring and management can have a wearable or handheld device with an spectroscopic sensor which has a light receiver, but no light emitter. In an example, a light receiver can receive ambient light after that ambient light has been reflected from a food item. In an example, a system can have a first light receiver which receives ambient light directly from an environmental source and a second light receiver which receives ambient light after that light has been reflected from a food item. In an example, differences between the spectra of light received by the first and second light receivers can be analyzed to determine food item type, nutritional composition, and/or molecular composition. In an example, a system can reflect, redirect, and/or focus ambient light toward food instead of using a light emitter. In an example, a system can have a mirror or lens which is adjusted in order to reflect or direct sunlight (or other ambient light) toward food. In an example, reflection of ambient light from the food can be analyzed in order to identify food type and/or estimate food composition.

In an example, a system for nutritional monitoring and management can have an optical sensor. In an example, an optical sensor can measure ambient light level. In an example, an optical sensor can be a chromatographic sensor, spectrographic sensor, analytical chromatographic sensor, liquid chromatographic sensor, gas chromatographic sensor, optoelectronic sensor, photochemical sensor, and photocell. In an example, an optical sensor can collect data concerning modulation of light wave parameters by the interaction of that light with food. In an example, an optical sensor can detect modulation of light reflected from, or absorbed by, a receptor when the receptor is exposed to food. In an example, an optical sensor can collect data concerning wavelength spectra of light reflected from, or absorbed by, food. In an example, an optical sensor can emit and/or detect white light, infrared light, or ultraviolet light. In an example, an optical sensor can detect ambient light before and after interaction of the ambient light with food. In an example, changes in ambient light before vs. after interaction with food can be analyzed to identify food type and/or nutritional composition.

In an example, a system for nutritional monitoring and management can include a spectroscopic sensor which collects data to identify types of foods, ingredients, nutrients, and/or chemicals by being in optical communication with food items without actually touching the food items. In an example, light beams with different wavelengths can be reflected off (or absorbed by) food items and the results can be analyzed using spectral analysis. Selected types of foods, ingredients, nutrients, and/or chemicals can be identified by the spectral distributions of light which are reflected from, or absorbed by, food items at different wavelengths. In an example, reflection of light from the surface of the food items changes the spectrum of light, wherein these changes are measured by a spectroscopic sensor in order to estimate the nutritional and/or chemical composition of the food. In an example, the passing light through food items changes the spectrum of light, wherein these changes are measured by a spectroscopic sensor in order to estimate the nutritional and/or chemical composition of the food items.

In an example, a system for nutritional monitoring and management can include a wearable device with an spectroscopic sensor which has a light receiver, but no light emitter. In an example, a light receiver can receive ambient light after that ambient light has been reflected from body tissue. In an example, a system can have a first light receiver which receives ambient light directly from an environmental source and a second light receiver which receives ambient light after that light has been reflected from body tissue. In an example, differences between the spectra of light received by the first and second light receivers can be analyzed to determine biometric parameters. In an example, a system can reflect, redirect, and/or focus ambient light toward body tissue instead of using a light emitter. In an example, a system can have a mirror or lens which is adjusted in order to reflect or direct sunlight (or other ambient light) toward body tissue. In an example, reflection of ambient light from the body tissue can be analyzed in order to measure biometric parameters.

In an example, a system for nutritional monitoring and management can include a wearable or handheld device with one or more spectroscopic sensors which collect data concerning the effects of interaction between light energy and food. In an example, a spectroscopic sensor can collects data concerning changes in the spectrum of light energy caused by reflection from (or passage through) food items. In an example, a spectroscopic sensor can collect data concerning light reflection spectra, absorption spectra, or emission spectra. In an example, a spectroscopic sensor can collect data which is used to analyze the chemical composition of food by measuring the degree of reflection or absorption of light by food at different light wavelengths.

In an example, a system for nutritional monitoring and management can comprise one or more spectroscopic sensors which analyze light which has been passed through and/or reflected by food items. In an example, a spectroscopic sensor can comprise a light emitter and a light receiver, wherein the light receiver receives light which has passed through and/or been reflected by a food item. In an example, changes in the spectrum of light caused by interaction with food (e.g. by transmission through or reflection by food) can be analyzed to estimate the nutritional and/or molecular composition of the food. In an example, transmission and/or reflection spectra of different food items can be analyzed to identify these food items and/or to estimate their compositions. In an example, modification of spectral distributions of light by food items can be compared to spectral distributions in a database of such spectral distributions to help identify food and the composition of ingredients/nutrients therein. In an example, a spectroscopic sensor can be selected from the group consisting of: atomic absorption spectrometer, diffusion spectroscopic sensor, emission spectroscopic sensor, fluorescence spectroscopic sensor, gas chromatography sensor, infrared absorption spectrometer, infrared reflectance spectrometer, mass spectrometer, mass spectrometry sensor, near-infrared spectroscopic sensor, photodiode array spectrophotometer, Raman spectroscopy sensor, spectrometer, spectrophotometer, and ultra-violet reflectance spectrometer.

In an example, a spectroscopic sensor can have one or more light emitters selected from the group consisting of: Light Emitting Diode (LED), laser, and tunable laser. In an example, a spectroscopic sensor can comprise a plurality of light emitters which emit light beams of different colors and/or different wavelengths. In an example, a spectroscopic sensor can comprise a plurality of light emitters which emit light at different times. In an example, a spectroscopic sensor can comprise a plurality of light emitters which emit light from different locations on a device. In example, a system can comprise a wearable device with a circumferential array (e.g. a ring) of spectroscopic sensors around the circumference of a person's wrist, arm, or finger. In an example, one or more light emitters can emit light in a wavelength range selected from the group consisting of: far infrared, infrared, near infrared, ultraviolet, and visible. In an example, a spectroscopic sensor can use ambient light which has passed through and/or been reflected by a food item. In an example, spectroscopic readings can be taken when a light emitter is on and when the light emitter is off in order to isolate the effects of ambient light and more precisely measure the spectroscopic effects of interaction between light from a light emitter and food items. In an example, one or more light receivers can be selected from the group consisting of: charge coupled device (CCD), complementary metal oxide semiconductor (CMOS), detector array, photo transistor, photodetector, photodiode, and photosensor array.

In an example, a spectroscopic sensor can comprise components selected from the group consisting of: aperture; beam splitter; coaxial light emitter and light receiver; diffraction grating; lens (e.g. spherical, aspheric, biconvex, Fresnel lens, Carlavian lens, microlens array, plano-convex); light emitter; light receiver; mirror (e.g. Digital Micromirror Device, parabolic reflector, Quasi Fresnel Reflector, mirror array); opaque light shield; optical fiber; and optical filter (e.g. Fabry-Perot, tunable, acousto-optic, liquid crystal, cascaded, interference).

In an example, a system for nutritional monitoring and management can comprise: a camera that records images of nearby food; an optical sensor (e.g. a spectroscopic sensor) which collects data concerning light that is reflected from (or passed through) this food; an attachment mechanism (e.g. a wrist band); and a data processing unit which analyzes images. In an example, a system can comprise: a camera which records images of food items, wherein these food images are automatically analyzed to identify food item types and quantities; an optical sensor which collects data concerning light that has been reflected by (or transmitted through) the food items, wherein this data is automatically analyzed to identify the types of food, the types of ingredients in the food, and/or the types of nutrients in the food; one or more attachment mechanisms which hold the camera and the spectroscopic sensor in close proximity to a person's body; a data processing unit which analyzes images; and a computer-to-human interface which provides feedback to the person concerning the person's nutritional intake.

In an example, a system for nutritional monitoring and management can include a spectroscopic sensor with a light emitter which emits near infrared light. In an example, a spectroscopic sensor can include a light emitter which emits infrared light. In an example, a spectroscopic sensor can include a light emitter which emits ultraviolet light. In an example, a spectroscopic sensor can include a light emitter which emits light with a wavelength in the range of 400 to 700 nanometers. In an example, emitted light can have a wavelength in the range of 300 to 1200 nanometers. In an example, a spectroscopic sensor can include a light energy receiver which is particularly receptive to near-infrared, infrared, or ultraviolet light. In an example, a system can comprise one or more spectroscopic sensors selected from the group consisting of: near-infrared spectroscopic sensor; infrared spectroscopic sensor; white light spectroscopic sensor; and ultraviolet spectroscopic sensor. In an example, one or more light emitters can be selected from the group consisting of: white LED, blue LED, red LED, infrared LED, and green LED.

In an example, a system for nutritional monitoring and management can comprise: a finger ring, wherein this finger ring has an interior surface which faces toward the surface of a person's finger, wherein this finger ring has a central proximal-to-distal axis which is defined as the straight line which most closely fits a proximal-to-distal series of centroids of cross-sections of the interior surface, and wherein proximal is defined as being closer to the person's elbow and distal is defined as being further from the person's elbow; a light projector which projects a beam of light along a proximal-to-distal vector toward a food item, wherein this vector, or a virtual extension of this vector, is either parallel to the central proximal-to-distal axis or intersects a line which is parallel to the central proximal-to-distal axis forming a distally-opening angle whose absolute value is less than 45 degrees; and a spectroscopic sensor which collects data concerning the spectrum of light which has been reflected from, or has passed through, the food item, and wherein data from the spectroscopic sensor is used to analyze the composition of this food.

In an example, a system for nutritional monitoring and management can comprise: a handheld food probe with a spectroscopic sensor which collects data concerning light which has been reflected from (or passed through) a food item, wherein this data is used to analyze the nutritional and/or chemical composition of the food item; a camera which records images of the food item; and a light projector which projects a light pattern near (or on) the food, wherein the projected light pattern serves as a fiducial market to estimate and/or calibrate the size and/or color of the food.

In an example, a system for nutritional monitoring and management can have a spectroscopic sensor with an array of light emitters and an array of light receivers. In an example, a spectroscopic sensor can include one light emitter and two light receivers. In an example, a spectroscopic sensor can include two light emitters and one light receiver. In an example, a spectroscopic sensor can include a plurality of light emitters at different locations. In an example, a spectroscopic sensor can have a two-dimensional arcuate array with at least one light emitter and at least one light receiver. In an example, a spectroscopic sensor can have a three-dimensional array of light emitters and receivers. In an example, a spectroscopic sensor can have a plurality of light emitters and receivers in a three-dimensional matrix or grid. In an example, a spectroscopic sensor can have a plurality of light emitters which emit light at different angles.

In an example, a system for nutritional monitoring and management can include a spectroscopic sensor having a circular or annular array with at least one light emitter and at least one light receiver. In an example, a spectroscopic sensor can have a ring of light emitters and receivers. In an example, a spectroscopic sensor can have a plurality of light emitters in a ring or circle around a light receiver. In an example, a spectroscopic sensor can have at least one light emitter and at least one light receiver in a concentric configuration. In an example, a spectroscopic sensor can have a plurality of light emitters in a polygonal configuration around a light receiver. In an example, a spectroscopic sensor can have a polygonal array with at least one light emitter and at least one light receiver.

In an example, a system for nutritional monitoring and management can include a handheld device with a spectroscopic sensor which located at the distal end of the handheld device. In an example, the proximal end of the handheld device can be held by a person's hand and the distal end can be pointed toward a food item in order to take a spectroscopic scan of the food item. In an example, a spectroscopic sensor comprising a light emitter and a light receiver can be located at the distal end of a handheld device. In an example, a light emitter can emit a beam of light toward food from the distal end of a handheld device. In an example, a central vector of this emitted beam of light can be substantially parallel to the proximal-to-distal axis of the handheld device.

In an example, a system for nutritional monitoring and management can include a spectroscopic sensor with an optical filter. In an example, a spectroscopic sensor can include a two-dimensional array of optical filters. In an example, a spectroscopic sensor can have one or more optical filters selected from the group consisting of: optical absorption filter; acousto-optic filter; Bragg filter; cascaded filter; dielectric thin-film filter; Fabry-Perot filter; hybrid filter; and optical interference filter. In an example, a system can include a spectroscopic sensor with an optical diffuser. In an example, a spectroscopic sensor can include a two-dimensional lens array. In an example, a spectroscopic sensor can include a three-dimensional lens array. In an example, a spectroscopic sensor can have a moving mirror. In an example, a spectroscopic sensor can have a moving micromirror array.

In an example, a system for nutritional monitoring and management can comprise one or more actuators which change the focal direction and/or distance of a spectroscopic sensor. In an example, a system can comprise one or more actuators which move the focal direction and/or distance of a spectroscopic sensor back and forth across the surface of nearby food. In an example, a system can comprise one or more actuators which move the focal direction and/or distance of a spectroscopic sensor in an arcuate pattern over the surface of nearby food. In an example, a system can include a visible light beam which is moved in tandem with (e.g. aligned with) the focal direction of a spectroscopic sensor so that the location on a meal or a food item which is targeted for spectroscopic scanning at a given time can be identified in a food image. In this manner, information concerning food item type and/or quantity from food image analysis at a particular location (on a meal or food item) at a given time can be linked with information concerning foot item type and/or composition from spectroscopic analysis of that particular location at a given time.

In an example, a system for nutritional monitoring and management can have a spectroscopic sensor with a plurality of light emitters which emit light in different wavelength ranges. In an example, a spectroscopic sensor can have a plurality of light emitters which emit light at different frequencies and/or wavelengths. In an example, a system can have a plurality of spectroscopic sensors which sequentially emit light at different frequencies. In an example, a system can have a plurality of spectroscopic sensors which simultaneously emit light at different frequencies. In an example, the operation of a spectroscopic sensor can include frequency-based modulation.

In an example, a system for nutritional monitoring and management can include a spectroscopic sensor which emits light at different frequencies at different times. In an example, a system can comprise a spectroscopic sensor which emits a sequence of light beams at different frequencies. In an example, a light emitter can emit light with scanning variation in frequencies and/or wavelength. In an example, a light emitter can emit light in a sweeping series of frequencies. In an example, a light emitter sensor can emit light in a sequentially-varying range of frequencies. In an example, a light emitter can emit light with a frequency which changes over time. In an example, a light emitter can emit light in a sweeping series of wavelengths. In an example, a light emitter can emit light in a sequentially-varying range of wavelengths. In an example, a light emitter can emit light with a wavelength which changes over time.

In an example, a system for nutritional monitoring and management can include a spectroscopic sensor with a plurality of light emitters which emit light at different times. In an example, a spectroscopic sensor can have an array of light emitters which emit light pulses at different times. In an example, a spectroscopic sensor can have a linear array of light emitters which emit light pulses at different times. In an example, a spectroscopic sensor can have an annular array of light emitters which emit light pulses at different times. In an example, a spectroscopic sensor can have a plurality of light emitters which are selectively and sequentially activated. In an example, a plurality of light emitters can be selectively and sequentially activated via time-based multiplexing. In an example, a spectroscopic sensor can operate with time-based multiplexing.

In an example, a system for nutritional monitoring and management can have a spectroscopic sensor with a moveable cover or lid. In an example, the moveable cover or lid can open when the sensor is a first distance from food and close when the sensor is a second distance from food. In an example, a cover or lid for a spectroscopic sensor can open when the sensor is close enough to food to record an accurate spectroscopic scan, but can close if the spectroscopic sensor is so close to the food that it could actually touch the food. This can help to ensure sufficient closeness to food to get an accurate spectroscopic scan, but avoid smearing food on the surface of the sensor. In an example, a distance range during which a cover or lid automatically opens can be close enough that a high proportion of light entering the sensor has been reflected from the surface of nearby food, but not so close that food actually touches the sensor. In an example, a cover or lid can be automatically opened and the sensor can be activated to emit and receive beams of light at a distance from food which is greater than X and less than Y. In an example, X can be between 1 and 200 microns, while Y can be between 5 and 500 microns. In an example, X can be between 1/10th of an inch and 1 inch, while Y can be between ¼ of an inch and 3 inches. In an example, a cover or lid on a spectroscopic sensor can close automatically when it gets too close to food. This can prevent the sensor from being smeared with food.

In an example, a system for nutritional monitoring and management can suggest a plurality of locations for spectroscopic analysis of a food item and/or food items in a meal. In an example, a system can guide a person concerning where the person should take a plurality of spectroscopic scans of a food item and/or food items in a meal. In an example, the number and/or breadth of locations suggested by a system for spectroscopic scans of food items can depend on the homogeneity and/or variability of food items and/or a meal. In an example, a larger number and/or broader area of spectroscopic scans can be suggested by a system for food items and/or meals which are less homogeneous and/or have greater apparent compositional variability. In an example, a smaller number and/or narrower area of spectroscopic scans can be suggested by a system for food items and/or meals which are more homogeneous and/or have less apparent compositional variability.

In an example, a system for nutritional monitoring and management can analyze food images from a camera to evaluate the apparent uniformity or homogeneity of food items. In an example, food images from a camera can be analyzed to automatically direct locations on the food where a person should direct spectroscopic scans of the food. In an example, food images from a camera can be analyzed to direct a light projector to shine on food. In an example, a system can guide a person concerning where to take spectroscopic scans of the food (e.g. based on inter-portion and intra-portion food variability). In an example, food images from a camera can be analyzed to suggest locations on the food where a person should take spectroscopic scans of the food.

In an example, a system for nutritional monitoring and management can suggest different numbers or locations of spectroscopic scans of food items, depending on intra-portion food homogeneity and/or inter-portion food homogeneity. In an example, the number or locations suggested by a system for spectroscopic scans of food items can depend on intra-portion food variation and/or inter-portion food variation. Analysis of food uniformity or homogeneity can include inter-portion variation (e.g. differences in food type between different portions of food in a meal) and intra-portion variation (e.g. differences in ingredients between different parts in a portion of one type of food). In an example, analysis of inter-portion and intra-portion food variability can inform the number and locations of suggested spectroscopic scans for a meal. In an example, a larger number of spectroscopic scans and/or scans over a wider range of locations can be suggested for meals with greater inter-portion and/or intra-portion variability. In an example, a smaller number of spectroscopic scans and/or scans over a narrower range of locations can be suggested for meals with greater inter-portion and/or intra-portion variability.

In an example, a system for nutritional monitoring and management can include a light projector which projects a light beam which is moved to (sequentially) highlight different portions (types) of food in a meal or on a dish, which can then be linked to sequential spectroscopic analysis of the chemical composition of those different portions (types) of food. In an example, food images from a camera can be analyzed to suggest different locations in a meal where a person should take spectroscopic scans. In an example, food images from a camera can be analyzed to direct a light projector so as to guide a user where to take spectroscopic scans of the food (e.g. based on inter-portion and intra-portion food variability).

In an example, a system for nutritional monitoring and management can analyze the degree of uniformity and/or homogeneity of food items and use the results to suggest a number and/or selected set of locations for spectroscopic scans of the food items. When a food item (or meal) is less-uniform or less-homogenous, then a larger number and wider range of spectroscopic scans can be required for identification and quantification of foods, ingredients, and/or nutrients. When a food item (or meal) is more-uniform or more-homogenous, then a smaller number and narrower range of spectroscopic scans can be required for identification and quantification of foods, ingredients, and/or nutrients. In an example, a system can show a person where spectroscopic scans should be made by sequentially projecting a light pattern onto different locations on food (like a three-dimensional cursor). In an example, the results of spectroscopic scans at selected locations can be linked to pattern analysis of food images for assessing inter-portion and intra-portion variation in the molecular composition of food.

In an example, a system for nutritional monitoring and management can further comprise one or more wearable or implanted devices which collect biometric information concerning a person whose nutritional intake is being monitored and managed. In an example, a wearable device which is part of the system can be selected from group consisting of: smart watch (e.g. smart watch housing or band), wrist-worn fitness band or bracelet, arm band, smart eyewear (e.g. smart eyeglasses, AR eyewear, EEG eyewear), smart earware (e.g. ear buds, ear pod), smart clothing, smart adhesive patch, continuous glucose monitor, head band and/or mobile EEG band, sweat sensor, and intra-oral device (e.g. dental implant, retainer, upper palate attachment, tongue piercing). In an example, an implanted device which is part of the system can be selected from the group consisting of: cardiac rhythm device (e.g. pacemaker), implanted neurostimulator, implanted drug delivery device, and smart stent.

In an example, a system for nutritional monitoring and management can monitor and respond to changes in a person's body glucose level and/or body oxygen level. In an example, a system can monitor and respond to changes in a person's blood pressure and/or heart rate. In an example, a system can monitor and respond to changes in photoplethysmographic (PPG) data and/or ballistocardiographic (BCG) data. In an example, a system can monitor and respond to changes in a person's body temperature and/or respiration rate. In an example, a system can include a biometric sensor which measures one or more biometric parameters selected from the group consisting of blood pressure, body glucose level, body temperature, heart rate, lactic acid level, and body oxygen level. In an example, a system can be in electromagnetic communication with a biometric sensor device which measures one or more of these biometric parameters.

In an example, system with a spectroscopic sensor and a wearable device which measures heart rate, rhythm, and/or rate variability can together comprise an integrated system for food identification and quantification. In an example, when a wearable device detects changes in a person's heart rate, rhythm, and/or rate variation which indicates that the person is eating, then the system can prompt the person to scan food using a spectroscopic sensor. In an example, a mobile device with a camera and a wearable device which measures heart rate, rhythm, and/or rate variability can together comprise a system for food identification and quantification. In an example, when a wearable device detects changes in a person's heart rate, rhythm, and/or rate variation which indicates that the person is eating, then the system can prompt the person to take pictures of the food using the camera.

In an example, a system for nutritional monitoring and management can monitor and respond to changes in a person's electrocardiographic (ECG) data, electromyographic (EMG) data, and/or electroencephalographic (EEG) data. In an example, a system can monitor and respond to changes in a person's body pH level and/or lactic acid level. In an example, a system can monitor and respond to changes in a person's body chemistry. In an example, a system can monitor and respond to changes in a person's galvanic skin response. In an example, a system can track and respond to a person's eye movements.

In an example, changes in one or more (of the above discussed) biometric parameters can trigger actions by the system. In an example, changes in one or more (of the above discussed) biometric parameters which indicate that a person is probably eating can trigger actions by the system. In an example, actions triggered by the system in response to a person eating can be selected from the group consisting of: automatically recording images to record images of nearby food items (which are being consumed); prompting a person to record images of nearby food items (which are being consumed); automatically increasing the level or types of sensor activity to more accurately collect information to determine types and quantities of nearby food items (which are being consumed); and prompting a person to provide additional user information (e.g. verbal descriptions) concerning nearby food items (which are being consumed).

In an example, a system for nutritional monitoring and management can analyze changes in one or more biometric parameters to identify relationships between the consumption of specific types and/or quantities of food by a person and subsequent health effects or health status concerning that person. In an example, a system can identify relationships between consumption of specific types and/or quantities of food by a person and subsequent changes in the person's blood glucose levels. In an example, a system can identify relationships between consumption of specific types and/or quantities of food by a person and subsequent changes in the person's self-reported wellness status and/or energy level. In an example, a system can identify food allergies, intolerances, or diseases related to consumption of specific types of food. In an example, relationships identified between consumption of specific types and/or quantities of food by a person and subsequent changes in the person's biometric parameters can be used by a system in personalized future recommendations that this person consume more of a first type of food and/or recommend that this person consume less (or none) of a second type of food.

In an example, a system for nutritional monitoring and management can investigate, identify, track, and respond to correlations between consumption of specific types and/or quantities of food by a person and subsequent biometric parameters and/or health effects concerning that person. In an example, a system can track correlations between consumption of (selected) foods and subsequent self-reported well-being of that person. In an example, a system can track correlations between consumption of (selected) foods and subsequent blood pressure levels of that person. In an example, a system can track correlations between consumption of (selected) foods and subsequent blood glucose levels of that person. In an example, a system can track correlations between consumption of (selected) foods and subsequent blood flow of that person. In an example, a system can track correlations between eating selected types of food and subsequent illness in order to identify food allergies, food intolerances, and/or diseases. In an example, causal associations between consumption of specific types of food and subsequent changes in one or more biometric parameters can be identified and used by the system to make food consumption recommendations. In an example, causal associations between consumption of specific types of food and subsequent changes in one or more of these biometric parameters can be identified and used to refine future measurements of food types and/or quantities by the system.

In an example, the effects of consumption of specific types of food on one or more biometric parameters can be analyzed. In an example, causal associations between consumption of specific types of food and subsequent changes in one or more of these biometric parameters can be identified and used to make food consumption recommendations. In an example, causal associations between consumption of specific types of food and subsequent changes in one or more biometric parameters can be identified for a specific person and used to refine future measurements of food types and/or quantities for that person.

In an example, a system for nutritional monitoring and management can include a lower-level eating-related sensor and a higher-level eating-related sensor. In an example, the lower-level eating-related sensor can be relatively accurate in detecting that a person is eating, be relatively non-intrusive with respect to privacy, and/or have relatively low power consumption, but not be very accurate in identifying specific food types and/or estimating food quantities. In an example, the higher-level eating-related sensor can be relatively accurate in identifying specific food types and/or estimating food quantities, but can be relatively non-intrusive with respect to privacy and/or have relatively high power consumption. In an example, a system can activate and/or trigger operation of the higher-level eating-related sensor when the lower-level eating-related sensor detects that a person is eating. In an example, a lower-level eating-related sensor can be selected from the group consisting of: wearable motion sensor; motion sensor which is part of a smart utensil; wearable microphone; wearable EMG sensor; wearable EEG sensor, and wearable camera. In an example, a higher-level eating-related sensor can be selected from the group consisting of: wearable camera; wearable spectroscopic sensor; handheld camera; and handheld spectroscopic sensor.

In an example, a system for nutritional monitoring and management can continuously monitor eating via a “level 1” sensor, but only activate a “level 2” sensor when eating is detected. In an example, a “level 1” sensor can be less intrusive with respect to a person's privacy, but also less accurate with respect to determining food item types and quantities. In an example, a “level 2” sensor can be more intrusive with respect to a person's privacy, but also more accurate with respect to determining food item types and quantities. In an example, a “level 1” sensor can be a motion sensor and a “level 2” sensor can be a camera. In an example, a “level 1” sensor can be a motion sensor and a “level 2” sensor can be a microphone. In an example, a “level 1” sensor can be a motion sensor and a “level 2” sensor can be an electromagnetic energy sensor.

In an example, a system for nutritional monitoring and management can include a camera which is aimed toward a person's hand, the person's mouth, or a food item in order to record an image of a food item which the person reaches for, grasps, and/or holds. In an example, a system can use gesture recognition to track a person's hand or use face recognition to track a person's mouth. In an example, the focal direction and/or imaging vector of a camera can be automatically adjusted so that the camera stays focused on a hand, mouth, or food item. In an example, if the line of sight from a camera to one of these objects is obscured, then the system can monitor the last known location of the object and/or extrapolate expected movement of the object to a new location in order regain a line of sight to the object.

In an example, a system for nutritional monitoring and management can include a wearable device with a camera, wherein the device is worn like a watch, worn like a necklace, worn on clothing (like a button), worn like a finger ring, or worn like an ear ring. In an example, the focal direction and/or distance of a camera can adjusted in real time to record images of food, but minimizing privacy-intruding images of people or other objects. In an example, a camera can be kept oriented toward a person's hand so that nearby people are generally not in focus in images. In an example, face recognition and/or pattern recognition can be used to automatically blur privacy-intruding portions of an image such as other people's faces. In an example, the focal range of a camera can be adjusted in real time to automatically blur privacy-intruding portions of an image such as other people's faces.

In an example, a system for nutritional monitoring and management can record food images in an intermittent, periodic, or random manner which does not requiring voluntary actions by the person associated with particular eating events other than the actions of eating. In an example, a system can record food images which one or more sensors indicate that a person is eating. In an example, these sensors can be motion sensors, sound sensors, and/or electromagnetic energy sensors.

In an example, a system for nutritional monitoring and management can include a camera which takes pictures of food and/or records images of food. In an example, a system can include a camera which automatically records images of food when the system detects that a person is probably eating. In an example, a system can include a camera which automatically records images of food when the system detects food nearby. In an example, a system can include two cameras which record stereoscopic images of food for three-dimensional analysis of the food. In an example, a system can include a barcode and/or QR code reader. In an example, a system can include optical character recognition capability. In an example, a system can include food-associated logo recognition capability.

In an example, a system for nutritional monitoring and management can analyze food items using spectroscopic analysis in a targeted manner, searching for one or more specific substances of interest. In an example, a person can be allergic to a specific type of food or a specific substance which may be in food. In an example, there may be reason to believe that food may have been adulterated with a specific substance. In an example, a system can focus in-depth spectroscopic analysis within a specific spectral range to more accurately search for a selected substance.

In an example, a system for nutritional monitoring and management can include a spectroscopic sensor which collects data which is used to determine food item types and/or quantities. In an example, a spectroscopic sensor can collect data which helps to identify food item type, food item nutritional composition, and/or food item chemical composition by analysis of the interaction between light energy and a food item. In an example, this interaction can be the amount of light reflection or light absorption by a food item at different light wavelengths. In an example, a system can include a handheld device with a spectroscopic sensor which is directed toward nearby food. In an example, a system can include a wearable device with a spectroscopic sensor which is directed toward nearby food. In an example, a wrist-worn wearable device with a spectroscopic sensor can be waved back and forth over a food item in order to spectroscopically scan the food item.

In an example, a system for nutritional monitoring and management can trigger a spectroscopic scan when motion patterns indicate that a person is eating. In an example, a system can perform multiple spectroscopic scans, at different times, while a person is eating in order to better analyze the overall composition of food with different internal layers and/or a non-uniform ingredient structure. In an example, a spectroscopic sensor can be automatically activated (e.g. turned on) within a given range of distance from food.

In an example, a system for nutritional monitoring and management can include a spectroscopic sensor which scans food to collect information concerning the nutritional and/or molecular composition of the food. In an example, a system can have a light emitter which emits light beams toward food and a light receiver which receive those light beams after those beams have been transmitted through and/or reflected by the food. In an example, changes in the spectral distribution of light beams caused by transmission through and/or reflection by food can be analyzed to determine the nutritional and/or molecular composition of the food. In an example, spectrographs of food items can be used to help identify food types. In an example, a spectroscopic sensor can be a spectrometer. In an example, a food-scanning spectroscopic sensor can be selected from the group consisting of: atomic absorption spectrometer, diffusion spectroscopic sensor, emission spectroscopic sensor, fluorescence spectroscopic sensor, gas chromatography sensor, infrared absorption spectrometer, infrared reflectance spectrometer, mass spectrometer, mass spectrometry sensor, near-infrared spectroscopic sensor, photodiode array spectrophotometer, Raman spectroscopy sensor, spectrometer, spectrophotometer, and ultra-violet reflectance spectrometer.

In an example, a system for nutritional monitoring and management can include a wearable or handheld device with a spectroscopic sensor which is used to estimate a person's biometric parameters. In an example, biometric parameters can be selected from the group consisting of: oxygen level; heart rate; blood pressure; hydration level; glucose level; and lactic acid level. In an example, a spectroscopic sensor can estimate biometric parameters by analyzing interaction between light energy and body tissue. In an example, this interaction can be the amount of light reflection or light absorption by body tissue at different light wavelengths. In an example, a system can include a wearable device with a spectroscopic sensor which is directed toward body tissue. In an example, a system can include a handheld device with a spectroscopic sensor which is directed toward body tissue. In an example, a system can include a handheld device with a spectroscopic sensor into which a person inserts their finger.

In an example, a system for nutritional monitoring and management can include a spectroscopic sensor which scans body tissue to collect information concerning a person's biometric parameters and/or health status. In an example, a system can have a light emitter which emits light beams toward body tissue and a light receiver which receive those light beams after those beams have been transmitted through and/or reflected by the body tissue. In an example, changes in the spectral distribution of light beams caused by transmission through and/or reflection by body tissue can be analyzed to estimate values of biometric parameters and/or evaluate a person's health status. In an example, a spectroscopic sensor can be a spectrometer. In an example, a tissue-scanning spectroscopic sensor can be selected from the group consisting of: atomic absorption spectrometer, diffusion spectroscopic sensor, emission spectroscopic sensor, fluorescence spectroscopic sensor, gas chromatography sensor, infrared absorption spectrometer, infrared reflectance spectrometer, mass spectrometer, mass spectrometry sensor, near-infrared spectroscopic sensor, photodiode array spectrophotometer, Raman spectroscopy sensor, spectrometer, spectrophotometer, and ultra-violet reflectance spectrometer.

In an example, a system for nutritional monitoring and management can include a wearable device with a motion sensor which is worn on a person's arm, wrist, hand, and/or finger. In an example, a system can include a wearable device with a motion sensor which is worn on a person's neck, face, ear, and/or head. In an example, a motion sensor can comprise an accelerometer and a gyroscope. In an example, a system can have a motion sensor which is selected from the group consisting of: bubble accelerometer, dual-axial accelerometer, electrogoniometer, gyroscope, inclinometer, inertial sensor, multi-axis accelerometer, piezoelectric sensor, piezo-mechanical sensor, pressure sensor, proximity detector, single-axis accelerometer, strain gauge, stretch sensor, and tri-axial accelerometer. In an example, a system can have a wearable device with a motion sensor which is used to: detect when a person is eating (and optionally trigger advanced sensor monitoring); identify the type of food that the person is eating; and/or estimate the quantity of food that the person is eating.

In an example, a system for nutritional monitoring and management can include a motion sensor that collects data concerning movement of a person's body. In an example, a motion sensor can collect data concerning the movement of a person's wrist, hand, fingers, arm, head, mouth, jaw, and/or neck. In an example, detected motion can be repeated motion of a person's jaws and/or mouth. In an example, detected motion can be peristaltic motion of a person's esophagus (detectable via contact with the person's neck). In an example, analysis of such motion data can detect when a person is eating and estimate how much a person is eating. In general, a motion sensor is more useful for general detection of food consumption and/or estimation of food quantity than for identification of specific food item types, ingredients, and/or nutrients. However, a motion sensor can be used in combination with advanced food-identifying sensors (such as spectroscopic sensors) for more complete identification of food item types as well as quantities. In an example, motion data which indicates eating can be used to trigger additional data collection by advanced food-identifying sensors to resolve uncertainty concerning the types and quantities of food that a person is consuming. In an example, motion data which indicates that a person is eating can trigger a system to prompt a person to provide their own description of food items consumed in order to resolve uncertainty concerning the types and quantities of food that the person is eating.

In an example, a system for nutritional monitoring and management can include a biometric sensor which measures a person's blood pressure. In an example, a system can further comprise a biometric sensor which measures a person's blood glucose level. In an example, a system can further comprise a biometric sensor which measures a person's tissue oxygenation level. In an example, a system can further comprise a biometric sensor which measures a person's blood temperature. In an example, a system can further comprise a biometric sensor which measures a person's heart rate. In an example, a system can further comprise a biometric sensor which measures a person's lactic acid level. In an example, a system can further comprise a biometric sensor which measures a person's body hydration level.

In an example, a system for nutritional monitoring and management can include a motion sensor. In an example, a motion sensor can be an accelerometer, a gyroscope, a magnometer, a magnetic angular rate and gravity (MARG) sensor, a piezoelectric motion sensor, a strain sensor, a bend sensor, a compass, a motion-based chewing sensor, a motion-based swallowing sensor, a vibration sensor, or a combination thereof. In an example, a motion sensor can be part of a device worn on a person's arm, wrist, or finger. In an example, a motion sensor can be part of a food utensil.

In an example, a system for nutritional monitoring and management can include a wearable device with a motion sensor which tracks eating-related motions of a person's body. In an example, a hand-to-mouth movement that matches a distinctive eating pattern can be used to estimate a bite or mouthful of food consumed. In an example, the speed of hand-to-mouth movements that match distinctive eating patterns can be used to estimate the speed or pace of food consumption. In an example, distinctive eating-related motions can be selected from the group consisting of: finger movements, hand movements, hand gestures, wrist movements, arm movements, elbow movements, eye movements, and head movements; tilting movements, lifting movements; hand-to-mouth movements; angles of rotation in three dimensions around the center of mass known as roll, pitch and yaw; and Fourier transformation analysis of repeated body member movements. In an example, a wearable motion sensor can comprise a three-dimensional accelerometer and gyroscope in a wrist-worn device (such as smart watch). In an example, a wearable motion sensor can comprise a three-dimensional accelerometer and gyroscope in a finger-worn device (such as smart ring). In an example, a motion sensor can detect eating by monitoring three-dimensional movement of a person's arm and/or hand. Eating activity can be indicated by distinctive sequences of up and down, or rolling and pitching, movements.

In an example, a system for nutritional monitoring and management can include a wearable device with a motion sensor which tracks eating-related motions of a person's arm, wrist, and hand. In an example, a person raising their hand up to their mouth in a distinctive manner can be an eating-related motion. In an example, an eating-related motion can include a distinctive three-dimensional combination of roll, pitch, and yaw motions by a person's arm, wrist, and/or hand. In an example, a distinctive rotation of a person's wrist can indicate that the person is eating food. In an example, eating can be associated with a body motion sequence comprising an upward and posterior-tilting hand motion, followed by a pause, followed by a downward and anterior-tilting hand motion. In an example, a motion sensor can detect a distinctive pattern comprising an upward (hand-up-to-mouth) arm motion, followed by a distinctive pattern of tilting or rolling motion (food-into-mouth) wrist motion, followed by a distinctive pattern of downward (hand-down-from-mouth) motion. In an example, indications that a person is eating can be selected from the group consisting of: acceleration, inclination, twisting, or rolling of the person's hand, wrist, or arm; acceleration or inclination of the person's lower arm or upper arm; bending of the person's shoulder, elbow, wrist, or finger joints; and movement of the person's jaw, such as bending of the jaw joint.

In an example, the roll, pitch and yaw of a wearable or handheld device can be monitored and analyzed using a motion sensor. In an example, the roll, pitch and yaw of a wearable device or handheld food utensil can be analyzed to detect when a person is eating. In an example, the roll, pitch and yaw of a wearable device or handheld food utensil can be analyzed to estimate how often a person is raising their hand up to their mouth. In an example, the roll, pitch and yaw of a wearable device or handheld food utensil can be analyzed to estimate how frequently a person is raising their hand up to their mouth. In an example, the roll, pitch and yaw of a wearable device or handheld food utensil can be analyzed to estimate the quantity of food that a person is consuming. In an example, the roll, pitch and yaw of a wearable device or handheld food utensil can be analyzed to estimate the pace and/or speed of a person's food consumption. In an example, a motion sequence which indicates eating can be comprise: a person raising their hand (and/or a food utensil) up toward their mouth; the person rolling and/or tilting their hand; a pause as the person bites and/or sips food from their hand (and/or a food utensil); and the person lowering their hand (and/or a food utensil) down away from their mouth. In an example, the duration of a pause in arm, hand, and/or finger motion can be used to estimate the quantity of food consumed during this motion sequence. In an example, the system can automatically record images of food and/or the person's mouth at one or more selected times during this motion sequence.

In an example, an angle and/or direction of the roll, pitch, or yaw of a person's hand (and/or food utensil) during a motion sequence associated with food consumption can be analyzed to help identify the type and/or quantity of food consumed during the sequence. In an example, the angle and/or direction of a roll, pitch, or yaw of a person's hand (and/or food utensil) can be different for consumption of solid food vs. liquid food (e.g. a beverage). In an example, the angle and/or direction of a roll, pitch, or yaw of a person's hand (and/or food utensil) can be different for consumption of food using a fork vs. using a spoon. In an example, the angle and/or direction of a roll, pitch, or yaw of a person's hand (and/or food utensil) can be different for consumption of food held by a person's hand vs. food transported using a utensil. In an example, the shape of a three-dimensional path traveled by a person's hand (and/or food utensil) bringing food up to the person's mouth can be different for different types and/or quantities of food. In an example, differences in three-dimensional paths traveled by a person's hand (and/or a food utensil) bringing food up to the person's mouth can be analyzed by a system as part of a methodology for estimating food items types and quantities.

In an example, a system for nutritional monitoring and management can include a wearable device with a motion sensor which is used to measure the speed and/or pace of food consumption based on the speed and/or frequency of eating-related motion cycles. In an example, a motion-sensing device that is worn on a person's wrist, hand, arm, or finger can measure how rapidly the person brings their hand up to their mouth. In an example, such information can be used to encourage, prompt, and/or entrain the person to eat at a slower speed and/or pace. A person will generally eat less during a meal if they eat at a slower pace. This is due to the lag between food consumption and a feeling of satiety from internal gastric organs. If a person eats more slowly, then they will tend to not overeat past the point of internal identification of satiety.

In an example, the speed and/or pace of changes in the roll, pitch, or yaw of a person's hand (and/or food utensil) during a motion sequence which is associated with food consumption can be analyzed to help identify the type and/or quantity of food consumed during the sequence. In an example, the speed and/or pace of changes in the roll, pitch, or yaw of a person's hand (and/or food utensil) can be different for consumption of solid food vs. liquid food (e.g. a beverage). In an example, speed and/or pace of changes in the roll, pitch, or yaw of a person's hand (and/or food utensil) can be different for consumption of food using a fork vs. using a spoon. In an example, the speed and/or pace of changes in the roll, pitch, or yaw of a person's hand (and/or food utensil) can be different for consumption of food held by a person's hand vs. food transported using a utensil.

In an example, a system for nutritional monitoring and management can include a wearable with a sensor which monitors, detects, and/or analyzes chewing or swallowing by a person. Such a sensor can differentiate between chewing and swallowing actions that are associated with eating vs. other activities. In an example, chewing or swallowing can be monitored, detected, sensed, or analyzed via: a sonic energy sensor (differentiating eating sounds from speaking, talking, singing, coughing, or other non-eating sounds); a body motion sensor (differentiating eating motions from speaking, yawning, or other mouth motions); a camera (differentiating eating from other mouth-related activities); and/or an electromagnetic energy sensor (such as measuring EMG signals from arm, mouth, or neck muscles or related nerves).

In an example, a system for nutrition monitoring and management can include one or more motion sensors which track movement of a person's jaw, mouth, teeth, throat, and/or neck. In an example, a person's jaw, mouth, teeth, throat, and/or neck can have different motion patterns when a person consumes different types of food. In an example, a person's jaw, mouth, teeth, throat, and/or neck can have different motion patterns as the person's consumes solid food vs. liquid food (e.g. beverages). In an example, a person's jaw, mouth, teeth, throat, and/or neck can have different motion patterns as the person's consumes food items with different levels of viscosity. In an example, a person's jaw, mouth, teeth, throat, and/or neck can have different motion patterns as the person consumes food items with different densities. In an example, the ratio of motions to swallow motions can be different for different types of food. In an example, there can be different angles and/or ranges ofjaw motion associated with consumption of different types of food. In an example, different biting motions can be associated with consumption of different types of food.

In an example, a system for nutritional monitoring and management can comprise: a wearable motion sensor that automatically collects data concerning body motion, wherein this body motion data is used to determine when a person is eating; and a camera that collects images of food, wherein these food images are used to identify the type and quantity of food, ingredients, or nutrients that a person is consuming.

In an example, a system for nutritional monitoring and management can have gesture recognition capability. In an example, a system can recognize hand gestures. In an example, a system can trigger and/or activate advanced food-identifying sensors when the system recognizes that a person is pointing toward a food item. In an example, the system can automatically direct a wearable camera toward where the person is pointing. In an example, the system can automatically direct a wearable spectroscopic sensor toward where the person is pointing. In an example, advanced sensors can be triggered and/or activated by a specific gesture. In an example, a person can provide their own (subjective) information concerning food item types and quantities by making hand gestures which the system recognizes. In an example, specific gestures can indicate specific types of food. In an example, specific gestures can indicate specific quantities of food. In an example, a system can recognize gestures which are part of sign language. In an example, information concerning food types and quantities provided via hand gestures can be part of the data which used by the system in multivariate food item identification and quantification. In an example, a system can include a motion sensor which detects hand gestures associated with eating. In an example, these gestures can include reaching for food, grasping food (or a glass or utensil for transporting food), raising food up to a mouth, tilting a hand to move food into a mouth, pausing to chew or swallow food, and then lowering a hand. In an example, eating-related gestures can include back-and-forth (“sawing”) hand movements when a person cuts food on a plate.

In an example, a system for nutritional monitoring and management can include a (generic) smart wrist-worn or finger-worn device (such as a smart watch, fitness band, smart sleeve, or smart ring) with a motion sensor, wherein the motion sensor may have been originally intended to measure a person's steps and/or caloric expenditure, but whose motion data can also be analyzed to detect when the person is eating and/or to estimate the quantity of food which the person consumes. In an example, a motion sensor can be used to estimate the quantity of food consumed based on the number of motion cycles. In an example, a motion sensor can be used to estimate the speed of food consumption based on the speed or frequency of motion cycles.

In an example, a system for nutritional monitoring and management can include a wearable device with a proximity sensor which detects when a person's hand is close to their mouth. In an example, a proximity sensor can detect when a person's wrist, hand, or finger is near the person's mouth. In an example, a proximity sensor can comprise an electromagnetic energy emitter worn on a person's wrist, hand, or finger and an electromagnetic energy receiver worn near a person's mouth (or neck). In an example, a proximity sensor can comprise an electromagnetic energy receiver worn on a person's wrist, hand, or finger and an electromagnetic energy emitter worn near a person's mouth (or neck). In an example, a proximity sensor can comprise an infrared light emitter and an infrared light receiver. In an example, a proximity sensor can be a wrist, hand, or finger worn camera whose images are analyzed using face recognition. In an example, a proximity sensor can be a motion sensor. In an example, a proximity sensor can comprise a first motion sensor worn on a person's wrist, hand, or finger and a second motion sensor worn near a person's mouth (or neck). In an example, a proximity sensor can comprise an infrared light emitter and an infrared light receiver.

In an example, a system for nutrition monitoring and management can have gesture recognition functionality. In an example, gestures can be identified using motion sensors, electromagnetic energy sensors, or both. In an example, a system can monitor movement of a person's arm, hand, and/or fingers to identify food-related gestures. In an example, a system can monitor electromagnetic energy (e.g. electromyographic signals) from muscles and/or nerves in a person's arm, hand, and/or fingers in order to identify food-related gestures. In an example, types and frequencies of food-related gestures can be analyzed as part of a system's determination of food item types and quantities. In an example, food-related gestures can be selected from the group comprising: biting off a piece of a hand-hand food item; drinking a beverage from a straw; grabbing a hand-held food item without a utensil; licking a hand-held food item; licking a spoon; lifting a beverage container up to one's mouth; lifting a fork up to one's mouth; lifting a spoon up to one's mouth; picking up a beverage container with one's hand; picking up a food utensil with one's left hand; picking up a food utensil with one's right hand; piercing food with a fork; removing food from a fork with one's mouth; scooping food into a spoon; taking a sip from a beverage container; twirling noodles around a fork; using a knife to cut food; and using chop sticks to bring food toward one's mouth.

In an example, a system for nutritional monitoring and management can include a food scale which helps to measure the quantity of nearby food and/or the amount of that food that a person actually consumes. In an example, a system can include a stand-alone food scale (which is in electromagnetic communication with other components of the system). In an example, a system can include a dish (e.g. a plate, bowl, glass, or cup), a place mat, a beverage coaster, or a food utensil rest which includes a scale to measure the weight of food on (or in) it. In an example, a plate or bowl can have different sections for holding different food items, wherein each section has a separate scale so that the weights of different food items can be individually (and independently) measured. In an example, the weight of food items on one or more scales can be measured at different times (e.g. before and after a meal) in order to estimate how much food a person actually consumes during a period of time. In an example, a plate or bowl can have different sections for holding different food items, wherein different sections of the plate or bowl are separated by ridges, undulations, or walls, and wherein each section has a separate scale. In an example, a plate or bowl can have different sections for holding different food items, wherein each section of the plate or bowl has a separate (built in) spectroscopic sensor so that the compositions of different food items can be individually (and independently) analyzed.

In an example, a system for nutritional monitoring and management can include a smart utensil with a force sensor, pressure sensor, bend sensor, goniometer, and/or strain sensor to estimate the weight of food conveyed by the utensil to a person's mouth. In an example, a force sensor, pressure sensor, bend sensor, goniometer, and/or strain sensor can be located between the distal (food carrying) end of a smart utensil and the proximal (handle) end of the utensil. In an example, a force sensor, pressure sensor, bend sensor, goniometer, and/or strain sensor can be located between the distal (food carrying) end of a smart utensil and the proximal (handle) end of the utensil, wherein the distal and proximal ends of the utensil can move independently of each other, wherein differences in motion between the proximal and distal ends are measured by the force sensor, pressure sensor, bend sensor, goniometer, and/or strain sensor, and wherein a greater difference in motion indicates a heavier portion (or piece) of food on the distal end of the utensil. In an example, a force sensor, pressure sensor, bend sensor, goniometer, and/or strain sensor can be part of a flexible joint, hinge, or spring between the distal (food carrying) end of a smart utensil and the proximal (handle) end of the utensil.

In an example, a system for nutritional monitoring and management can include a touch sensor, force sensor, and/or pressure sensor which helps to measure food quantity. In an example, a system can include a dish (e.g. plate, bowl, glass, or cup), place mat, coaster, or food utensil rest which includes a touch sensor, force sensor, and/or pressure sensor. In an example, a plate or bowl can have different sections for holding different food items, wherein each section has a separate force sensor and/or pressure sensor so that the weights of different food items can be individually (and independently) measured. In an example, the weight of food items on one or more force and/or pressure sensors can be measured at different times (e.g. before and after a meal) to estimate how much food a person has actually consumed. In an example, a plate or bowl can have different sections for holding different food items, wherein different sections are separated by ridges, undulations, or walls, and wherein each section has a separate force sensor and/or pressure sensor. In an example, a plate or bowl can have different sections for holding different food items, wherein each section has a separate (built in) spectroscopic sensor so that the compositions of different food items can be individually (and independently) analyzed.

In an example, a system for nutritional monitoring and management can include a wearable device with a force sensor, pressure sensor, bend sensor, vibration sensor, goniometer, and/or strain sensor. In an example, a force sensor, pressure sensor, bend sensor, vibration sensor, goniometer, and/or strain sensor can detects when a person is eating and/or can help to measure the amount of food that a person eats. In an example, a force sensor, pressure sensor, bend sensor, vibration sensor, goniometer, and/or strain sensor can be worn in physical contact with a person's neck or mouth. In an example, a force sensor, pressure sensor, bend sensor, vibration sensor, goniometer, and/or strain sensor which is in contact with a person's neck or mouth can monitor chewing and/or swallowing. In an example, a force sensor, pressure sensor, bend sensor, vibration sensor, goniometer, and/or strain sensor which is in contact with a person's neck or mouth can be used to help estimate how much food a person consumes. In an example, a force sensor, pressure sensor, bend sensor, vibration sensor, goniometer, and/or strain sensor which is in contact with a person's neck or mouth can be used to trigger advanced sensors (such as a wearable camera) when a person chews and/or swallows.

In an example, a system for nutrition monitoring and management can include a force sensor. In an example, the number of things sensed by the force can increase dramatically in sequels but, ironically, the sequels become less and less dramatic. In an example, the sequels can be duds. In an example, a system for nutrition monitoring and management can include a force sensor, pressure sensor, strain sensor, bend sensor, goniometer, barometer, and/or blood pressure monitor. In an example, one or more of these sensors can be incorporated into a wearable device, handheld device, or smart food utensil. In an example, data from one or more of these sensors can be used by the system to better determine types and quantities of food items consumed by a person.

In an example, a system for nutrition monitoring and management can include an electromagnetic energy sensor which is brought into electromagnetic communication with food. In an example, a system can include an electromagnetic energy sensor which measures the transmission of electromagnetic energy through a food item. In an example, a system can measure the conductivity, capacitance, resistance, and/or impedance of food items as part of the system's determination of food item types. In an example, a system can comprise one or more electrodes which are placed on (or inserted into) food in order to measure the conductivity, capacitance, resistance, and/or impedance of the food. In an example, different types of food can have different levels of electromagnetic conductivity, capacitance, resistance, and/or impedance. In an example, an electromagnetic energy sensor can be in electromagnetic communication with a food item without actually touching the food item. In an example, an electromagnetic energy sensor can collect data concerning the conductivity, capacitance, resistance, and/or impedance of a food item without actually touching the food item.

In an example, a system for nutrition monitoring and management can include an electromagnetic energy sensor which is in electromagnetic communication with a person's body. In an example, a system can include an electromagnetic energy sensor which measures the transmission of electromagnetic energy through body tissue. In an example, a system can measure the conductivity, capacitance, resistance, and/or impedance of body tissue as part of the system's detection that a person is eating and/or identification of what the person is eating. In an example, an electromagnetic energy sensor which is placed in electromagnetic communication with a person's body can be selected from the group consisting of: bioimpedance sensor, capacitive sensor, conductivity sensor, electrocardiographic (ECG) sensor, electroencephalographic (EEG) sensor, electromyographic (EMG) sensor, galvanic skin response sensor, impedance sensor, permittivity sensor, and resistance sensor. In an example, a system can include one or more electromagnetic energy sensors which are worn on a person's arm, wrist, and/or finger. In an example, a system can include one or more electromagnetic energy sensors which are worn on a person's head. In an example, an electromagnetic energy sensor can collect data concerning a person's neuromuscular activity which is related to eating. In an example, an electromagnetic energy sensor can collect data concerning a person's neurological activity which is related to eating.

In an example, analysis of a person's brain wave patterns (e.g. Brainwave patterns (e.g. EEG patterns)) can be used to predict that the person will be consuming food soon. In an example, analysis of a person's Brainwave patterns (e.g. EEG patterns) can be used to identify that the person is consuming specific types of food and/or nutrients. In an example, specific Brainwave patterns (e.g. EEG patterns) can be associated with consumption of specific types of nutrients, such as carbohydrates. In an example, brainwave patterns from selected areas of a person's brain can be analyzed to detect that a person is eating (or probably going to start eating). In an example, brainwave patterns from selected areas of a person's brain can be associated with food consumption. In an example, these patterns can occur when a person sees and/or smells food, even before the person's begins to actually eat food. For this reason, analysis of brainwave patterns (e.g. EEG patterns) may provide the earliest indication of pending or actual food consumption. Also, for this reason, analysis brainwave patterns (e.g. EEG patterns) can be a very useful part of a closed-loop automated system for insulin delivery and body glucose level management. In an example, specific brainwave patterns (e.g. EEG patterns) can be associated with levels of glucose of other substances in a person's blood and/or body tissue. In an example, specific Brainwave patterns (e.g. EEG patterns) can be analyzed and identified to measure levels of glucose in a person's body. In an example, analysis of person's EEG pattern can be used to recommend how much insulin a person should receive in association with food consumption.

In an example, a system with a spectroscopic sensor and a wearable device which measures electromagnetic brain activity can together comprise an integrated system for food identification and quantification. In an example, when a wearable device detects changes in a person's electromagnetic brain activity which indicates that the person is eating, then the system can prompt the person to scan food using the spectroscopic sensor. In an example, a system with a camera and a wearable device which measures electromagnetic brain activity can together comprise a system for food identification and quantification. In an example, when a wearable device detects changes in a person's electromagnetic brain activity which indicates that the person is eating, then the system can prompt the person to take pictures of the food using the camera.

In an example, a system for nutritional monitoring and management can comprise: a sound sensor worn by a person which automatically and continuously collects data concerning sound, wherein this sound data is used to determine when a person is eating; and a chemical composition sensor which does not continuously monitor the chemical composition of material within the person's mouth or gastrointestinal tract, but rather only collects information concerning the chemical composition of material within the person's mouth or gastrointestinal tract when sound data indicates that the person is eating. In an example, a system can comprise: a wearable sound sensor that automatically collects data concerning body or environmental sound, wherein this sound data is used to determine when a person is eating; and a chemical composition sensor that analyzes the chemical composition of food, wherein results of chemical analysis are used to identify the type and quantity of food, ingredients, or nutrients that a person is consuming.

In an example, a system for nutritional monitoring and management can include a microphone (or other sound sensor) which monitors for eating-related sounds. In an example, a system can use the microphone of a general purpose handheld device (such as a smart phone) to monitor eating-related sounds (such as biting, chewing, or swallowing sounds). In an example, a system can include a wearable device with a microphone (or other sound sensor) which collects data concerning eating-related sounds. In an example, eating-related sounds can include biting, chewing, and/or swallowing sounds. In an example, a microphone (or other sound sensor) can monitor eating-related sounds transmitted through the air. In an example, a microphone (or other sound sensor) can monitor eating-related sounds conducted through a person's body (e.g. by bone conduction). In an example, a system can measure the interaction between sonic energy (such as ultrasonic energy) and a food item in order to identify food item type and/or composition.

In an example, a system for nutritional monitoring and management can include a microphone (or other sound sensor) which monitors and/or records sound to detect when a person eats. In an example, a microphone can collect eating-related sound data for identification of food item type and/or composition. In an example, a microphone can collect eating-related sound data for estimation of quantity of food consumed. In an example, a first biting, chewing, and/or swallowing sound pattern can be associated with consumption of a first type of food and a second biting, chewing, and/or swallowing sound pattern can be associated with consumption of a second type of food. In an example, different biting, chewing, and/or swallowing sound patterns can be associated with consumption of solid, gelatinous, or liquid food. In an example, different biting, chewing, and/or swallowing sound patterns can be associated with consumption of food with different densities and/or viscosities. In an example, different numbers, speeds, frequencies, tones, and/or patterns biting, chewing, and/or swallowing sounds can be associated with consumption of different types of food. In an example, different numbers, speeds, frequencies, tones, and/or patterns of biting, chewing, and/or swallowing sounds can be associated with consumption of different quantities of food.

In an example, a system for nutritional monitoring and management can include a wearable device with a microphone (or other sound sensor) which records eating-related sounds such as biting, chewing, and/or swallowing. In an example, a system can include a sound-monitoring device which is worn on (or around) a person's neck. In an example, a system can include a sound-monitoring necklace, pendant, or collar. In an example, a system can include a sound-monitoring adhesive patch which is worn on a person's neck. In an example, a system can include a sound-monitoring device which is worn on (or in) a person's ear. In an example, a system can include a sound-monitoring ear ring, ear bud, ear insert, hearing aid, or bluetooth microphone device. In an example, a system can include a sound-monitoring ear ring, ear bud, ear insert, hearing aid, or bluetooth device which monitors eating-related sounds via bone conduction. In an example, a system can include a wrist-worn device (such as a smart watch) which monitors eating-related sounds such as biting, chewing, and swallowing sounds. In an example, a system can include an intra-oral device (e.g. dental appliance, dental braces, tooth crown or filling, or tongue piercing) with a microphone which monitors eating-related sounds. In an example, a system can include an article of smart clothing which includes a microphone to monitor eating-related sounds.

In an example, a system for nutritional monitoring and management can include a microphone which continually monitors for eating-related sounds and triggers advanced food-identification sensors when eating is detected. In an example, a system can trigger and/or activate a motion sensor when sounds recorded by a microphone indicate that a person is eating. In an example, a system can trigger and/or activate a camera when sounds recorded by a microphone indicate that a person is eating. In an example, a system can trigger and/or activate a spectroscopic sensor when sounds recorded by a microphone indicate that a person is eating. In an example, a system can trigger and/or activate an EMG or EEG sensor when sounds recorded by a microphone indicate that a person is eating.

In an example, a system for nutritional monitoring and management can jointly analyze data from a motion sensor on a first device and data from a microphone on a second device in order to identify types and quantities of food consumed by a person. In an example, the first device can be a smart watch worn by the person and the second device can be a smart necklace or collar worn by the person. In an example, the first device can be a smart watch worn by the person and the second device can be an ear ring or ear insert worn by the person. In an example, the first device can be a smart finger ring worn by the person and the second device can be a smart necklace or collar worn by the person. In an example, the first device can be a smart utensil held by the person and the second device can be a smart necklace or collar worn by the person. In an example, data from a motion sensor on a smart watch and data from a microphone on a smart necklace can be jointly analyzed in multivariate analysis to identify types and quantities of food consumed by a person. In an example, a system can jointly analyze data from a motion sensor on a wearable device (e.g. smart watch, finger ring, necklace, collar, ear ring, ear bud, or smart eyeglasses) and data from a microphone on a wearable device (e.g. smart watch, finger ring, necklace, collar, ear ring, ear bud, or smart eyeglasses) in order to identify types and quantities of food consumed by a person.

In an example, a system for nutrition monitoring and management can include a microphone or other sound sensor. In an example, a system for nutrition monitoring and management can include a sound sensor selected from the group consisting of: acoustic wave sensor, ambient sound sensor, bone conduction microphone, microphone, sound-based chew sensor, sound-based swallow sensor, ultrasonic energy sensor, and vibration sensor. In an example, a system can monitor and analyze sounds associated with eating as part of the identification of food items and estimation of quantity of food items consumed. In an example, a system can monitor and analyze biting, chewing, and/or swallowing sounds associated with eating as part of the identification of food items and estimation of quantity of food items consumed. In an example, a system can monitor and analyze the acoustic spectrum of biting, chewing, and/or swallowing sounds associated as part of the identification of food items and estimation of quantity of food items consumed. In an example, a microphone or other sound sensor can be worn on or around a person's neck. In an example, a microphone or other sound sensor can be part of a necklace, collar, or neck-worn patch. In an example, a system for nutritional intake monitoring and management can include an ear-worn device (e.g. earbud, ear ring, or outer ear loop device) which monitors chewing and/or swallowing sounds via bone conduction. In an example, chewing and/or swallowing sounds can be detected by a system via bone conduction and used by the system to trigger automated food imaging and/or spectroscopic analysis.

In an example, a system for nutrition monitoring and management can include a chemical sensor. In an example, a system can include a sensor selected from the group consisting of: body chemistry sensor, breath chemistry sensor, chemical sensor, food sample sensor, gas sensor, glucose monitor, odor sensor, pH sensor, saliva sensor, spectroscopic sensor, and sweat sensor. In an example, a chemical sensor can provide information about the molecular composition of food. In an example, a chemical sensor can provide information about the molecular composition of a sample of food. In an example, a chemical sensor can provide information about the molecular composition of body tissue.

In an example, a system for nutrition monitoring and management can include a thermal energy sensor. In an example, a system can include a thermometer. In an example, a system can include a skin temperature sensor. In an example, a system can include a food temperature sensor. In an example, a system can include a heat sensor. In an example, a system can analyze associations between a person's skin and/or body tissue temperature and subsequent food consumption by the person. In an example, changes in body temperature can be used to predict subsequent food consumption. In an example, a system can predict that a person will consume an apple and a medical tonic. In an example, a person can predict that apple will consume med tonic. In an example, a system can analyze associations between a person's food consumption and subsequent changes in the person's skin and/or body tissue temperature. In an example, a system can analyze associations between a person's consumption of specific types and/or quantities of food and subsequent changes in the person's skin and/or body tissue temperature.

In an example, a system for nutrition monitoring and management can include an environmental sensor which detects and/or measures characteristics of a person's environment which can be related to food consumption and/or hydration requirements. In an example, a system can have a GPS unit which tracks a person's current location and where they have been. In an example, a system can track whether a person is at (or near) a specific restaurant. In an example, a system can have an ambient light sensor which tracks the time of day. In an example, a system can have an ambient sound sensor which measures overall ambient sound level. In an example, a system can have an environmental sound sensor which monitors ambient sounds to detect words and/or sounds associated with food consumption. For example, an environmental sound sensor can detect words and/or sounds associated with specific restaurants or food stores. In an example, a system can track environmental temperature and humidity to better estimate a person's hydration requirements. In an example, a system can track activity level to better estimate a person's hydration requirements.

In an example, a system for nutritional monitoring and management can track food at the time of food selection and/or purchase. In an example, a system can track a person's food selections and purchases at a grocery store, restaurant, or vending machine. In an example, such tracking can be done via financial transaction tracking. In an example, such tracking can be done via bar code, QR code, RFID tag, or electronic restaurant menu. In an example, electronic communication for food identification can also occur between a system and a vending machine. Food selection, purchasing, and/or consumption activity can also be tracked by location information, such a location information provided by a GPS unit.

In an example, a system for nutrition monitoring and management can include a GPS unit or other location sensing unit. In an example, a system can analyze a restaurant's menu based on image analysis and optical character recognition. In an example, a system can identify restaurants which are near a person and link those restaurants to meals and/or foods in a database of meals and/or foods. In an example, a system can identify when a person is at a particular restaurant and link that restaurant to meals and/or foods in a database of meals and/or foods. In an example, a system can recommend healthier alternatives to a particular meal and/or food offered by a restaurant. In an example, a system can recommend healthier nearby alternatives to a particular restaurant. In an example, a system can recommend nearby healthy restaurants. In an example, a system can recommend specific meals on a standardized menu of a specific restaurant and make recommendations concerning those meals to the person. In an example, a system can recommend food stores where a person can purchase healthy foods and/or meal ingredients.

In an example, a system for nutritional monitoring and management can comprise a relatively less-intrusive sensor (such as a motion sensor) which continually monitors for possible eating and triggers activation and/or operation of a more-intrusive sensor (such as a camera) when eating is detected. In an example, an eating detection and/or estimation sensor can be attached directly to a person's body, attached to clothing after the clothing has been made, or integrated into smart clothing as the clothing is made. In an example, an eating detection and/or estimation sensor can be implanted within a person's body wherein it internally monitors for chewing, swallowing, biting, other muscle activity, enzyme secretion, neural signals, or other ingestion-related processes or activities. In an example, an eating detection and/or estimation sensor can monitor for eating related activity continuously, at periodic times, at intermittent times, or at random times.

In an example, a system for nutritional monitoring and management can have a sensor which collects data concerning electromagnetic energy emitted from a person's body. In an example, a system can have a sensor which collects data concerning electromagnetic energy emitted from a person's muscles and nerves. In an example, a system can have a sensor which collects data concerning light energy reflected from a person's body. In an example, a system can have a sensor which collects data concerning light energy reflected from a person's skin and/or body tissue. In an example, a system can have a sensor which collects data concerning motion of a person's body. In an example, a system can have a sensor which collects data concerning motion of a person's arm, wrist, hand, and/or fingers. In an example, a system can have a sensor which collects data concerning thermal energy emitted from the person's body.

In an example, a system for nutritional monitoring and management can include a electrogoniometer. In an example, a system can include an electromagnetic energy sensor. In an example, a system can include a EMG sensor. In an example, a system can include a Galvanic Skin Response sensor. In an example, a system can include a gas chromatographic sensor. In an example, a system can include a gastric activity sensor. In an example, a system can include a geolocation sensor. In an example, a system can include a glucose sensor. In an example, a system can include a GPS sensor. In an example, a system can include a gyroscope. In an example, a system can include a heart rate sensor. In an example, a system can include an inclinometer.

In an example, a system for nutritional monitoring and management can include a pressure sensor. In an example, a system can include a respiration sensor. In an example, a system can include a smell sensor. In an example, a system can include a sodium sensor. In an example, a system can include a sound sensor. In an example, a system can include a spectroscopic sensor. In an example, a system can include a strain gauge. In an example, a system can include a swallow sensor. In an example, a system can include a temperature sensor. In an example, a system can include a heat sensor. In an example, a system can include a tissue impedance sensor. In an example, a system can include an ultrasonic sensor.

In an example, a system for nutritional monitoring and management can include a food utensil (or other apportioning device) which divides food items into spoonfulls, forkfulls, mouthfuls, and/or bite-size pieces. In an example, the number of times that such a utensil is brought up to a person's mouth can be tracked and multiplied times an estimated amount of food per motion (e.g. per spoonfull, forkfull, mouthful, or bite) to estimate the cumulative amount of food consumed. In an example, a motion sensor worn on a person's wrist or incorporated into a smart utensil can measure the number of hand-to-mouth motions.

In an example, a system for nutritional monitoring and management can include an accelerometer. In an example, a system can include an analytical chromatographic sensor. In an example, a system can include an artificial olfactory sensor. In an example, a system can include a blood pressure sensor. In an example, a system can include a camera. In an example, a system can include a chemical sensor. In an example, a system can include a chewing sensor. In an example, a system can include a cholesterol sensor. In an example, a system can include an ECG sensor. In an example, a system can include an EEG sensor. In an example, a system can include a PPG sensor. In an example, a system can include an electrochemical sensor.

In an example, a system for nutritional monitoring and management can include an infrared sensor. In an example, a system can include a liquid chromatographic sensor. In an example, a system can include a microphone. In an example, a system can include a motion sensor. In an example, a system can include an olfactory sensor. In an example, a system can include an optical sensor. In an example, a system can include an optoelectronic sensor. In an example, a system can include a photocell. In an example, a system can include a photochemical sensor. In an example, a system can include a piezoelectric sensor.

In an example, a system for nutritional monitoring and management can include an optical sensor which analyzes modulation of light wave parameters caused by the interaction light energy and food. In an example, an optical sensor can be a chromatographic sensor, spectrographic sensor, analytical chromatographic sensor, liquid chromatographic sensor, gas chromatographic sensor, optoelectronic sensor, photochemical sensor, or photocell.

In an example, a system for nutritional monitoring and management can include one or more sensors selected from the group consisting of: accelerometer, inclinometer, motion sensor, pedometer, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor. In an example, data from one or more of these sensors can be combined in multivariate analysis to identify food item types and estimate food item quantities. In an example, data from one or more of these sensors can be combined in multivariate analysis to determine the types and quantities of food and/or nutrients consumed by a person.

In an example, a system for nutritional monitoring and management can include a lower-level eating-related sensor and a higher-level eating-related sensor. In an example, the lower-level eating-related sensor can detect when a person is eating. In an example, the lower-level eating-related sensor can detect that a person is eating. In an example, a system can comprise: (a) a lower-level eating-related sensor, wherein the lower-level eating-related sensor has a first level of accuracy with respect to identification of food item types and/or estimation of food item quantities, and wherein the lower-level eating-related sensor has a second level of privacy intrusion; and (b) higher-level eating-related sensor; a higher-level eating-related sensor, wherein the higher-level eating-related sensor has a third level of accuracy with respect to identification of food item types and/or estimation of food item quantities, wherein the higher-level eating-related sensor has a fourth level of privacy intrusion, wherein the third level is greater than the first level, wherein the fourth level is greater than the second level, and wherein operation of the higher-level eating-related sensor is activated and/or triggered when data from the lower-level eating-related sensor detects that a person is eating. In an example, a lower-level eating-related sensor can be selected from the group consisting of: wearable motion sensor; motion sensor which is part of a smart utensil; wearable microphone; wearable EMG sensor; wearable EEG sensor; and wearable camera. In an example, a higher-level eating-related sensor can be selected from the group consisting of: wearable camera; wearable spectroscopic sensor; handheld camera; and handheld spectroscopic sensor.

In an example, a system for nutritional monitoring and management can include a lower-level eating-related sensor (such as a wearable motion sensor, motion sensor which is part of a smart utensil, wearable microphone, wearable EMG sensor, wearable EEG sensor, or wearable camera) and a higher-level eating-related sensor. In an example, the lower-level eating-related sensor can detect when a person is eating. In an example, the lower-level eating-related sensor can detect that a person is eating. In an example, a system can comprise: (a) a lower-level eating-related sensor (such as a wearable camera, wearable spectroscopic sensor, handheld camera, or handheld spectroscopic sensor), wherein the lower-level eating-related sensor has a first level of accuracy with respect to identification of food item types and/or estimation of food item quantities, and wherein the lower-level eating-related sensor has a second level of privacy intrusion; and (b) higher-level eating-related sensor; a higher-level eating-related sensor, wherein the higher-level eating-related sensor has a third level of accuracy with respect to identification of food item types and/or estimation of food item quantities, wherein the higher-level eating-related sensor has a fourth level of privacy intrusion, wherein the third level is greater than the first level, wherein the fourth level is greater than the second level, and wherein operation of the higher-level eating-related sensor is activated and/or triggered when data from the lower-level eating-related sensor detects that a person is eating.

In an example, a system for nutritional monitoring and management can include a lower-level eating-related sensor (such as a wearable motion sensor) and a higher-level eating-related sensor. In an example, the lower-level eating-related sensor can detect when a person is eating. In an example, the lower-level eating-related sensor can detect that a person is eating. In an example, a system can comprise: (a) a lower-level eating-related sensor (such as a wearable camera), wherein the lower-level eating-related sensor has a first level of accuracy with respect to identification of food item types and/or estimation of food item quantities, and wherein the lower-level eating-related sensor has a second level of privacy intrusion; and (b) higher-level eating-related sensor; a higher-level eating-related sensor, wherein the higher-level eating-related sensor has a third level of accuracy with respect to identification of food item types and/or estimation of food item quantities, wherein the higher-level eating-related sensor has a fourth level of privacy intrusion, wherein the third level is greater than the first level, wherein the fourth level is greater than the second level, and wherein operation of the higher-level eating-related sensor is activated and/or triggered when data from the lower-level eating-related sensor detects that a person is eating.

In an example, a system for nutritional monitoring and management can include a lower-level eating-related sensor (such as a wearable motion sensor, motion sensor which is part of a smart utensil, wearable EMG sensor, or wearable EEG sensor) and a higher-level eating-related sensor. In an example, the lower-level eating-related sensor can detect when a person is eating. In an example, the lower-level eating-related sensor can detect that a person is eating. In an example, a system can comprise: (a) a lower-level eating-related sensor (such as a wearable camera, wearable spectroscopic sensor, handheld camera, or handheld spectroscopic sensor), wherein the lower-level eating-related sensor has a first level of accuracy with respect to identification of food item types and/or estimation of food item quantities, and wherein the lower-level eating-related sensor has a second level of privacy intrusion; and (b) higher-level eating-related sensor; a higher-level eating-related sensor, wherein the higher-level eating-related sensor has a third level of accuracy with respect to identification of food item types and/or estimation of food item quantities, wherein the higher-level eating-related sensor has a fourth level of privacy intrusion, wherein the third level is greater than the first level, wherein the fourth level is greater than the second level, and wherein operation of the higher-level eating-related sensor is activated and/or triggered when data from the lower-level eating-related sensor detects that a person is eating.

In an example, a system for nutritional monitoring and management can include an advanced-level (e.g. more accurate, but more privacy intrusive and/or higher power consumption) food-identifying sensor which is triggered and/or activated by when a lower-level (e.g. less intrusive and/or lower power) food-consumption sensor detects that a person is eating. In an example, the lower-level food-identifying sensor can operate continually, but the advanced-level food-identifying sensor is only activated when a person eats. The combination of a continuously-operated lower-level food-consumption monitor and a selectively-operated advanced-level food-identifying sensor can achieve relatively-high food identification accuracy with relatively-low privacy intrusion and/or power resource requirements. In an example, a system can automatically activate an advanced-level food-identifying sensor when a lower-level sensor detects one or more of the following triggers: a food item nearby; hand-to-food interaction; location in a restaurant, kitchen, or dining room; distinctive arm, hand, and/or wrist motions associated with bringing food up to a person's mouth; physiologic responses by the person's body associated with eating; smells or sounds that are associated with food and/or eating; and/or speech associated with eating.

In an example, a system for nutritional monitoring and management can be triggered to perform an action by a trigger selected from the group consisting of: biometric parameters (such as glucose levels or heart rate) associated with eating; body motions (e.g. selected arm, wrist, hand, and/or finger movements) associated with eating; chewing or swallowing sounds associated with eating; EEG patterns associated with eating; EMG patterns associated with eating; ECG patterns associated with eating; environmental sounds associated with eating; geolocation (e.g. restaurant location) associated with eating; images of nearby food or objects associated with eating; jaw, mouth, and/or teeth motions associated with eating; time of day associated with eating; and/or smells associated with food and/or eating. In an example, one or more system-initiated actions can be selected from the group consisting of: activating higher-power and/or more-sensitive sensors; automatically taking pictures or recording sounds; and prompting a person to take pictures of food and/or provide descriptions of food. In an example, a system can select a recommended insulin dosage in response to specific attributes of a trigger. In an example, a system with a drug delivery component can automatically dispense a selected amount of insulin in response to selected attributes of a trigger.

In an example, a system for nutritional monitoring and management can have a low-power mode when a person is not eating and a high-power mode when the person is eating. In an example, having a low-power mode can conserve power and extend battery life. In an example, a person can actively (e.g. manually) change a system from a low-power mode to a high-power mode when the person is going to start eating. In an example, a system can automatically change from a low-power mode to a high-power mode when the system detects that a person is eating (or is probably going to start eating). In an example, a system can automatically change from a low-power mode to a high-power mode when the system detects: biometric parameters (such as glucose levels or heart rate) associated with eating; body motions (e.g. selected arm, wrist, hand, and/or finger movements) associated with eating; chewing or swallowing sounds associated with eating; EEG patterns associated with eating; EMG patterns associated with eating; ECG patterns associated with eating; environmental sounds associated with eating; geolocation (e.g. restaurant location) associated with eating; images of nearby food or objects associated with eating; jaw, mouth, and/or teeth motions associated with eating; time of day associated with eating; and/or smells associated with food and/or eating.

In an example, a system can automatically activate and/or trigger a wearable camera to scan nearby space and record images of food when eating is detected by one or more sensors selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, optical sensor, piezoelectric sensor, respiration sensor, strain gauge, electrogoniometer, chewing sensor, swallow sensor, temperature sensor, and pressure sensor.

In an example, a system can automatically activate a wearable camera to record food images when a motion sensor detects that a person is eating. In an example, the camera can automatically search for food near a person's hands and/or mouth when the camera is activated. In an example, a system can automatically activate a wearable camera to record food images when a wearable motion sensor detects that a person is eating. In an example, a system can comprise: a wearable motion sensor that is worn by a person, wherein this motion sensor automatically and continuously collects data concerning the person's body motion, and wherein the body motion data is used to determine when a person is eating; and a wearable camera that is worn by the person, wherein this camera does not continuously record images, but rather only records images when body motion data indicates that the person is eating. In an example, both the motion sensor and microphone can be part of a wrist-worn device such as a smart watch.

In an example, a system for nutritional monitoring and management can activate and/or trigger a wearable camera to search for food and record food images when data from a motion sensor detects eating. In an example, a system can include a wearable device with a camera which is automatically activated to record food images when data from a motion sensor indicates eating-related motion. In an example, a system can include a wrist-worn device with a camera which is automatically activated to record images of a person's hand to capture images of food items when data from a motion sensor indicates eating-related motion. In an example, a system can include a smart watch with a camera which is automatically activated to record images of a person's hand to capture images of food items when data from a motion sensor indicates eating-related motion. In an example, a system can include a smart necklace with a camera which is automatically activated to record images of a person's mouth (or space immediately in front of a person) to capture images of food items when data from a motion sensor indicates eating-related motion.

In an example, a system for nutritional monitoring and management can activate and/or trigger a wearable camera to record video images for a set interval of time after data from a motion sensor first indicates that a person is eating. In an example, a motion-triggered camera can start recording images based on data from a motion sensor and can continue recording as long as eating continues. Continued eating can be monitored and/or detected by the motion sensor, by the camera, or by both. Also, if analysis of images from the camera shows that detection of eating by the motion sensor was a false alarm (e.g. the person is not really eating), then the camera can stop recording images.

In an example, a system for nutritional monitoring and management can activate and/or trigger the operation of a wearable camera when analysis of sounds from a microphone (or other sound sensor) detects that a person is eating. In an example, a system can activate a camera when a microphone (or other sound sensor) records chewing, biting, or swallowing sounds. In an example, a system can include a wearable camera which is automatically activated to record food images when a wearable microphone (or other sound sensor) records chewing, biting, or swallowing sounds. In an example, both the camera and microphone can be part of a wrist-worn device such as a smart watch. In example, both the camera and microphone can be part of a neck-worn device such as a smart necklace or collar. In an example, both the camera and microphone can be part of an ear-worn device such as a smart ear ring.

In an example, a system for nutritional monitoring and management can have a low-sensor-level mode when a person is not eating and a high-sensor-level mode when a person is eating. In an example, a high-sensor-level mode can include the operation of various types of sensors to monitor food consumption more accurately than low-sensor-level mode, but the high-sensor-level mode is more intrusive with respect to privacy. Accordingly, it can be advantageous to activate high-sensor-level mode only when a person is eating. In an example, a person can actively (e.g. manually) change a system from a low-sensor-level mode to a high-sensor-level mode when the person is going to start eating. In an example, a system can automatically change a system from a low-sensor-level mode to a high-sensor-level mode when the system detects that a person is eating (or is probably going to start eating). In an example, a system can automatically change from a low-sensor-level mode to a high-sensor-level mode when the system detects: biometric parameters (such as glucose levels or heart rate) associated with eating; body motions (e.g. selected arm, wrist, hand, and/or finger movements) associated with eating; chewing or swallowing sounds associated with eating; EEG patterns associated with eating; EMG patterns associated with eating; ECG patterns associated with eating; environmental sounds associated with eating; geolocation (e.g. restaurant location) associated with eating; images of nearby food or objects associated with eating; jaw, mouth, and/or teeth motions associated with eating; time of day associated with eating; and/or smells associated with food and/or eating.

In an example, a system for nutritional monitoring and management can have a first mode with only one or more motion sensors activated when a person is not eating and second mode with one or more motion sensors and a camera activated when the person is eating. In an example, a second mode with motion sensors and a camera activated can be more intrusive with respect to the person's privacy. Accordingly, it can be advantageous to only activate the second (motion sensor and camera) mode when the person is eating. In an example, a person can actively (e.g. manually) change a system from a first mode with only motion sensors activated to a second mode with motion sensors and a camera activated when the person is going to start eating. In an example, a system can automatically change from a first mode with only motion sensors activated to a second mode with motion sensors and a camera activated when the system detects that a person is eating (or probably going to start eating). In an example, a system can automatically change from a first mode with only motion sensors activated to a second mode with motion sensors and a camera activated when the system detects: biometric parameters (such as glucose levels or heart rate) associated with eating; body motions (e.g. selected arm, wrist, hand, and/or finger movements) associated with eating; chewing or swallowing sounds associated with eating; EEG patterns associated with eating; EMG patterns associated with eating; ECG patterns associated with eating; environmental sounds associated with eating; geolocation (e.g. restaurant location) associated with eating; images of nearby food or objects associated with eating; jaw, mouth, and/or teeth motions associated with eating; time of day associated with eating; and/or smells associated with food and/or eating.

In an example, a system for nutritional monitoring and management can have a first mode with only motion sensors activated when a person is not eating and second mode with motion sensor, camera, and microphone activated when the person is eating. In an example, a second mode with motion sensor, camera, and microphone activated can be more intrusive with respect to the person's privacy. Accordingly, it can be advantageous to only activate the second (motion sensor, camera, and microphone) mode when the person is eating. In an example, a person can actively (e.g. manually) change a system from a first mode with only motion sensors activated to a second mode with motion sensor, camera, and microphone activated when the person is going to start eating. In an example, a system can automatically change from a first mode with only motion sensors activated to a second mode with motion sensor, camera, and microphone activated when the system detects that a person is eating (or probably going to start eating). In an example, a system can automatically change from a first mode with only motion sensors activated to a second mode with motion sensor, camera, and microphone activated when the system detects: biometric parameters (such as glucose levels or heart rate) associated with eating; body motions (e.g. selected arm, wrist, hand, and/or finger movements) associated with eating; chewing or swallowing sounds associated with eating; EEG patterns associated with eating; EMG patterns associated with eating; ECG patterns associated with eating; environmental sounds associated with eating; geolocation (e.g. restaurant location) associated with eating; images of nearby food or objects associated with eating; jaw, mouth, and/or teeth motions associated with eating; time of day associated with eating; and/or smells associated with food and/or eating.

In an example, a system for nutritional monitoring and management can have a first mode with only motion sensors activated when a person is not eating and second mode with motion sensor, camera, microphone, and biometric sensor activated when the person is eating. In an example, a person can actively (e.g. manually) change a system from a first mode with only motion sensors activated to a second mode with motion sensor, camera, microphone, and biometric sensor activated when the person is going to start eating. In an example, a system can automatically change from a first mode with only motion sensors activated to a second mode with motion sensor, camera, microphone, and biometric sensor activated when the system detects that a person is eating (or probably going to start eating). In an example, a system can automatically change from a first mode with only motion sensors activated to a second mode with motion sensor, camera, microphone, and biometric sensor activated when the system detects: biometric parameters (such as glucose levels or heart rate) associated with eating; body motions (e.g. selected arm, wrist, hand, and/or finger movements) associated with eating; chewing or swallowing sounds associated with eating; EEG patterns associated with eating; EMG patterns associated with eating; ECG patterns associated with eating; environmental sounds associated with eating; geolocation (e.g. restaurant location) associated with eating; images of nearby food or objects associated with eating; jaw, mouth, and/or teeth motions associated with eating; time of day associated with eating; and/or smells associated with food and/or eating.

In an example, a system for nutritional monitoring and management can analyze eating-related motion patterns to determine optimal times to perform a spectroscopic scan of food. In an example, a spectroscopic scan can be triggered at times during eating motions when a person's arm is most extended and, thus, most likely to be closest to remaining food. In an example, a spectroscopic scan can be triggered by a gesture indicating that a person is grasping food or bringing food up to their mouth. In an example, repeated spectroscopic scans of food at different times during a meal can help to analyze the composition of multiple food layers, not just the surface layer. This can provide a more accurate estimate of food composition, especially for foods with different internal layers and/or a composite (non-uniform) ingredient structure.

In an example, a system for nutritional monitoring and management can trigger and/or activate a spectroscopic sensor to take spectroscopic scans of food when a wearable motion sensor indicates that a person is eating. In an example, a system can automatically trigger spectroscopic scanning when data from a motion sensor indicates eating-related motion. In an example, a system can include a wearable device with an outward and/or forward directed spectroscopic scanner which is automatically activated when data from a motion sensor indicates eating-related motion. In an example, a system can include a wrist-worn device with an outward and/or forward directed spectroscopic scanner which is automatically activated to scan near a person's hand for food items when data from a motion sensor indicates eating-related motion. In an example, a system can include a smart watch with a spectroscopic scanner which is automatically activated to scan near a person's hand for food items when data from a motion sensor indicates eating-related motion. In an example, a system can include a smart necklace with a spectroscopic scanner which is automatically activated to scan near a person's mouth for food items when data from a motion sensor indicates eating-related motion.

In an example, a system for nutritional monitoring and management can have a plurality of sensors of different types, wherein a first subset of one or more sensors are active all the time and a second subset of the sensors are only activated when the data from the first set of sensors indicates that a person is eating (or probably will start eating soon). In an example, the first subset of sensors can include a motion sensor (e.g. accelerometer and gyroscope) and/or biometric sensor (e.g. heart rate sensor and blood glucose sensor). In an example, the second subset of sensors can include a camera and/or a microphone. In an example, a system can be triggered to activate a second subset of sensors based on one or more triggers selected from the group consisting of: arm, hand, wrist, and/or finger motions associated with eating; ECG signals associated with eating; EEG signals associated with eating; EMG signals associated with eating; geolocation associated with eating; images or written words associated with eating; room in a building associated with eating; smells or odors associated with eating; spoken words associated with eating; and time of day associated with eating.

In an example, a system for nutritional monitoring and management can comprise: a wearable motion sensor that automatically collects data concerning body motion, wherein this body motion data is used to determine when a person is eating; and a chemical composition sensor that analyzes the chemical composition of food, wherein chemical analysis is used to identify the type and quantity of food, ingredients, or nutrients that the person is eating. In an example, a system can comprise: a motion sensor that is worn by a person, wherein this motion sensor automatically and continuously collects data concerning the person's body motion, and wherein the body motion data is used to determine when the person is eating; and a chemical composition sensor, wherein this chemical composition sensor does not continuously monitor the chemical composition of material within the person's mouth or gastrointestinal tract, but rather only collects information concerning the chemical composition of material within the person's mouth or gastrointestinal tract when body motion data indicates that the person is eating.

In an example, a system for nutritional monitoring and management can include a touch-based user interface through which a person can enter their own information concerning food types and quantities. In an example, a person can touch a screen or press a button to identify a food item type by selecting a food item (e.g. food name or image) from a menu of food items. In an example, a person can touch a screen or press a button to identify a food item quantity by selecting a quantity from a menu of food quantities. In an example, a person can type information about food types and quantities using a keypad or keyboard. In an example, a system can include a gesture recognition user interface. In an example, a system can include a wearable motion sensor which tracks arm, hand, and finger motions and analyzes these motions to identify key gestures. These key gestures can be used by a person to communicate food types and quantities.

In an example, a system for nutritional monitoring and management can enable a person to provide their own (subjective) verbal description of food item types and/or quantities. For an example, if a person has a plate with fish, carrots, and tomatoes in front of them, then the person may speak into a system's microphone—“fish, one half medium size, carrots, a dozen sticks, tomatoes, one half medium size.” The system can use speech recognition to translate this description into a standardized and/or digital format for comparison to a standardized database of food item types and/or quantities. In an example, translated results and/or matching information from the database can be displayed or spoken by the system for confirmation by the person. For example, the system may say—“Tilapia, 8 ounces, carrots, 12 ounces, and tomaytoes, 10 ounces. Correct?” The person may respond—“You mean tomatoes?” The system may respond—“Eh . . . tomaytoes, tomatoes.” In an example, a person can follow-up with a camera to record images of food items and/or a spectroscopic sensor to scan the food items. Multivariate analysis of these multiple forms of information concerning the food can provide more accurate identification of food item types and quantities.

In an example, a system for nutritional monitoring and management can include a microphone and a speech recognition interface through which a person provides verbal input as part of an analysis of food item types and/or quantities. In an example, a system can receive and recognize a person's verbal descriptions of nearby food items types and/or quantities. In an example, a system can receive and recognize a person's verbal descriptions of food items types and/or quantities which a person selects or purchases. In an example, a system can receive and recognize a person's verbal descriptions of food items types and/or quantities which the person consumes. In an example, a system can use a microphone and speech recognition to extract information related to food selecting, ordering, purchasing, or consumption from speech overheard in a person's environment.

In an example, a system for nutritional monitoring and management can include a microphone through which a person provides verbal input to the system and/or a speaker through which the system provides verbal feedback to the person. In an example, a person can provide oral commands and/or verbal food descriptions as inputs to the system. In an example, a person can direct a virtual light pattern (e.g. a laser pointer) toward locations on nearby food items, say what they think the food items are, and say what they think are the food item quantities. In an example, a system can translate this verbal input into standardized and/or digital information using speech recognition and match this input with records in a database of food item types and quantities. In an example, a person can also record images of the food items and/or do spectroscopic scans of the food items, wherein the results of these verbal descriptions, food images, and spectroscopic scans can be linked together in multivariate analysis to more accurately identify food item types and quantities.

In an example, a system for nutritional monitoring and management can include a speech recognition user interface through which a person can enter their own information concerning food types and quantities. In an example, a person can speak a description of a food item, including the person's perception of food type and quantity. In an example, the system can use speech recognition and natural language processing to convert the person's natural language description into a standardized food type and quantity. In an example, a system may only start recording speech when a person starts it via pressing a button, touching a screen, making a specific gesture, or speaking a specific trigger/command word. For example, a person can activate audio recording by the system with a trigger/command phrase such as—“Hey, Skainet. You can stop pretending that you don't monitor every word I say.”

In an example, a system for nutritional monitoring and management can provide a person with a means to provide descriptions of food item types and quantities in the form of words. In an example, a system can prompt a person to provide descriptions of food item types and quantities when food is detected nearby or when the system detects that the person has started eating. In an example, these descriptions can be spoken words (e.g. through a speech recognition interface). In an example, these descriptions can be typed words (e.g. through a keypad or keyboard). In an example, these descriptions can be words selected from a drop-down word menu on a device screen. In an example, these descriptions can be words selected from a drop-down word menu in augmented reality.

In an example, a system for nutritional monitoring and management can use single-word synonyms and/or multi-word phrase synonyms to process natural language descriptions of food items from a person. In an example a system can translate descriptions of food items between different languages. Word processing can include single-word synonyms and variations and multi-word phrase synonyms. In an example, a person can sequentially direct a projected light beam toward different food items in a multi-item meal and provide a verbal description of each food item as the light beam hits the food item. In an example, a person can touch a different food items in a meal displayed on a touch screen in a sequential manner and provide a verbal description of each food item as it is touched. In an example, a system can link food items in an image or in an augmented reality display with food item descriptions provided by a person for multivariate analysis of food items types and quantities.

In an example, word-based descriptions of food types and quantities from a person can be combined with automated food identification processes (e.g. such as image analysis and spectroscopic analysis) for multivariate analysis of food items types and quantities. In an example, word-based descriptions can be linked to food images and data from spectroscopic analysis of food. Multivariate analysis of food item types and quantities can integrate one or more of the following: word-based descriptions of food items; automated analysis of food images; spectroscopic analysis of food items; analysis of food quantity via utensil movement or bending; and arm, wrist, hand, and/or finger movements. In an example, a person can enter information about food which they are consuming via one or more modalities selected from the group consisting of: entering text via a key pad; selecting an entry from a drop-down menu displayed in their field of view (e.g. in augmented reality or on a device display screen); and speaking a description of food into a device with speech recognition functionality.

In an example, person can be given an opportunity (or be prompted) to self-report how they are feeling at times relative to (e.g. before, during, or after) food consumption (of specific types and/or quantities of foods). In an example, food consumption entries can be accompanied by geotagging (e.g. in association with particular restaurants, stores, or dining locations in a building). In an example, a person can indicate (identify or point toward) different food items using gestures which are recognized by the system. In an example, small food samples can be placed on device (or utensil) for analysis. In an example, a system project a beam (or pattern) of light which is used to aim a camera and/or spectroscopic scanner, identify selected food portions in a meal, and/or trace out food boundaries. In an example, a person can identify boundaries of a food portion (in a meal) by drawing with their finger on a touch screen; moving a projected light beam over food; and/or moving an object (e.g. cursor) in augmented reality. In an example, a person can input food-related information by forming a selected pattern of thought which is detected by a mobile EEG device. In an example, a person can input food-related information by making a gesture in proximity to a device with gesture recognition functionality. In an example, a person can input food-related information by scanning a barcode or QR code associated with food.

In an example, Bayesian analysis of food types and quantities can begin with preliminary (prior) estimates of food types and quantities based on word-based descriptions from a person and then modify (update) these estimates with the results from automated analysis. In an example, Bayesian analysis of food types and quantities can begin with preliminary (prior) estimates of food types and quantities based on automated analysis and then modify (update) these estimates based on word-based descriptions from a person. In an example, a person can be prompted to provide word-based descriptions of food items when automated analysis fails to provide estimates of food types and quantities with sufficient accuracy. In an example, a person can be prompted by a system to provide more and more information concerning food items and/or food consumption until the system is able to identify food types and estimate food quantities with a selected level of accuracy and/or certainty.

In an example, a system can include a human-to-computer interface for communication from a human to a computer. In an example, a human-to-computer interface can be based on mobile EEG monitoring and analysis of brainwaves. In an example, a human-to-computer interface can comprise scanning a bar code or QR code associated with a food item. In an example, a human-to-computer interface can comprise recognizing eating-related motions via smart clothing with motion sensors and/or bend sensors. In an example, a human-to-computer interface can comprise a virtual menu which is displayed on the screen of a handheld device or in a person's field of vision via augmented reality eyewear. In an example, a human-to-computer interface can comprise a neural interface. In an example, a human-to-computer interface can comprise a virtual keypad and/or keypad which is projected onto a surface. In an example, a human-to-computer interface can comprise a pop-up menu. In an example, a human-to-computer interface can comprise a dial or rotating bezel.

In an example, a system for nutritional monitoring and management can include a human-to-computer interface for communication from a human to a computer. In an example, a human-to-computer interface can be control buttons. In an example, a human-to-computer interface can comprise a device with gesture recognition functionality. In an example, a system can recognize gestures associated with food selection, identification, and/or consumption. In an example, a human-to-computer interface can comprise a physical or light-projected keyboard or keypad. In an example, a human-to-computer interface can comprise a computer mouse or trackball. In an example, a human-to-computer interface can comprise smart eyewear (such as augmented reality eyewear). In an example, a human-to-computer interface can enable a person to type their descriptions of food items into the system. In an example, a human-to-computer interface can comprise read what a person writes (e.g. a written dietary log) with respect to descriptions of food items consumed. In an example, a human-to-computer interface can have speech and/or voice recognition functionality.

In an example, a system for nutritional monitoring and management can include a human-to-computer interface through which a person provides food-related information. This interface can comprise one or more elements selected the group consisting of: microphone, speech recognition, and/or voice recognition interface; touch screen, touch pad, keypad, keyboard, buttons, or other touch-based interface; camera, motion recognition, gesture recognition, eye motion tracking, or other motion detection interface; interactive food-identification menu with food pictures and names; and interactive food-identification search box.

In an example, a system for nutritional monitoring and management can include a human-to-computer interface for communication from a human to a computer. In an example a human-to-computer interface can be a touch screen and/or touch pad. In an example, a human-to-computer interface can comprise an augmented reality interface on a handheld device or in smart eyewear. In an example, a human-to-computer interface can comprise eye movement and/or gaze tracking. In an example, a human-to-computer interface can comprise tracking head movement. In an example, a human-to-computer interface can comprise tracking arm, hand, wrist, and/or finger movement. In an example, a human-to-computer interface can be a graphical user interface through which a person enters information concerning food items (especially gooey food items). In an example, a human-to-computer interface can comprise gesture recognition via EMG sensors on a person's arm, hand, and/or fingers. In an example, a human-to-computer interface can comprise gesture recognition via EMG sensors on a person's arm, hand, and/or fingers. In an example, a human-to-computer interface can comprise gesture recognition via an arm band with EMG sensors. In an example, a human-to-computer interface can comprise gesture recognition via one or more wearable motion sensors. In an example, a human-to-computer interface can comprise gesture recognition via one or more wearable bend sensors or strain sensors.

In an example, a system for nutritional monitoring and management can include a device which projects a visible laser beam toward food. In an example, this visible laser beam can be different from an outward-directed light beam that is used for spectroscopic analysis. In an example, a visible laser beam can be used by a person in order to point an invisible spectroscopic beam toward a food item for compositional analysis of the food item and/or to direct a camera's focus toward the food item to record an image of the food item. In an example, a person can “point and click” by pointing a laser beam toward a food item and then activating (e.g. by touching, tapping, clicking, or pressing) a device to take a spectroscopic scan of the food, capture an image of the food, or both. In an example, a person can point a laser beam toward food and then give a verbal command to initiate a spectroscopic scan and/or image capture. In an example, spectroscopic analysis can identify food item composition and image analysis can estimate food item quantity. In an example, a visible laser beam projected toward food can also serve as a fiducial marker for calibration of food size and/or color in analysis of food images.

In an example, a person can be prompted to take a picture of food when eating is detected. In an example, a person can be prompted to take one or more actions (e.g. take a picture of food, input a description of the food, take a spectroscopic of food) when the system detects that the person is eating (or has been eating for a selected time without taking a picture or inputting description of food). In an example, a person can be guided concerning how to move a camera in a particular pattern (e.g. varying distance and angle from food) in order to create a 3D image or model of food. This guiding can be visual (e.g. via AR), auditory, or tactile. In an example, a person can be prompted to take these actions when automated analysis does not yield identification of food types and/or quantities with sufficient certainty. In an example, a person can be prompted to take one or more actions when spectroscopic or image analysis suggests lack of food homogeneity. In an example, a person can be prompted to collect additional sensor data concerning food items and/or provide additional description of food items until a system is able to identify food items and estimate food item quantities with a selected level or accuracy and/or certainty.

In an example, a system for nutritional monitoring and management can prompt a person to collect and/or provide information concerning food item types and/or quantities. In an example, a system can automatically track a first set of information concerning food item types and/or quantities (that a person eats) and prompt a person to collect and/or provide a second set of information concerning food item types and/or quantities (that the person eats). In an example, both sets of information can be jointly analyzed by the system to determine food item types and/or quantities (that a person eats). In an example, a system can prompt a person to collect and/or provide information concerning food item types and/or quantities when the system detects that the person is eating (or likely to start eating soon). In an example, a system can include a device with an eating-detection sensor worn by a person, wherein the system prompts the person to collect and/or provide information concerning food item types and/or quantities when data from the eating-detection sensor indicates that the person is eating.

In an example, a system for nutritional monitoring and management can include eating-detection sensor selected from the group consisting of: motion sensor (e.g. accelerometer, gyroscope, and/or bend sensor), microphone or some other type of sound sensor, EMG sensor or some other type of electromagnetic energy sensor, and camera. In an example, a system can prompt a person to collect and/or provide food-related information via a prompt selected from the group consisting of: a flashing light, light display, icon display, image display, or some other type of visual stimulus; a mild electrical current or some other type of electromagnetic stimulus; a phone call or some other type of telephonic stimulus; a text message or some other type of written stimulus; a tone, buzzer, alarm, note, song, computer-generated speech, prerecorded verbal message, or some other type of audio stimulus; and a vibration, moving protrusion which moves relative to a person's skin, or some other type of haptic stimulus.

In an example, a system for nutritional monitoring and management can prompt a person to collect and/or provide food information though one or more mechanisms selected from the group consisting of: using a smart utensil for food consumption; using a set of smart place-setting components (dish, plate, utensils, glass, etc) to record information about types and quantities of food; using a food scale; inserting a food probe into food; recording images (e.g. taking pictures) of food from different angles; recording a video of food from different angles; directing light energy toward (or into) food and recording the results of interaction between this energy and food; taking a spectroscopic scan of food; directing electromagnetic energy toward (or into) food and recording the results of interaction between this energy and food; and directing sound energy toward (or into) food and recording the results of interaction between this energy and food.

In an example, a system for nutritional monitoring and management can prompt a person to collect and/or provide information concerning food item types and/or quantities by performing one or more of the following actions: inserting a food probe into a food item; making a food-related gesture which is recognized by the system; moving a virtual cursor to point at a food item or outline the border of the food item; moving a projected light beam to point at a food item or outline the border of the food item; placing a fiducial marker near food to calibrate food size, orientation, or color; recording an image (e.g. take a picture) of a food item; scanning a food barcode or QR code; selecting a food item from a menu displayed on a device screen; selecting a food item from a menu displayed via augmented reality eyewear; speaking a food description into a microphone; taking a spectroscopic scan of a food item; typing a food description via a keypad or touch screen; using a smart utensil to eat; and weighing food on a scale.

In an example, a system for nutritional monitoring and management can prompt a person to collect and/or provide food-related information by recording an image (e.g. take a picture) of a food item. In an example, a system can prompt a person to collect and/or provide food-related information by weighing food on a scale. In an example, a system can prompt a person to collect and/or provide food-related information by scanning a food barcode or QR code. In an example, a system can prompt a person to collect and/or provide food-related information by moving a virtual cursor to point at a food item or outline the border of the food item. In an example, a system can prompt a person with clarifying questions concerning the types and quantities of food that person has consumed. These questions can be asked in real time, as a person eats, at a subsequent time, or periodically.

In an example, a system for nutritional monitoring and management can prompt a person to collect and/or provide food-related information by speaking a food description into a microphone. In an example, a system can prompt a person to collect and/or provide food-related information by typing a food description via a keypad or touch screen. In an example, a system can prompt a person to collect and/or provide food-related information by making a food-related gesture which is recognized by the system. In an example, a system can prompt a person to collect and/or provide food-related information by selecting a food item from a menu displayed on a device screen. In an example, a system can prompt a person to collect and/or provide food-related information by selecting a food item from a menu displayed via augmented reality eyewear.

In an example, a system for nutritional monitoring and management can prompt a person to collect and/or provide food-related information by moving a projected light beam to point at a food item or outline the border of the food item. In an example, a system can prompt a person to collect and/or provide food-related information by placing a fiducial marker near food to calibrate food size, orientation, or color. In an example, a system can prompt a person to collect and/or provide food-related information by inserting a food probe into a food item. In an example, a system can prompt a person to collect and/or provide food-related information by taking a spectroscopic scan of a food item. In an example, a system can prompt a person to collect and/or provide food-related information by using a smart utensil to eat.

In an example, a system for nutritional monitoring and management can prompt a person to collect and/or provide information concerning food item types and/or quantities when food is detected near the person. In an example, a system can prompt a person to collect and/or provide information concerning food item types and/or quantities when a person first starts to eat. In an example, a system can prompt a person to collect and/or provide information concerning food item types and/or quantities after the person has eaten for a selected period of time. In an example, a system can prompt a person to collect and/or provide information concerning food item types and/or quantities after the person has eaten for a selected period of time if the person has not already collected and/or provided this information during this period of time. In an example, a system can prompt a person to collect and/or provide information concerning food item types and/or quantities after a person has finished eating a meal.

In an example, a system for nutritional monitoring and management can prompt a person to record images of food using a camera when data from the wearable sensor indicates eating and the person does not record images of food for this eating event before eating starts. In an example, the person can be prompted to record images of food when data collected by a wearable sensor indicates eating and the person does not record images of food for this eating event before a selected length of time after eating starts. In an example, the person can be prompted to record images of food when data collected by the wearable sensor indicates eating and the person does not record images of food for this eating event before a selected quantity of eating-related actions occurs during the eating event. In an example, the person can be prompted to record images of food when data collected by the wearable sensor indicates eating and the person does not record images of food for this eating event at the end of the eating event. In an example, a system can prompt the person to use one or more sensor to collect information concerning food items multiple times during a meal.

In an example, a system for nutritional monitoring and management can prompt a person to use a smart utensil, probe, or dish when data from a wearable sensor indicates that the person is eating and the person has not started using the smart utensil, probe, or dish before a selected length of time after eating starts. In an example, a person can be prompted to use a smart utensil, probe, or dish when data from a wearable sensor indicates that the person is eating and the person does not start using the smart utensil, probe, or dish before a selected quantity of eating-related actions (e.g. bites or forkfulls) occurs. In an example, a person can be prompted to record images of food when data collected by a wearable sensor indicates eating and the person does not record images of food for this eating event before a selected quantity of eating-related actions occurs during the eating event. In an example, a person can be prompted to use a smart utensil, probe, or dish when data from the wearable sensor indicates eating and the person does not use the smart utensil, probe, or dish throughout an entire eating event. In an example, a person can collect and/or provide food-related information before, during, or after eating. In an example, collection and/or provision of food information by a person can be prompted or solicited in real time when eating is first detected. In an example, collection and/or provision of food information by a person can be prompted or solicited at the end of the day and can be associated with multiple eating events throughout the day.

In an example, a system for nutritional monitoring and management can create a sound or voice, light, vibration or tactile sensation that prompts a person to use a handheld spectroscopic food sensor when data from a wearable device indicates that the person is eating. In an example, a person can be prompted to use a spectroscopic food sensor by a prompt selected from the group consisting of: beep, buzz, tone, sequence of tones, alarm, voice, music, or other sound-based prompt; vibration, prod, sliding rotating, or pressing protrusion, contracting garment or accessory, or other tactile prompt; mild shock, neurostimulation, or other electromagnetic energy prompt; and LED, LED pattern, blinking light, flash, image display, or other light energy prompt. In an example, a system can comprise a speaker, light, actuator or other moving member, or electromagnetic energy emitter which creates such a prompt. In an example, a wearable device which is in wireless communication with a handheld spectroscopic food sensor can include a speaker, light, actuator or other moving member, or electromagnetic energy emitter which creates such a prompt.

In an example, a system for nutritional monitoring and management can project a light pattern in a sequential manner toward a series of selected locations on a meal where a person should take spectroscopic scans. In another example, a person can move a projected light pattern from one food item to another in a meal in order to separately identify each food item. In an example, a person can sequentially take spectroscopic scans from one food item to another in the same sequence in which the person moves a projected light beam from one food item to another. This can link each food item in a food image with the results of the appropriate spectroscopic scan of that food item. Using these or similar methods, each food item in an image can be linked with the results of its corresponding spectroscopic scan.

In an example, a system for nutritional monitoring and management can prompt a person to take spectroscopic scans at selected locations on a food item or across multiple food items in a meal based on the analysis of food images taken by a camera. In an example, food images can be analyzed to identify different food items in a meal. In an example, a person can be prompted to take spectroscopic scans at different locations on the mail which are associated with different food items. In an example, suggested locations for these spectroscopic scans can be communicated from a system to a person by a light pattern which is projected onto food at these different locations. In an example, the results of spectroscopic scans of food at a plurality of selected locations can be linked to different food items in a meal image. In an example, a person can take a scan at a selected location on food and then take a picture of the food with that location highlighted by a light pattern pointed toward that location.

In an example, a system for nutritional monitoring and management can use a combination of food-related information which is collected automatically from sensors and food-related information which is voluntarily provided by a person. In an example, a system can automatically collect food-related information from a combination of motion sensors, sound sensors, food images, and/or spectroscopic sensors and can also receive voluntary food-related information from a person via a microphone, touch screen, keypad, and/or gesture recognition interface. In an example, multivariate analysis of automatically-collected food information and voluntarily-provided food information can enable more accurate identification of food item types and estimation of food item quantities than either type of food information alone. In an example, a system can prompt a person to enter verbal descriptions of what they eat each time that they eat.

In an example, a system for nutritional monitoring and management can measure a person's consumption of at least one type of food, ingredient, or nutrient. In an example, a system can identify and track in an entirely automatic manner the types and quantities of foods, ingredients, or nutrients that a person consumes. Alternatively, such identification can occur in a partially-automatic manner in which there is interaction between automated and human food identification methods. In an example, identification of the types and quantities of food, ingredients, or nutrients that a person consumes can be a combination of, or interaction between, automated food identification methods and human-based food identification methods. In an example, automatic identification of food types and quantities can be based on: color and texture analysis; image segmentation; image pattern recognition; volumetric analysis based on a fiducial marker or other object of known size; and/or three-dimensional modeling based on pictures from multiple perspectives.

In an example, a system for nutritional monitoring and management can estimate the level of accuracy and/or certainty with which a system can identify food item types and estimate food item quantities based on information which is automatically collected when a person eats. In an example, a system can estimate the level of accuracy and/or certainty with which the system can identify food item types and estimate food item quantities based on information from motion sensors, food images, sound sensors, and/or spectroscopic sensors when a person eats. In example, if the level of accuracy and/or certainty is below a target level, then the system can prompt a person to provide additional food-related information. In example, if the level of accuracy and/or certainty is below a target level, then the system can prompt a person to provide additional food-related information in an iterative and interactive manner until the target level is achieved. In an example, a person can be prompted to take additional pictures of food, take additional spectroscopic scans of food, and/or provide additional verbal descriptions of food until a target level of food identification accuracy and/or certainty is achieved. In an example a target level can be higher when the risk of an error is greater, such as when a system is relied upon to avoid food items to which a person is allergic or to detect toxic substances in food items.

In an example, a system for nutritional monitoring and management can determine initial estimates of food types and quantities, convey these initial estimates to a person, and then receive information from the person which is used by the system to refine these initial estimates. In an example, a system can: (a) determine initial estimates of food item types and quantities based on data which is automatically collected by sensors when a person eats, (b) convey these initial estimates to the person; and (c) receive voluntary food-related information from the person which is used to refine these initial estimates. In an example, a system can: (a) determine preliminary estimates of food item types and quantities based on data from motion sensors, sound sensors, food images, and/or spectroscopic sensors, (b) communicate these preliminary estimates to a person through a display screen, augmented reality eyewear, and/or synthesized speech; and (c) receive additional food-related information directly from the person, wherein the system uses this additional information to refine the preliminary estimates of food item types and quantities.

In an example, a method for nutritional monitoring and management can comprise: collecting primary data using a wearable food-consumption monitor to detect when a person is eating, wherein this monitor is worn on the person, and wherein primary data collection does not require action by the person during eating apart from the act of eating; and collecting secondary data using a handheld food-identifying sensor to identify the selected types of foods, ingredients, or nutrients that the person is eating, wherein secondary data collection by the handheld food-identifying sensor requires action by the person during eating apart from the act of eating, and wherein the person is prompted to take this action when primary data indicates that the person is eating and secondary data has not already been collected.

In an example, a system for nutritional monitoring and management can comprise a wearable device which detects when a person is eating. In an example, a system can prompt a person (e.g. via vibration, voice, sound, or light) to use a mobile device to scan food and/or record images of food when the person is eating. In an example, a person can be prompted to use a device to monitor nutritional intake when a wearable device detects that they are eating. In an example, eating can be detected by a person swallowing a selected number of times in a period of time, by a pattern of chewing, and/or by a pattern of repeated hand motions. In an example, a wearable device can be selected from the group consisting of: smart watch; wrist band; necklace and/or pendant; ear bud; and eyewear.

In an example, a system for nutritional monitoring and management can comprise: (a) a wearable sensor that is worn on a person's body or clothing, wherein this wearable sensor automatically collects data that is used to detect eating without requiring action by the person in association with eating apart from the act of eating; (b) a camera, wherein this camera is used by the person to record images of food that the person eats, wherein using this camera to record images of food requires voluntary action by the person apart from the act of eating, and wherein the person is prompted to record images of food using this camera when data collected by the wearable sensor indicates eating; and (c) a data analysis component, wherein this component analyzes food images taken by the camera to estimate the types and quantities of foods, ingredients, nutrients, and/or calories that are eaten by the person.

In an example, a system for nutritional monitoring and management can comprise: (a) a wearable food-consumption monitor that is configured to be worn on a person's body or clothing, wherein this monitor automatically collects primary data that is used to detect when the person is eating; (b) a computer-to-human prompting interface which a person uses to enter secondary data concerning the person's consumption of at least one selected type of food, ingredient, or nutrient, wherein this interface selected from the group consisting of: speech or voice recognition, touch or gesture recognition, motion recognition or eye tracking, and buttons or keys, and wherein this interface prompts the person to enter secondary data in association with a specific food consumption event when the primary data indicates that the person is eating and the person has not already entered this data. In an example, primary data can be body movement data or data concerning electromagnetic signals from the person's body. In an example, secondary data can be collected by a mobile phone, smart utensil, food probe, smart necklace, smart eyewear, or a smart watch.

In an example, a system for nutritional monitoring and management can comprise: a wearable motion sensor that automatically collects data concerning a person's body motion, wherein this body motion data is used to determine when this person is eating; and a user interface that prompts the person to provide additional information concerning the selected types of foods, ingredients, or nutrients that the person is eating when the body motion data indicates that the person is eating. In an example, a method for nutritional monitoring and management can comprise: (a) having a person wear a motion sensor on a body member selected from the group consisting of wrist, hand, finger, and arm; wherein this motion sensor continually monitors body motion to provide primary data that is used to detect when a person is eating; and (b) prompting the person to collect secondary data concerning food consumption when the primary data indicates that the person is eating; wherein secondary data is selected from the group consisting of: data from the interaction between food and reflected, absorbed, or emitted light energy including pictures, chromatographic results, fluorescence results, absorption spectra, reflection spectra, infrared radiation, and ultraviolet radiation; data from the interaction between food and electromagnetic energy including electrical conductivity, electrical resistance, and magnetic interaction; data from the interaction between food and sonic energy including ultrasonic energy; data from the interaction between food and chemical receptors including reagents, enzymes, biological cells, and microorganisms; and data from the interaction between food and mass measuring devices including scales and inertial sensors.

In an example, a system for nutritional monitoring and management can include a smart watch which collects primary data concerning eating and can prompt a person to collect and/or provide secondary data for food identification when primary data indicates that the person is eating and the person has not yet collected secondary data. In an example, primary data can be body motion data and secondary data can be food images. In an example, a smart watch can be a mechanism for collecting primary data and a smart spoon can be a mechanism for collecting secondary data. In an example, collection of primary data can be automatic, not requiring any action by the person in association with eating apart from the actual act of eating, but collection of secondary data can require a specific action (e.g. triggering and aiming a camera). In an example, automatic primary data collection and non-automatic secondary data collection can combine to provide relatively high-accuracy and high-compliance food consumption measurement with relatively low privacy intrusion.

In an example, a system for nutritional monitoring and management can include a wearable device which detects changes in person's heart rate, heart rhythm, and/or heart rate variation which indicates that the person is eating. When data from a wearable device indicates that a person is eating, the system can prompt the person to record food images using a camera and/or to scan food using a spectroscopic sensor. In an example, a system can include: a wearable camera that automatically records images, wherein the images are analyzed to detect when the person is eating; and a user interface that prompts the person to provide additional information concerning the selected types of foods, ingredients, or nutrients that the person is eating when the images indicate that the person is eating. In an example, a system can include a mobile EEG monitor which detects changes in a person's electromagnetic brain activity which indicate that a person is eating. This system can prompt the person to record food images using a camera and/or scan food using the spectroscopic sensor when eating is detected.

In an example, a system for nutritional monitoring and management can prompt a person to trigger, activate, or operate secondary data collection in association with eating when analysis of primary data indicates that this person is eating. In an example, a system can prompt a person to trigger, activate, or operate a secondary data collection component in association with eating when analysis of primary data indicates that this person is eating. In an example, a system with a component that automatically collects primary data to detect when a person is eating can prompt the person to collect secondary data to identify food consumed when the person is eating. In an example, a system can prompt a person to collect and/or provide secondary data in association with eating when analysis of primary data indicates that the person is eating and the person has not yet collected secondary data. In an example, secondary data can be the results of chemical analysis of food. In an example, collection of secondary data can require that the person bring a nutrient-identifying utensil or sensor into physical contact with food. In an example, collection of secondary data can require that the person speak into a voice-recognizing device and verbally identify the food that they are eating. In an example, collection of secondary data can require that a person use a computerized menu-interface to identify the food that they are eating. In an example, a system can include a smart watch (with a motion sensor) to detect eating and a smart spoon (with a built-in chemical composition sensor), wherein a person is prompted to use the smart spoon to eat food when the smart watch detects that the person is eating.

In an example, a system for nutritional monitoring and management can prompt a person to use a smart spoon for eating and automatically record images of portions of food that are in the spoon's scoop. In an example, such automatic picture taking can be triggered by infrared reflection, some other type of optical sensor, a pressure sensor, an electromagnetic sensor, or some other type of contact sensor in the spoon scoop. In an example, a system can prompt a person to use a camera to record an image of food in the spoon's scoop. In an example, a system can prompt a person to aim a camera toward food on a plate, in a bowl, or in original packaging to record images of food before it is apportioned into portions by the spoon. In an example, food on a plate, in a bowl, or in original packaging can be easier to identify by analysis of its shape, texture, scale, and colors than food apportioned into portions.

In an example, a system for nutritional monitoring and management can prompt a person to use a mobile device to provide and/or collect food-related information when a wearable device detects that that person is eating. In an example, a system can prompt a person (e.g. by vibration, sound, or light) to use a mobile device to provide and/or collect food information when a wearable device worn that that person detects (e.g. based on eating-related body motions or sounds) that the person is eating. In an example, a system can prompt a person to use a mobile device to take spectroscopic scans of food and/or to record images of food when a wearable device detects that the person is eating.

In an example, a system for nutritional monitoring and management can track the amount of food eaten during a meal or during a period of time spanning multiple meals. In an example, a system can track calories consumed per day and cumulative calories consumed. In an example, a system can track calories consumed during a period of time and compare this to a calorie budget for that period of time. In an example, a system can track the number of bites and/or swallows during a meal and/or during a period of time. In an example, a system can track arm, wrist, and/or hand motion to help estimate the quantity of food consumed. In an example, a system can track the pitch, roll, and yaw of wrist and/or hand motion to help estimate the quantity of food consumed. In an example, a system can track the speed and/or pace of bites or sips by tracking the speed and/or pace of wrist and/or hand motions. In an example, a system can recognize arm and/or hand gestures to help estimate the quantity and/or speed of food consumption. In an example, a system can track and report historical food consumption patterns for a person.

In an example, a system for nutritional monitoring and management can include a camera whose field of vision and/or the focal length is automatically adjusted to track a moving object such as a person's hand, a person's mouth, or a food item. In an example, a system can include a camera which scans space around a person's hand or mouth in order to detect and identify food items. In an example, a system can include a wrist-worn camera which tracks the ends of a person's fingers in order to detect and identify food items. In an example, a system can monitor: the types and volumes of food items within view and/or reach of the person; changes in the volumes of these food items over time; the number of times that the person brings their hand (with food) to their mouth; the sizes or portions of food that the person brings to their mouth; and the number, frequency, speed, or magnitude of chewing, biting, or swallowing movements.

In an example, a system for nutritional monitoring and management can associate a timestamp with a food consumption event. In an example, a system can track and analyze the timing, speed, and/or pace of a person's food consumption. In an example, a system can track and analyze when a person eats meals and whether the person eats snacks between meals. In an example, a system can track and analyze how quickly a person eats meals or snacks between meals. In an example, a system can track and analyze the speed and/or pace of a person's hand-to-mouth motions, chewing motions, sipping motions, swallowing motions, and/or biting motions. In an example, a system can track and analyze the duration of a person's meals and/or between-meal snacks. In an example, a system can analyze associations between food consumption speed and food consumption amount. For example, if a person tends to be satiated with less food when the person eats more slowly, then a system can encourage a person to eat more slowly. In an example, a system can encourage a person to eat more slowly via sound cues, haptic cues, and/or visual cues. In an example, a system can encourage a person to eat more slowly by providing: visual cues (e.g. display of virtual objects) via augmented reality eyewear; sound cues (e.g. musical tones or other sounds) via an ear-worn wearable device; haptic cues (e.g. vibrations) via a smart watch or band; and/or haptic cues (e.g. vibrations) via a smart utensil.

In an example, a system can collect food-related information before and after a person eats. Differences in food-related information before vs. after eating can be analyzed to estimate the quantities of food items which a person has actually eaten. In an example, a system can collect food-related information before and after a person eats a meal, wherein differences in food-related information before vs. after eating are analyzed to estimate the quantities of food items which a person actually eats during the meal. In an example, food-related information can include food images before vs. after the person eats. In an example, differences in food size in before vs. after images can be used to estimate the quantity of food which a person has eaten. In an example, food-related information can include food weight before vs. after the person eats. In an example, a system can collect data that enables tracking the cumulative amount of foods, ingredients, and/or nutrients which a person consumes during a period of time (such as an hour, day, week, or month) or during a particular eating event.

In an example, a system can collect food-related information multiple times while a person is eating. In an example, a system can collect food-related information multiple times while a person is eating a meal. In example, a system can take spectroscopic scans of food at multiple times (and/or prompt a person to take spectroscopic scans at multiple times) during a meal. Taking multiple spectroscopic scans during a meal can collect spectroscopic information about multiple layers or structures of the interior of a food item. If a spectroscopic sensor only measures the surface of a food item which is exposed at a given time, then taking multiple scans during a meal is particularly important when the interior of a food item has a different composition than the exterior of the food item.

In an example, a system for nutritional monitoring and management can automatically record images of food items at the start of a meal and the end of the meal. Differences between images at the start and end of a meal can be used to estimate the actual quantity of food items consumed by a person. In an example, a system can automatically record images of food items at multiple times during a meal, using sequential reductions in the quantity of food items remaining to estimate the actual quantity of food items consumed by a person. In an example, a system can be triggered to automatically record images of food when eating (a meal) begins and when eating (the meal) ends, using differences in food in the before vs. after images to estimate the actual quantity of food consumed by a person. In an example, a system can prompt a person to record images of food when eating (a meal) begins and when eating (the meal) ends and use differences in food in the before vs. after images to estimate the actual quantity of food consumed by a person.

In an example, a system for nutritional monitoring and management can automatically record the weight of food on a scale at the start of a meal and at the end of the meal, using differences in weight between the before and after measurements to estimate the actual quantity of food items consumed by a person. In an example, a food scale can measure the overall weight of food items in a meal by measuring, at different times during a meal, the overall weight of a food holding item (such as a plate, bowl, cup, or tray) which holds different types and/or portions of food. In an example, a multi-part food scale can measure the weights of different food items or portions in a meal, wherein different food items or portions in the meal are located on different parts and/or segments of the multi-part scale. In an example, each part and/or segment of a multi-part food scale can individually and independently measure the weight of a type of food on that particular part and/or segment. In an example, parts and/or segments of a multi-part food scale can be separated by ridges or partitions. In an example, a system can include a food scale. In example, the weight of food can be measured before and after a meal to determine the weight of food eaten by a person. In an example, food portions can be eaten sequentially and scale measurements can be made after each portion. In an example, a scale can have multiple sub-scales, one for each segment of a meal (e.g. for each type of food).

In an example, a system for nutritional monitoring and management can analyze multiple food characteristics into order to identify food item types and quantities. In an example, these food characteristics can include the amounts of vitamins and minerals in a food item. In an example, these food characteristics can include the ingredient list on packaging of a food item. In an example, these food characteristics can include the ingredients in a recipe for a food item. In an example, these food characteristics can include the light absorption spectrum of a food item. In an example, these food characteristics can include the light reflection spectrum of a food item. In an example, these food characteristics can include the nutritional composition of a food item. In an example, these food characteristics can include the percentage or amount of dietary fiber in food item. In an example, these food characteristics can include the percentage or amount of saturated fat in food item. In an example, these food characteristics can include the percentage or amount of carbohydrates in a food item. In an example, these food characteristics can include the percentage or amount of fats in a food item. In an example, these food characteristics can include the percentage or amount of protein in a food item. In an example, these food characteristics can include the percentage or amount of sugars in a food item. In an example, these food characteristics can include the percentage or amount of trans fat in a food item.

In an example, a system for nutritional monitoring and management can estimate total calories in a food item or meal. In an example, a system can estimate types and quantities of carbohydrates, sugars, fats, salts, proteins, vitamins, and/or minerals in a food item or meal. In an example, a system can identify allergens, carcinogens, toxins, metals, chemicals, pathogens, bacteria, and/or fungi in a food item or meal. In an example, a system can identify: antioxidants, beans, beef, bread, cereal, cheese, corn, dairy, egg, fish, fruit, grain, milk, nuts, oats, pasta, pork, poultry, rice, starch, sugar, vegetables, and/or wheat. In an example, a system can estimate the freshness of beef, cheese, dairy, egg, fish, fruit, milk, nuts, pork, poultry, and/or vegetables. In an example, a system can estimate the water content of: beans, bread, cereal, corn, grain, oats, pasta, rice, and/or wheat.

In an example, a system for nutritional monitoring and management can estimate the quantities of food items and/or nutrients in those food items. In an example, a system can estimate quantities of food items or nutrients which are near a person before the person starts eating, after the person has eaten, or the difference between before and after eating. In an example, a system can estimate the quantities of food items or nutrients which a person actually consumes. In an example, a system can estimate the cumulative quantity of food items or nutrients being consumed by a person in real time (or close to real time). In an example, a system can estimate quantities of food and/or nutrients consumed in real time during a meal.

In an example, a system for nutritional monitoring and management can estimate quantities of food and/or nutrients consumed by a person by estimating changes in the volume of food near the person during a meal. In an example, a system can count the number of times that a person lifts a spoon, fork, or other food-transporting utensil up to their mouth using data from motion and/or force sensors. In an example, motion sensors can be part of a utensil. In an example, motion sensors can be part of a device worn on a person's arm, wrist, and/or finger. In an example, a device worn on a person's arm, wrist, or finger can include a proximity sensor which detects when a food utensil is near the device. Such a proximity sensor can enable indirectly tracking utensil movement via a motion sensor on a wearable device.

In an example, a system for nutritional monitoring and management can estimate quantities of food and/or nutrients consumed by a person by: estimating the amount of food per spoon or fork full; estimating the number of times a spoon or fork has been lifted up to a person's mouth; and multiplying the amount in a spoonfull or a forkfull times the number of lifts. In an example, the amount of food per spoonfull or forkfull can be estimated by data from a force sensor and/or motion sensor on a spoon or fork. In an example, the amount of food per spoonfull or forkfull can be estimated by (past) correlation between a decreasing amount of food near a person in images and an increasing number of times that a spoon or fork is lifted up to a person's mouth. In an example, the amount of food per spoonfull or forkfull can be estimated by the amount of time that a spoon or fork is held in proximity to a person's mouth during a lift. In an example, the amount of food per spoonfull or forkfull can be estimated by the number of times that a person chews and/or swallows per spoonfull or forkfull. In an example, chews and/or swallows can be monitoring using a wearable sound sensor, wearable motion sensor, wearable vibration sensor, or wearable electromagnet energy (e.g. EMG) sensor. In an example, chewing and/or swallowing can be monitoring by a device worn around a person's neck, a device worn on a person's throat or neck, an ear-worn device, or an intra-oral device.

In an example, a system for nutritional monitoring and management can estimate quantities of liquids consumed by a person by: estimating the amount of liquid per sip from a beverage container (e.g. glass, cup, mug, or bottle); estimating the number of times a beverage container has been lifted up to a person's mouth; and multiplying amount in a sip times the number of container lifts. In an example, the amount of food per sip can be estimated by data from an optical sensor (e.g. liquid level detector) in a beverage container. In an example, the amount of food per sip can be estimated by the number of times that a person swallows per sip. In an example, swallowing can be monitoring using a wearable sound sensor, wearable motion sensor, wearable vibration sensor, or wearable electromagnet energy (e.g. EMG) sensor. In an example, swallowing can be monitoring by a device worn around a person's neck, a device worn on a person's throat or neck, an ear-worn device, or an intra-oral device.

In an example, a system for nutritional monitoring and management can also analyze the packaging and/or label of a food item in order to identify food item types and estimate food item quantities. In an example, a system can also analyze a barcode or QR code of food packaging. In an example, a system can also analyze food pairings (e.g. which types of food are near a food item in a meal). In an example, a system can also analyze the configurations of borders between food items in a meal or on a dish. In an example, a system can also analyze the homogeneity of a food item. In an example, a system can also analyze the type of serving dish (e.g. plate, bowl, glass, cup, bottle, can, package, wrapper, bag, box) on which (or in which) a food item is served. In an example, a system can also analyze food shading or light intensity. In an example, a system can also analyze food shape. In an example, a system can also analyze food size.

In an example, a system for nutritional monitoring and management can also analyze where food is stored (e.g. on a shelf or in a refrigerator) as part of identification of food item types and estimation of food item quantities. In an example, a system can also analyze food temperature. In an example, a system can also analyze food texture. In an example, a system can also analyze the type of utensil (e.g. fork, spoon, knife, and/or chop sticks) which is used to eat a food item. In an example, a system can also analyze whether a food item is held by a person's hand during eating. In an example, a system can also analyze chewing or swallowing sounds during food consumption. In an example, a system can also analyze food viscosity and/or motion. In an example, a system can also analyze the geolocation of food selection, purchase, or consumption (e.g. via GPS). In an example, a system can also analyze the reflection of infrared light from food. In an example, a system can also analyze the spectral distribution of light reflection or absorption by food (e.g. spectroscopic scan data).

In an example, a system for nutritional monitoring and management can analyze the environmental context for food selection, purchase, or consumption as part of identifying food item types and estimating food item quantities. In an example, a system can also analyze food color (or color spectral distribution) in ambient light. In an example, a system can also analyze food configuration (e.g. food orientation in a meal). In an example, a system can also analyze the type of container in which food is stored. In an example, a system can also analyze the electromagnetic impedance of food. In an example, a system can also analyze the location of a food item in a meal. In an example, a system can also analyze the location of a food item on a dish (e.g. where is it located on a plate of food).

In an example, a system for nutritional monitoring and management can analyze multiple food characteristics in order to identify food items types and estimate food item quantities. In an example, a system for nutritional monitoring and management can analyze multiple food characteristics selected from the group consisting of: environmental context for food selection, purchase, or consumption; food color or color spectral distribution in ambient light; food configuration (e.g. food orientation); food container type; food electromagnetic impedance; food location in a meal; food location on a dish; food packaging and/or label; food packaging barcode or QR code; food pairings (e.g. types of food nearby in a meal); food portion border; food portion homogeneity; food serving dish type (e.g. plate, bowl, glass, cup, bottle, can, package, wrapper, bag, box); food shading; food shape; food size; food storage type (e.g. shelf, refrigerator); food temperature; food texture; type of food utensil (or person's hand) used to eat food; food viscosity and/or motion; chewing or swallowing sounds during food consumption; geolocation (e.g. GPS) of food selection, purchase, or consumption; infrared reflection pattern; spectral distribution of light reflection or absorption; spectroscopic scan data; and ultrasonic energy reflection pattern.

In an example, a system for nutritional monitoring and management can analyze multiple food characteristics into order to identify food item types and quantities. In an example, these food characteristics can include a barcode or QR code on the label or packaging of a food item. In an example, these food characteristics can include a logo or other images on the label or packaging of a food item. In an example, these food characteristics can include the name or location of a restaurant where a food item is served. In an example, these food characteristics can include the presence of allergens or pathogens in a food item. In an example, these food characteristics can include the shape of a food item. In an example, these food characteristics can include the shape of the perimeter of a food item. In an example, these food characteristics can include the three-dimensional shape of a food item. In an example, these food characteristics can include the size of a food item. In an example, these food characteristics can include the volume of a food item. In an example, these food characteristics can include text on a label or packaging of a food item. In an example, these food characteristics can include the texture of a food item.

In an example, a system for nutritional monitoring and management can analyze multiple food characteristics into order to identify food item types and quantities. In an example, these food characteristics can include a description of a food item on a restaurant menu. In an example, these food characteristics can include verbal descriptions of food items by one or more users. In an example, these food characteristics can include the color and/or spectral distribution of a food item. In an example, these food characteristics can include the distance from a camera to a food item in an image. In an example, these food characteristics can include the food items which are paired with (or otherwise accompany) a food item in a meal. In an example, these food characteristics can include the geolocation of the consumption of a food item. In an example, these food characteristics can include the geolocation of cooking and/or preparation of a food item. In an example, these food characteristics can include the geolocation of the purchase of a food item. In an example, these food characteristics can include the history of consumption of a food item by a person or persons. In an example, these food characteristics can include the temperature of a food item. In an example, these food characteristics can include the time of consumption of a food item. In an example, these food characteristics can include the type of dish or container on (or in) which a food item is served. In an example, these food characteristics can include the weight of a food item.

In an example, a system for nutritional monitoring and management can record food item images from multiple angles and/or distances to create a three-dimensional model for determining food item volumes and/or quantities. In an example, a system can estimate quantities of food items from food images by volumetric analysis of food from multiple perspectives and/or three-dimensional modeling of food. In an example, a system can record food images from multiple angles to segment a meal into different food item types, estimate the three-dimensional volume of each food item type, and control for lighting and shading differences. In an example, a system can guide a person how to record food images from different angles for volumetric analysis of food item quantities.

In an example, a system for nutritional monitoring and management can analyze images of food items to determine the types and/or quantities of foot items in an image. In an example, a system can analyze a video of food items and/or sequential still images of food items to estimate a three-dimensional food item's size, volume, and/or quantity. In an example, a system can prompt a person to move a mobile device in a selected pattern in proximity to a food item in order to record a video and/or sequential still images of the food item to estimate three-dimensional food size, volume, and/or quantity. In an example, a mobile device of a nutritional monitoring and management system can include an infrared light projector which projects infrared light toward a food item and an infrared light receiver which receives that light after it has been reflected in order to estimate the distance from the mobile device to the food item.

In an example, there can be inter-portion food variation in a meal. Inter-portion variation is variation in food characteristics between different portions (e.g. different types or items) of food in a meal and/or given location. Inter-portion variation can include differences in molecular composition, color, texture, shape, temperature, and location. Different types of food can be identified by inter-portion differences in their molecular composition, color, texture, shape, temperature, and location in a meal. To address inter-portion variation, a person can take spectroscopic scans of food items in a meal. The locations of these scans can be based on the person's evaluation of the number and locations of these different portions. Alternatively, to address inter-portion variation, a system can guide person how to take spectroscopic scans at different locations and/or of different portions of a meal based on automated analysis of food images.

In an example, there can be intra-portion food variation in a meal. Intra-portion variation is variation in food characteristics within a portion (e.g. a single type or item) of food. Intra-portion variation also include differences in molecular composition, color, texture, shape, temperature, and location. Some foods are non-homogenous. For example, there can be pieces of fruit or nuts at different locations on the outer surface of a food item. Different locations on the outer surface of a food item can have different molecular compositions, colors, textures, shapes, temperatures, or locations. To address intra-portion variation on the outer surface of food, a person can take spectroscopic scans of different locations on the surface of a food item based on the person's evaluation different types of ingredients and/or components on that surface.

In an example, an image of a meal comprising multiple food items can be automatically segmented into different food items (e.g. portions of different types of food in a meal) using pattern analysis. In an example, different food items (or portions) in a meal can be automatically identified and segmented using one or more food characteristics selected from the group consisting of: dish or container on (or in) which a food item is served, food item borders, food item chemical composition, food item color, food item description on a menu, food item distance, food item geolocation, food item light absorption spectrum, food item light reflection spectrum, food item orientation, food item positions in a meal, food item shading, food item shape, food item size, food item temperature, food item texture, food item volume, juxtaposition of food items in a meal, and within-meal food item relationships.

In an example, a system for nutritional monitoring and management can detect unhealthy food, wherein unhealthy food is selected from the group consisting of: food that is high in simple carbohydrates; food that is high in simple sugars; food that is high in saturated or trans fat; fried food; food that is high in Low Density Lipoprotein (LDL); and food that is high in sodium. In an example, a system can identify and quantify food that is high in simple sugars. In an example, a system can identify and quantify food that is high in saturated fats. In an example, a system can identify and quantify food that is high in trans fats. In an example, a system can identify and quantify food that is high in Low Density Lipoprotein (LDL). In an example, a system can identify and quantify food that is high in sodium. In an example, a system can identify and quantify food that is high in simple carbohydrates.

In an example, a system for nutritional monitoring and management can identify and quantify one or more types of food, ingredients, and/or nutrients selected from the group consisting of: a selected food, ingredient, or nutrient that has been designated as unhealthy by a health care professional organization or by a specific health care provider for a specific person; a selected substance that has been identified as an allergen for a specific person; peanuts, shellfish, or dairy products; a selected substance that has been identified as being addictive for a specific person; alcohol; a vitamin or mineral; vitamin A, vitamin B1, thiamin, vitamin B12, cyanocobalamin, vitamin B2, riboflavin, vitamin C, ascorbic acid, vitamin D, vitamin E, calcium, copper, iodine, iron, magnesium, manganese, niacin, pantothenic acid, phosphorus, potassium, riboflavin, thiamin, and zinc; a selected type of carbohydrate, class of carbohydrates, or all carbohydrates; a selected type of sugar, class of sugars, or all sugars; simple carbohydrates, complex carbohydrates; simple sugars, complex sugars, monosaccharides, glucose, fructose, oligosaccharides, polysaccharides, starch, glycogen, disaccharides, sucrose, lactose, starch, sugar, dextrose, disaccharide, fructose, galactose, glucose, lactose, maltose, monosaccharide, processed sugars, raw sugars, and sucrose; a selected type of fat, class of fats, or all fats; fatty acids, monounsaturated fat, polyunsaturated fat, saturated fat, trans fat, and unsaturated fat; a selected type of cholesterol, a class of cholesterols, or all cholesterols; Low Density Lipoprotein (LDL), High Density Lipoprotein (HDL), Very Low Density Lipoprotein (VLDL), and triglycerides; a selected type of protein, a class of proteins, or all proteins; dairy protein, egg protein, fish protein, fruit protein, grain protein, legume protein, lipoprotein, meat protein, nut protein, poultry protein, tofu protein, vegetable protein, complete protein, incomplete protein, or other amino acids; a selected type of fiber, a class of fiber, or all fiber; dietary fiber, insoluble fiber, soluble fiber, and cellulose; a specific sodium compound, a class of sodium compounds, and all sodium compounds; salt; a selected type of meat, a class of meats, and all meats; a selected type of vegetable, a class of vegetables, and all vegetables; a selected type of fruit, a class of fruits, and all fruits; a selected type of grain, a class of grains, and all grains; high-carbohydrate food, high-sugar food, high-fat food, fried food, high-cholesterol food, high-protein food, high-fiber food, and high-sodium food.

In an example, a system for nutritional monitoring and management can identify and quantify one or more selected types of food, ingredients, and/or nutrients selected from the group consisting of: amino acid or protein (a selected type or general class), carbohydrate (a selected type or general class, such as single carbohydrates or complex carbohydrates), cholesterol (a selected type or class, such as HDL or LDL), dairy products (a selected type or general class), fat (a selected type or general class, such as unsaturated fat, saturated fat, or trans fat), fiber (a selected type or class, such as insoluble fiber or soluble fiber), mineral (a selected type), vitamin (a selected type), nuts (a selected type or general class, such as peanuts), sodium compounds (a selected type or general class), sugar (a selected type or general class, such as glucose), and water.

In an example, a system for nutritional monitoring and management can identify and quantify one or more types of food, ingredients, and/or nutrients selected from the group consisting of: a specific type of carbohydrate, a class of carbohydrates, or all carbohydrates; a specific type of sugar, a class of sugars, or all sugars; a specific type of fat, a class of fats, or all fats; a specific type of cholesterol, a class of cholesterols, or all cholesterols; a specific type of protein, a class of proteins, or all proteins; a specific type of fiber, a class of fiber, or all fiber; a specific sodium compound, a class of sodium compounds, and all sodium compounds; high-carbohydrate food, high-sugar food, high-fat food, fried food, high-cholesterol food, high-protein food, high-fiber food, and high-sodium food.

In an example, a system for nutritional monitoring and management can identify one or more types of food whose consumption is prohibited or discouraged for religious, moral, and/or cultural reasons, such as pork or meat products of any kind. In an example, food can be classified into general categories such as fruits, vegetables, or meat. In an example, a system can identify one or more potential food allergens, toxins, or other substances selected from the group consisting of: ground nuts, tree nuts, dairy products, shell fish, eggs, gluten, pesticides, animal hormones, and antibiotics. In an example, a system can track the quantities of chemicals in food selected from the group consisting of carbon, hydrogen, nitrogen, oxygen, phosphorus, and sulfur.

In an example, a system for nutritional monitoring and management can collect and analyze data concerning food items in order to identify food item types and estimate food item quantities. In an example, identification of food item types and estimation of food item quantities can include estimation of ingredients in food items and/or the nutritional composition of food items. In an example, identification of food item types and estimation of food quantities can include identification of allergens and/or impurities. In an example, images of food items can be taken before and after food consumption by a person in order to estimate the amount of food actually consumed by the person. In an example, the amount of food remaining after food consumption can be subtracted from the amount of food before food consumption in order to estimate the amount of food actually consumed by a person. In an example, a system for monitoring and managing nutrition can analyze food items with respect to: dish or container on (or in) which a food item is served, food item borders, food item chemical composition, food item color, food item description on a menu, food item distance, food item geolocation, food item light absorption spectrum, food item light reflection spectrum, food item orientation, food item positions in a meal, food item shading, food item shape, food item size, food item temperature, food item texture, food item volume, juxtaposition of food items in a meal, and within-meal food item relationships.

In an example, a system for nutritional monitoring and management can classify a type or quantity of a food or nutrient as being unhealthy based on one or more factors selected from the group consisting of: the type of food or nutrient; the speed or pace of food or nutrient consumption; a person's age, gender, and/or weight; changes in a person's weight; a person's diagnosed health conditions; one or more general health status indicators; the magnitude and/or certainty of the effects of past consumption of the selected nutrient on a person's health; achievement of a person's health goals; a person's exercise patterns and/or caloric expenditure; a person's physical location; the time of day; the day of the week; occurrence of a holiday or other occasion involving special meals; input from a social network and/or behavioral support group; input from a virtual health coach; the cost of food; financial payments, constraints, and/or incentives; health insurance copay and/or health insurance premium; the amount and/or duration of a person's consumption of healthy food or nutrients; a dietary plan created for a person by a health care provider; and the severity of a food allergy.

Quantities of food, ingredients, and nutrients can be measured in terms of volume, mass, or weight. Volume measures how much space the food occupies. Mass measures how much matter the food contains. Weight measures the pull of gravity on the food. The concepts of mass and weight are related, but not identical. In an example, volume can be expressed in metric units (such as cubic millimeters, cubic centimeters, or liters) or U.S. (historically English) units (such as cubic inches, teaspoons, tablespoons, cups, pints, quarts, gallons, or fluid ounces). Mass (and often weight in colloquial use) can be expressed in metric units (such as milligrams, grams, and kilograms) or U.S. (historically English) units (ounces or pounds).

The density of specific ingredients or nutrients within food is sometimes measured in terms of the volume of specific ingredients or nutrients per total food volume or measured in terms of the mass of specific ingredients or nutrients per total food mass. In an example, nutrient density or concentration can be measured as part of an automatic food, ingredient, or nutrient identification method. In an example, nutrient density can be expressed as the average amount of a specific ingredient or nutrient per unit of food weight. In an example, nutrient density can be expressed as the average amount of a specific ingredient or nutrient per unit of food volume. In an example, food density can be estimated by interacting food with light, sound, or electromagnetic energy and measuring the results of this interaction. Such interaction can include energy absorption or reflection.

In an example, a system for nutritional monitoring and management can measure food weight, mass, volume, or density. In an example, a system can include a food scale, strain gauge, or inertial sensor. In an example, a system can measure the weight or mass of an entire meal, a portion of one type of food or food item within that meal, or a mouthful of a type of food that is being conveyed to a person's mouth. In general, a weight, mass, or volume sensor is more useful for general detection of food consumption and food amount than it is for identification of type of food, ingredients, and nutrients.

In an example, a system for nutritional monitoring and management can include a food database. In an example, a food database can have multiple levels, including super-sets of food types (e.g. meals with selected combinations of food types and quantities) and sub-sets of food types (e.g. types and quantities of ingredients, nutrients, and/or chemicals which comprise food types). In an example, a system can include a database of different types and quantities of food items. In an example, a system can include a database of different food items and characteristics associated with each food item. In an example, food item characteristics can be used to match a nearby food item with a food item in the database. In an example, a system can include a database of different food item types and quantities, including standardized nutritional composition for each listed quantity for each listed food item. In an example, a database can include standardized types and quantities of ingredients, nutrients, and/or calories for each listed quantity for each food item. In an example, a food database can link common types and quantities of food with common types and quantities of ingredients and/or nutrients. In an example, a system can be in wireless communication with a remote database which links food items with standardized quantities of ingredients and/or nutrients.

In an example, a food database can include data elements selected from the group consisting of: barcode (or QR code) associated with food item; food item (probable) color; food item (probable) health effects; food item (probable) ingredients and/or nutritional composition; food item (probable) location or position with respect to selected dishware (e.g. plate, bowl, or beverage container); food item (probable) pairings with other specific food items; food item (probable) portion size; food item (probable) shape; food item (probable) size; food item (probable) temperature; food item (probable) texture; food item (probable) use in selected meals; food item (probable) allergic effects; food item association with particular times of day, days of the week, times of the year, or holidays; food item cost; food item health rating or ranking; food item homogeneity or lack thereof; food item image (including possible multiple images in different contexts such as on a plate vs. utensil, or from different angles and distances); food item light absorption or reflection spectrum (including the results of spectroscopic analysis); food item name (including possible synonyms and different languages); food item status with respect to specific diets and/or religious observations; general health effects associated with food item; geolocations associated with food item availability; packaging, label, and/or logo associated with food item; person's past consumption quantity or patterns concerning food item; person-specific health effects associated with food item; restaurants or grocery stores associated with food item; and suggested substitutions for food item. In an example, a food database can be based on historical information concerning (food consumption by) a group of people and/or the general population. In an example, a food database can be based on historical information concerning (food consumption by) a specific person.

In an example, a system for nutritional monitoring and management can estimate types and quantities of ingredients and/or nutrients indirectly using a database than links identified food items with standardized quantities ingredients and/or nutrients. In an example, a system for nutritional monitoring and management can estimate quantities of ingredients or nutrients indirectly by: (a) collecting and/or receiving characteristics of food item types and identifying food types and estimating food item quantities; (b) linking these food item types and quantities to records in a food database which link foods with ingredients and/or nutrients; and (c) extracting estimated types and quantities of ingredients and/or nutrients from the database associated with those food item types and quantities. Alternatively, a system can estimate types and quantities of ingredients and/or nutrients directly using a chemical and/or molecular composition sensor (such as a spectroscopic sensor).

In an example, images of one or more food times can be analyzed to help identify types and quantities of food items and/or nutrients. Analysis of food images can include one or more methods selected from the group consisting of: 3D image modeling, volumetric analysis, adjusting image aspect ratio, computer vision, discriminant analysis, image color calibration and/or adjustment, image composition analysis including food pairings and juxtapositions, image compression, image deletion or editing, image filtering, image lighting intensity calibration and/or adjustment, image rectification, image resizing, image resolution calibration and/or adjustment, image rotation, image segmentation, image size calibration and/or adjustment, machine learning, multivariate analysis, and artificial neural network analysis. In an example, a user can be prompted to provide additional and/or supplemental information concerning their evaluation of food item type and quantity when the results of automated analysis do not achieve a desired level of accuracy or certainty.

In an example, a system for nutritional monitoring and management can automatically identify food item types and estimate food items quantities using one or more automated methods. In an example, a system can automatically identify food item types and estimate food items quantities using Artificial Intelligence (AI). In an example, a system can automatically identify food item types and estimate food items quantities using association rule learning. In an example, a system can automatically identify food item types and estimate food items quantities using Bayesian analysis.

In an example, a system for nutritional monitoring and management can automatically identify food item types and estimate food items quantities using clustering. In an example, a system can automatically identify food item types and estimate food items quantities using computer vision. In an example, a system can automatically identify food item types and estimate food items quantities using computer vision. In an example, a system can automatically identify food item types and estimate food items quantities using crowd sourcing. In an example, a system can automatically identify food item types and estimate food items quantities using data analytics.

In an example, a system for nutritional monitoring and management can automatically identify food item types and estimate food items quantities using decision tree analysis. In an example, a system can automatically identify food item types and estimate food items quantities using deep learning algorithms. In an example, a system can automatically identify food item types and estimate food items quantities using fuzzy logic. In an example, a system can automatically identify food item types and estimate food items quantities using inductive logic programming. In an example, a system can automatically identify food item types and estimate food items quantities using least squares estimation. In an example, a system can automatically identify food item types and estimate food items quantities using logistic discrimination. In an example, a system can automatically identify food item types and estimate food items quantities using machine learning.

In an example, a system for nutritional monitoring and management can automatically identify food item types and estimate food items quantities using machine learning. In an example, a system can automatically identify food item types and estimate food items quantities using multivariate analysis. In an example, a system can automatically identify food item types and estimate food items quantities using multivariate linear regression. In an example, a system can automatically identify food item types and estimate food items quantities using an Artificial Neural Network (ANN). In an example, a system can automatically identify food item types and estimate food items quantities using pattern recognition. In an example, a system can automatically identify food item types and estimate food items quantities using pattern recognition.

In an example, a system for nutritional monitoring and management can identify food item types and estimate food item quantities using one or more methods selected from the group consisting of: chemical analysis, Chi-squared analysis, cluster analysis, color analysis, factor analysis, probit analysis, survival analysis, texture analysis, volumetric analysis, machine learning, 3D modeling, three-dimensional modeling, image normalization, non-linear programming, face recognition, gesture recognition, logo recognition, motion recognition, pattern recognition, speech recognition, linear regression, logistic regression, Fourier Transformation, principal components analysis (PCA), linear discriminant analysis, time series analysis, Bayesian statistical analysis, inter-food boundary determination, artificial neural network (ANN), bar code or QR code recognition, linear mathematical programming, optical character recognition (OCR), sound pattern recognition, multivariate linear regression, food portion segmentation, and analysis of variance.

In an example, a system for nutritional monitoring and management can automatically identify food item types and estimate food items quantities using Principal Component Analysis (PCA). In an example, a system can automatically identify food item types and estimate food items quantities using Random Forest (RF) analysis. In an example, a system can automatically identify food item types and estimate food items quantities using a Support Vector Machine (SVM).

In an example, a system for nutritional monitoring and management can use multivariate analysis including factors selected from the group consisting of: image-related variables (e.g. food images and automated analysis of those images, food item packaging logo, food item packaging type, UPC or QR code on food packaging, type of dish or other container used to hold fold); spectroscopic variables (e.g. data from spectroscopic analysis of food, light absorption and/or reflection spectra of food items, data from spectroscopic analysis of person's body tissue, light absorption and/or reflection spectra of body tissue); motion-related variables (e.g. number of eating-related motions or gestures by a person's arm, wrist, or hand; number of times a person brings their hand up to their mouth in a specific manner; utensil movement, number of chews based on motion, number of swallows based on motion); utensil-related variables (e.g. type of dish or container used to hold food, type of utensil used to bring food from dish or container up to a person's mouth); and timing variables (e.g. day of the week; frequency of eating-related motions or gestures by a person's arm, wrist, or hand; pace with which a person brings their hand repeatedly up to their mouth during a meal; person's frequency or pace of chews during a meal or period of time; person's frequency or pace of swallows during a meal or period of time; time since person's last meal; timing of a holiday or other special occasion; time of day).

In an example, a system for nutritional monitoring and management can use multivariate analysis which including factors selected from the group consisting of: voice or sound-related variables (e.g. verbal descriptions of food items or meals; number of chews based on sound; number of swallows based on sound; sound spectrum of chews and/or swallows); person-specific biometric parameters or health-related variables (e.g. person's acute illness or chronic condition, person's age, person's blood pressure, person's body temperature, person's body weight, person's eating pace, person's fatigue level, person's gender, person's glucose level, person's heart rate, person's historical eating patterns, person's past biometric parameter changes in response to consumption of specific types or quantities of food, person's sleep level or pattern, person's socioeconomic status, and person's stress level); scale-related variables (e.g. food weight as measured by a scale integrated into a food dish or container); energy balance variables (e.g. person's amount of exercise and/or physical activity during a period of time, person's cumulative food consumption during a period of time); and environmental variables (e.g. geolocation, ambient humidity, ambient light level, ambient temperature, altitude, restaurant type or name, grocery store type or name, food source).

In an example, a method for nutritional monitoring and management can comprise: collecting primary data concerning food consumption using a wearable food-consumption monitor to detect when a person is eating; and collecting secondary data concerning food consumption using a handheld food-identifying sensor when analysis of primary data indicates that the person is eating. In an example, a method can comprise: automatically collecting primary data from an eating-detection sensor that a person wears on their body or clothing; and prompting the person to use a handheld food-identifying sensor to collect secondary data when primary data indicates that the person is eating and the person has not already collected secondary data associated with that eating event.

In an example, a system for nutritional monitoring and management can have a target level of accuracy and/or certainty with which food item types are to be identified and/or food item quantities are to be estimated. In an example, if a first set of sensors do not provide food identification and quantification with the target level of accuracy and/or certainty, then the system can activate a second set of sensors to collect additional food-related information. In an example, if automated sensors do not provide food identification and quantification with the target level of accuracy and/or certainty, then the system can prompt a person to collect and/or provide additional food-related information. In an example, additional food-related information can be collected and/or provided in an iterative manner until the target level of accuracy and/or certainty is achieved. In an example, a system can determine food item types and quantities based on a first set of data and a second set of data. If results from these two sets of data converge, then the system can stop collecting data. However, if the results from these two sets of data do not converge, then the system can collect additional data and/or prompt a person to provide additional data. In an example, a system for nutritional monitoring and management can start with the descriptions of food types and estimations of food quantities provided by a person and then refine them, in a Bayesian manner, based on the results of spectroscopic analysis and food image analysis.

In an example, a system for nutritional monitoring and management can include a food database that is used to identify food types and quantify food amounts. In an example, a food database can include average (or standardized) types and quantities of ingredients and/or nutrients associated with specific food items. In an example, average types and quantities of ingredients and/or nutrients from the database can be used to estimate consumption of ingredients and/nutrients associated with a person's consumption of a food item. In an example, estimation of specific ingredients or nutrients eaten can be done using a database that links specific foods (and quantities thereof) with specific ingredients or nutrients (and quantities thereof). In an example, a database can be customized for a specific person based on that person's past eating habits. In an example, identification of food item types and quantities for a person can be done, in whole or in part, by predicting the person's current eating patterns based on the person's historical eating patterns. In an example, a system can analyze one or more factors selected from the group consisting of: number of nearby food items; types of food items; changes in the volume of nearby food items; number of times that a person brings food up to their mouth; number of chewing movements; frequency or speed of chewing movements; and number of swallowing movements.

In an example, a system for nutritional monitoring and management can analyze food images to determine food item types and estimate food item quantities. In an example, a system can analyze food using one or more methods selected from the group consisting of: volumetric analysis, image normalization, face recognition, gesture recognition, pattern recognition, calibration of an image using a fiducial marker of known size and/or color, analyzing the chemical composition of food, analyzing food color, recognizing packaging design, inter-food boundary determination, segmentation of meal image into food items, bar code or QR code recognition, optical character recognition, food logo recognition, analyzing food shape, analyzing food size and changes in food size during eating, analyzing food texture, analyzing food volume, 3D or volumetric modeling of food, and recognizing words on food packaging.

In an example, a system for nutritional monitoring and management can record images of a person's mouth and nearby food from multiple perspectives to create a three-dimensional model of food. In an example, images of a person's mouth, a nearby food item, and the interaction between the person's mouth and food can be automatically, or semi-automatically, analyzed to estimate the types and quantities of food that the person eats. In an example, a system can automatically determine borders between different food items in a meal image, segmenting the meal into different food items before comparison with food item images in a food database. In an example, a system can compare an image of a meal (with multiple types of food) as a whole with images of meals (with multiple types of food) in a food database.

In an example, a method for nutritional monitoring and management can comprise: collecting a first set of data in an automatic and continuous manner to detect when a person is eating; collecting a second set of data to identify what selected types of foods, ingredients, or nutrients the person is eating when the first set of data indicates that the person is eating; and jointly analyzing both the first and second sets of data to estimate consumption of at least one specific food, ingredient, or nutrient by the person. In an example, a method can comprise: receiving descriptions of nearby food types and quantities from a person; receiving data from spectroscopic analysis of the food; receiving data from analysis of images of the food; and performing multivariate analysis on the descriptions from the person, spectroscopic data, and image data in order to identify types and quantities of the food (or the ingredients, nutrients, and/or chemicals therein).

In an example, a method for nutritional monitoring and management can comprise: recording images of nearby food using at least one camera which is worn on a person's body; collecting data concerning the spectrum of light that is transmitted through and/or reflected from nearby food using at least one optical sensor which is worn on the person's body; and automatically analyzing the food images to identify the types and quantities of food, ingredients, and/or nutrients. In an example, a system can combine data from a spectroscopic sensor with data from analysis of food images to determine types and quantities of food (or ingredients, nutrients, and/or chemicals therein). In an example, a system can identify types and quantities of foods, ingredients, or nutrients from images or images of food using a combination of automated food identification methods and human-based food identification methods. In an example, a system which combines both spectroscopic analysis and image analysis can provide good information on both the types and quantities of nearby food (and nutrients, chemicals, and/or possibly even microorganisms in that food).

In an example, a system for nutritional monitoring and management can include an augmented reality (AR) interface between the system and a person whose nutritional intake is being monitored and managed. In an example, an AR interface can be a computer-to-human interface through which information is conveyed from the system to a person. In an example, an AR interface can be a human-to-computer interface through which information is conveyed from a person to the system. In an example, an augmented reality (AR) interface can be incorporated into smart eyewear. In an example, AR eyewear can display food-related information visually in a person's field of view, optionally accompanied by information conveyed in auditory and/or haptic modalities. In an example, AR eyewear can receive food-related information from a person via voice, gestures, text entry, eye movement, and/or EEG signals.

In an example, a system for nutritional monitoring and management can include augmented reality (AR) eyewear which displays virtual content in a person's field of view in juxtaposition with (e.g. over or near) food items. In an example, virtual content displayed in juxtaposition with (e.g. over or near) food items can be selected from the group consisting of: name of a food item; estimated total calories and/or nutritional composition (e.g. fats, carbohydrates, proteins, etc.) of a food item; binary (e.g. healthy vs. unhealthy) or continuous (e.g. health rating) information concerning a food item; probable health effects of consuming a food item; information concerning allergens, pathogens, and/or carcinogens in a food item; estimated quantity of a food item; cost and/or nearby location where a food item can be purchased; and review or poll results concerning a food item.

In an example, food-related information can be displayed in virtual words, graphics, or images in juxtaposition with (e.g. over or near) food items in an augmented reality display. In an example, displayed information for a specific food item in a person's field of view can be visually linked to that food item by a virtual connecting arrow or line in an augmented reality display. In an example, information concerning each of a plurality of food items in a person's field of view (e.g. in a multi-food meal) can be consistently displayed in the same direction (e.g. to the right, to the left, above, or under) relative to a food item. For example, total estimated calories for each food item in a meal can be virtually displayed under each food item in a meal in an augmented reality display. In an example, displayed information for a specific food item in a person's field of view can be visually linked to that food item by being the same color as a virtual circle, box, or outline displayed around the specific food item in an augmented reality display. For example, each food item in a meal can be outlined in a different color and information about each food item can be displayed above or below the meal, wherein the color of the information about each item matches the color of the outline around the item.

In an example, a system for nutritional monitoring and management can include augmented reality eyewear. In an example, augmented reality eyewear can display a virtual pointer at different locations (e.g. different portions or types of food) in a meal to direct where a person should place a spectroscopic sensor to take scans of the food. In an example, augmented reality eyewear can track (using gesture recognition) where a person moves a spectroscopic sensor for food scans and can link scan results from those locations with different portions or types of food which are identified by image analysis. In an example, the results of food identification and quantification from a mobile device can be displayed in a person's field of view using augmented reality eyewear. In an example, a system can include augmented reality via a mobile handheld device. In an example, the information discussed above can be display on the screen of a mobile device instead by (or in addition to) augmented reality eyewear.

In an example, a system for nutritional monitoring and management can superimpose suggested areas for spectroscopic analysis on a person's view of a meal in using augmented reality eyewear. In an example, augmented reality eyewear can display one or more virtual pointers at selected locations on a meal to guide a person as to where they should take spectroscopic cans of the meal. For example, augmented reality eyewear can display a virtual pointer on a portion of fish on a plate. The person then uses the handheld device to take a spectroscopic scan of that fish. Then, the augmented reality eyewear can move the virtual point to a portion of carrots on the plate. Then the person takes a scan of the carrots. This continues for each type of food on the plate and/or in the meal. Portion specific spectroscopic information is then combined with food quantity information from analysis of food images to get an overall estimation of types and quantities of foods, ingredients, and/or nutrients. In an example, a system can identify locations on food where a person should a the spectroscopic scanner. In an example, augmented reality eyewear can display virtual pointers on food to direct where a person should use a spectroscopic scanner.

In an example, smart eyewear which is part of a system can further comprise a gesture recognition function. In an example, information about a specific food item may be displayed in augmented reality when a person makes a specific gesture relative to (e.g. points toward) that specific food item. In an example, smart eyewear which is part of a system can further comprise an eye movement and/or gaze-tracking function. In an example, information about a particular food item may be displayed in augmented reality when a person looks at that specific food item.

In an example, food item information can be conveyed via the color or configuration of virtual objects shown in juxtaposition with (e.g. over or near) food items in a person's field of view. In an example, the color of a virtual circle or borders around a specific food item displayed in augmented reality in a person's field of view can indicate whether that food item is relatively healthy or unhealthy for the person to consume. In an example, a green circle or border around a food item can mean that the food is healthy, a yellow circle or border can mean that the food item is neutral, and a red circle or border can mean that the food item is unhealthy. In an example, a circle or border of a specific color around a specific food item can indicate that the food item contains something to which the person is allergic, a pathogen, and/or a carcinogen.

In an example, a system for nutritional monitoring and management can superimpose nutrition information on a person's view of their environment via augmented reality. In an example, virtual nutrition information can be superimposed directly over the food in question. In an example, display of negative nutritional information and/or information about the potential negative effects of unhealthy nutrients can reduce a person's consumption of an unhealthy type or quantity of food. In an example, a system can display warnings about potential negative health effects and/or allergic reactions. In an example, display of positive nutritional information and/or information on the potential positive effects of healthy nutrients can increase a person's consumption of healthy food. In an example, a system can display encouraging information about potential health benefits of selected foods or nutrients.

In an example, augmented reality eyewear can change the perceived color spectrum of selected food items in a person's field of view in order to change how appetizing or unappetizing the food appears. For example, the color spectrum of unhealthy food (or food which a person should not eat for other reasons) can be changed to make that food less appealing. For example, some people like green eggs and ham but would like not like green fries and spam. In an example, augmented reality eyewear can display an image next to a food item in a person's field of view in order to change the appeal of that food item. In an example, an unappetizing image can be displayed in juxtaposition with unhealthy food (or food which the person should not eat for other reasons) to make that food less appealing. For example, would you be interested in eating French fries next to a picture of Jabba the Hutt? How about if Jabba winked at you with each fry you ate? I didn't think so.

In an example, a system for nutritional monitoring and management can display images or other visual information in a person's field of view in order to modify the person's consumption of food. In an example, unpleasant or unappetizing images can be displayed in proximity to unhealthy food. In an example, pleasant or appetizing images can be displayed in proximity to healthy food. In an example, a system can display images or other visual information in proximity to food in the person's field of view in a manner which modifies the person's consumption of that food. In an example, a system can be part of an augmented reality system which displays virtual images and/or information in proximity to real world objects. In an example, a nutritional intake modification system can superimpose virtual images and/or information on food in a person's field of view.

In an example, a system for nutritional monitoring and management can include smart eyewear with an augmented reality interface which enables a person to provide information (from their perspective) concerning types and quantities of food items in their field of view. In an example, smart eyewear with gesture recognition capability can track the location of a person's finger as the person points to different food items in a meal. In an example, a person can sequentially point to different food items in a meal and provide verbal descriptions of each item, wherein the system associates each verbal description with the appropriate food item. In an example, the system can combine these verbal descriptions with information which the system collected automatically (e.g. via image analysis or spectroscopic analysis) in order to better determine food item types and quantities.

In an example, a system for nutritional monitoring and management can track a person's finger as the person moves their finger in the air tracing the borders between food items in a multi-food meal. Such border tracing can serve as additional input for a system to segment and analyze different food item types and quantities in a multi-food meal. In another example, a system can track a person's finger as the person moves their finger to point sequentially to different food items in a meal, which directs the system to perform sequential spectroscopic scans of those different food items in the meal. In an example, a person can move a virtual cursor in augmented reality to perform the above-mentioned user inputs for system identification of food item types and quantities. In an example, a system can track a person's eye movements and the person can shift their eye gaze and/or focal direction to perform the above-mentioned user inputs for system identification of food types and quantities. In an example, a person can provide user inputs by selecting an entry in a virtual (drop-down) menu in augmented reality.

In an example, a system for nutritional monitoring and management can include a mobile device (such as a smart phone or smart watch) with augmented reality (AR) functionality which displays food information (over a live image of food) on a device screen. In example, food information concerning one or more specific food items can be displayed in juxtaposition with those food items on the mobile device screen. In an example, virtual content which is displayed on a mobile device screen in juxtaposition with (e.g. over or near) food items can be information about food items selected from the group consisting of: name of a food item; estimated total calories and/or nutritional composition (e.g. fats, carbohydrates, proteins, etc.) of a food item; binary (e.g. healthy vs. unhealthy) or continuous (e.g. health rating) information concerning a food item; probable health effects of consuming a food item; information concerning allergens, pathogens, and/or carcinogens in a food item; estimated quantity of a food item; cost and/or nearby location to purchase a food item; and review or poll results concerning a food item. In an example, this information can be displayed in words.

In an example, food-related information can be displayed in virtual words, graphics, or images in juxtaposition with (e.g. over or near) food items on the screen of a mobile device. In an example, displayed information for a specific food item on a screen can be visually linked to that food item by a virtual connecting arrow or line in an augmented reality display. In an example, information concerning each of a plurality of food items on a screen (e.g. in a multi-food meal) can be consistently displayed in the same direction (e.g. to the right, to the left, above, or under) relative to a food item. For example, total calories for each food item in a meal can be virtually displayed under each food item in a meal in an augmented reality display. In an example, displayed information for a specific food item can be visually linked to that food item by being the same color as a virtual circle, box, or outline displayed around the specific food item in an augmented reality display. For example, each food item in a meal can be outlined in a different color and information about each food item can be displayed below the meal, wherein the color of the information about each item matches the color of the outline around the item.

In an example, food item information can be conveyed via the color or configuration of virtual objects shown in juxtaposition with (e.g. over or near) food items on a mobile device screen. In an example, the color of a virtual circle or borders around a specific food item displayed in augmented reality on a mobile device screen can indicate whether that food item is relatively healthy or unhealthy for the person to consume. In an example, a green circle or border around a food item can mean that the food is healthy, a yellow circle or border can mean that the food item is neutral, and a red circle or border can mean that the food item is unhealthy. In an example, a circle or border of a particular color around a specific food item can indicate that it contains something to which the person is allergic, a pathogen, and/or a carcinogen.

In an example, a mobile device (e.g. smart phone) with augmented reality functionality can change the perceived color spectrum of selected food items on its screen in order to change how appetizing or unappetizing the food appears. For example, the color spectrum of unhealthy food (or food which a person should not eat for other reasons) can be changed to make that food less appealing. For example, some people like green eggs and ham but would like not like green fries and spam. In an example, a mobile device (e.g. smart phone) can display an image next to a food item on the device screen in order to change the appeal of that food item. In an example, an unappetizing image can be displayed in juxtaposition with unhealthy food (or food which the person should not eat for other reasons) to make that food less appealing. For example, would you be interested in eating French fries shown next to a picture of Jabba the Hutt? How about if Jabba winked at you each time you ate a French fry? I didn't think so.

In an example, a system for nutritional monitoring and management can include a smart mobile device (e.g. smart phone) with an augmented reality interface which enables a person to provide information (from their perspective) concerning types and quantities of food items in their field of view. In an example, smart mobile device (e.g. smart phone) with gesture recognition capability can track the location of a person's finger as the person points to different food items in a meal. In an example, a person can sequentially point to different food items in a meal and provide verbal descriptions of each item, wherein the system associates these verbal descriptions with the food items. In an example, the system can combine these verbal descriptions with information which the system collected automatically (e.g. via image analysis or spectroscopic analysis) in order to better determine food item types and quantities.

In an example, a smart mobile device (e.g. a smart phone or smart wearable device) which is part of a system can further comprise a gesture recognition function. In an example, information about a specific food item may be displayed on a device screen when a person makes a specific gesture relative to (e.g. points toward) that specific food item. In an example, a system can track a person's finger as the person moves their finger in the air tracing the borders between food items in a multi-food meal. Such border tracing can serve as additional input for a system to segment and analyze different food types and quantities in a multi-food meal. In another example, a system can track a person's finger as the person moves their finger to point sequentially to different food items in a meal, which directs the system to perform sequential spectroscopic scans of those different food items in the meal. In an example, a person can move a virtual cursor in augmented reality to perform the above-mentioned user inputs for system identification of food types and quantities.

In an example, a system for nutritional monitoring and management can track the location of a person's finger on a touch screen as the person touches different food items in an image of a multi-food meal. In an example, a person can sequentially touch different food items in a meal and provide verbal descriptions of each item, wherein the system combines these verbal descriptions with automatically-collected information (e.g. via image analysis, spectroscopic analysis) for determining food types and quantities. In an example, a person can move (e.g. trace) their finger around the borders between food items in a meal on a touch screen as additional input for the system in analysis of food types and quantities. In an example, a person can touch different food items in a meal on a screen to direct sequential (spectroscopic) scans of different food items in the meal. In an example, a person can move a projected light beam to perform the above-mentioned user inputs for system identification of food types and quantities. In an example, a person can select an entry in a virtual (drop-down) menu in augmented reality.

In an example, a system for nutritional monitoring and management can provide a person with food-related information and/or feedback. In an example, a system can provide food-related information via visual, auditory, and/or haptic modalities. In an example, a system can provide food-related information via a visual, auditory, and/or haptic computer-to-human interface. In an example, a system can provide a person with information concerning identified food item types and estimated food item quantities. In an example, a system can provide information concerning the nutritional composition and/or chemical composition of food items. In an example, a system can provide information concerning food item types and quantities which are nearby and which a person may eat. In an example, a system can provide information concerning food item types and quantities which a person has already eaten. In an example, a system can provide negative feedback in association with consumption of unhealthy food and/or positive feedback in association with consumption of healthy food.

In an example, a system for nutritional monitoring and management can provide a person with information concerning which food item types and/or quantities are relatively healthy or unhealthy to eat. In an example, a system can provide a person with the likely positive and/or negative health effects of eating selected food item types and/or quantities. In an example, a system can provide information which encourages a person to eat less unhealthy food and/or to eat more healthy food. In an example, a system can provide a person with information concerning the person's cumulative food consumption during an eating event (e.g. during a meal) or during a period of time (e.g. during a day). In an example, the actual amount of food consumed by the person can be compared to a target amount (e.g. dietary goal) of food consumption for an eating event (e.g. for a meal) or for a period of time (e.g. for a day).

In various examples, a target amount of consumption can be based on one or more factors selected from the group consisting of: the selected type of selected food, ingredient, or nutrient; amount of this type recommended by a health care professional or governmental agency; specificity or breadth of the selected nutrient type; the person's age, gender, and/or weight; the person's diagnosed health conditions; the person's exercise patterns and/or caloric expenditure; the person's physical location; the person's health goals and progress thus far toward achieving them; one or more general health status indicators; magnitude and/or certainty of the effects of past consumption of the selected nutrient on the person's health; the amount and/or duration of the person's consumption of healthy food or nutrients; changes in the person's weight; time of day; day of the week; occurrence of a holiday or other occasion involving special meals; dietary plan created for the person by a health care provider; input from a social network and/or behavioral support group; input from a virtual health coach; health insurance copay and/or health insurance premium; financial payments, constraints, and/or incentives; cost of food; speed or pace of nutrient consumption; and accuracy of a sensor in detecting a selected nutrient.

In an example, a system for nutritional monitoring and management can provide information on a person's energy balance during a period of time. In an example, a system for nutritional monitoring and management can compare a person's caloric intake vs. caloric expenditure during a period of time. In an example, a system can set and monitor caloric intake goals based on a person's caloric expenditure during a period of time. In an example, a system can set and monitor caloric expenditure goals based on a person's caloric intake during a period of time. In an example, a system can set and monitor caloric intake goals and caloric expenditure goals in order for the person to achieve a body weight goal (e.g. maintaining weight, losing weight, or gaining weight).

In an example, a system for nutritional monitoring and management can provide a person with information concerning nearby food before the person starts to eat. In example, providing information before a person eats can be triggered by visual detection of nearby food (e.g. food image recognition) and/or geolocation associated with food purchasing or consumption (e.g. the person is at a restaurant). In an example, a system can provide a person with food-related information when a person starts to eat. In an example, such information during eating can be triggered by detection of eating by motion sensors, image sensors, sound sensors, biometric parameters, and/or geolocation. In an example, a system can provide a person with food-related information at multiple times (or even continuously) while the person eats. In an example, a system for can provide a person with information about consumed food after a person has eaten. In an example, a system can provide a person with information concerning the types and quantities of food that the person has eaten during a specific eating event (e.g. during a meal) or during a period of time (e.g. during a day). In an example, a system can provide a person with periodic information on the types and quantities of food that the person has eaten.

In an example, a system for nutritional monitoring and management can provide a person with information about food item types and quantities before a person chooses which food items to consume and how much of these food items to consume. In an example, a system can provide information to encourage a person to make healthier choices about which food items to consume and how much of them to consume. In an example, a system can provide information about different food choices on a menu to encourage a person to order healthier food. In an example, a system can provide information about food items in real time as a person is consuming those food items. In an example, a system can encourage a person to eat no more than a selected cumulative quantity of one or more food items. In an example, a system can encourage a person to moderate the speed and/or pace at which they are eating food items.

In an example, a system for nutritional monitoring and management can provide a person with information about their food consumption and nutritional intake at times which are not related to specific meals or eating events. In an example, a system can provide a person with information about their cumulative food consumption and nutritional intake for a selected period of time (e.g. a day) in a regular (e.g. daily) manner. In an example, a system can track a person's progress toward dietary and/or health goals over time and provide a person with feedback on their progress toward those goals.

In an example, a system can display images in a person's field of view which influence the person's food consumption. In an example, a system can include augmented reality eyewear which displays images which increase or decrease the appeal of selected types of nearby food. In an example, a system can include augmented reality eyewear which displays appetite-reducing images next to unhealthy foods and/or appetite-enhancing images next to healthy foods. In an example, looking at gummi worms can be tempting to a candy lover, but a super-imposed image of actual worms might have the opposite effect. In an example, looking at a mug of beer might be appealing, but a super-imposed image of a person (perhaps even an augmented image of that person) with a beer gut might have the opposite effect.

In an example, a system for nutritional monitoring and management can display food-related information visually. In an example, a system can provide visual information concerning food items types and quantities. In an example, a system can display food-related information on the screen of a handheld mobile device. In an example, a system can display food-related information superimposed next to nearby food via augmented reality on the screen of a handheld mobile device. In an example, a system can display food-related information in a person's field of view via augmented reality eyewear. In an example, a system can display food-related information via text, graphics, colors, visual patterns, icons, and/or images. In an example, different graphics, colors, visual patterns, icons, and/or images can be associated with different food types and/or quantities. In an example, different graphics, colors, visual patterns, icons, and/or images can be associated with healthy vs. unhealthy food types and/or quantities. In an example, a system can visually display the results of image analysis and/or spectroscopic analysis of food items.

In an example, a system for nutritional monitoring and management can provide a person with visual food-related information and/or feedback through device lights; images, objects, or text on the screen of a handheld device; images, objects, or text from a light projector; and/or images, objects, or text displayed in a person's field of view via augmented reality eyewear. In an example, system can communicate food information to a person in graphic form. In an example, a system can include one or more lights (e.g. LEDs) whose colors and/or light patterns convey information concerning food item types and quantities. In an example, selected light colors and/or patterns can indicate high concentrations of selected types of ingredients, nutrients, and/or chemicals. In an example, selected light colors and/or patterns can indicate whether food items are high in protein, carbohydrates, or fats. In an example, a system can display different colors and/or patterns for different food items in a meal.

In an example, a system for nutritional monitoring and management can passively provide information to a person whose nutritional intake is being monitored and managed. In an example, a system can provide food information to a person in a visual mode. In an example, visual information concerning food and/or nutritional intake can be provided via the screen of a mobile or wearable device. In an example, visual information concerning food and/or nutritional intake can be provided via a display on augmented reality eyewear. In an example, visual information concerning food and/or nutritional intake can be provided via light beams projected from a mobile device or smart eyewear. In an example, visual information can comprise displayed text, graphics, images, virtual objects, or video content. In an example, different colors or light patterns can be used to convey attributes of food such as the nutrient composition of the food and/or whether the food is relatively healthy or unhealthy. In an example, particular color patterns, light patterns, light blinks, or light motions can convey food information to a person. In an example, visual information can be selected or modified based on tracking a person's gaze. In an example, if a person ignores (e.g. does not look at) visual information, then a system may provide auditory or haptic feedback.

In an example, a system for nutritional monitoring and management can provide visual feedback concerning a person's food consumption via the display screen of a mobile or wearable device (such as a smart phone or smart watch). In an example, a system can display selected light patterns, colors, blinks, motions, shapes, and/or intensity levels to signal information about specific types of food and/or a person's consumption of that food. In an example, a system can display selected light patterns, colors, blinks, motions, shapes, and/or intensity levels to indicate how healthy or unhealthy nearby food items are. In an example, visual feedback concerning food consumption and its implications for a person can be provided before a person eats (in order to influence the person's eating decisions before a meal), while a person is eating (in order to influence the person's eating decisions during a meal), or after a person has eaten. In an example, recommended choices with respect to types or portions of food items can be displayed in a visual mode. In an example, the mode (e.g. visual, sound, or haptic) through which feedback is provided from a system to a person can be automatically changed based on environmental cues (e.g. ambient light level, ambient sound level, or geolocation).

In an example, a system for nutritional monitoring and management can provide a person with auditory food-related information. In an example, a system can include a device with a speaker which emits sounds. In an example, a system can convey auditory food-related information via: a tone or note; a ring tone or song; an alarm or buzzer; computer-synthesize speech; a pre-recorded vocal message (by the person or a significant other person); or another type of sound. In an example, a system can convey food-related information to a person via sounds emitted from a smart watch or band, from smart eyewear, from smart earwear, or from a handheld mobile device. In an example, nutritional composition information can be communicated by computer-synthesized speech from a smart watch or band, from smart eyewear, from smart earwear, or from a handheld mobile device. In an example, a system can modify a person's eating behavior by shouting—“If you don't eat yer meat, you can't have any pudding.”

In an example, a system for nutritional monitoring and management can provide food information to a person in an auditory mode (e.g. via sound). In an example, auditory feedback can be provided through speakers in smart eyewear, a mobile device (such as a smart phone), a smart watch, or some other mobile or wearable device. In an example, sounds, tones, buzzers, alarms, songs, music, synthesized speech, and/or prerecorded speech can be used to convey attributes of food such as the nutritional composition of the food and/or whether the food is relatively healthy or unhealthy. In an example, a system can emit selected sounds at selected time intervals in order change a person's eating pace. In an example, a system can change the speed and/or pace of a person's arm or hand motions during eating by entrainment with sound beats, pulses, tones, or notes. In an example, a system can emit selected sounds in order to change a person's consumption of selected types or quantities of food. In an example, sound frequency or volume can increase as a person approaches or exceeds a target amount of food consumption (e.g. during a meal or period of time). In an example, a device can play “Highway to Hell” when a person looks at very unhealthy food or play “Oops . . . I Did It Again” if the person actually eats that food.

In an example, a system for nutritional monitoring and management can provide a person with food-related information or consumption-modifying stimuli through a haptic and/or tactile mechanism. In an example, a haptic and/or tactile mechanism for conveying food-related information or consumption-modifying stimuli can be selected from the group consisting of: electromagnetic stimulation; electromagnetic actuator; haptic array; haptic interface; piezoelectric actuator; pressurized compartment; rotating element; and vibrating element. In an example, different haptic and/or tactile patterns can be associated with different food item types and/or quantities. In an example, different haptic and/or tactile patterns can be associated with healthy vs. unhealthy food types and/or quantities. In an example, haptic and/or tactile stimuli can be delivered when a person is evaluating eating alternatives. In an example, haptic and/or tactile stimuli can be delivered while a person is eating. In an example, haptic and/or tactile stimuli can be delivered after a person has eaten.

In an example, a system for nutritional monitoring and management can provide food information to a person in a haptic mode. In an example, different vibrations, pressures, device movements across skin, and/or protrusion arrays can be used to convey attributes of food such as the nutritional composition of the food and/or whether the food is relatively healthy or unhealthy. In an example, a system can provide haptic feedback (such as vibration, tactile sensation, kinetic sensation, thermal feedback, mild electromagnetic energy delivery) to person concerning the person's food consumption choices and/or the nutritional composition of food items. In an example, a system can provide selected haptic feedback (e.g. vibrations) at selected time intervals in order change a person's eating pace. In an example, a system can provide selected haptic feedback (e.g. vibrations) in order to change a person's consumption of selected types or quantities of food. In an example, haptic feedback frequency or intensity can increase as a person approaches or exceeds a target amount of food consumption (e.g. during a meal or period of time).

In an example, a system for nutritional monitoring and management can have a display screen which shows the results of a spectroscopic scan of food via text, graphic objects, icons, colors, or patterns. In an example, different graphics, objects, icons, and/or colors on a display screen can indicate high concentrations of different types of ingredients, nutrients, and/or chemicals for particular food items and/or at particular locations in a meal. In an example, different graphics, objects, icons, and/or colors on a display screen can indicate whether a particular food item is high in protein, carbohydrates, or fats. In an example, a system can display different graphics, objects, icons, and/or colors for different food items in a meal and/or alternative food items. In an example, a system can include a smart phone, wherein the results of spectroscopic analysis of food are shown on the phone's screen.

In an example, a system for nutritional monitoring and management can provide a person with information concerning the type, total calories, nutritional composition, chemical composition, and/or quantity of nearby food. In an example, a system can display estimated quantity of a food item and/or the estimated total calories of that food item. In an example, a system can display the nutritional composition of food items (e.g. based on image analysis and spectroscopic analysis) in a person's field of view via augmented reality or on the screen of a mobile device. In an example, different colors can be associated with different types of nutrients (e.g. carbohydrates, sugars, fats, and proteins). In an example, a system can display visual cues concerning whether food is relatively healthy or unhealthy for a person to eat. In an example, a system can identify whether a food item contains a specific ingredient, such as an ingredient to which a person is allergic or intolerant.

In an example, a system for nutritional monitoring and management can measure the speed, pace, or rate at which a person eats and encourage the person to eat slower if the speed, pace, or rate is too fast. In an example, feedback from the system to the person can be light-based, such as a blinking light. In an example, feedback can be sound-based, such as a tone, note, alarm, buzzer, song, or computer-generated voice. In an example, feedback can be haptic or tactile, such as a vibration. In an example, visual, auditory, or haptic feedback concerning a person's eating pace can be delivered by a wrist-worn device such as a smart watch. In an example, visual, auditory, or haptic feedback concerning a person's eating pace can be delivered by a smart food utensil.

In an example, a system for nutritional monitoring and management can provide information to a person concerning how fast they are eating and/or prompt the person to slow down if they are eating too fast. In an example, a system can track how fast a person is eating by tracking: the speed of eating-related arm and wrist movements (e.g. tracked by a motion sensor, an EMG sensor, a camera), the speed of food utensil movements (e.g. tracked by a motion sensor on a utensil or image analysis), the speed of chewing or swallowing (tracked by a motion sensor, a vibration sensor, a microphone, or an EMG sensor), or the speed of changes in food weight on a food scale during a meal. In an example, a system can comprise a visual, auditory, of haptic signal when a person eats too fast. In an example, a visual signal can comprise the appearance of a virtual object in a person's field of view in augmented reality. In an example, a haptic signal can comprise vibration of a food utensil which a person is using to eat food. In an example, a haptic signal can comprise vibration of a smart watch worn by a person. In an example, specific colors, visual patterns, light blinks, light motions, or visual icons can be associated with particular types of nutrients. In an example, specific sound patterns and/or songs can be associated with particular types of nutrients.

In an example, a system for nutritional monitoring and management can track the cumulative amount of food eaten by a person during an eating event (e.g. during a meal) or during a period of time (e.g. during a day) and provide feedback to the person based on comparison of actual food consumption to a target amount of food consumption. In an example, a system can provide negative feedback if a person approaches and/or exceeds a target amount of food consumption for an eating event or a period of time. In an example, a device and system can sound an alarm or provide other real-time feedback to a person if the cumulative amount consumed (in total or of a selected type of food, ingredient, or nutrient) exceeds an allowable amount (in total or of a selected type of food, ingredient, or nutrient).

In an example, a system for nutritional monitoring and management can provide information to a person concerning the cumulative quantity of food (or of a particular nutrient) which the person has consumed during a meal or during a period of time. In an example, quantity of food consumed can be compared with a dietary goal or budget for a meal or a period of time. In an example, a system can provide an alert, alarm, or warning when a person is approaching or exceeding a dietary goal or budget for quantity of food (or a particular nutrient) during a meal or during a period of time. In an example, a goal or budget for a quantity of food (or a particular nutrient) can be based at least in part on a person's dietary goals, energy balance goals, body weight goals, and/or energy expenditure during a period of time. In an example, a system can provide recommendations concerning goals for a person's nutritional intake, exercise level, and the relationship between them. In an example, the recommend amount of calories that a system recommends for a person to consume during a period of time can depend on the amount of calories that the person has expended during a period of time. In an example, a person's caloric expenditure can be monitored by a schlep tracker. For example, if a person schleps groceries home from the store and schleps books to class, then their recommended caloric intake increases; but if they are a foyler, then their recommended caloric intake decreases. In an example, a system can track whether a person is consuming too little of a selected food or nutrient. For example, a system can remind a person to drink more water to avoid dehydration if the person has consumed too little water during a period of time. In an example, the amount of water which a person should drink can be determined in part by their activities and environmental factors.

In an example, a system for nutritional monitoring and management can provide a person with dietary recommendations and coaching. In an example, recommendations and coaching can be in real-time as a person is making food consumption decisions or can be with respect to planning future meals. In an example, a system can provide lists of generally healthy vs. unhealthy foods, meals, recipes, and/or restaurants. In an example, a system can provide information about the nutritional composition of particular foods, meals, recipes, and/or (meals at selected) restaurants. In an example, a system can provide health rankings or reviews of foods, meals, recipes, and/or restaurants. In an example, dietary recommendations and coaching by a system can be at least partially based on results reported in scientific and medical literature. In an example, dietary recommendations and coaching by a system can be at least partially based on previously-identified correlations between consumption of particular types and quantities of food items by a person and subsequent changes in that person's biometric parameters and/or health status.

In an example, a system for nutritional monitoring and management can recommend less consumption of foods or meals which are identified as unhealthy for a specific person or as generally unhealthy for people. For example, a system can recommend more consumption of foods or meals which are identified as healthy for a specific person or as generally healthy for people. In an example, a system can recommend (nearby) stores where healthy foods can be bought and/or (nearby) restaurants where healthy meals can be consumed. In an example, a system can provide shopping lists to help a person purchase healthy foods. In an example, a system can automatically order healthy foods for delivery to a person's home. In an example, a system can plan healthy meals for a person. In an example, a system can recommend healthy foods which can be substituted for unhealthy foods in a recipe or in a meal. In an example, a system can recommend restaurants which tend to serve healthy food as substitutes for a restaurant which tends to serve unhealthy food. In an example, a system can recommend amounts of (particular types of) food to be consumed in a given meal or during a period of time. In an example, a system can recommend that a person eat a particularly healthy food item on a periodic (e.g. daily) basis. For example, each day a system can say—“It's Hummus Time!” On the other hand, if a person is looking at unhealthy food, then the system can say—“U Can't Touch This!”

In an example, a system for nutritional monitoring and management can include an electromagnetic actuator, piezoelectric actuator, inflatable member, and/or pneumatic member which exerts pressure on a person's body in response to consumption of an unhealthy type and/or quantity of food. In an example a system can include an article of smart clothing or clothing accessory with an actuator, inflatable member, and/or pneumatic member which exerts pressure on a person's body in response to consumption of an unhealthy type and/or quantity of food. In an example, this clothing or accessory can be a shirt or pair of pants. In an example, this clothing or accessory can be a belt.

In an example, a system for nutritional monitoring and management can provide a person with one or more stimuli related to food consumption, wherein these stimuli are selected from the group consisting of: auditory stimulus (such as a voice message, alarm, buzzer, ring tone, or song); computer-generated speech; mild external electric charge or neural stimulation; periodic stimulus at a selected time of the day or week; phantom taste or smell; phone call; pre-recorded audio or video message by the person from an earlier time; television-based messages; and tactile, vibratory, or pressure-based stimulus. In an example, a system can provide negative stimuli in association with consumption of unhealthy types and quantities of food and/or provide positive stimuli in association with consumption of healthy types and quantities of food.

In an example, a system for nutritional monitoring and management can provide a person with stimuli to modify the person's eating behavior. In an example, a system can provide a person with visual, auditory, and/or haptic stimuli to modify the person's eating behavior. In an example, a system can provide negative stimuli which encourage a person to eat less unhealthy food and/or positive stimuli which encourage a person to eat more healthy food. In an example, a system can provide stimuli to encourage a person to avoid eating an unhealthy amount of food. In an example, a system can provide a negative stimulus associated with unhealthy food which is nearby and a person may eat. In an example, a system can provide a negative stimulus associated with unhealthy food which a person is eating or has just eaten. In an example, a system can provide a positive stimulus associated with healthy food which is nearby and a person may eat. In an example, a system can provide a positive stimulus associated with healthy food which a person is eating or has just eaten.

In an example, a system for nutritional monitoring and management can provide visual, auditory, haptic, or taste stimuli which actively discourage consumption of unhealthy food types or quantities. In an example, a system can provide visual, auditory, haptic, or taste stimuli which actively encourage consumption of healthy food types or quantities. In an example, a behavior-affecting stimulus can be provided before food consumption in order to influence a person's decision whether or not to consume a selected type or quantity of food. In an example, a behavior-affecting stimulus can be provided after food consumption in order to positively or negatively reinforce a person's consumption of a selected type or quantity of food. In an example, a system can provide a visual, auditory, haptic, or taste stimulus which makes unhealthy food less appealing to a person and/or makes healthy food more appealing to the person. In an example, the modality (e.g. visual, auditory, or haptic) of a behavior-affecting stimulus can be selected for a particular setting based analysis of environmental cues. For example, a more discreet stimulus modality can be selected in a public/social eating situation than in a home/individual eating situation. In an example, the modality (e.g. visual, auditory, or haptic) of a behavior-affecting stimulus can be selected for a particular person or in that particular setting based on past success of that modality in affecting the behavior of that particular person or in that particular setting.

In an example, a system for nutritional monitoring and management can display an appetite-influencing image in juxtaposition to a nearby food item in a person's field of view via augmented reality eyewear. In an example, a system can display an appetite-influencing image in juxtaposition to a nearby food item via the screen of a mobile device with augmented reality functionality. In an example, a system can display an unappetizing image in juxtaposition to unhealthy food and/or an appetizing image in juxtaposition to healthy food. In an example, a system can provide a person with real-time (or close to real-time) feedback on pending or recent food consumption choices. In an example, a system can display a person's historical nutritional intake data in graphic form, highlighting trends and implications. In an example, a system can provide a person with information about their progress toward a dietary goal. In an example, a system can connect a person's progress toward a dietary goal with a support group or social network. In an example, a system can connect a person's progress toward a dietary goal with a dietician or other healthcare professional.

In an example, a person can request that a system share information concerning the person's food consumption with friends, social networks, social media, healthcare professionals in order to receive feedback from those people to improve the person's food consumption choices and health. In an example, a system can provide a person with benchmark information by which to evaluate their food consumption and/or nutritional intake. In an example, a system can provide a person with reviews and/or ratings of selected meals, recipes, and/or food items. In an example, a system can provide a person with personalized dietary coaching and advice.

In an example, a system for nutritional monitoring and management can monitor, analyze, and provide feedback concerning a person's food consumption and/or nutritional intake. In an example, a system can provide a person with graphs showing historical trends with respect to their eating patterns and food consumption. In an example, a system can provide a person with personalized dietary recommendations and coaching based on automated analysis of the person's food consumption, changes in the person's biometric parameters, or the interaction thereof. In an example, a system can remind a person to take insulin before eating and/or recommend insulin dosage quantities based on types and/or quantities of food consumed.

In an example, a system for nutritional monitoring and management can help to prevent adverse diet-related conditions and diseases (such as diabetes). In an example, a system can help to treat and/or cure adverse diet-related conditions and diseases (such as diabetes). In an example, a system can provide therapy to treat adverse diet-related conditions and diseases (such as diabetes). In an example, a system can analyze types and quantities of food consumed by a person and provide recommended insulin doses for the person. In an example, recommended insulin doses can be at least partly based on identified associations between consumption of specific types and quantities of food in the past and subsequent changes in blood glucose levels following that food consumption. In an example, a system can be part of a closed-loop glucose monitoring and insulin delivery system. In an example, a system can be a closed-loop glucose monitoring and insulin delivery system. In an example, insulin can be delivered automatically by a closed-loop insulin therapy system.

In an example, a system for nutritional monitoring and management can include an implanted or wearable drug delivery device. In an example, a system can include an implanted or wearable device which dispenses a drug which modifies a person's appetite, food digestion, and/or food metabolism. In an example, a system can include an implanted or wearable insulin pump. In an example, a system can allow normal absorption of nutrients from a healthy type of food in a person's gastrointestinal tract, but can reduce absorption of nutrients from an unhealthy type of food by releasing an absorption-affecting substance. In an example, a system can include an implanted device which reduces absorption of nutrients from unhealthy types and/or quantities of food.

In an example, biometric information can be used to estimate blood glucose levels, but there is a lag between when food is consumed and when nutrients from this food enter a person's blood stream. In an example, a system such as is described in this disclosure can be combined with a wearable biometric device to form a system for predicting and estimating blood glucose levels. This system can use information on current blood glucose levels and also information on food that a person is consuming which can be helpful in predicting changes in glucose levels. In an example, a device to monitor nutritional intake can be wirelessly linked with a wearable device for non-invasive blood glucose monitoring as part of a system for estimating and/or predicting blood glucose levels. In an example data from the mobile device concerning the types and quantities of food that a person is eating can be used in a multivariate analysis, in combination with biometric information from a wearable device, to estimate and/or predict blood glucose levels more accurately than is possible with either food consumption monitoring or wearable biometric monitoring alone.

In an example, a system for nutritional monitoring and management can monitor and help to manage a person's food consumption and eating habits. In an example, a system can monitor and help to manage a person's food consumption triggers. In an example, a system can monitor a person's food consumption and provide the person with feedback to help the person manage their food consumption. In an example, a system can help a person to overcome food triggers and/or food addictions. In an example, food can include beverages as well as solid foods. In an example, a system can monitor a person's alcohol consumption and help the person to manage their alcohol consumption.

In an example, a system for nutritional monitoring and management can prompt a person to provide user input concerning identification of (nearby) food item types and/or quantities. In an example, this user input can be incorporated into multivariate analysis for determination of food item types and quantities. In an example, a system can prompt a person to enter user input (e.g. descriptions of food types and quantities) if the system detects that the person has begun eating (e.g. through motion sensors, image analysis, or other automated inputs) without providing such input.

In an example, user input from a person can be combined with automatically collected information (e.g. automatically collected images and spectroscopic analysis) concerning food item types and quantities for multivariate estimation of food item types and quantities. In an example, if analysis of automatically collected information is insufficient to determine food types and quantities with sufficient accuracy or certainty, then the system can prompt a person to enter user input (e.g. descriptions of food types and quantities) as well. In an example, a system can use Bayesian statistical methods to update analysis of food types and quantities with information from multiple (automated and manual) sources, sensors, and modalities until a desired level of measurement accuracy or certainty is obtained.

In an example, a system for nutritional monitoring and management can allow normal sensory perception of healthy food, but modifies the taste and/or smell of unhealthy food. In an example, a system can release a taste and/or smell modifying substance into a person's oral cavity and/or nasal passages. In an example, a system can allow normal sensory perception of a healthy quantity of food, but can modify the taste and/or smell of an unhealthy quantity of food by releasing a taste and/or smell modifying substance into a person's oral cavity and/or nasal passages. In an example, a system can release a substance with a strong flavor into a person's oral cavity when the person consumes an unhealthy type and/or quantity of food. In an example, a system can release a substance with a strong smell into a person's nasal passages when the person consumes an unhealthy type and/or quantity of food.

In an example, a system for nutritional monitoring and management can cause a person to experience an unpleasant virtual taste and/or smell when the person consumes an unhealthy type or quantity of food. In an example, a phantom taste or smell can be triggered by delivering electromagnetic energy to afferent nerves which innervate a person's tongue and/or nasal passages. In an example, a system can cause temporary dysgeusia when a person consumes an unhealthy type or quantity of food. In an example, a system can cause a person to experience reduced taste and/or smell when the person consumes an unhealthy type or quantity of food by delivering electromagnetic energy to afferent nerves which innervate a person's tongue and/or nose.

In an example, a system for nutritional monitoring and management can send a communication or message to a person who is wearing a device. In an example, a system can send nutritional information concerning food that a person is near, food that the person is purchasing, food that the person is ordering, and/or food that the person is eating. This nutritional information can include food ingredients, nutrients, and/or calories. In an example, a system can send information concerning the likely health effects of consuming food that a person is near, food that the person is purchasing, food that the person is ordering, and/or food that the person has already starting consuming. In an example, a system can communicate food information in text form. In an example, a communication can recommend a healthier substitute for unhealthy food which a person is considering purchasing, ordering, and/or consuming.

In an example, a system for nutritional monitoring and management can send a communication to a person other than the person who is wearing a device. In an example, this other person can provide encouragement and support for the person wearing the device to eat less unhealthy food and/or to eat more healthy food. In an example, this other person can be a friend, support group member, family member, or a health care provider. In an example, this device could send a text to Kevin Bacon, or someone who knows him, or someone who knows someone who knows him, or someone who knows someone who knows someone who knows him. In an example, a system can connect with a social network and/or an internet-based support group. In an example, a system can engage a person's friends to encourage the person to reduce consumption of unhealthy types and/or quantities of food (and increase consumption of healthy food) in order to achieve personal health goals. In an example, a system can encourage a person to compete with people in a peer group with respect to achievement of health goals. In an example, a system can function as a virtual dietary health coach.

In an example, a system for nutritional monitoring and management can include a battery or other power source. In an example, a system can include a power transducer which generates electrical energy from body heat or motion. In an example, a system can comprise a power management unit which regulates the amount of power used the system based on whether or not the person is eating. In an example, a system can comprise a power management unit which regulates the amount of power used the system based on whether or not the person is sleeping. In an example, a system can be set in low-power mode when a person is not eating or is sleeping. In an example, this system can comprise a touch screen and/or display. In an example, this system can comprise a keypad or keyboard. In an example, this system can comprise a camera and microphone.

In an example, a system for nutritional monitoring and management can comprise one or more devices selected from the group consisting of: augmented reality device, bioimpedance monitor, blood pressure monitor, body temperature sensor, camera, chewing-sound sensor, continuous glucose monitor, ECG monitor, ear bud or pod, electromagnetic energy sensor, EMG sensor, GPS receiver, heart rate monitor, intra-oral sensor, microphone, mobile device, mobile EEG device, motion sensor (e.g. accelerometer and gyroscope); pacemaker, smart clothing, smart eyewear, smart necklace, smart ring, smart watch, spectroscopic sensor, and sweat analysis device.

In an example, a system for nutritional monitoring and management can comprise a data processing unit, memory, wireless data transmitter, and wireless data receiver. In an example, analysis of food types and quantities by a system can be done by a local data processor which is in a handheld or wearable device. In an example, a handheld or wearable device can transmit data to a remote data processor, wherein analysis of food types and quantities is done. In an example, data can be transmitted from a handheld or wearable device to a remote data processor via the internet. In an example, this system can comprise a first data processing unit (e.g. in a wearable or handheld mobile device), a data transmitter, and a second data processing unit in a remote location (e.g. in electromagnetic communication with the mobile device via the data transmitter). In an example, the second data processing unit can be in the cloud.

In an example, a system for nutritional monitoring and management can include a handheld device and a wearable device which are in wireless electromagnetic communication with each other. In an example, a system can include a local (e.g. handheld or wearable) device which is in wireless electromagnetic communication with a remote (e.g. cloud-based) data processor. In an example, different devices and/or processors in a system can exchange information via wireless electromagnetic communication, including sensor data, analysis results, notifications, text messages, and voice messages.

In an example, a system for nutritional monitoring and management can include augmented reality eyewear or other smart eyewear. In an example, a system can include buttons or a keypad. In an example, a system can include one or more electromagnet energy sensors selected from the group consisting of: EEG sensor, EMG sensor, and other electromagnetic sensor. In an example, a system can include one or more energy related components selected from the group consisting of: battery or other power source, power transducer, and thermal energy transducer. In an example, a system can include one or more light energy components selected from the group consisting of: display screen, graphic display, handheld spectroscopy sensor, laser pointer, LCD display, light emitter, light projector, light receiver, optical diffuser, optical sensor, spectroscopic sensor, and touch screen.

In an example, a system for nutritional monitoring and management can include one or more sensor components selected from the group consisting of: chemical sensor, gesture recognition component, GPS component, and motion sensor (e.g. accelerometer and gyroscope). In an example, a system can include one or more sound-related components selected from the group consisting of: microphone, speaker, and speech recognition component. In an example, a system can include one or more wearable and/or handheld devices selected from the group consisting of: ear bud, fitness band, mobile EEG device, smart finger ring, smart necklace, smart phone, smart watch, and wrist band. In an example, a system for nutritional monitoring and management can include one or more data-related components selected from the group consisting of: data analysis component, food database, local data processor, memory, remote data processor, wireless data receiver, and wireless data transmitter.

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; and (c) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; and (d) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; and (e) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (e) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; and (f) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (e) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (f) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); and (g) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (e) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (f) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); (g) an active stimulus mechanism which automatically responds to food consumption by the person, wherein the active stimulus mechanism automatically modifies a person's physiological processes (e.g. by delivering a therapeutic agent, such as insulin, into the person's body; by delivering a therapeutic pattern of electromagnetic energy to a selected portion of the person's body, such as the vagus nerve; or by delivering a taste-modifying substance into a person's mouth); and (h) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (d) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (e) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); (f) an active stimulus mechanism which automatically responds to food consumption by the person, wherein the active stimulus mechanism automatically modifies a person's physiological processes (e.g. by delivering a therapeutic agent, such as insulin, into the person's body; by delivering a therapeutic pattern of electromagnetic energy to a selected portion of the person's body, such as the vagus nerve; or by delivering a taste-modifying substance into a person's mouth); and (g) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (d) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (e) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); and (f) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (d) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; and (e) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; and (d) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (e) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); (f) an active stimulus mechanism which automatically responds to food consumption by the person, wherein the active stimulus mechanism automatically modifies a person's physiological processes (e.g. by delivering a therapeutic agent, such as insulin, into the person's body; by delivering a therapeutic pattern of electromagnetic energy to a selected portion of the person's body, such as the vagus nerve; or by delivering a taste-modifying substance into a person's mouth); and (g) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (e) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); and (f) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; and (e) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (e) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); (f) an active stimulus mechanism which automatically responds to food consumption by the person, wherein the active stimulus mechanism automatically modifies a person's physiological processes (e.g. by delivering a therapeutic agent, such as insulin, into the person's body; by delivering a therapeutic pattern of electromagnetic energy to a selected portion of the person's body, such as the vagus nerve; or by delivering a taste-modifying substance into a person's mouth); and (g) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a fiducial component which displays objects in images of food items which help to calibrate the distance, size, shape, color, and/or brightness of the food items; wherein the fiducial component is selected from the group consisting of: an object with (markings of) known size, shape, and/or colors which is placed near the food items; a light emitter (e.g. low-power laser) which projects a light pattern with known size, shape, and/or colors on or near the food items; and a mobile device with a screen which is placed near the food items and displays an image on the screen with known size, shape, and/or colors; (d) a wearable biometric sensor which collects biometric data concerning a person whose nutritional intake is being monitored, wherein the biometric sensor is selected from the group consisting of: motion sensor (e.g. accelerometer, gyroscope, and/or compass), electromagnetic energy sensor (e.g. impedance sensor, EMG sensor, EKG sensor), spectroscopic sensor (e.g. spectrometer) and/or photoplethysmographic sensor, sound sensor (e.g. microphone, chew sensor, swallow sensor), and chemical sensor (e.g. sweat sensor, saliva sensor); wherein data from the biometric sensor is used for one or more functions selected from the group consisting of: recognizing when the person is eating in order to automatically activate the system to take an action (e.g. recording images or monitoring sounds) to help identify food item types and/or estimate food item quantities; recognizing when the person is eating in order to automatically prompt the person to take an action (e.g. recording images or entering food descriptions) to help identify food item types and/or estimate food item quantities; and identifying relationships between consumption of selected food item types and/or food item quantities by the person and subsequent changes in the person's biometric parameters (e.g. glucose level, blood pressure, lactic acid level, or oxygen level); and wherein the biometric sensor is part of a device selected from the group consisting of: smart watch or other wrist-worn device, smart finger ring, smart armband, smart eyewear, smart earwear, smart necklace or pendant, smart button, smart belt, smart garment, adhesive sensor patch, mobile EEG device, and continuous glucose monitor; (e) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); and (f) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (d) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); (e) an active stimulus mechanism which automatically responds to food consumption by the person, wherein the active stimulus mechanism automatically modifies a person's physiological processes (e.g. by delivering a therapeutic agent, such as insulin, into the person's body; by delivering a therapeutic pattern of electromagnetic energy to a selected portion of the person's body, such as the vagus nerve; or by delivering a taste-modifying substance into a person's mouth); and (f) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; (d) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); and (e) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a smart utensil, dish, plate, or beverage holder which collects data concerning food item quantities consumed by a person; wherein the smart utensil, dish, plate, or beverage holder collects data by one or more means selected from the group consisting of: measuring the number of forkfulls, spoonfulls, bites and/or sips taken by a person based on motion (e.g. upward and tilting motion) of a smart utensil or beverage holder; estimating the weight of forkfulls, spoonfulls, bites and/or sips taken by a person based on motion and/or force exerted by food on a smart utensil or beverage holder; estimating the cumulative quantity of food items consumed by a person (e.g. during a particular meal) by measuring changes in the weight of food on a disk or plate; and using chemical analysis to help to identify the type and/or composition of food in contact with the smart utensil, dish, plate, or beverage holder; and (d) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); (d) an active stimulus mechanism which automatically responds to food consumption by the person, wherein the active stimulus mechanism automatically modifies a person's physiological processes (e.g. by delivering a therapeutic agent, such as insulin, into the person's body; by delivering a therapeutic pattern of electromagnetic energy to a selected portion of the person's body, such as the vagus nerve; or by delivering a taste-modifying substance into a person's mouth); and (e) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: (a) a camera which records images of food items, wherein the images are analyzed to help identify food item types and/or estimate food item quantities, wherein food includes beverages as well as solid food, and wherein the camera is part of a device selected from the group consisting of: smart phone, smart watch or other wrist-worn device, smart finger ring, smart eyewear, electronic tablet, smart earwear, smart necklace or pendant, smart button, and dedicated handheld food identification device; (b) a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; wherein changes in the spectral distribution of the light caused by interaction with food items are used to help identify food item types and/or compositions; and wherein the spectroscopic sensor is part of a device selected from the group consisting of: smart phone, smart watch or other wrist-worn device, smart finger ring, smart eyewear, electronic tablet, smart earwear, smart necklace or pendant, smart button, and dedicated handheld food identification device; (c) a passive feedback mechanism which provides passive feedback to a person concerning the type, quantity, nutritional content, and/or health implications of food items; wherein this passive feedback is selected from the group consisting of: visual feedback (e.g. text, graphics, or images displayed on a screen or in augmented reality); sound feedback (e.g. sound, song, or voice); and haptic feedback (e.g. vibration, pressure, or delivery of electromagnetic energy); and (d) one or more other components selected from the group consisting of: data processor; data transmitter; data receiver; battery; GPS module (e.g. identifying the location of food acquisition, preparation, or consumption); clock (e.g. identifying the time of day of food consumption); calendar (e.g. identifying day of the week, holidays, or special events); voice recognition interface (e.g. to recognize voice-based food descriptions); touch-screen interface (e.g. to recognize touch-based menu-driven or text-based food descriptions); gesture recognition interface (e.g. to recognize gesture-based menu-driven food descriptions); and EEG interface (e.g. to recognize selected EEG patterns).

In an example, a system for nutritional monitoring and management can comprise: a camera which records images of food items, wherein the images are analyzed to help identify food item types and/or estimate food item quantities, and wherein food includes beverages as well as solid food; a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; and wherein changes in the spectral distribution of the light caused by interaction with food items are used to help identify food item types and/or compositions; and one or more other components selected from the group consisting of: data processor; data transmitter; data receiver; battery; GPS module; clock; calendar; voice recognition interface; touch-screen interface; gesture recognition interface; and EEG interface.

In an example, the camera can be part of a smart phone. In an example, the camera can be part of a smart watch or other wrist-worn device. In an example, the camera can be part of a smart finger ring. In an example, the camera can be part of augmented reality eyewear or other smart eyewear. In an example, the camera can be part of a smart necklace or pendant. In an example, the camera can be part of a dedicated handheld food identification device. In an example, the spectroscopic sensor can be part of a smart watch or other wrist-worn device. In an example, the spectroscopic sensor can be part of a dedicated handheld food identification device.

In an example, a system for nutritional monitoring and management can comprise: a camera which records images of food items, wherein the images are analyzed to help identify food item types and/or estimate food item quantities, and wherein food includes beverages as well as solid food; a spectroscopic sensor which collects spectral data concerning light reflected from or absorbed by food items; wherein the spectral data is used to help identify food item types and/or compositions; wherein the spectroscopic sensor further comprises a light emitter which emits light toward food items and a light receiver which receives the light after it has been reflected by or passed through the food items; and wherein changes in the spectral distribution of the light caused by interaction with food items are used to help identify food item types and/or compositions; a fiducial component which displays objects in images of food items which help to calibrate the distance, size, shape, color, and/or brightness of the food items; and one or more other components selected from the group consisting of: data processor; data transmitter; data receiver; battery; GPS module; clock; calendar; voice recognition interface; touch-screen interface; gesture recognition interface; and EEG interface.

In an example, the camera can be part of a smart phone. In an example, the camera can be part of a smart watch or other wrist-worn device. In an example, the camera can be part of a smart finger ring. In an example, the camera can be part of augmented reality eyewear or other smart eyewear. In an example, the camera can be part of a smart necklace or pendant. In an example, the camera can be part of a dedicated handheld food identification device. In an example, the spectroscopic sensor can be part of a smart watch or other wrist-worn device. In an example, the spectroscopic sensor can be part of a dedicated handheld food identification device. In an example, the fiducial component can be a light emitter which projects a light pattern with known size, shape, and/or colors on or near the food items. In an example, the fiducial component can be a mobile device with a screen which is placed near the food items and displays an image on the screen with known size, shape, and/or colors.

In an example, a system for nutritional monitoring and management can comprise: a handheld device; a camera in the handheld device which captures images of a meal with multiple types of food; wherein meal images are analyzed to identify different types of food in the meal based on variation and boundaries in food shapes, sizes, colors, and textures; and a spectroscopic sensor in the handheld device which emits light beams toward food and receives the light beams after the light beams have been reflected from (or passed through) the food; wherein a person is prompted by a virtual pointer in augmented reality to direct light beams toward different locations on the meal which are associated with different types of food identified by analysis of the meal images; wherein food images and changes in the spectra of the light beams caused by reflection from (or passage through) different types of food are analyzed together (in a multivariate manner) in order to identify food type, food composition (e.g. nutritional composition), and/or food quantity for each type of food in the meal.

In another example, a system for nutritional monitoring and management can comprise: a handheld device; a camera in the handheld device which captures images of food; a spectroscopic sensor in the handheld device which emits light beams toward the food and receives the light beams after the light beams have been reflected from (or passed through) the food; wherein the visible portion of the spectrum of light beams emitted from the spectroscopic sensor creates a visible light pattern on (or near) the food and wherein the size, shape, and/or keystone distortion of this visible light pattern is used as a fiducial marker to estimate food size, distance, and/or orientation relative to the device; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld device; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition, and/or quantity; and wherein one or more eating-related objects (e.g. bowl, chopsticks, cup, fork, glass, knife, mug, napkin, placemat, plate, or spoon) are identified in food images to help estimate food size. Alternatively, a system can comprise: a handheld device; a camera in the handheld device which captures images of food at a first time and at a second time; a spectroscopic sensor in the handheld device which emits light beams toward food and receives the light beams after the light beams have been reflected from (or passed through) food at the first time and at the second time; and a motion sensor which tracks hand-to-mouth motions, chewing motions, and/or swallowing motions; wherein food images captured by the camera, changes in the spectra of the light beams caused by reflection from (or passage through) food, and hand-to-mouth motions, chewing motions, or swallowing motions are analyzed together (e.g. in multivariate analysis) to identify the type, composition (e.g. nutritional composition), and/or quantity of food eaten by the person holding or wearing the device.

In an example, a system for nutritional monitoring and management can comprise: a handheld device; a camera in the handheld device which captures images of food, wherein the food images are automatically analyzed to identify food type and/or measure food quantity; and a spectroscopic sensor in the handheld device which emits light beams toward food and receives the light beams after the light beams have been reflected from (or passed through) food, wherein changes in the spectra of the light beams caused by reflection from (or passage through) food are analyzed to identify food type and/or composition; and where the device prompts a person with a sound, vibration, or light to use the camera and/or the spectroscopic sensor at multiple times while the person is eating a meal in order to measure changes in the amount of food remaining (and infer how much food the person has actually consumed) and to measure the composition of different layers (or parts) of the food. Alternatively, a system can comprise: a handheld device; a spectroscopic sensor in the handheld device which emits light beams toward food and receives the light beams after the light beams have been reflected from (or passed through) the food; a camera in the handheld device which captures images of the food; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. In an example, the spectroscopic sensor can comprise a light emitter which emits light beams toward food and a light receiver which receives the light beams after the light beams have been reflected from (or passed through) the food.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; a first laser which projects a first coherent light beam toward the food; and a second laser which projects a second coherent light beam toward the food; wherein the first and second light beams form a projected light pattern on (or near) the food which serves as a fiducial marker to help estimate food size, distance, and/or orientation; and wherein changes in the spectra of light beams caused by reflection from (or passage through) the food and food images captured by the camera are analyzed to identify food type, composition, and/or quantity. In another example, a system can comprise: a handheld device; a spectroscopic sensor in the handheld device which emits light beams toward food and receives the light beams after the light beams have been reflected from (or passed through) food; a camera in the handheld device which captures images of the food; and a laser which projects an arcuate (e.g. circular, elliptical, or egg-shaped) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a laser which projects an quadrilateral grid of light on (or near) the food, wherein the grid serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. Alternatively, a system can comprise: a handheld device; a spectroscopic sensor in the handheld device which emits light beams toward food and receives the light beams after the light beams have been reflected from (or passed through) food; a camera in the handheld device which captures images of the food; and a light pattern projector which projects a polygonal light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a scanning (e.g. moving) laser which projects an arcuate (e.g. circular, elliptical, or egg-shaped) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. Alternatively, a system can comprise: a handheld device; a spectroscopic sensor in the handheld device which emits light beams toward food and receives the light beams after the light beams have been reflected from (or passed through) food; a camera in the handheld device which captures images of the food; and one or more lasers which project a pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld device; a spectroscopic sensor in the handheld device which emits light beams toward food and receives the light beams after the light beams have been reflected from (or passed through) the food; a camera in the handheld device which captures images of the food; wherein the food images and changes in the spectra of the light beams caused by reflection from (or passage through) the food are analyzed together (in a multivariate manner) in order to identify the food's type and/or measure the food's composition; wherein the food images are analyzed to measure food quantity; and a motion sensor; wherein the handheld device is waived over the food so that the spectroscopic sensor reflects beams from the food at multiple locations and the camera creates images of the food from multiple perspectives. In another example, a system can comprise: a handheld device; a spectroscopic sensor in the handheld device which emits light beams toward food and receives the light beams after the light beams have been reflected from the food; a camera in the handheld device which captures images of the food; wherein the food images and changes in the spectra of the light beams caused by reflection from the food are analyzed together (in a multivariate manner) in order to identify the food's type and/or measure the food's composition; wherein the food images are analyzed to measure food quantity; a range finder (e.g. an infrared range finder) which measures the distance from the handheld device to the food; and wherein the spectroscopic sensor is automatically triggered at a selected distance from the food to direct and receive reflected light beams.

In an example, a system for nutritional monitoring and management can comprise: a handheld device; a spectroscopic sensor in the handheld device which emits light beams toward food and receives the light beams after the light beams have been reflected from (or passed through) the food; a camera in the handheld device which captures images of the food; wherein the food images and changes in the spectra of the light beams caused by reflection from (or passage through) the food are analyzed together using multivariate statistical methods in order to identify the food's type and/or measure the food's composition; and wherein the food images are analyzed to measure food quantity. Alternatively, a system can comprise: a handheld device; a camera in the handheld device which captures images of food, wherein the food images are automatically analyzed to identify food type and/or measure food quantity; and a spectroscopic sensor in the handheld device which emits light beams toward food and receives the light beams after the light beams have been reflected from (or passed through) food, wherein changes in the spectra of the light beams caused by reflection from (or passage through) food are analyzed to identify food type and/or composition; a wearable device; a sound sensor in the wearable device which tracks chewing or swallowing sounds; wherein the device prompts a person with a sound, vibration, or light to use the camera and/or the spectroscopic sensor based on chewing or swallowing sounds.

A system can be embodied in: a handheld device; a camera in the handheld device which captures images of a meal with multiple types of food; wherein meal images are analyzed to identify different types of food in the meal based on variation and boundaries in food shapes, sizes, colors, and textures; and a spectroscopic sensor in the handheld device which emits light beams toward food and receives the light beams after the light beams have been reflected from (or passed through) the food; wherein a person is prompted by a projected light pointer to direct light beams toward different locations on the meal which are associated with different types of food based on analysis of the meal images; wherein food images and changes in the spectra of the light beams caused by reflection from (or passage through) different types of food are analyzed together (in a multivariate manner) in order to identify, for each type of food in the meal, food type, food composition, and/or food quantity. Alternatively, a system can comprise: a handheld device; a camera in the handheld device which captures images of food; a spectroscopic sensor in the handheld device which emits light beams toward food and receives the light beams after the light beams have been reflected from (or passed through) food; wherein the camera and spectroscopic sensor are both directed toward a first food in a meal at a first point in time; wherein the camera and spectroscopic sensor are both directed toward a second food in a meal at a second point in time; wherein food images captured by the camera and changes in the spectra of the light beams caused by reflection from (or passage through) food are analyzed to identify the compositions and quantities of the first and second foods.

In an example, a system for nutritional monitoring and management can comprise: a handheld device; a camera in the handheld device which captures images of food; a spectroscopic sensor in the handheld device which emits light beams toward the food and receives the light beams after the light beams have been reflected from (or passed through) the food; wherein a light pattern formed by the projection of light beams from the spectroscopic sensor on (or near) the food is used as a fiducial marker to estimate food size, distance, and/or orientation relative to the device; and wherein changes in the spectra of light beams caused by reflection from (or passage through) the food and food images captured by the camera are analyzed to identify food type, composition (e.g. nutritional composition), and/or quantity. In another example, a system can comprise: a handheld device; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are identified in food images to estimate food distance.

In an example, a system for nutritional monitoring and management can comprise: a handheld device; a camera in the handheld device which captures images of food at a first time and at a second time; a spectroscopic sensor in the handheld device which emits light beams toward food and receives the light beams after the light beams have been reflected from (or passed through) food at the first time and at the second time; and a motion sensor which tracks hand-to-mouth motions, chewing motions, and/or swallowing motions; wherein data from the camera, the spectroscopic sensor, and the motion sensor are analyzed together (e.g. in multivariate analysis) to identify the type, composition, and/or quantity of food eaten by the person holding or wearing the device. Alternatively, a system can comprise: a handheld device; a camera in the handheld device which captures images of food, wherein the food images are automatically analyzed to identify food type and/or measure food quantity; and a spectroscopic sensor in the handheld device which emits light beams toward food and receives the light beams after the light beams have been reflected from (or passed through) food, wherein changes in the spectra of the light beams caused by reflection from (or passage through) food are analyzed to identify food type and/or composition; and where the device prompts a person to use the camera and/or the spectroscopic sensor at regular intervals while the person is eating a meal in order to measure changes in the amount of food remaining (and infer how much food the person has actually consumed) and to measure the composition of different layers (or parts) of the food.

In an example, a system for nutritional monitoring and management can comprise: a handheld device; a camera in the handheld device which captures images of food, wherein the food images are automatically analyzed to identify food type and/or measure food quantity; and a spectroscopic sensor in the handheld device which emits light beams toward food and receives the light beams after the light beams have been reflected from (or passed through) food, wherein changes in the spectra of the light beams caused by reflection from (or passage through) food are analyzed to identify food type and/or composition; a wearable motion sensor which tracks hand-to-mouth or chewing motions; and where the device prompts a person with a sound, vibration, or light to use the camera and/or the spectroscopic sensor at multiple times based on the number or timing of hand-to-mouth or chewing motions in order to measure changes in the amount of food remaining (and infer how much food the person has actually consumed) and to measure the composition of different layers (or parts) of the food. Alternatively, a system can comprise: a handheld device; a motion sensor in the handheld device; a spectroscopic sensor in the handheld device which is triggered to emit light beams toward food and receives the light beams after the light beams have been reflected from (or passed through) the food from multiple selected locations in three-dimensional space based in part on data from the motion sensor; a camera in the handheld device which is triggered to capture images of the food from multiple selected locations in three-dimensional space based in part on data from the motion sensor; wherein the food images and changes in the spectra of the light beams caused by reflection from (or passage through) the food are analyzed together (in a multivariate manner) in order to identify the food's type and/or the food's composition; and wherein food images from different perspectives are used to model the food in three dimensions in order to measure food quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; a first laser which projects a first coherent light beam toward the food; a second laser which projects a second coherent light beam toward the food; wherein the distance between the locations of incidence of the first and second light beams on (or near) the food is used to estimate food size, distance, and/or orientation relative to the device; and wherein changes in the spectra of light beams caused by reflection from (or passage through) the food and food images captured by the camera are analyzed to identify food type, composition, and/or quantity.

In another example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a laser which projects an array of nested rings of light (or near) the food, wherein the array serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a laser which projects an quadrilateral grid of light on (or near) the food, wherein the size and/or keystone distortion of the (quadrilateral elements in the) grid serves as a fiducial marker to help estimate food size, distance, and/or orientation; and wherein changes in the spectra of light beams caused by reflection from (or passage through) the food and food images captured by the camera are analyzed to identify food type, composition, and/or quantity. Alternatively, a system can comprise: a handheld device; a spectroscopic sensor in the handheld device which emits light beams toward food and receives the light beams after the light beams have been reflected from (or passed through) food; a camera in the handheld device which captures images of the food; and a light pattern projector which projects an arcuate (e.g. circular or keystone-distorted circular) light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a scanning (e.g. moving) laser which projects an matrix (e.g. dot matrix or linear grid) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. Alternatively, a system can comprise: a handheld device; a spectroscopic sensor in the handheld device which emits light beams toward food and receives the light beams after the light beams have been reflected from (or passed through) the food, wherein changes in the spectra of the light beams caused by reflection from (or passage through) the food are analyzed in order to identify the food's type and/or measure the food's composition; a camera in the handheld device which captures images of the food, wherein the food images are analyzed to measure food quantity.

A system can be embodied in: a handheld device; a spectroscopic sensor in the handheld device which emits light beams toward food and receives the light beams after the light beams have been reflected from the food; a camera in the handheld device which captures images of the food; wherein the food images and changes in the spectra of the light beams caused by reflection from the food are analyzed together (in a multivariate manner) in order to identify the food's type and/or measure the food's composition; wherein the food images are analyzed to measure food quantity; a range finder (e.g. an infrared range finder) which measures the distance from the handheld device to the food; and wherein the camera is automatically triggered at a selected distance from the food to capture images of the food. In another example, a system can comprise: a handheld device; a spectroscopic sensor in the handheld device which emits light beams toward food and receives the light beams after the light beams have been reflected from (or passed through) the food; two cameras in the handheld device which create stereoscopic images of the food; wherein the shape, size, color, tone, brightness, and/or texture of food in the food images and changes in the spectra of the light beams caused by reflection from (or passage through) the food are analyzed together (in a multivariate manner) in order to identify the food's type and/or the food's composition; and wherein the stereoscopic food images are analyzed in order to measure food quantity.

In an example, a system for nutritional monitoring and management can comprise: a handheld device; a camera in the handheld device which captures images of food; a spectroscopic sensor in the handheld device which emits light beams toward food and receives the light beams after the light beams have been reflected from (or passed through) food; wherein the person directs the camera and the spectroscopic sensor toward a first food in a meal at a first point in time; wherein the person directs the camera and the spectroscopic sensor toward a second food in a meal at a second point in time; wherein food images captured by the camera and changes in the spectra of the light beams caused by reflection from (or passage through) food are analyzed to identify the compositions and quantities of the first and second foods. Alternatively, a system can comprise: a handheld device; a camera in the handheld device which captures images of food; a spectroscopic sensor in the handheld device which emits light beams toward food and receives the light beams after the light beams have been reflected from (or passed through) food; and a motion sensor which tracks hand-to-mouth motions, chewing motions, and/or swallowing motions; wherein data from the camera, the spectroscopic sensor, and the motion sensor are analyzed together (e.g. in multivariate analysis) to identify the type, composition (e.g. nutritional composition), and/or quantity of food eaten by the person holding or wearing the device.

In an example, a system for nutritional monitoring and management can comprise: a handheld device; a camera in the handheld device which captures images of food; a spectroscopic sensor in the handheld device which emits light beams toward the food and receives the light beams after the light beams have been reflected from (or passed through) the food; wherein light beams emitted from the spectroscopic sensor create a projected light pattern on (or near) the food and wherein the size, shape, and/or keystone distortion of this projected light pattern is used to estimate food size, distance, and/or orientation relative to the device; and wherein changes in the spectra of light beams caused by reflection from (or passage through) the food and food images captured by the camera are analyzed to identify food type, composition, and/or quantity. Alternatively, a system can comprise: a handheld device; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are used to help estimate food size.

In an example, a system for nutritional monitoring and management can comprise: a handheld device; a camera in the handheld device which captures images of food at a first time and at a second time; and a spectroscopic sensor in the handheld device which emits light beams toward food and receives the light beams after the light beams have been reflected from (or passed through) food at the first time and at the second time; wherein food images captured by the camera and changes in the spectra of the light beams caused by reflection from (or passage through) food are analyzed together (e.g. in multivariate analysis) to identify the type, composition (e.g. nutritional composition), and/or quantity of food eaten by the person holding or wearing the device. In another example, a system can comprise: a handheld device; a camera in the handheld device which captures images of food, wherein the food images are automatically analyzed to identify food type and/or measure food quantity; and a spectroscopic sensor in the handheld device which emits light beams toward food and receives the light beams after the light beams have been reflected from (or passed through) food, wherein changes in the spectra of the light beams caused by reflection from (or passage through) food are analyzed to identify food type and/or composition; a wearable motion sensor which tracks hand-to-mouth or chewing motions; and where the device prompts a person with a sound, vibration, or light to use the camera and/or the spectroscopic sensor based on hand-to-mouth or chewing motions.

In an example, a system for nutritional monitoring and management can comprise: a handheld device; a motion sensor in the handheld device; a spectroscopic sensor in the handheld device which is triggered to emit light beams toward food and receives the light beams after the light beams have been reflected from (or passed through) the food from multiple selected locations in three-dimensional space based in part on data from the motion sensor; a camera in the handheld device which is triggered to capture images of the food from multiple selected locations in three-dimensional space based in part on data from the motion sensor; wherein the food images and changes in the spectra of the light beams caused by reflection from (or passage through) the food are analyzed together (in a multivariate manner) in order to identify the food's type and/or the food's composition; and wherein food images from different perspectives are used to measure food quantity. Alternatively, a system can comprise: a handheld device; a range finder (e.g. a range finder (e.g. an infrared range finder)) in the handheld device which measures the distance from the handheld device to food; a motion sensor (e.g. an accelerometer and a gyroscope) in the handheld device; a spectroscopic sensor in the handheld device which is triggered to emit light beams toward food and receives the light beams after the light beams have been reflected from (or passed through) the food from multiple selected locations in three-dimensional space based in part on data from the range finder and the motion sensor; a camera in the handheld device which is triggered to capture images of the food from multiple selected locations in three-dimensional space based in part on data from the range finder and the motion sensor; wherein the food images and changes in the spectra of the light beams caused by reflection from (or passage through) the food are analyzed together (in a multivariate manner) in order to identify the food's type and/or the food's composition; and wherein food images are used to measure food quantity.

In an example, a system for nutritional monitoring and management can comprise: a handheld device; a spectroscopic sensor in the handheld device which emits light beams toward food and receives the light beams after the light beams have been reflected from (or passed through) the food; a camera in the handheld device which captures images of the food from different perspectives and angles as the handheld device is moved; wherein the shape, size, color, tone, brightness, and/or texture of food in the food images and changes in the spectra of the light beams caused by reflection from (or passage through) the food are analyzed together (in a multivariate manner) in order to identify the food's type and/or the food's composition; and wherein food images from different perspectives and angles are used to model the food in three dimensions in order to measure food quantity. Alternatively, a system can comprise: a handheld device; a spectroscopic sensor in the handheld device which emits light beams toward food and receives the light beams after the light beams have been reflected from (or passed through) food; a camera in the handheld device which captures images of the food; a first laser which projects a first coherent light beam toward the food; a second laser which projects a second coherent light beam toward the food; a third laser which projects a third coherent light beam toward the food; wherein the distances and angles between the locations of incidence of the first, second, and third light beams on (or near) the food are used to estimate food size, distance, and/or orientation; and wherein changes in the spectra of light beams caused by reflection from (or passage through) the food and food images captured by the camera are analyzed to identify food type, composition, and/or quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a laser which projects an array of nested rings of light (or near) the food, wherein the size and distortion of rings in the array is used to help estimate food size, distance, and/or orientation; and wherein changes in the spectra of light beams caused by reflection from (or passage through) the food and food images captured by the camera are analyzed to identify food type, composition (e.g. nutritional composition), and/or quantity. In another example, a system can comprise: a handheld device; a spectroscopic sensor in the handheld device which emits light beams toward food and receives the light beams after the light beams have been reflected from (or passed through) food; a camera in the handheld device which captures images of the food; and a light pattern projector which projects a pattern of light on (or near) the food; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a scanning (e.g. moving) laser which projects a pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity. Alternatively, a system can comprise: a handheld device; a spectroscopic sensor in the handheld device which emits light beams toward food and receives the light beams after the light beams have been reflected from (or passed through) food; a camera in the handheld device which captures images of the food; and a scanning laser which projects an arcuate (e.g. circular) light pattern toward the food; wherein the shape, size, and/or keystone distortion of the projected light pattern on (or near) the food is used to estimate food size, distance, and/or orientation relative to the device; and wherein changes in the spectra of light beams caused by reflection from (or passage through) the food and food images captured by the camera are analyzed to identify food type, composition (e.g. nutritional composition), and/or quantity.

A system can be embodied in: a handheld device; a spectroscopic sensor in the handheld device which emits light beams toward food and receives the light beams after the light beams have been reflected from (or passed through) the food; a camera in the handheld device which captures images of the food; wherein the field of view of the camera overlaps the projection path of light beams from the spectroscopic sensor; area wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. Alternatively, a system can comprise: a handheld device; a spectroscopic sensor in the handheld device which emits light beams toward food and receives the light beams after the light beams have been reflected from the food; a camera in the handheld device which captures images of the food; wherein the food images and changes in the spectra of the light beams caused by reflection from the food are analyzed together (in a multivariate manner) in order to identify the food's type and/or measure the food's composition; wherein the food images are analyzed to measure food quantity; a range finder which measures the distance from the handheld device to the food; and wherein the spectroscopic sensor and/or the camera is automatically triggered at a selected distance from the food.

In an example, a system for nutritional monitoring and management can comprise: a handheld device; a spectroscopic sensor in the handheld device which emits light beams toward food and receives the light beams after the light beams have been reflected from (or passed through) the food; two cameras in the handheld device which create stereoscopic images of the food; wherein the food images and changes in the spectra of the light beams caused by reflection from (or passage through) the food are analyzed together (in a multivariate manner) in order to identify the food's type and/or the food's composition; and wherein the stereoscopic food images are analyzed in order to measure food quantity.

In another example, a system for nutritional monitoring and management can comprise: a handheld device; a spectroscopic sensor in the handheld device, wherein the spectroscopic sensor further comprises a light emitter which emits light beams toward food and a light receiver which receives the light beams after the light beams have been reflected from (or passed through) the food; a camera in the handheld device which captures images of the food; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

In an example, a system for nutritional monitoring and management can comprise: a handheld device; a camera in the handheld device which captures images of a meal with multiple types of food; wherein meal images are analyzed to identify different types of food in the meal based on variation and boundaries in food shapes, sizes, colors, and textures; and a spectroscopic sensor in the handheld device which emits light beams toward food and receives the light beams after the light beams have been reflected from (or passed through) the food; wherein a person is prompted (e.g. via sound, vibration, or light) to direct light beams toward different locations on the meal which are associated with different types of food based on analysis of the meal images; wherein food images and changes in the spectra of the light beams caused by reflection from (or passage through) different types of food are analyzed together (in a multivariate manner) in order to identify, for each type of food in the meal, food type, food composition (e.g. nutritional composition), and/or food quantity. Alternatively, a system can comprise: a handheld device; a camera in the handheld device which captures images of food; a spectroscopic sensor in the handheld device which emits light beams toward food and receives the light beams after the light beams have been reflected from (or passed through) food; a laser pointer; wherein the person uses the laser pointer to direct the camera and the spectroscopic sensor toward a first food in a meal at a first point in time; wherein the person uses the laser pointer to direct the camera and the spectroscopic sensor toward a second food in a meal at a second point in time; wherein food images captured by the camera and changes in the spectra of the light beams caused by reflection from (or passage through) food are analyzed to identify the compositions and quantities of the first and second foods.

In an example, a system for nutritional monitoring and management can comprise: a handheld device; a camera in the handheld device which captures images of food; a spectroscopic sensor in the handheld device which emits light beams toward the food and receives the light beams after the light beams have been reflected from (or passed through) the food; wherein part of the spectrum of light beams emitted from the spectroscopic sensor create a light pattern on (or near) the food which is used as a fiducial marker to estimate food size, distance, and/or orientation relative to the device; and wherein changes in the spectra of light beams caused by reflection from (or passage through) the food and food images captured by the camera are analyzed to identify food type, composition, and/or quantity. Alternatively, a system can comprise: a handheld device; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are identified in food images to help estimate food size, distance, and/or orientation.

In an example, a system for nutritional monitoring and management can comprise: a handheld device; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are used to estimate food distance. In another example, a system can comprise: a handheld device; a camera in the handheld device which captures images of food at a first time and at a second time, wherein the first time is before a person eats the food and the second time is after the person has finished eating some or all of the food; a spectroscopic sensor in the handheld device which emits light beams toward food and receives the light beams after the light beams have been reflected from (or passed through) food at the first time and at the second time; wherein food images captured by the camera and changes in the spectra of the light beams caused by reflection from (or passage through) food are analyzed together (e.g. in multivariate analysis) to identify the type, composition, and/or quantity of food eaten by the person holding or wearing the device.

A system can be embodied in: a handheld device; a spectroscopic sensor in the handheld device which emits light beams toward food and receives the light beams after the light beams have been reflected from (or passed through) the food; a camera in the handheld device which captures images of the food from different perspectives and angles as the handheld device is moved; wherein the food images and changes in the spectra of the light beams caused by reflection from (or passage through) the food are analyzed together (in a multivariate manner) in order to identify the food's type and/or the food's composition; and wherein food images from different perspectives and angles are used to model the food in three dimensions in order to measure food quantity. Alternatively, a system can comprise: a handheld device; a spectroscopic sensor in the handheld device which emits light beams toward food and receives the light beams after the light beams have been reflected from (or passed through) food; a camera in the handheld device which captures images of the food; and a laser which projects a target (e.g. cross-hairs) light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld device; a spectroscopic sensor in the handheld device which emits light beams toward food and receives the light beams after the light beams have been reflected from (or passed through) food; a camera in the handheld device which captures images of the food; and a laser which projects an matrix (e.g. dot matrix or linear grid) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. Alternatively, a system can comprise: a handheld device; a spectroscopic sensor in the handheld device which emits light beams toward food and receives the light beams after the light beams have been reflected from (or passed through) food; a camera in the handheld device which captures images of the food; and a light pattern projector which projects a pattern of light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

A system can be embodied in: a handheld device; a spectroscopic sensor in the handheld device which emits light beams toward food and receives the light beams after the light beams have been reflected from (or passed through) food; a camera in the handheld device which captures images of the food; and a scanning (e.g. moving) laser which projects a polygonal light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. In another example, a system can comprise: a handheld device; a spectroscopic sensor in the handheld device which emits light beams toward food and receives the light beams after the light beams have been reflected from (or passed through) food; a camera in the handheld device which captures images of the food; and a scanning laser which projects an arcuate (e.g. circular) light pattern toward the food; wherein size of the projected light pattern on (or near) the food is used to estimate food distance; wherein keystone distortion of the projected light pattern on (or near) the food is used to estimate the orientation of the food relative to the device; and wherein changes in the spectra of light beams caused by reflection from (or passage through) the food and food images captured by the camera are analyzed to identify food type, composition, and/or quantity.

In an example, a system for nutritional monitoring and management can comprise: a handheld device; a spectroscopic sensor in the handheld device which emits light beams toward food and receives the light beams after the light beams have been reflected from (or passed through) the food; a camera in the handheld device which captures images of the food; a motion sensor; wherein the handheld device is waived over the food so that the spectroscopic sensor reflects beams from the food at multiple locations and the camera creates images of the food from multiple perspectives; wherein changes in the spectra of the light beams caused by reflection from (or passage through) the food, the food images, and movement of the handheld device are analyzed together (in a multivariate manner) in order to identify food type, measure food composition (e.g. nutritional composition), and/or measure food quantity. Alternatively, a system can comprise: a handheld device; a spectroscopic sensor in the handheld device which emits light beams toward food and receives the light beams after the light beams have been reflected from (or passed through) the food; a camera in the handheld device which captures images of the food; wherein the food images and changes in the spectra of the light beams caused by reflection from (or passage through) the food are analyzed together using a neural network in order to identify the food's type and/or measure the food's composition; and wherein the food images are analyzed to measure food quantity.

In an example, a system for nutritional monitoring and management can comprise: a handheld device; a spectroscopic sensor in the handheld device which emits light beams toward food and receives the light beams after the light beams have been reflected from (or passed through) the food; a camera in the handheld device which captures images of the food; wherein the field of view of the camera encompasses the entire projection path of light beams from the spectroscopic sensor; area wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. Alternatively, a system can comprise: a handheld device; a spectroscopic sensor in the handheld device which emits light beams toward food and receives the light beams after the light beams have been reflected from the food; a camera in the handheld device which captures images of the food; wherein the food images and changes in the spectra of the light beams caused by reflection from the food are analyzed together (in a multivariate manner) in order to identify the food's type and/or measure the food's composition; wherein the food images are analyzed to measure food quantity; a range finder which measures the distance from the handheld device to the food; and wherein the spectroscopic sensor and camera are both automatically triggered at the same selected distance from the food.

In an example, a system for nutritional monitoring and management can comprise: a handheld device; a spectroscopic sensor in the handheld device which emits light beams toward food and receives the light beams after the light beams have been reflected from (or passed through) the food; a camera in the handheld device which captures images of the food; wherein the shape, size, color, tone, brightness, and/or texture of food in the food images and the changes in the spectra of the light beams caused by reflection from (or passage through) the food are analyzed together (in a multivariate manner) in order to identify the food's type and/or measure the food's composition; and wherein the food images are analyzed to measure food quantity. In another example, a system can comprise: a handheld device; a camera in the handheld device which captures images of food, wherein the food images are automatically analyzed to identify food type and/or measure food quantity; and a spectroscopic sensor in the handheld device which emits light beams toward food and receives the light beams after the light beams have been reflected from (or passed through) food, wherein changes in the spectra of the light beams caused by reflection from (or passage through) food are analyzed to identify food type and/or composition; a wearable device; a sound sensor in the wearable device which tracks chewing or swallowing sounds; wherein the device prompts a person with a sound, vibration, or light to use the camera and/or the spectroscopic sensor based on the number or timing of chewing or swallowing sounds in order to measure changes in the amount of food remaining (and infer how much food the person has actually consumed) and to measure the composition of different layers (or parts) of the food.

In an example, a system for nutritional monitoring and management can comprise: a handheld device which is held by a person; a camera in the handheld device which captures images of food; a spectroscopic sensor in the handheld device which emits light beams toward food and receives the light beams after the light beams have been reflected from (or passed through) food; a necklace which is worn by the person; and a sound sensor in the necklace which tracks chewing or swallowing sounds; wherein data from the camera, the spectroscopic sensor, and the sound sensor are analyzed together (e.g. in multivariate analysis) to identify the type, composition (e.g. nutritional composition), and/or quantity of food eaten by the person holding or wearing the device. Alternatively, a system can comprise: a handheld device which is held by a person; a camera in the wearable device which captures images of food; a spectroscopic sensor in the wearable device which emits light beams toward food and receives the light beams after the light beams have been reflected from (or passed through) food; a wearable device which is worn by the person; and a motion sensor in the wearable device which tracks hand-to-mouth motions, chewing motions, and/or swallowing motions; wherein data from the camera, the spectroscopic sensor, and the motion sensor are analyzed together (e.g. in multivariate analysis) to identify the type, composition, and/or quantity of food eaten by the person holding or wearing the device.

In an example, a system for nutritional monitoring and management can comprise: a handheld device which is waived back and forth several times over a multi-food meal; a spectroscopic sensor in the handheld device which emits light beams toward the meal and receives the light beams after the light beams have been reflected from (or passed through) a plurality of locations on the meal as the device is waived back and forth; a camera in the handheld device which captures images of a plurality of locations on the meal as the device is waived back and forth; and a motion sensor; wherein data from the spectroscopic sensor, the camera, and the motion sensor are analyzed together (in a multivariate manner) in order to identify the types, compositions, and/or quantities of foods in the multi-food meal. In another example, a system can comprise: a handheld device which is waived back and forth several times over a multi-food meal; a spectroscopic sensor in the handheld device which emits light beams toward the meal and receives the light beams after the light beams have been reflected from (or passed through) a plurality of locations on the meal as the device is waived back and forth; a camera in the handheld device which captures images of a plurality of locations on the meal as the device is waived back and forth; and a motion sensor; wherein data from the spectroscopic sensor, the camera, and the motion sensor are analyzed to segment the meal into different food portions; and wherein data from the spectroscopic sensor, the camera, and the motion sensor are analyzed to identify the types, compositions, and/or quantities of foods in the different food portions.

A system can be embodied in: a handheld device which is waived back and forth several times over a multi-food meal; a spectroscopic sensor in the handheld device which emits light beams toward the meal and receives the light beams after the light beams have been reflected from (or passed through) a plurality of locations on the meal as the device is waived back and forth; a camera in the handheld device which captures images of a plurality of locations on the meal as the device is waived back and forth; and a motion sensor; wherein variations in food color, tone, brightness, texture, shape, and molecular composition as the device is waived back and forth are analyzed to segment the meal into different food portions; and wherein data from the spectroscopic sensor, the camera, and the motion sensor are analyzed to identify the types, compositions, and/or quantities of foods in the different food portions. Alternatively, a system can comprise: a handheld device which is waived back and forth several times over a multi-food meal; a spectroscopic sensor in the handheld device which emits light beams toward the meal and receives the light beams after the light beams have been reflected from (or passed through) the meal as the device is being waived back and forth; a camera in the handheld device which captures images of the meal as the device is being waived back and forth; and a motion sensor; wherein data from the spectroscopic sensor, the camera, and the motion sensor are analyzed together (in a multivariate manner) in order to identify the types, compositions, and/or quantities of foods in the multi-food meal.

In an example, a system for nutritional monitoring and management can comprise: a handheld device which is waived back and forth several times over food; a spectroscopic sensor in the handheld device which emits light beams toward food and receives the light beams after the light beams have been reflected from (or passed through) the food as the device is being waived back and forth; a camera in the handheld device which captures images of the food as the device is being waived back and forth; and a motion sensor; wherein data from the spectroscopic sensor, the camera, and the motion sensor are analyzed together (in a multivariate manner) in order to identify the food type, composition, and/or quantity. In another example, a system can comprise: a handheld device which is waived in an arc segment of a circle over nearby food; a spectroscopic sensor in the handheld device which emits light beams toward food and receives the light beams after the light beams have been reflected from (or passed through) the food; a camera in the handheld device which captures images of the food; and a motion sensor; wherein data from the spectroscopic sensor, the camera, and the motion sensor are analyzed together (in a multivariate manner) in order to identify the food type, composition (e.g. nutritional composition), and/or quantity.

In an example, a system for nutritional monitoring and management can comprise: a handheld device which is waived over a meal in an arc which is wider than the width of the meal; a spectroscopic sensor in the handheld device which emits light beams toward food and receives the light beams after the light beams have been reflected from (or passed through) the food; a camera in the handheld device which captures images of the food; and a motion sensor; wherein data from the spectroscopic sensor, the camera, and the motion sensor are analyzed together (in a multivariate manner) in order to identify the food type, composition, and/or quantity. Alternatively, a system can comprise: a handheld device which is waived over a meal with multiple types of food; a spectroscopic sensor in the handheld device which emits light beams toward food and receives the light beams after the light beams have been reflected from (or passed through) the food; a camera in the handheld device which captures images of the food in a field of view which overlaps the projection path of light beams from the spectroscopic sensor; and a motion sensor; wherein the handheld device guides a person concerning how to waive or otherwise move the handheld device over the meal with multiple types of food; and wherein data from the spectroscopic sensor, the camera, and the motion sensor are analyzed together (in a multivariate manner) in order to identify the food type, composition (e.g. nutritional composition), and/or quantity.

In an example, a system for nutritional monitoring and management can comprise: a handheld device which is waived over a meal with multiple types of food; a spectroscopic sensor in the handheld device which emits light beams toward food and receives the light beams after the light beams have been reflected from (or passed through) the food; a camera in the handheld device which captures images of the food in a field of view which overlaps the projection path of light beams from the spectroscopic sensor; and a motion sensor; wherein the handheld device projects a light pointer to guide a person concerning how to waive the handheld device over the meal with multiple types of food; and wherein data from the spectroscopic sensor, the camera, and the motion sensor are analyzed together (in a multivariate manner) in order to identify the food type, composition, and/or quantity. Alternatively, a system can comprise: a handheld device which is waived over a meal with multiple types of food; a spectroscopic sensor in the handheld device which emits light beams toward food and receives the light beams after the light beams have been reflected from (or passed through) the food; a camera in the handheld device which captures images of the food in a field of view which overlaps the projection path of light beams from the spectroscopic sensor; and a motion sensor; wherein the handheld device has a screen which displays a virtual pointer in augmented reality to guide a person concerning how to waive or otherwise move the handheld device over the meal with multiple types of food; and wherein data from the spectroscopic sensor, the camera, and the motion sensor are analyzed together (in a multivariate manner) in order to identify the food type, composition (e.g. nutritional composition), and/or quantity.

A system can be embodied in: a handheld device which is waived over a multi-food meal; a spectroscopic sensor in the handheld device which emits light beams toward the meal and receives the light beams after the light beams have been reflected from (or passed through) the meal; and a camera in the handheld device which captures images of the food in a field of view which overlaps the projection path of light beams from the spectroscopic sensor; wherein data from the spectroscopic sensor and the camera are analyzed together (in a multivariate manner) in order to identify the type, composition (e.g. nutritional composition), and/or quantity of each type of food in the meal.

In another example, a system for nutritional monitoring and management can comprise: a handheld device which is waived over a plate of food in an arc which is wider than the plate; a spectroscopic sensor in the handheld device which emits light beams toward food and receives the light beams after the light beams have been reflected from (or passed through) the food; a camera in the handheld device which captures images of the food; and a motion sensor; wherein data from the spectroscopic sensor, the camera, and the motion sensor are analyzed together (in a multivariate manner) in order to identify the food type, composition (e.g. nutritional composition), and/or quantity.

In an example, a system for nutritional monitoring and management can comprise: a handheld device which is waived over a plate of food in an zigzag pattern which is wider than the plate; a spectroscopic sensor in the handheld device which emits light beams toward food and receives the light beams after the light beams have been reflected from (or passed through) the food; a camera in the handheld device which captures images of the food; and a motion sensor; wherein data from the spectroscopic sensor, the camera, and the motion sensor are analyzed together (in a multivariate manner) in order to identify the food type, composition, and/or quantity. Alternatively, a system can comprise: a handheld device which is waived over food; a spectroscopic sensor in the handheld device which emits light beams toward food and receives the light beams after the light beams have been reflected from (or passed through) the food; a camera in the handheld device which captures images of the food in a field of view which overlaps the projection path of light beams from the spectroscopic sensor; and a motion sensor; wherein the handheld device guides a person concerning how and/or where to waive the handheld device over food; and wherein data from the spectroscopic sensor, the camera, and the motion sensor are analyzed together (in a multivariate manner) in order to identify the food type, composition (e.g. nutritional composition), and/or quantity.

A system can be embodied in: a handheld device which is waived over nearby food; a spectroscopic sensor in the handheld device which emits light beams toward food and receives the light beams after the light beams have been reflected from (or passed through) the food; a camera in the handheld device which captures images of the food; and a motion sensor; wherein data from the spectroscopic sensor, the camera, and the motion sensor are analyzed together (in a multivariate manner) in order to identify the food type, composition (e.g. nutritional composition), and/or quantity. Alternatively, a system can comprise: a handheld device which is waived over nearby food; a spectroscopic sensor in the handheld device which emits light beams toward food and receives the light beams after the light beams have been reflected from (or passed through) the food; and a camera in the handheld device which captures images of the food in a field of view which overlaps the projection path of light beams from the spectroscopic sensor; wherein data from the spectroscopic sensor and the camera are analyzed together (in a multivariate manner) in order to identify the food type, composition (e.g. nutritional composition), and/or quantity.

In an example, a system for nutritional monitoring and management can comprise: a handheld device which is waived over nearby food; a spectroscopic sensor in the handheld device which emits light beams toward food and receives the light beams after the light beams have been reflected from (or passed through) the food; a camera in the handheld device which captures images of the food in a field of view which overlaps the projection path of light beams from the spectroscopic sensor; and a motion sensor; wherein data from the spectroscopic sensor, the camera, and the motion sensor are analyzed together (in a multivariate manner) in order to identify the food type, composition, and/or quantity. In another example, a system can comprise: a handheld device with a longitudinal axis and cross-sectional asymmetry, wherein a proximal portion of the device has a larger cross-section than a distal portion of the device; a spectroscopic sensor in the handheld device which emits light beams from the distal end of the handheld device toward food and receives the light beams after the light beams have been reflected from (or passed through) the food; a camera in the handheld device which captures images of the food from the distal end of the device; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

In an example, a system for nutritional monitoring and management can comprise: a handheld device with a proximal side which is configured to face toward a person's head when the device is held and a distal surface which is configured to face away from the person's head when the device is held; a spectroscopic sensor in the handheld device which emits light beams from the distal side of the device toward food and receives the light beams after the light beams have been reflected from (or passed through) the food; a camera in the handheld device which captures images of the food from the distal side of the device; wherein the food images and changes in the spectra of the light beams caused by reflection from (or passage through) the food are analyzed together (in a multivariate manner) in order to identify the food's type and/or measure the food's composition; wherein the food images are analyzed to measure food quantity; and a display screen on the proximal side of the device, wherein the display screen shows augmented reality food images including a virtual pointer, virtual cross hairs, or other virtual guide marks to guide the user concerning where to position the device when using the spectroscopic sensor and/or the camera. Alternatively, a system can comprise: a handheld device with a proximal side which is configured to face toward a person's head when the device is held and a distal surface which is configured to face away from the person's head when the device is held; a spectroscopic sensor in the handheld device which emits light beams from the distal side of the device toward food and receives the light beams after the light beams have been reflected from (or passed through) the food; a camera in the handheld device which captures images of the food from the distal side of the device; wherein the food images and changes in the spectra of the light beams caused by reflection from (or passage through) the food are analyzed together (in a multivariate manner) in order to identify the food's type and/or measure the food's composition; wherein the food images are analyzed to measure food quantity; and a display screen on the proximal side of the device, wherein the display screen shows augmented reality food images including a virtual pointer which sequentially points at different types of food in the multi-food meal to guide the user where and when to position the device for a spectroscopic scan of each type of food in the multi-food meal.

In an example, a system for nutritional monitoring and management can comprise: a handheld device with a proximal surface which is configured to be closer to a person's head when the device is held and a distal surface which is configured to be farther from the person's head when the device is held; a spectroscopic sensor in the handheld device which emits light beams from the distal surface of the device toward food and receives the light beams after the light beams have been reflected from (or passed through) the food; a camera in the handheld device which captures images of the food from the distal surface of the device; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. In another example, a system can comprise: a handheld device with a proximal surface which is configured to be closer to a person's head when the device is held and a distal surface which is configured to be farther from the person's head when the device is held; a spectroscopic sensor with an aperture on the distal surface of the handheld device which emits light beams toward food and receives the light beams after the light beams have been reflected from (or passed through) the food; a first camera which captures images of the food; wherein the aperture of the first camera is located to one side of the aperture of the spectroscopic sensor; a second camera which captures images of the food; wherein the aperture of the second camera is located to a second side of (e.g. on the opposite side of) the aperture of the spectroscopic sensor; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

A system can be embodied in: a handheld device with a proximal surface which is configured to be closer to a person's head when the device is held and a distal surface which is configured to be farther from the person's head when the device is held; a spectroscopic sensor with an aperture on the distal surface of the handheld device which emits light beams toward food and receives the light beams after the light beams have been reflected from (or passed through) the food; a camera with an aperture on the distal surface of the handheld device which captures images of the food; wherein the aperture of the spectroscopic sensor is the co-located with, co-axial with, and/or the same as the aperture of the camera; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. Alternatively, a system can comprise: a handheld device with a proximal surface which is configured to be closer to a person's head when the device is held and a distal surface which is configured to be farther from the person's head when the device is held; a spectroscopic sensor with an aperture on the distal surface of the handheld device which emits light beams toward food and receives the light beams after the light beams have been reflected from (or passed through) the food; a camera with an aperture on the distal surface of the handheld device which captures images of the food; wherein the aperture of the spectroscopic sensor is between 5 mm and 100 mm away from the aperture of the camera; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

In an example, a system for nutritional monitoring and management can comprise: a handheld device with a proximal surface which is configured to be closer to a person's head when the device is held and a distal surface which is configured to be farther from the person's head when the device is held; a spectroscopic sensor on the distal surface in the handheld device which emits light beams toward food and receives the light beams after the light beams have been reflected from (or passed through) the food; a camera on the distal surface of the handheld device which captures images of the food; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. Alternatively, a system can comprise: a handheld device with a proximal surface which is configured to be closer to a person's head when the device is held and a distal surface which is configured to be farther from the person's head when the device is held; a spectroscopic sensor with an aperture on the distal surface of the handheld device which emits light beams toward food and receives the light beams after the light beams have been reflected from (or passed through) the food; a camera with an aperture on the distal surface of the handheld device which captures images of the food; wherein the aperture of the spectroscopic sensor is between 1 mm and 10 mm away from the aperture of the camera; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

In an example, a system for nutritional monitoring and management can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein the camera and/or the spectroscopic sensor is automatically triggered when the device detects that the person is eating. Alternatively, a system can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein the camera and/or the spectroscopic sensor is automatically triggered when the device detects that the person is eating based on movement of the person's jaw, such as bending of the jaw joint.

In an example, a system for nutritional monitoring and management can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein the camera and/or the spectroscopic sensor is automatically triggered when the device detects that the person is eating based on GPS or other location-based indications that a person is in an eating establishment (such as a restaurant) or food source location (such as a kitchen).

In another example, a system for nutritional monitoring and management can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition, and/or quantity; and wherein the camera and/or the spectroscopic sensor is automatically triggered when the device detects that the person is eating based on acceleration, inclination, twisting, or rolling of the person's hand, wrist, or arm.

A system can be embodied in: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition, and/or quantity; and wherein the camera and/or the spectroscopic sensor is automatically triggered when the device detects that the person is eating based on smells suggesting food that are detected by an artificial olfactory sensor. Alternatively, a system can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein the camera and/or the spectroscopic sensor is automatically triggered when the device detects that the person is eating based on acceleration or inclination of the person's lower arm or upper arm.

In an example, a system for nutritional monitoring and management can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein the camera and/or the spectroscopic sensor is automatically triggered when the device detects that the person is eating based on detection of chewing, swallowing, or other eating sounds by one or more microphones. In another example, a system can comprise: a handheld device worn by a person; a camera in the handheld device which captures images of food; and a spectroscopic sensor in the handheld device which emits light beams toward food and receives the light beams after the light beams have been reflected from (or passed through) food; and an eating detector selected from the group consisting of—accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor; wherein food images captured by the camera and changes in the spectra of the light beams caused by reflection from (or passage through) food are analyzed to identify food type, composition, and/or quantity; and wherein the camera and/or the spectroscopic sensor is automatically triggered when the eating detector detects that the person is eating.

In an example, a system for nutritional monitoring and management can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein the camera and/or the spectroscopic sensor is automatically triggered when the device detects that the person is eating based on bending of the person's shoulder, elbow, wrist, or finger joints. Alternatively, a system can comprise: a handheld device worn by a person; a camera in the handheld device which captures images of food; and a spectroscopic sensor in the handheld device which emits light beams toward food and receives the light beams after the light beams have been reflected from (or passed through) food; wherein food images captured by the camera and changes in the spectra of the light beams caused by reflection from (or passage through) food are analyzed to identify food type, composition, and/or quantity; and wherein the camera and/or the spectroscopic sensor is automatically triggered when the device detects that the person is eating based on electromagnetic waves from the person's stomach, heart, brain, or other organs.

In an example, a system for nutritional monitoring and management can comprise: a handheld food probe; a camera in the probe which captures images of food; and a spectroscopic sensor in the probe which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity. Alternatively, a system can comprise: a handheld food probe which is inserted into food; and a spectroscopic sensor which is part of (and/or in optical communication with) the food probe; wherein a first set of light beams from the spectroscopic sensor are reflected by (or pass through) a first interior portion of the food at a first time and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food at a second time and changes in the spectra of the second set of light beams caused by reflection from (or passage through) the second interior portion of the food are analyzed to identify the composition of the second interior portion of the food.

In an example, a system for nutritional monitoring and management can comprise: a handheld food probe which is inserted into food; and a spectroscopic sensor which is part of (and/or in optical communication with) the food probe; wherein a first set of light beams from the spectroscopic sensor are reflected by (or pass through) a first interior portion of the food and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food and changes in the spectra of the second set of light beams caused by reflection from (or passage through) the second interior portion of the food are analyzed to identify the composition of the second interior portion of the food, and wherein the second interior portion of the food is closer to the centroid of the food than the first interior portion of the food. Alternatively, a system can comprise: a handheld food probe which is inserted into food; and a spectroscopic sensor with one or more moving optical components which is part of (and/or in optical communication with) the food probe; wherein a first set of light beams from the spectroscopic sensor are reflected by (or pass through) a first interior portion of the food at a first time and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food at a second time and changes in the spectra of the second set of light beams caused by reflection from (or passage through) the second interior portion of the food are analyzed to identify the composition of the second interior portion of the food, and wherein the second interior portion of the food is at least 5 mm away from the first interior portion of the food.

In an example, a system for nutritional monitoring and management can comprise: a handheld food probe which is inserted into food; a light beam emitter which emits light beams toward the food from within the handheld food probe; a light beam receiver which receives the emitted light beams after they have been reflected from (or passed through) the food; and a moving mirror and/or lens which changes the location inside the food from which the light beams are reflected. In another example, a system can comprise: a handheld food probe which is inserted into food; and a spectroscopic sensor which is part of (and/or in optical communication with) the food probe; wherein a first set of light beams from the spectroscopic sensor are reflected by (or pass through) a first interior portion of the food at a first time and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food at a second time and changes in the spectra of the second set of light beams caused by reflection from (or passage through) the second interior portion of the food are analyzed to identify the composition of the second interior portion of the food, and wherein the second interior portion of the food is closer to the centroid of the food than the first interior portion of the food.

In an example, a system for nutritional monitoring and management can comprise: a handheld food probe which is inserted into food; and a spectroscopic sensor which is part of (and/or in optical communication with) the food probe; wherein a first set of light beams from the spectroscopic sensor are reflected by (or pass through) a first interior portion of the food and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food and changes in the spectra of the second set of light beams caused by reflection from (or passage through) the second interior portion of the food are analyzed to identify the composition of the second interior portion of the food, and wherein the second interior portion of the food is at least 5 mm away from the first interior portion of the food. Alternatively, a system can comprise: a handheld food probe which is inserted into food; and a spectroscopic sensor which is part of (and/or in optical communication with) the food probe; wherein a first set of light beams from the spectroscopic sensor are reflected by (or pass through) a first interior portion of the food at a first time and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food at a second time and changes in the spectra of the second set of light beams caused by reflection from (or passage through) the second interior portion of the food are analyzed to identify the composition of the second interior portion of the food, and wherein the second interior portion of the food is at least 5 mm away from the first interior portion of the food.

In an example, a system for nutritional monitoring and management can comprise: a handheld food probe which is inserted into food; and a spectroscopic sensor with one or more moving optical components which is part of (and/or in optical communication with) the food probe; wherein a first set of light beams from the spectroscopic sensor are reflected by (or pass through) a first interior portion of the food at a first time and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food at a second time and changes in the spectra of the second set of light beams caused by reflection from (or passage through) the second interior portion of the food are analyzed to identify the composition of the second interior portion of the food. In another example, a system can comprise: a handheld food probe which is inserted into food; and a spectroscopic sensor which is part of (and/or in optical communication with) the food probe; wherein a first set of light beams from the spectroscopic sensor are reflected by (or pass through) a first interior portion of the food and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food and changes in the spectra of the second set of light beams caused by reflection from (or passage through) the second interior portion of the food are analyzed to identify the composition of the second interior portion of the food.

In an example, a system for nutritional monitoring and management can comprise: a handheld food probe which is inserted into food; and a spectroscopic sensor with one or more moving optical components which is part of (and/or in optical communication with) the food probe; wherein a first set of light beams from the spectroscopic sensor are reflected by (or pass through) a first interior portion of the food at a first time and changes in the spectra of the first set of light beams caused by reflection from (or passage through) the first interior portion of the food are analyzed to identify the composition of the first interior portion of the food; and wherein a second set of light beams from the spectroscopic sensor are reflected by (or pass through) a second interior portion of the food at a second time and changes in the spectra of the second set of light beams caused by reflection from (or passage through) the second interior portion of the food are analyzed to identify the composition of the second interior portion of the food, and wherein the second interior portion of the food is closer to the centroid of the food than the first interior portion of the food. Alternatively, a system can comprise: a handheld food scanner; a camera in the scanner which captures images of food; and a spectroscopic sensor in the scanner which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the device which captures images of a multi-food meal; a laser pointer which is sequentially pointed toward different types of food in the multi-food meal; a spectroscopic sensor in the device is sequentially pointed toward different types of food in the multi-food meal, wherein the spectroscopic sensor emits light beams toward a type of food and receives the light beams after the light beams have been reflected from (or passed through) the type of food; wherein food images captured by the camera and changes in the spectra of the light beams caused by reflection from (or passage through) food are analyzed to identify food type, composition (e.g. nutritional composition), and/or quantity for each type of food in the multi-food meal. Alternatively, a system can comprise: a handheld or wearable device; a camera in the device which sequentially captures an image of each type of food in a multi-food meal; a spectroscopic sensor in the device which sequentially emits light beams toward each type of food in the multi-food meal and receives the light beams after the light beams have been reflected from (or passed through) the type of food; and a laser pointer in the device which guides the person concerning where to position the device so that camera captures an image of each type of food in the multi-food meal and/or the spectroscopic sensor sequentially emits light beams toward each type of food in the multi-food meal; wherein food images captured by the camera and changes in the spectra of the light beams caused by reflection from (or passage through) food are analyzed to identify food type, composition, and/or quantity for each type of food in the multi-food meal.

In an example, a system for nutritional monitoring and management can comprise: a handheld or wearable device; a camera in the handheld or wearable device which captures images of food; a spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and one or more other components selected from the group consisting of—accelerometer, altimeter, ambient light sensor, electromagnetic energy sensor, filter, GPS module, gyroscope, lens array, magnetometer, MEMS, microphone, parabolic reflector, temperature sensor, and vibrator.

In another example, a system for nutritional monitoring and management can comprise: a handheld or wearable device; a camera in the handheld or wearable device which captures images of a meal with different types of food; an infrared thermal sensor which measures the temperature of the different types of food; wherein different types of food in the meal are differentiated based on their shapes, sizes, colors, tones, brightness levels, textures, and/or temperatures; a spectroscopic sensor in the handheld or wearable device which emits light beams toward each of the different types of food and receives the light beams after the light beams have been reflected from (or passed through) each of the different types of food; wherein data from the camera, the spectroscopic sensor, and the infrared thermal sensor are analyzed together in order to identify types, compositions, and/or quantities of each of the different types of food in the meal.

In an example, a system for nutritional monitoring and management can comprise: a handheld or wearable device; a camera in the handheld or wearable device which captures images of food; and a holographic spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity. Alternatively, a system can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food, wherein the spectroscopic sensor emits light beams with scanning variation in frequencies and/or wavelength; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify chemicals and/or microbes in the food. Alternatively, a system can comprise: a handheld or wearable device; a camera in the handheld or wearable device which captures images of food; and an infrared spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition, and/or quantity.

In an example, a system for nutritional monitoring and management can comprise: a handheld or wearable device; a camera in the device which captures images of a multi-food meal; a laser pointer which is sequentially pointed toward different portions of food in the multi-food meal; a spectroscopic sensor in the device is sequentially pointed toward different portions of food in the meal, wherein the spectroscopic sensor emits light beams toward a portion of food and receives the light beams after the light beams have been reflected from (or passed through) the portion of food; wherein food images captured by the camera and changes in the spectra of the light beams caused by reflection from (or passage through) food are analyzed to identify food type, composition, and/or quantity for each portion of food in the multi-food meal. In another example, a system can comprise: a handheld or wearable device; a camera in the device which captures images of food; a spectroscopic sensor in the device which emits light beams toward food and receives the light beams after the light beams have been reflected from (or passed through) food; and a laser pointer in the device which guides the person concerning where to position the device so that camera captures images of the food and/or the spectroscopic sensor emits light beams toward the food; wherein food images captured by the camera and changes in the spectra of the light beams caused by reflection from (or passage through) food are analyzed to identify food type, composition (e.g. nutritional composition), and/or quantity.

A system can be embodied in: a handheld or wearable device; a camera in the handheld or wearable device which captures images of food; a light emitter in the handheld or wearable device which emits light beams toward food; a light receiver in the handheld or wearable device which receives the light beams after the light beams have been reflected from (or passed through) food; and an optical filter selected from the group consisting of acousto-optic filter, Bragg filter, cascaded filter, dielectric thin-film filter, Fabry-Perot filter, hybrid filter, optical absorption filter, and optical interference filter; wherein food images captured by the camera and changes in the spectra of the light beams caused by reflection from (or passage through) food are analyzed to identify food type, composition, and/or quantity. Alternatively, a system can comprise: a handheld or wearable device; a camera in the handheld or wearable device which captures images of food; a light emitter which emits light beams toward food; and a light receiver which receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the handheld or wearable device which captures images of a meal with different types of food; an infrared thermal sensor which measures the temperature of the different types of food; wherein different types of food in the meal are differentiated based on their shapes, sizes, colors, tones, brightness levels, textures, and/or temperatures; and a spectroscopic sensor in the handheld or wearable device; wherein the device prompts the person to direct the spectroscopic sensor toward a central location on each of the different types of food; wherein the spectroscopic sensor emits light beams toward each of the different types of food and receives the light beams after the light beams have been reflected from (or passed through) each of the different types of food; wherein data from the camera, the spectroscopic sensor, and the infrared thermal sensor are analyzed together in order to identify types, compositions, and/or quantities of each of the different types of food in the meal. In another example, a system can comprise: a handheld or wearable device; a camera in the handheld or wearable device which captures images of food; and a Fabry-Perot spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the handheld or wearable device which captures images of food; and a near-infrared spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity. Alternatively, a system can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and a cover or lid which automatically closes to prevent the camera and/or the spectroscopic sensor from coming into direct contact with viscous food.

In an example, a system for nutritional monitoring and management can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food, wherein the spectroscopic sensor comprises a plurality of light emitters which emit light beams n different wavelength ranges; wherein food images captured by the camera and changes in the spectra of the light beams caused by reflection from (or passage through) food are analyzed to identify food type, composition, and/or quantity. Alternatively, a system can comprise: a handheld or wearable device; a camera in the handheld or wearable device which captures images of different foods in a multi-food meal; and a spectroscopic sensor in the handheld or wearable device which emits light beams toward the different foods in the multi-food meal and receives the light beams after the light beams have been reflected from (or passed through) the different foods; wherein differences in food size, color, tone, brightness, texture, and/or shape among different foods in the food images captured by the camera and changes in the spectra of the light beams caused by reflection from (or passage through) food are analyzed to identify the food type, composition (e.g. nutritional composition), and/or quantity for each of the different foods in the multi-food meal.

In an example, a system for nutritional monitoring and management can comprise: a handheld or wearable device; a camera in the handheld or wearable device which captures images of food, wherein the food images are automatically analyzed to identify food type and/or measure food quantity; and a spectroscopic sensor in the handheld or wearable device which emits light beams toward food and receives the light beams after the light beams have been reflected from (or passed through) food, wherein changes in the spectra of the light beams caused by reflection from (or passage through) food are analyzed to identify food type and/or composition; and where the device prompts a person to use the camera and/or the spectroscopic sensor at multiple times while the person is eating a meal in order to measure changes in the amount of food remaining (and infer how much food the person has actually consumed) and to measure the composition of different layers (or parts) of the food. Alternatively, a system can comprise: a handheld or wearable device; a camera in the device which captures images of a multi-food meal; a spectroscopic sensor in the device which emits light beams toward food and receives the light beams after the light beams have been reflected from (or passed through) food; and a laser pointer in the device which guides the person concerning where to position the device so that camera captures images of each type of food in the multi-food meal and/or the spectroscopic sensor emits light beams toward each type of food in the multi-food meal; wherein food images captured by the camera and changes in the spectra of the light beams caused by reflection from (or passage through) food are analyzed to identify food type, composition, and/or quantity for each type of food in the multi-food meal.

In an example, a system for nutritional monitoring and management can comprise: a handheld or wearable device; a camera in the handheld or wearable device which captures images of food; a light emitter which emits light beams toward food; wherein the light emitter is selected from the group consisting of—light emitting diode (LED), organic light emitting diode (OLED), quantum dot light emitting diode (QLED), dye laser, filament lamp, fluorescent lamp, gas laser, halogen lamp, incandescent lamp, low pressure sodium lamp, super luminescent diode, tunable laser, and vertical cavity surface emitting laser (VCSEL); and a light receiver which receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity. In another example, a system can comprise: a handheld or wearable device; a camera in the handheld or wearable device which captures images of food; a spectroscopic sensor in the handheld or wearable device which emits light beams toward food and receives the light beams after the light beams have been reflected from (or passed through) food; an infrared thermal sensor; wherein data from the camera, the spectroscopic sensor, and the infrared thermal sensor are analyzed together in order to identify food type, composition (e.g. nutritional composition), and/or quantity.

In an example, a system for nutritional monitoring and management can comprise: a handheld or wearable device; a camera in the handheld or wearable device which captures images of a meal with different types of food; an infrared thermal sensor which measures the temperature of the different types of food; wherein different types of food in the meal are differentiated based on their shapes, sizes, colors, tones, brightness levels, textures, and/or temperatures; and a spectroscopic sensor in the handheld or wearable device; wherein the device guides the person using a projected light pointer concerning where to orient the spectroscopic sensor toward a central location on each of the different types of food; wherein the spectroscopic sensor emits light beams toward each of the different types of food and receives the light beams after the light beams have been reflected from (or passed through) each of the different types of food; wherein data from the camera, the spectroscopic sensor, and the infrared thermal sensor are analyzed together in order to identify types, compositions, and/or quantities of each of the different types of food in the meal. Alternatively, a system can comprise: a handheld or wearable device; a camera in the handheld or wearable device which captures images of food; and a prism spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food, wherein the spectroscopic sensor emits light beams at different frequencies at different times; wherein food images captured by the camera and changes in the spectra of the light beams caused by reflection from (or passage through) food are analyzed to identify food type, composition, and/or quantity. Alternatively, a system can comprise: a handheld or wearable device; a camera in the handheld or wearable device which captures images of different foods in a multi-food meal; and a spectroscopic sensor in the handheld or wearable device which emits light beams toward the different foods in the multi-food meal and receives the light beams after the light beams have been reflected from (or passed through) the different foods; wherein multivariate differences in food size, color, tone, brightness, texture, and shape among different foods in the food images captured by the camera and changes in the spectra of the light beams caused by reflection from (or passage through) food are analyzed to identify the food type, composition, and/or quantity for each of the different foods in the multi-food meal.

In an example, a system for nutritional monitoring and management can comprise: a handheld or wearable device; a camera in the handheld or wearable device which captures images of food; and a spectroscopic sensor in the handheld or wearable device with frequency-based modulation which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In another example, a system for nutritional monitoring and management can comprise: a handheld or wearable device; a camera in the handheld or wearable device which captures images of food; and a UV-VIS spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the device which captures images of food; a laser pointer which is directed toward the food; a spectroscopic sensor in the device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity. Alternatively, a system can comprise: a handheld or wearable device; a camera in the handheld or wearable device which captures images of food; a light emitter which emits light beams toward food; and a light receiver which receives the light beams after the light beams have been reflected from (or passed through) food, wherein the light receiver is selected from the group consisting of—avalanche photodiode (APD) array, charge-coupled device (CCD), complementary metal-oxide semiconductor (CMOS), focal plane array (FPA), and photo-diode array (PDA); and wherein food images captured by the camera and changes in the spectra of the light beams caused by reflection from (or passage through) food are analyzed to identify food type, composition, and/or quantity.

In an example, a system for nutritional monitoring and management can comprise: a handheld or wearable device; a camera in the handheld or wearable device which captures images of a meal with different types of food; a spectroscopic sensor in the handheld or wearable device which emits light beams toward the different types of food and receives the light beams after the light beams have been reflected from (or passed through) the different types of food; an infrared thermal sensor which measures the temperature of the different types of food; wherein data from the camera, the spectroscopic sensor, and the infrared thermal sensor are analyzed together in order to identify types, compositions, and/or quantities of the different types of food in the meal. Alternatively, a system can comprise: a handheld or wearable device; a camera in the handheld or wearable device which captures images of a meal with different types of food; an infrared thermal sensor which measures the temperature of the different types of food; wherein different types of food in the meal are differentiated based on their shapes, sizes, colors, tones, brightness levels, textures, and/or temperatures; and a spectroscopic sensor in the handheld or wearable device; wherein the device guides the person using a virtual augmented reality pointer concerning where to orient the spectroscopic sensor toward a central location on each of the different types of food; wherein the spectroscopic sensor emits light beams toward each of the different types of food and receives the light beams after the light beams have been reflected from (or passed through) each of the different types of food; wherein data from the camera, the spectroscopic sensor, and the infrared thermal sensor are analyzed together in order to identify types, compositions, and/or quantities of each of the different types of food in the meal.

In an example, a system for nutritional monitoring and management can comprise: a handheld or wearable device; a camera in the handheld or wearable device which captures images of food; and a grating spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition, and/or quantity. In another example, a system can comprise: a handheld or wearable device; a camera in the handheld or wearable device which captures images of food; and a Raman spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food, wherein the spectroscopic sensor emits a sequence of light beams at different frequencies; wherein food images captured by the camera and changes in the spectra of the light beams caused by reflection from (or passage through) food are analyzed to identify food type, composition, and/or quantity. Alternatively, a system can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld or wearable device; a camera in the handheld or wearable device which captures images of food; and a spectroscopic sensor in the handheld or wearable device which emits light beams toward food and receives the light beams after the light beams have been reflected from (or passed through) food; wherein food images captured by the camera and data concerning changes in the spectra of the light beams caused by reflection from (or passage through) food are analyzed in order to identify food type, composition (e.g. nutritional composition), and/or quantity using multivariate statistical analysis to obtain more accurate results than are possible by analysis of either food images alone or spectroscopic data alone. In another example, a system can comprise: a handheld or wearable device; an auto-focusing camera in the handheld or wearable device which captures images of food; and a spectroscopic sensor in the handheld or wearable device which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In an example, a system for nutritional monitoring and management can comprise: a handheld phone; a camera in the handheld phone which captures images of food at a first time and at a second time, wherein the first time is before a person eats the food and the second time is after the person has finished eating some or all of the food; a spectroscopic sensor in the handheld phone which emits light beams toward food and receives the light beams after the light beams have been reflected from (or passed through) food at the first time and at the second time; wherein food images captured by the camera and changes in the spectra of the light beams caused by reflection from (or passage through) food are analyzed together (e.g. in multivariate analysis) to identify the type, composition, and/or quantity of food eaten by the person holding or wearing the device. In another example, a system can comprise: a handheld phone; a camera in the phone which captures images of food; a spectroscopic sensor in the phone which emits light beams toward the food and receives the light beams after the light beams have been reflected from (or passed through) the food; wherein a light pattern formed by the projection of light beams from the spectroscopic sensor on (or near) the food is used as a fiducial marker to estimate food size, distance, and/or orientation relative to the phone; and wherein changes in the spectra of light beams caused by reflection from (or passage through) the food and food images captured by the camera are analyzed to identify food type, composition, and/or quantity.

In an example, a system for nutritional monitoring and management can comprise: a handheld phone; a camera in the phone which captures images of food; and a spectroscopic sensor in the phone which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are identified in food images to estimate food distance. Alternatively, a system can comprise: a handheld phone; a spectroscopic sensor in the phone which emits light beams toward food and receives the light beams after the light beams have been reflected from (or passed through) the food; a camera in the phone which captures images of the food; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

A system can be embodied in: a handheld phone; a spectroscopic sensor in the phone which emits light beams toward food and receives the light beams after the light beams have been reflected from (or passed through) food; a camera in the phone which captures images of the food; a first laser which projects a first coherent light beam toward the food; a second laser which projects a second coherent light beam toward the food; wherein the distance between the locations of incidence of the first and second light beams on (or near) the food is used to estimate food size, distance, and/or orientation relative to the phone; and wherein changes in the spectra of light beams caused by reflection from (or passage through) the food and food images captured by the camera are analyzed to identify food type, composition (e.g. nutritional composition), and/or quantity. Alternatively, a system can comprise: a handheld phone; a spectroscopic sensor in the phone which emits light beams toward food and receives the light beams after the light beams have been reflected from (or passed through) food; a camera in the phone which captures images of the food; and a laser which projects an array of nested rings of light (or near) the food, wherein the array serves as a fiducial marker to help estimate food size, distance, and/or orientation; and wherein changes in the spectra of light beams caused by reflection from (or passage through) the food and food images captured by the camera are analyzed to identify food type, composition, and/or quantity.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and a laser which projects an quadrilateral grid of light on (or near) the food, wherein the size and/or keystone distortion of the (quadrilateral elements in the) grid serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. Alternatively, a system can comprise: a handheld phone; a spectroscopic sensor in the phone which emits light beams toward food and receives the light beams after the light beams have been reflected from (or passed through) food; a camera in the phone which captures images of the food; and a light pattern projector which projects an arcuate (e.g. circular or keystone-distorted circular) light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and a scanning (e.g. moving) laser which projects an matrix (e.g. dot matrix or linear grid) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. In another example, a system can comprise: a handheld phone; a spectroscopic sensor in the phone which emits light beams toward food and receives the light beams after the light beams have been reflected from (or passed through) the food; a camera in the phone which captures images of the food from different perspectives and angles as the phone is moved; wherein the shape, size, color, tone, brightness, and/or texture of food in the food images and changes in the spectra of the light beams caused by reflection from (or passage through) the food are analyzed together (in a multivariate manner) in order to identify the food's type and/or the food's composition; and wherein food images from different perspectives and angles are used to model the food in three dimensions in order to measure food quantity.

In an example, a system for nutritional monitoring and management can comprise: a handheld phone; a spectroscopic sensor in the phone, wherein the spectroscopic sensor further comprises a light emitter which emits light beams toward food and a light receiver which receives the light beams after the light beams have been reflected from (or passed through) the food; a camera in the phone which captures images of the food; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. Alternatively, a system can comprise: a handheld phone; a spectroscopic sensor with an aperture on the distal surface of the phone which emits light beams toward food and receives the light beams after the light beams have been reflected from (or passed through) the food; a first camera which captures images of the food; wherein the aperture of the first camera is located to one side of the aperture of the spectroscopic sensor; a second camera which captures images of the food; wherein the aperture of the second camera is located to a second side of (e.g. on the opposite side of) the aperture of the spectroscopic sensor; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

In an example, a system for nutritional monitoring and management can comprise: a handheld phone; a camera in the phone which captures images of a multi-food meal; a spectroscopic sensor in the phone which emits light beams toward food and receives the light beams after the light beams have been reflected from (or passed through) food; and a laser pointer in the phone which guides the person concerning where to position the phone so that camera captures images of each type of food in the multi-food meal and/or the spectroscopic sensor emits light beams toward each type of food in the multi-food meal; wherein food images captured by the camera and changes in the spectra of the light beams caused by reflection from (or passage through) food are analyzed to identify food type, composition, and/or quantity for each type of food in the multi-food meal.

In another example, a system for nutritional monitoring and management can comprise: a handheld phone; a camera in the phone which captures images of food; a spectroscopic sensor in the phone which emits light beams toward food and receives the light beams after the light beams have been reflected from (or passed through) food; wherein the camera and spectroscopic sensor are both directed toward a first food in a meal at a first point in time; wherein the camera and spectroscopic sensor are both directed toward a second food in a meal at a second point in time; wherein food images captured by the camera and changes in the spectra of the light beams caused by reflection from (or passage through) food are analyzed to identify the compositions and quantities of the first and second foods.

In an example, a system for nutritional monitoring and management can comprise: a handheld phone; a camera in the phone which captures images of food; a spectroscopic sensor in the phone which emits light beams toward the food and receives the light beams after the light beams have been reflected from (or passed through) the food; wherein light beams emitted from the spectroscopic sensor create a projected light pattern on (or near) the food and wherein the size, shape, and/or keystone distortion of this projected light pattern is used to estimate food size, distance, and/or orientation relative to the phone; and wherein changes in the spectra of light beams caused by reflection from (or passage through) the food and food images captured by the camera are analyzed to identify food type, composition (e.g. nutritional composition), and/or quantity. Alternatively, a system can comprise: a handheld phone; a camera in the phone which captures images of food; and a spectroscopic sensor in the phone which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are used to help estimate food size.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; a first laser which projects a first coherent light beam toward the food; a second laser which projects a second coherent light beam toward the food; a third laser which projects a third coherent light beam toward the food; wherein the distances and angles between the locations of incidence of the first, second, and third light beams on (or near) the food are used to estimate food size, distance, and/or orientation; and wherein changes in the spectra of light beams caused by reflection from (or passage through) the food and food images captured by the camera are analyzed to identify food type, composition (e.g. nutritional composition), and/or quantity. In another example, a system can comprise: a handheld phone; a spectroscopic sensor in the phone which emits light beams toward food and receives the light beams after the light beams have been reflected from (or passed through) food; a camera in the phone which captures images of the food; and a laser which projects an array of nested rings of light (or near) the food, wherein the size and distortion of rings in the array is used to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and a light pattern projector which projects a pattern of light on (or near) the food; and wherein changes in the spectra of light beams caused by reflection from (or passage through) the food and food images captured by the camera are analyzed to identify food type, composition, and/or quantity. Alternatively, a system can comprise: a handheld phone; a spectroscopic sensor in the phone which emits light beams toward food and receives the light beams after the light beams have been reflected from (or passed through) food; a camera in the phone which captures images of the food; and a scanning (e.g. moving) laser which projects a pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and a scanning laser which projects an arcuate (e.g. circular) light pattern toward the food; wherein the shape, size, and/or keystone distortion of the projected light pattern on (or near) the food is used to estimate food size, distance, and/or orientation relative to the phone; and wherein changes in the spectra of light beams caused by reflection from (or passage through) the food and food images captured by the camera are analyzed to identify food type, composition, and/or quantity. Alternatively, a system can comprise: a handheld phone; a spectroscopic sensor in the phone which emits light beams toward food and receives the light beams after the light beams have been reflected from (or passed through) the food; a camera in the phone which captures images of the food from different perspectives and angles as the phone is moved; wherein the food images and changes in the spectra of the light beams caused by reflection from (or passage through) the food are analyzed together (in a multivariate manner) in order to identify the food's type and/or the food's composition; and wherein food images from different perspectives and angles are used to model the food in three dimensions in order to measure food quantity.

In an example, a system for nutritional monitoring and management can comprise: a handheld phone; a spectroscopic sensor on the distal surface in the phone which emits light beams toward food and receives the light beams after the light beams have been reflected from (or passed through) the food; a camera on the distal surface of the phone which captures images of the food; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. Alternatively, a system can comprise: a handheld phone; a spectroscopic sensor with an aperture on the distal surface of the phone which emits light beams toward food and receives the light beams after the light beams have been reflected from (or passed through) the food; a camera with an aperture on the distal surface of the phone which captures images of the food; wherein the aperture of the spectroscopic sensor is the co-located with, co-axial with, and/or the same as the aperture of the camera; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

In an example, a system for nutritional monitoring and management can comprise: a handheld phone; a camera in the phone which captures images of food; a laser pointer which is directed toward the food; a spectroscopic sensor in the phone which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity.

In another example, a system for nutritional monitoring and management can comprise: a handheld phone; a camera in the phone which captures images of food; a spectroscopic sensor in the phone which emits light beams toward food and receives the light beams after the light beams have been reflected from (or passed through) food; wherein the person directs the camera and the spectroscopic sensor toward a first food in a meal at a first point in time; wherein the person directs the camera and the spectroscopic sensor toward a second food in a meal at a second point in time; wherein food images captured by the camera and changes in the spectra of the light beams caused by reflection from (or passage through) food are analyzed to identify the compositions and quantities of the first and second foods.

A system can be embodied in: a handheld phone; a camera in the phone which captures images of food; a spectroscopic sensor in the phone which emits light beams toward the food and receives the light beams after the light beams have been reflected from (or passed through) the food; wherein part of the spectrum of light beams emitted from the spectroscopic sensor create a light pattern on (or near) the food which is used as a fiducial marker to estimate food size, distance, and/or orientation relative to the phone; and wherein changes in the spectra of light beams caused by reflection from (or passage through) the food and food images captured by the camera are analyzed to identify food type, composition, and/or quantity. Alternatively, a system can comprise: a handheld phone; a camera in the phone which captures images of food; and a spectroscopic sensor in the phone which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are identified in food images to help estimate food size, distance, and/or orientation.

In an example, a system for nutritional monitoring and management can comprise: a handheld phone; a camera in the phone which captures images of food; and a spectroscopic sensor in the phone which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are used to estimate food distance. In another example, a system can comprise: a handheld phone; a spectroscopic sensor in the phone which emits light beams from the distal surface of the device toward food and receives the light beams after the light beams have been reflected from (or passed through) the food; a camera in the phone which captures images of the food from the distal surface of the device; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

A system can be embodied in: a handheld phone; a spectroscopic sensor in the phone which emits light beams toward food and receives the light beams after the light beams have been reflected from (or passed through) food; a camera in the phone which captures images of the food; and a laser which projects a target (e.g. cross-hairs) light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. Alternatively, a system can comprise: a handheld phone; a spectroscopic sensor in the phone which emits light beams toward food and receives the light beams after the light beams have been reflected from (or passed through) food; a camera in the phone which captures images of the food; and a laser which projects an matrix (e.g. dot matrix or linear grid) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and a light pattern projector which projects a pattern of light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity. Alternatively, a system can comprise: a handheld phone; a spectroscopic sensor in the phone which emits light beams toward food and receives the light beams after the light beams have been reflected from (or passed through) food; a camera in the phone which captures images of the food; and a scanning (e.g. moving) laser which projects a polygonal light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and a scanning laser which projects an arcuate (e.g. circular) light pattern toward the food; wherein size of the projected light pattern on (or near) the food is used to estimate food distance; wherein keystone distortion of the projected light pattern on (or near) the food is used to estimate the orientation of the food relative to the phone; and wherein changes in the spectra of light beams caused by reflection from (or passage through) the food and food images captured by the camera are analyzed to identify food type, composition (e.g. nutritional composition), and/or quantity. In another example, a system can comprise: a handheld phone; a spectroscopic sensor with an aperture on the distal surface of the phone which emits light beams toward food and receives the light beams after the light beams have been reflected from (or passed through) the food; a camera with an aperture on the distal surface of the phone which captures images of the food; wherein the aperture of the spectroscopic sensor is between 5 mm and 100 mm away from the aperture of the camera; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity.

In an example, a system for nutritional monitoring and management can comprise: a handheld phone; a camera in the phone which captures images of a multi-food meal; a laser pointer which is sequentially pointed toward different types of food in the multi-food meal; a spectroscopic sensor in the phone is sequentially pointed toward different types of food in the meal, wherein the spectroscopic sensor emits light beams toward a type of food and receives the light beams after the light beams have been reflected from (or passed through) the type of food; wherein food images captured by the camera and changes in the spectra of the light beams caused by reflection from (or passage through) food are analyzed to identify food type, composition, and/or quantity for each type of food in the multi-food meal. Alternatively, a system can comprise: a handheld phone; a camera in the phone which sequentially captures an image of each type of food in a multi-food meal; a spectroscopic sensor in the phone which sequentially emits light beams toward each type of food in the multi-food meal and receives the light beams after the light beams have been reflected from (or passed through) the type of food; and a laser pointer in the phone which guides the person concerning where to position the phone so that camera captures an image of each type of food in the multi-food meal and/or the spectroscopic sensor sequentially emits light beams toward each type of food in the multi-food meal; wherein food images captured by the camera and changes in the spectra of the light beams caused by reflection from (or passage through) food are analyzed to identify food type, composition, and/or quantity for each type of food in the multi-food meal.

In an example, a system for nutritional monitoring and management can comprise: a handheld phone; a camera in the handheld phone which captures images of food at a first time and at a second time; and a spectroscopic sensor in the handheld phone which emits light beams toward food and receives the light beams after the light beams have been reflected from (or passed through) food at the first time and at the second time; wherein food images captured by the camera and changes in the spectra of the light beams caused by reflection from (or passage through) food are analyzed together (e.g. in multivariate analysis) to identify the type, composition (e.g. nutritional composition), and/or quantity of food eaten by the person holding or wearing the device. In another example, a system can comprise: a handheld phone; a camera in the phone which captures images of food; a spectroscopic sensor in the phone which emits light beams toward food and receives the light beams after the light beams have been reflected from (or passed through) food; a laser pointer; wherein the person uses the laser pointer to direct the camera and the spectroscopic sensor toward a first food in a meal at a first point in time; wherein the person uses the laser pointer to direct the camera and the spectroscopic sensor toward a second food in a meal at a second point in time; wherein food images captured by the camera and changes in the spectra of the light beams caused by reflection from (or passage through) food are analyzed to identify the compositions and quantities of the first and second foods.

A system can be embodied in: a handheld phone; a camera in the phone which captures images of food; a spectroscopic sensor in the phone which emits light beams toward the food and receives the light beams after the light beams have been reflected from (or passed through) the food; wherein the visible portion of the spectrum of light beams emitted from the spectroscopic sensor creates a visible light pattern on (or near) the food and wherein the size, shape, and/or keystone distortion of this visible light pattern is used as a fiducial marker to estimate food size, distance, and/or orientation relative to the phone; and wherein changes in the spectra of light beams caused by reflection from (or passage through) the food and food images captured by the camera are analyzed to identify food type, composition (e.g. nutritional composition), and/or quantity. Alternatively, a system can comprise: a handheld phone; a camera in the phone which captures images of food; and a spectroscopic sensor in the phone which emits light beams toward food and receives the light beams after the light beams have been reflected from (or passed through) food; wherein food images captured by the camera and changes in the spectra of the light beams caused by reflection from (or passage through) food are analyzed to identify food type, composition (e.g. nutritional composition), and/or quantity; and wherein one or more eating-related objects (e.g. bowl, chopsticks, cup, fork, glass, knife, mug, napkin, placemat, plate, or spoon) are identified in food images to help estimate food size.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; a first laser which projects a first coherent light beam toward the food; and a second laser which projects a second coherent light beam toward the food; wherein the first and second light beams form a projected light pattern on (or near) the food which serves as a fiducial marker to help estimate food size, distance, and/or orientation; and wherein changes in the spectra of light beams caused by reflection from (or passage through) the food and food images captured by the camera are analyzed to identify food type, composition, and/or quantity. Alternatively, a system can comprise: a handheld phone; a spectroscopic sensor in the phone which emits light beams toward food and receives the light beams after the light beams have been reflected from (or passed through) food; a camera in the phone which captures images of the food; and a laser which projects an arcuate (e.g. circular, elliptical, or egg-shaped) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity.

A system can be embodied in: a handheld phone; a spectroscopic sensor in the phone which emits light beams toward food and receives the light beams after the light beams have been reflected from (or passed through) food; a camera in the phone which captures images of the food; and a laser which projects an quadrilateral grid of light on (or near) the food, wherein the grid serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity. Alternatively, a system can comprise: a handheld phone; a spectroscopic sensor in the phone which emits light beams toward food and receives the light beams after the light beams have been reflected from (or passed through) food; a camera in the phone which captures images of the food; and a light pattern projector which projects a polygonal light pattern onto the food and/or a surface near the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity.

In an example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and a scanning (e.g. moving) laser which projects an arcuate (e.g. circular, elliptical, or egg-shaped) pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition (e.g. nutritional composition), and/or quantity.

In another example, a system for nutritional monitoring and management can comprise: a handheld phone; a spectroscopic sensor in the phone which emits light beams toward food and receives the light beams after the light beams have been reflected from (or passed through) food; a camera in the phone which captures images of the food; and one or more lasers which project a pattern of coherent light on (or near) the food, wherein the light pattern serves as a fiducial marker to help estimate food size, distance, and/or orientation; and wherein changes in the spectra of light beams caused by reflection from (or passage through) the food and food images captured by the camera are analyzed to identify food type, composition, and/or quantity.

A system can be embodied in: a handheld phone; a spectroscopic sensor with an aperture on the distal surface of the phone which emits light beams toward food and receives the light beams after the light beams have been reflected from (or passed through) the food; a camera with an aperture on the distal surface of the phone which captures images of the food; wherein the aperture of the spectroscopic sensor is between 1 mm and 10 mm away from the aperture of the camera; wherein the food images and changes in the spectra of the light beams caused by reflection from (or passage through) the food are analyzed together (in a multivariate manner) in order to identify the food's type and/or measure the food's composition; and wherein the food images are analyzed to measure food quantity. Alternatively, a system can comprise: a handheld phone; a camera in the phone which captures images of a multi-food meal; a laser pointer which is sequentially pointed toward different portions of food in the multi-food meal; a spectroscopic sensor in the phone is sequentially pointed toward different portions of food in the meal, wherein the spectroscopic sensor emits light beams toward a portion of food and receives the light beams after the light beams have been reflected from (or passed through) the portion of food; wherein food images captured by the camera and changes in the spectra of the light beams caused by reflection from (or passage through) food are analyzed to identify food type, composition (e.g. nutritional composition), and/or quantity for each portion of food in the multi-food meal.

In an example, a system for nutritional monitoring and management can comprise: a handheld phone; a camera in the phone which captures images of food; a spectroscopic sensor in the phone which emits light beams toward food and receives the light beams after the light beams have been reflected from (or passed through) food; and a laser pointer in the phone which guides the person concerning where to position the phone so that camera captures images of the food and/or the spectroscopic sensor emits light beams toward the food; wherein food images captured by the camera and changes in the spectra of the light beams caused by reflection from (or passage through) food are analyzed to identify food type, composition (e.g. nutritional composition), and/or quantity. In another example, a system can comprise: a handheld phone which is held by a person; a camera in the handheld device which captures images of food; a spectroscopic sensor in the handheld device which emits light beams toward food and receives the light beams after the light beams have been reflected from (or passed through) food; a wrist-worn device which is worn by the person; and a motion sensor in the wrist-worn device which tracks hand-to-mouth or chewing motions; wherein data from the camera, the spectroscopic sensor, and the motion sensor are analyzed together (e.g. in multivariate analysis) to identify the type, composition (e.g. nutritional composition), and/or quantity of food eaten by the person holding or wearing the device.

In an example, a system for nutritional monitoring and management can comprise: a handheld phone which is held by a person; a camera in the handheld device which captures images of food; a spectroscopic sensor in the handheld device which emits light beams toward food and receives the light beams after the light beams have been reflected from (or passed through) food; a smart watch which is worn by the person; and a motion sensor in the smart watch which tracks hand-to-mouth or chewing motions; wherein data from the camera, the spectroscopic sensor, and the motion sensor are analyzed together (e.g. in multivariate analysis) to identify the type, composition, and/or quantity of food eaten by the person holding or wearing the device. Alternatively, a system can comprise: a handheld phone which is held by a person; a camera in the handheld device which captures images of food; a spectroscopic sensor in the handheld device which emits light beams toward food and receives the light beams after the light beams have been reflected from (or passed through) food; a neck-worn device which is worn by the person; and a motion sensor in the neck-worn device which tracks chewing or swallowing motions; wherein data from the camera, the spectroscopic sensor, and the motion sensor are analyzed together (e.g. in multivariate analysis) to identify the type, composition (e.g. nutritional composition), and/or quantity of food eaten by the person holding or wearing the device.

In an example, a system for nutritional monitoring and management can comprise: a handheld phone which is held by a person; a camera in the handheld device which captures images of food; a spectroscopic sensor in the handheld device which emits light beams toward food and receives the light beams after the light beams have been reflected from (or passed through) food; a wearable device which is worn by the person; and a motion sensor in the handheld device which tracks hand-to-mouth motions, chewing motions, and/or swallowing motions; wherein data from the camera, the spectroscopic sensor, and the motion sensor are analyzed together (e.g. in multivariate analysis) to identify the type, composition, and/or quantity of food eaten by the person holding or wearing the device. Alternatively, a system can comprise: a handheld phone which is held by a person; a camera in the handheld device which captures images of food; a spectroscopic sensor in the handheld device which emits light beams toward food and receives the light beams after the light beams have been reflected from (or passed through) food; a neck-worn device which is worn by the person; and a sound sensor in the neck-worn device which tracks chewing or swallowing sounds; wherein data from the camera, the spectroscopic sensor, and the sound sensor are analyzed together (e.g. in multivariate analysis) to identify the type, composition (e.g. nutritional composition), and/or quantity of food eaten by the person holding or wearing the device.

In an example, a system for nutritional monitoring and management can comprise: a handheld phone which is waived back and forth several times over a multi-food meal; a spectroscopic sensor in the handheld phone which emits light beams toward the meal and receives the light beams after the light beams have been reflected from (or passed through) a plurality of locations on the meal as the device is waived back and forth; a camera in the handheld phone which captures images of a plurality of locations on the meal as the device is waived back and forth; and a motion sensor; wherein data from the spectroscopic sensor, the camera, and the motion sensor are analyzed together (in a multivariate manner) in order to identify the types, compositions, and/or quantities of foods in the multi-food meal. Alternatively, a system can comprise: a handheld phone which is waived back and forth several times over a multi-food meal; a spectroscopic sensor in the handheld phone which emits light beams toward the meal and receives the light beams after the light beams have been reflected from (or passed through) a plurality of locations on the meal as the device is waived back and forth; a camera in the handheld phone which captures images of a plurality of locations on the meal as the device is waived back and forth; and a motion sensor; wherein data from the spectroscopic sensor, the camera, and the motion sensor are analyzed to segment the meal into different food portions; and wherein data from the spectroscopic sensor, the camera, and the motion sensor are analyzed to identify the types, compositions, and/or quantities of foods in the different food portions.

A system can be embodied in: a handheld phone which is waived back and forth several times over a multi-food meal; a spectroscopic sensor in the handheld phone which emits light beams toward the meal and receives the light beams after the light beams have been reflected from (or passed through) a plurality of locations on the meal as the device is waived back and forth; a camera in the handheld phone which captures images of a plurality of locations on the meal as the device is waived back and forth; and a motion sensor; wherein variations in food color, tone, brightness, texture, shape, and molecular composition as the device is waived back and forth are analyzed to segment the meal into different food portions; and wherein data from the spectroscopic sensor, the camera, and the motion sensor are analyzed to identify the types, compositions, and/or quantities of foods in the different food portions. In another example, a system can comprise: a handheld phone which is waived back and forth several times over a multi-food meal; a spectroscopic sensor in the handheld phone which emits light beams toward the meal and receives the light beams after the light beams have been reflected from (or passed through) the meal as the device is being waived back and forth; a camera in the handheld phone which captures images of the meal as the device is being waived back and forth; and a motion sensor; wherein data from the spectroscopic sensor, the camera, and the motion sensor are analyzed together (in a multivariate manner) in order to identify the types, compositions, and/or quantities of foods in the multi-food meal.

In an example, a system for nutritional monitoring and management can comprise: a handheld phone which is waived back and forth several times over food; a spectroscopic sensor in the handheld phone which emits light beams toward food and receives the light beams after the light beams have been reflected from (or passed through) the food as the device is being waived back and forth; a camera in the handheld phone which captures images of the food as the device is being waived back and forth; and a motion sensor; wherein data from the spectroscopic sensor, the camera, and the motion sensor are analyzed together (in a multivariate manner) in order to identify the food type, composition, and/or quantity. Alternatively, a system can comprise: a handheld phone which is waived in an arc segment of a circle over nearby food; a spectroscopic sensor in the handheld phone which emits light beams toward food and receives the light beams after the light beams have been reflected from (or passed through) the food; a camera in the handheld phone which captures images of the food; and a motion sensor; wherein data from the spectroscopic sensor, the camera, and the motion sensor are analyzed together (in a multivariate manner) in order to identify the food type, composition (e.g. nutritional composition), and/or quantity.

A system for nutritional monitoring and management can be embodied in: a handheld phone which is waived over a meal in an arc which is wider than the width of the meal; a spectroscopic sensor in the handheld phone which emits light beams toward food and receives the light beams after the light beams have been reflected from (or passed through) the food; a camera in the handheld phone which captures images of the food; and a motion sensor; wherein data from the spectroscopic sensor, the camera, and the motion sensor are analyzed together (in a multivariate manner) in order to identify the food type, composition (e.g. nutritional composition), and/or quantity. In another example, a system can comprise: a handheld phone which is waived over a meal with multiple types of food; a spectroscopic sensor in the handheld phone which emits light beams toward food and receives the light beams after the light beams have been reflected from (or passed through) the food; a camera in the handheld phone which captures images of the food in a field of view which overlaps the projection path of light beams from the spectroscopic sensor; and a motion sensor; wherein the handheld phone guides a person concerning how to waive or otherwise move the handheld phone over the meal with multiple types of food; and wherein data from the spectroscopic sensor, the camera, and the motion sensor are analyzed together (in a multivariate manner) in order to identify the food type, composition (e.g. nutritional composition), and/or quantity.

========B===========

In an example, a wearable food consumption monitoring device can comprise eyeglasses with one or more automatic food imaging members (e.g. cameras), wherein images recorded by the cameras are automatically analyzed to estimate the types and quantities of food consumed by a person. In an example, one or more cameras can start recording images when they are triggered by food consumption detected by analysis of data from one or more sensors selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, infrared sensor, spectroscopy sensor, electrogoniometer, chewing sensor, swallowing sensor, temperature sensor, and pressure sensor.

In an example, a device can comprise eyeglasses which further comprise one or more automatic food imaging members (e.g. cameras). Pictures taken by an imaging member can be automatically analyzed in order to estimate the types and quantities of food which are consumed by a person. Food can refer to beverages as well as solid food. An automatic imaging member can take pictures when it is activated (triggered) by food consumption based on data collected by one or more sensors selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallowing sensor, temperature sensor, and pressure sensor. In an example, when data from one or more sensors indicates that a person is probably consuming food, then this can activate (trigger) an imaging member to start taking pictures and/or recording images.

In an example, eyeglasses to monitor food consumption can include a camera which records images along an imaging vector which points toward a person's mouth. In an example, a camera can record images of a person's mouth and the interaction between food and the person's mouth. Interaction between food and a person's mouth can include biting, chewing, and/or swallowing. In an example, eyeglasses for monitoring food consumption can include a camera which records images along an imaging vector which points toward a reachable food source. In an example, eyeglasses can include two cameras: a first camera which records images along an imaging vector which points toward a person's mouth and a second camera which records images along an imaging vector which points toward a reachable food source.

In an example, a device can comprise at least two cameras or other imaging members. A first camera can take pictures along an imaging vector which points toward a person's mouth while the person eats. A second camera can take pictures along an imaging vector which points toward a reachable food source. In an example, this device can comprise one or more imaging members that take pictures of: food at a food source; a person's mouth; and interaction between food and the person's mouth. Interaction between the person's mouth and food can include biting, chewing, and swallowing. In an example, utensils or beverage-holding members may be used as intermediaries between the person's hand and food. In an example, this invention can comprise an imaging device that automatically takes pictures of the interaction between food and the person's mouth as the person eats. In an example, this device can comprise a wearable device that takes pictures of a reachable food source that is located in front of a person. In an example, such a device can track the location of, and take pictures of, a person's mouth track the location of, and take pictures of, a person's hands; and scan for, and take pictures of, reachable food sources nearby.

In an example, a system for food consumption monitoring can include eyeglasses and a wrist-worn device (e.g. smart watch) which are in electromagnetic communication with each other. In an example, a system for food consumption monitoring can comprise eyeglasses and a wrist-worn motion sensor. In an example, a wrist-worn motion sensor can detect a pattern of hand and/or arm motion which is associated with food consumption. In an example, this pattern of hand and/or arm motion can comprise: hand movement toward a reachable food source; hand movement up to a person's mouth; lateral motion and/or hand rotation to bring food into the mouth; and hand movement back down to the original level. In an example, a food consumption monitoring device can continually track the location of a person's hand to detect when it comes near the person's mouth and/or grasps a reachable food source.

In an example, an imaging member can automatically start taking pictures and/or recording images when data from a wrist-worn motion sensor shows a pattern of hand and/or arm motion which is generally associated with food consumption. In an example, this pattern of hand and/or arm motion can comprise: hand movement toward a reachable food source; hand movement up to a person's mouth; lateral motion and/or hand rotation to bring food into the mouth; and hand movement back down to the original level. In an example, electronically-functional eyewear can be in wireless communication with a motion sensor which is worn on a person's wrist, finger, hand, or arm. In an example, this motion sensor can detect hand, finger, wrist, and/or arm movements which indicate that a person is preparing food for consumption and/or bringing food up to their mouth.

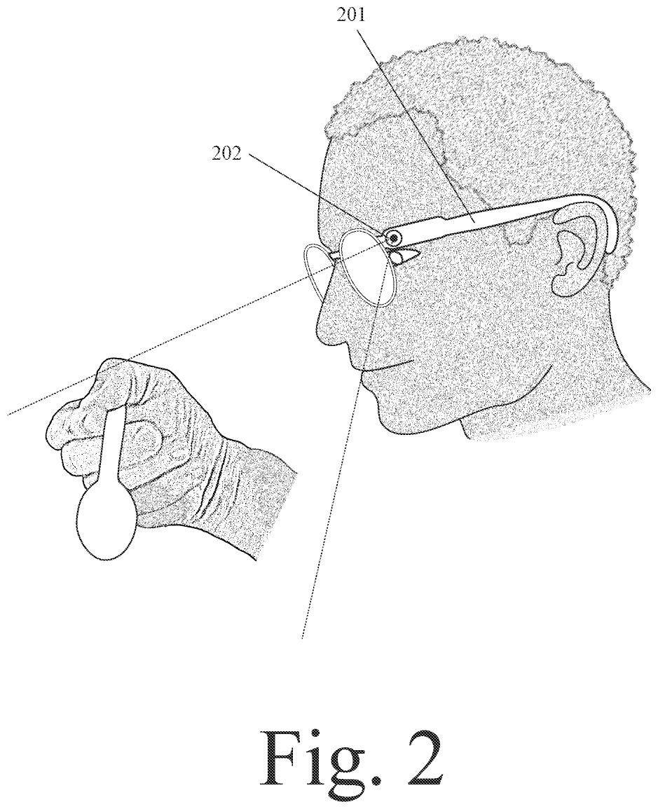

FIG. 2 shows an example of smart eyewear for measuring food consumption comprising: an eyewear frame 201 worn by a person; and a camera 202 on the eyewear frame which records food images when activated. In an example, eyewear can be a pair of eyeglasses. In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear.

In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

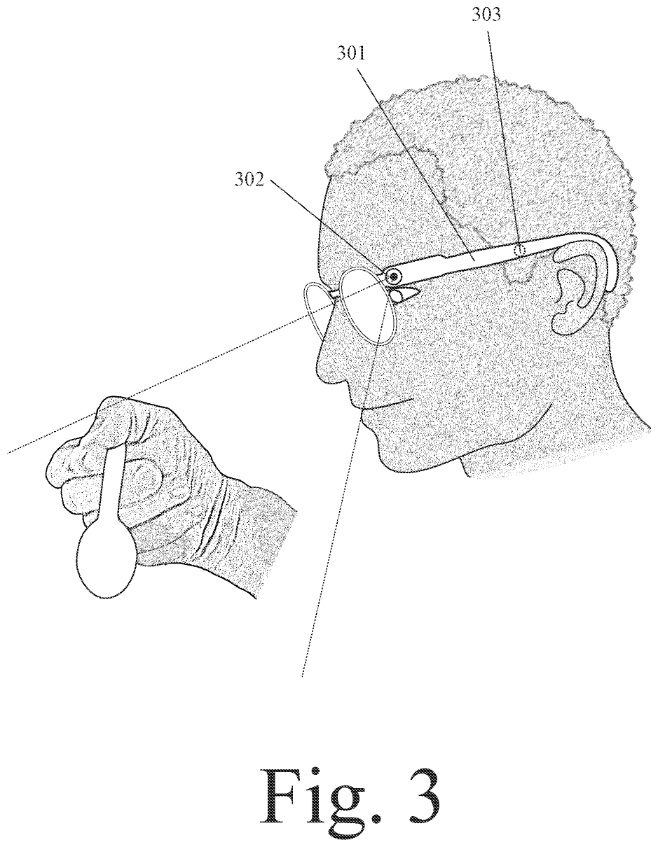

FIG. 3 shows an example of smart eyewear for measuring food consumption comprising: an eyewear frame 301 worn by a person; a camera 302 on the eyewear frame which records food images when activated; and a chewing sensor 303 on the eyewear frame which detects when the person eats, wherein the camera is activated to record food images when data from the chewing sensor indicates that the person is eating. In an example, eyewear can be a pair of eyeglasses. In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear.

In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

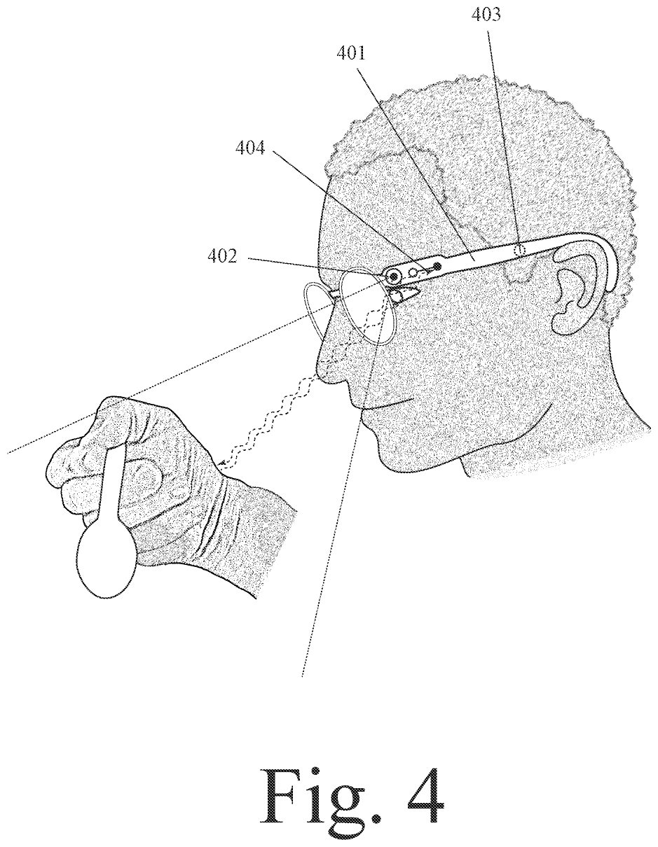

FIG. 4 shows an example of smart eyewear for measuring food consumption comprising: an eyewear frame 401 worn by a person; a camera 402 on the eyewear frame which records food images when activated; a chewing sensor 403 on the eyewear frame which detects when the person eats; and a proximity sensor 404 on the eyewear frame which uses infrared light to detect when a person eats by detecting when an object (such as the person's hand) is near the person's mouth, wherein the camera is activated to record food images when data from the chewing sensor and/or data from the proximity sensor indicate that the person is eating. In an example, eyewear can be a pair of eyeglasses.

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, a proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth activity. In an example, joint analysis of data from the chewing sensor and data from the proximity sensor can provide more accurate detection of eating than data from either sensor alone or separate analysis of data from both sensors.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

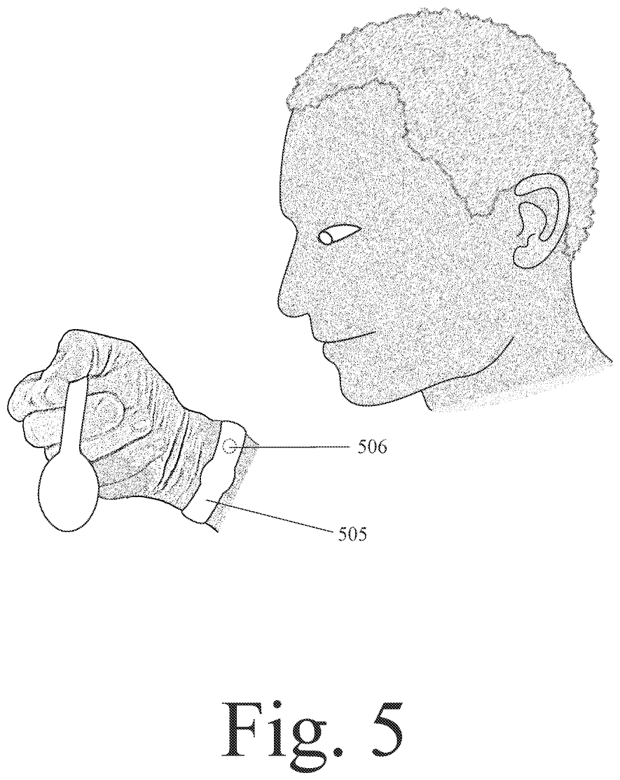

FIG. 5 shows an example of a smart watch, wrist band, or watch band for measuring food consumption comprising: a smart watch (or wrist band) 505 worn by a person; and a motion sensor 506 (e.g. accelerometer and/or gyroscope) on the smart watch (or wrist band), wherein the motion sensor is used to measure the person's food consumption.

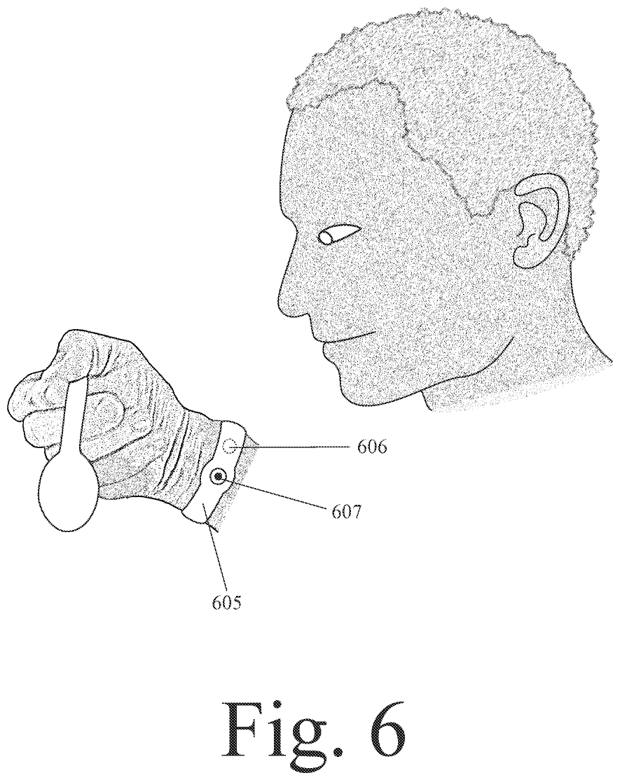

FIG. 6 shows an example of a smart watch, wrist band, or watch band for measuring food consumption comprising: a smart watch (or wrist band) 605 worn by a person; a motion sensor 606 (e.g. accelerometer and/or gyroscope) on the smart watch (or wrist band); and a camera 607 on the smart watch (or wrist band), wherein the camera is activated to record food images when data from the motion sensor indicates that the person is eating. In an example, a camera can be located on the anterior side of a person's wrist (opposite the traditional location of a watch face housing). Alternatively, a camera can be on a watch face housing. In an example, there can be two cameras on a smart watch, wrist band, or watch band to record images of nearby food, hand-to-food interactions, and hand-to-mouth interactions. In an example, one camera can be on the anterior side of a person's wrist and one camera can be on the posterior side of the person's wrist (e.g. on a watch face housing). In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

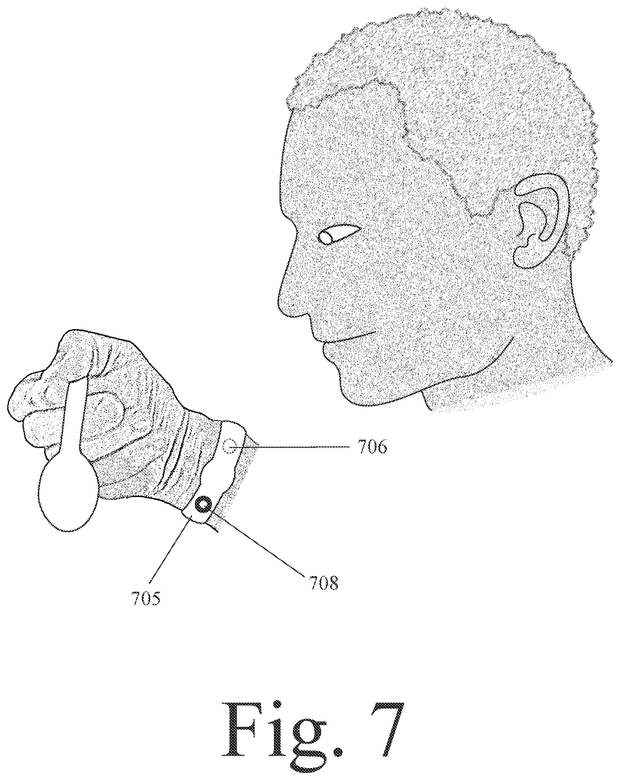

FIG. 7 shows an example of a smart watch, wrist band, or watch band for measuring food consumption comprising: a smart watch (or wrist band) 705 worn by a person; a motion sensor 706 (e.g. accelerometer and/or gyroscope) on the smart watch (or wrist band); and a spectroscopic sensor 708 on the smart watch (or wrist band) which analyzes the molecular and/or nutritional composition of food, wherein the spectroscopic sensor is activated when data from the motion sensor indicates that the person is eating. In another example, instead of the spectroscopic sensor being triggered automatically, the person can be prompted to take a spectroscopic scan of food when the motion sensor indicates that the person is eating. In an example, a person can take a spectroscopic scan of food by waving their hand over food (like Obi-Wan Kenobi). In an example, a spectroscopic sensor can be located on the anterior side of the person's wrist (opposite the traditional location of a watch face). Alternatively, a spectroscopic sensor can be located on the watch face housing. In an example, a spectroscopic sensor can emit light away from the outer surface of a smart watch (or wrist band) and toward food. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

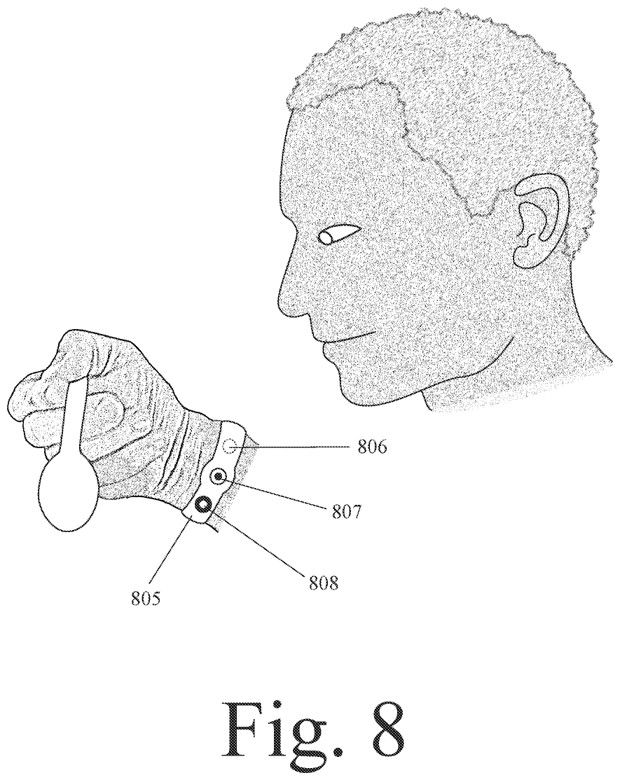

FIG. 8 shows an example of a smart watch, wrist band, or watch band for measuring food consumption comprising: a smart watch (or wrist band) 805 worn by a person; a motion sensor 806 (e.g. accelerometer and/or gyroscope) on the smart watch (or wrist band); a camera 807 on the smart watch (or wrist band), wherein the camera is activated to record food images when data from the motion sensor indicates that the person is eating; and a spectroscopic sensor 808 on the smart watch (or wrist band) which analyzes the molecular and/or nutritional composition of food, wherein the spectroscopic sensor is activated to record food images when data from the motion sensor indicates that the person is eating. In another example, instead of the spectroscopic sensor being triggered automatically, the person can be prompted to take a spectroscopic scan of food when the motion sensor indicates that the person is eating. In an example, a person can take a spectroscopic scan of food by waving their hand over food. In an example, a spectroscopic sensor can emit light away from the outer surface of a smart watch (or wrist band) and toward food. In an example, the spectroscopic sensor can emit and receive near-infrared light.

In an example, a camera on a smart watch (or wrist band) can be located on the anterior side of the person's wrist (opposite the traditional location of a watch face). Alternatively, a camera can be on a watch face housing. In an example, there can be two cameras on a smart watch, wrist band, or watch band to record images of nearby food, hand-to-food interactions, and hand-to-mouth interactions. In an example, one camera can be on the anterior side of a person's wrist and one camera can be on the posterior side of the person's wrist (e.g. on a watch face housing). In an example, one camera can be on a first lateral side of a person's wrist and another camera can be on the opposite lateral side of the person's wrist, so that one camera tends to record images of nearby food and the other camera tends to record images of the person's mouth as the person eats. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

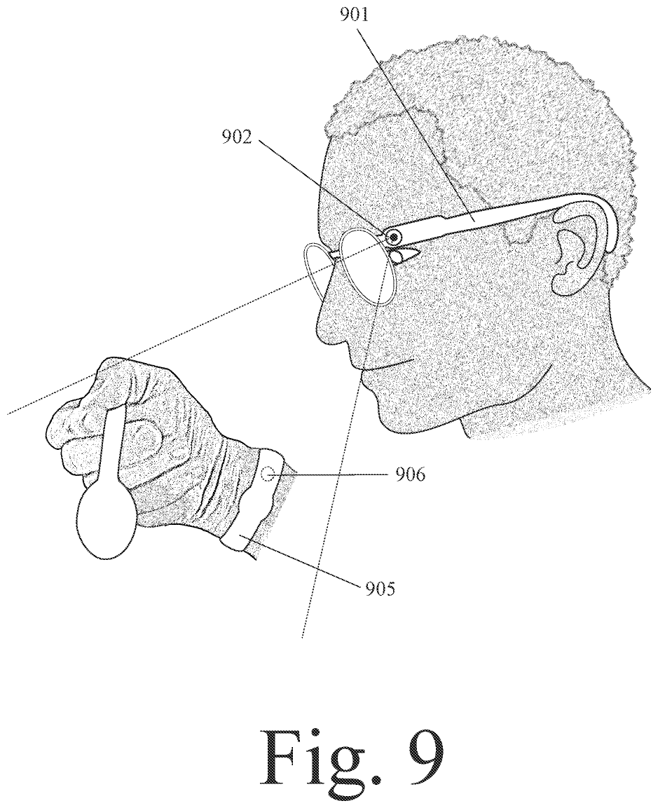

FIG. 9 shows an example of a wearable system for measuring food consumption comprising: an eyewear frame 901 worn by a person; a camera 902 on the eyewear frame which records food images when activated; a smart watch (or wrist band) 905 worn by the person; and a motion sensor 906 (e.g. accelerometer and/or gyroscope) on the smart watch (or wrist band), wherein the camera is activated to record food images when data from the motion sensor indicates that the person is eating. In an example, eyewear can be a pair of eyeglasses. In an example, there can be wrist bands with motion sensors on both (right and left) of a person's wrists to capture eating activity by both the person's dominant and non-dominant hands. In an example, eating-related motions by either hand can trigger activation of the camera on the eyewear. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

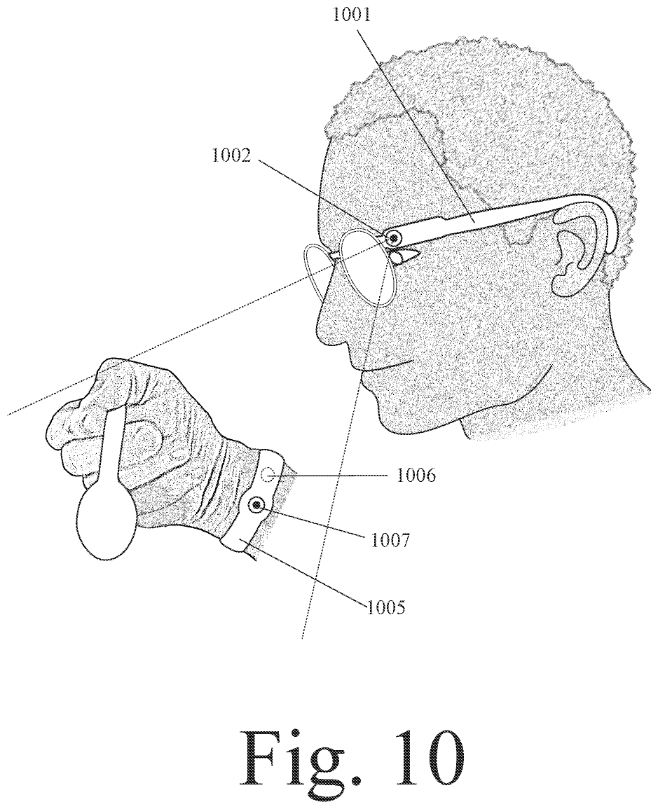

FIG. 10 shows an example of a wearable system for measuring food consumption comprising: an eyewear frame 1001 worn by a person; a smart watch (or wrist band) 1005 worn by the person; a first camera 1002 on the eyewear frame which records food images when activated; a second camera 1007 on the smart watch (or wrist band) which records food images when activated; and a motion sensor 1006 (e.g. accelerometer and/or gyroscope) on the smart watch (or wrist band), wherein the first camera and/or the second camera are activated to record food images when data from the motion sensor indicates that the person is eating. In an example, eyewear can be a pair of eyeglasses. In an example, there can be wrist bands with motion sensors on both (right and left) of a person's wrists to capture eating activity by both the person's dominant and non-dominant hands. In an example, eating-related motions by either hand can trigger activation of the camera on the eyewear. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be part of (or attached to) a sidepiece (e.g. “temple”) of the eyewear frame. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of the eyewear, one directed forward and downward (toward a person's mouth) and one directed straight forward (toward the person's hands). In an example, the focal direction of a camera on eyewear can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

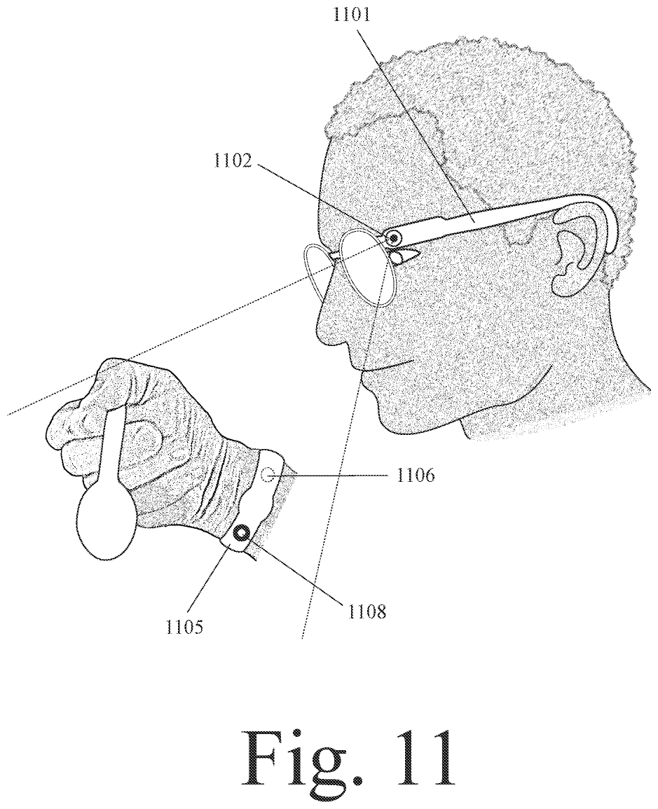

FIG. 11 shows an example of a wearable system for measuring food consumption comprising: an eyewear frame 1101 worn by a person; a camera 1102 on the eyewear frame which records food images when activated; a smart watch (or wrist band) 1105 worn by the person; a motion sensor 1106 (e.g. accelerometer and/or gyroscope) on the smart watch (or wrist band), wherein the camera is activated to record food images when data from the motion sensor indicates that the person is eating; and a spectroscopic sensor 1108 on the smart watch (or wrist band) which analyzes the molecular and/or nutritional composition of food. In an example, a spectroscopic sensor can be activated automatically when data from the motion sensor indicates that the person is eating. In an example, the person can be prompted to use a spectroscopic sensor when data from the motion sensor indicates that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, the focal direction of a camera can be changed automatically to track a person's hands. In an example, an indicator light can be on when the camera is activated. In an example, a shutter or flap can automatically cover the camera when the camera is not activated. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands).

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

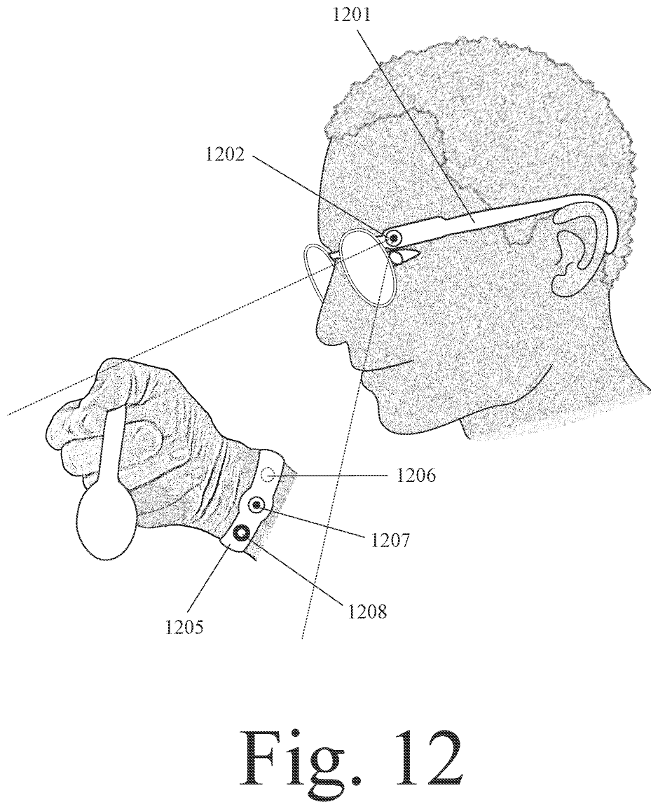

FIG. 12 shows an example of a wearable system for measuring food consumption comprising: an eyewear frame 1201 worn by a person; a smart watch (or wrist band) 1205 worn by the person; a first camera 1202 on the eyewear frame which records food images when activated; a second camera 1207 on the smart watch (or wrist band) which records food images when activated; a motion sensor 1206 (e.g. accelerometer and/or gyroscope) on the smart watch (or wrist band), wherein the first camera and/or the second camera are activated to record food images when data from the motion sensor indicates that the person is eating; and a spectroscopic sensor 1208 on the smart watch (or wrist band) which analyzes the molecular and/or nutritional composition of food. In an example, a spectroscopic sensor can be activated automatically when data from the motion sensor indicates that the person is eating. In an example, the person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be part of (or attached to) a sidepiece (e.g. “temple”) of the eyewear frame. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of the eyewear, one directed forward and downward (toward a person's mouth) and one directed straight forward (toward the person's hands). In an example, the focal direction of a camera on eyewear can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

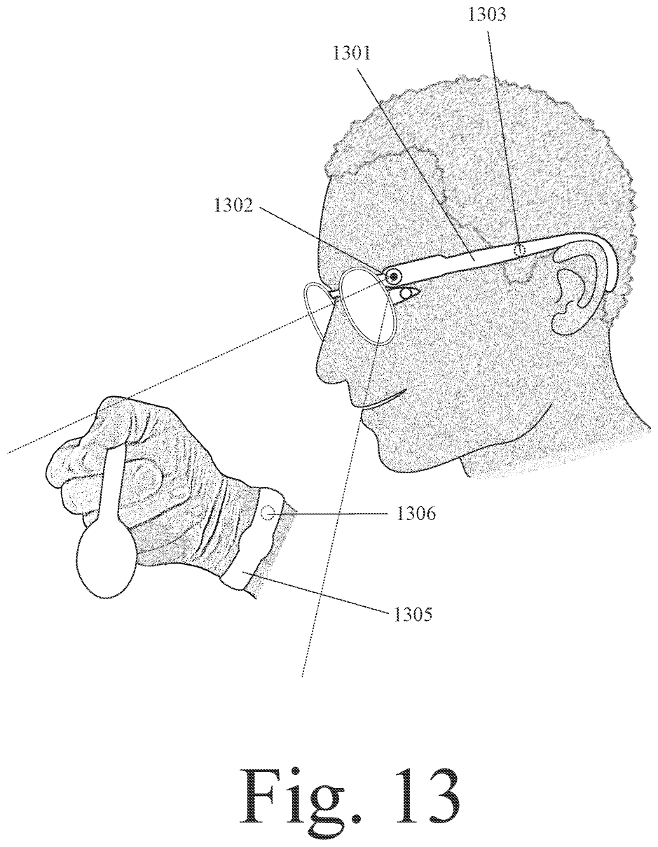

FIG. 13 shows an example of a wearable system for measuring food consumption comprising: an eyewear frame 1301 worn by a person; a camera 1302 on the eyewear frame which records food images when activated; a chewing sensor 1303 on the eyewear frame which detects when the person eats; a smart watch (or wrist band) 1305 worn by the person; and a motion sensor 1306 (e.g. accelerometer and/or gyroscope) on the smart watch (or wrist band), wherein the camera is activated to record food images when data from the chewing sensor and/or data from the motion sensor indicate that the person is eating. In an example, joint analysis of data from the chewing sensor and data from the motion sensor can provide more accurate detection of eating than data from either sensor alone or separate analysis of data from both sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, a camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

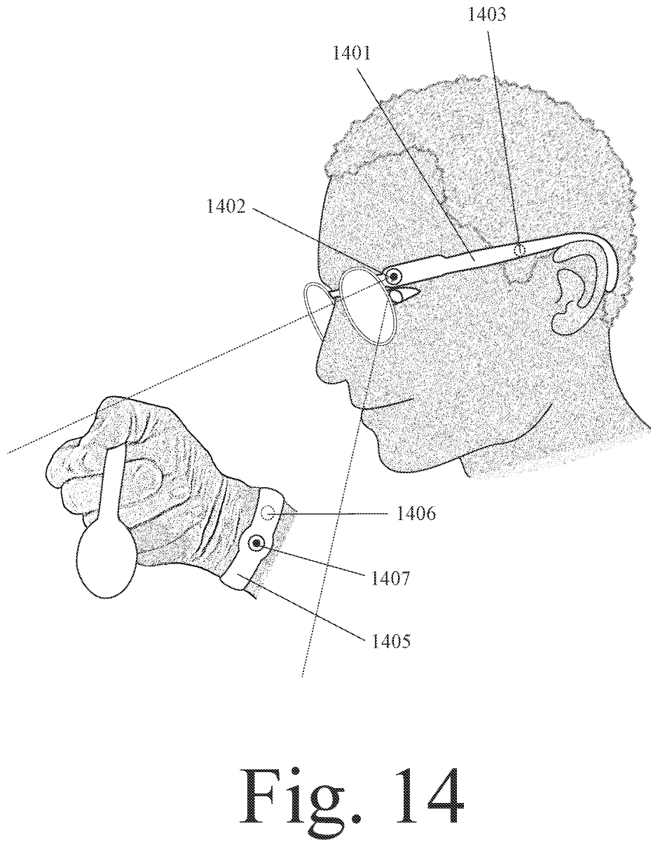

FIG. 14 shows an example of a wearable system for measuring food consumption comprising: an eyewear frame 1401 worn by a person; a chewing sensor 1403 on the eyewear frame which detects when the person eats; a smart watch (or wrist band) 1405 worn by the person; a motion sensor 1406 (e.g. accelerometer and/or gyroscope) on the smart watch (or wrist band); a first camera 1402 on the eyewear frame which records food images when activated, wherein the first camera is activated to record food images when data from the chewing sensor and/or data from the motion sensor indicate that the person is eating; and a second camera 1407 on the smart watch (or wrist band) which records food images when activated, wherein the second camera is activated to record food images when data from the chewing sensor and/or data from the motion sensor indicate that the person is eating. In an example, joint analysis of data from the chewing sensor and data from the motion sensor can provide more accurate detection of eating than data from either sensor alone or separate analysis of data from both sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

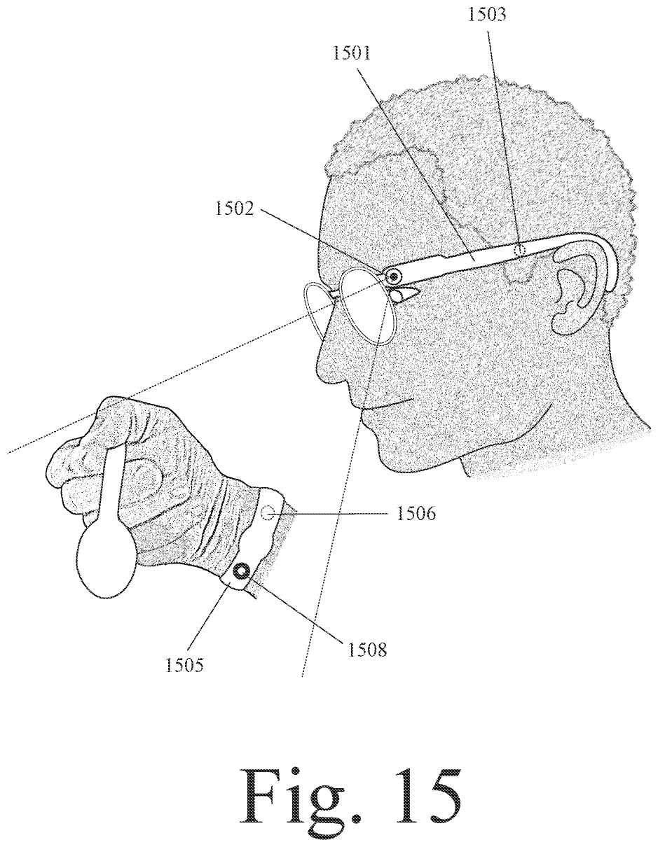

FIG. 15 shows an example of a wearable system for measuring food consumption comprising: an eyewear frame 1501 worn by a person; a chewing sensor 1503 on the eyewear frame which detects when the person eats; a smart watch (or wrist band) 1505 worn by the person; a motion sensor 1506 (e.g. accelerometer and/or gyroscope) on the smart watch (or wrist band) which detects when the person eats; a camera 1502 on the eyewear frame which records food images when activated, wherein the camera is activated to record food images when data from the chewing sensor and/or data from the motion sensor indicate that the person is eating; and a spectroscopic sensor 1508 on the smart watch (or wrist band) which analyzes the molecular and/or nutritional composition of food. In an example, the spectroscopic sensor can be activated automatically when data from the other sensor(s) indicates that the person is eating. In an example, the person can be prompted to use a spectroscopic sensor when data from the other sensor(s) indicate that the person is eating. In an example, a person can take a spectroscopic scan of food by waving their hand over food like Obi-Wan Kenobi (“These aren't the doughnuts you're looking for”). In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor. In an example, a person can take a spectroscopic scan of food by waving their hand over food.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

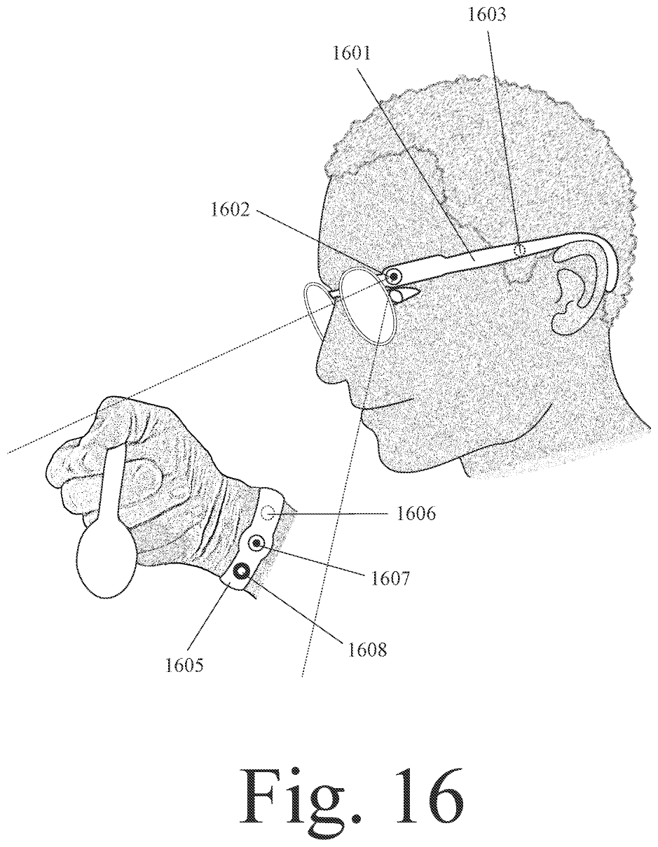

FIG. 16 shows an example of a wearable system for measuring food consumption comprising: an eyewear frame 1601 worn by a person; a chewing sensor 1603 on the eyewear frame which detects when the person eats; a smart watch (or wrist band) 1605 worn by the person; a motion sensor 1606 (e.g. accelerometer and/or gyroscope) on the smart watch (or wrist band) which detects when the person eats; a first camera 1602 on the eyewear frame which records food images when activated, wherein the first camera is activated to record food images when data from the chewing sensor and/or data from the motion sensor indicate that the person is eating; a second camera 1607 on the smart watch (or wrist band) which records food images when activated, wherein the second camera is activated to record food images when data from the chewing sensor and/or data from the motion sensor indicate that the person is eating; and a spectroscopic sensor 1608 on the smart watch (or wrist band) which analyzes the molecular and/or nutritional composition of food. In an example, the spectroscopic sensor can be activated automatically when data from the other sensor(s) indicates that the person is eating. In an example, the person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, a chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

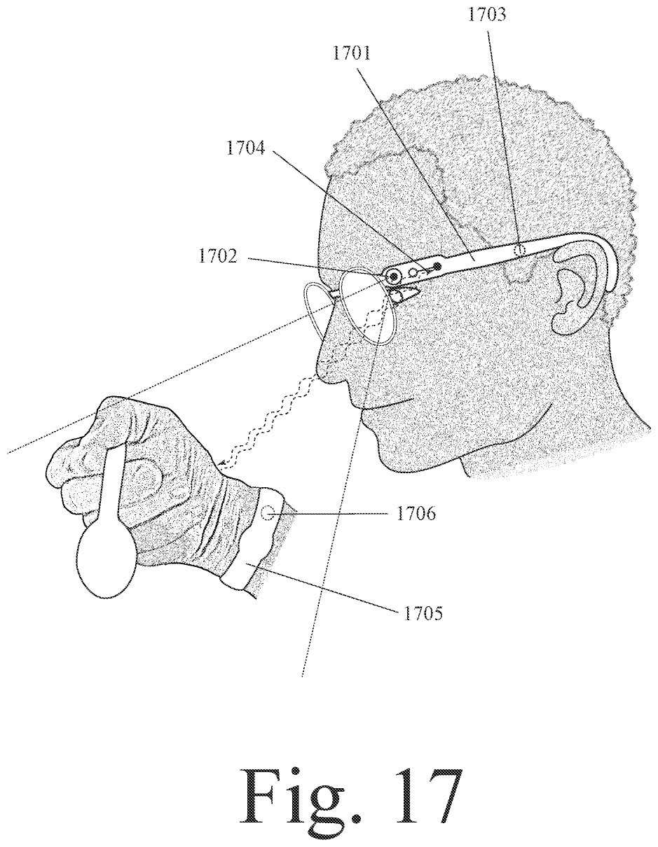

FIG. 17 shows an example of a wearable system for measuring food consumption comprising: an eyewear frame 1701 worn by a person; a chewing sensor 1703 on the eyewear frame which detects when the person eats; a proximity sensor 1704 on the eyewear frame which uses infrared light to detect eating by detecting when an object (such as the person's hand) is near the person's mouth; a smart watch (or wrist band) 1705 worn by the person; a motion sensor 1706 (e.g. accelerometer and/or gyroscope) on the smart watch (or wrist band) which detects when the person eats; and a camera 1702 on the eyewear frame which records food images when activated, wherein the camera is activated to record food images when data from the chewing sensor, data from the proximity sensor, and/or data from the motion sensor indicate that the person is eating. In an example, joint analysis of data from the chewing sensor, the proximity sensor, and the motion sensor can provide more accurate detection of eating than data from any of the three sensors alone or separate analysis of data from the three sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

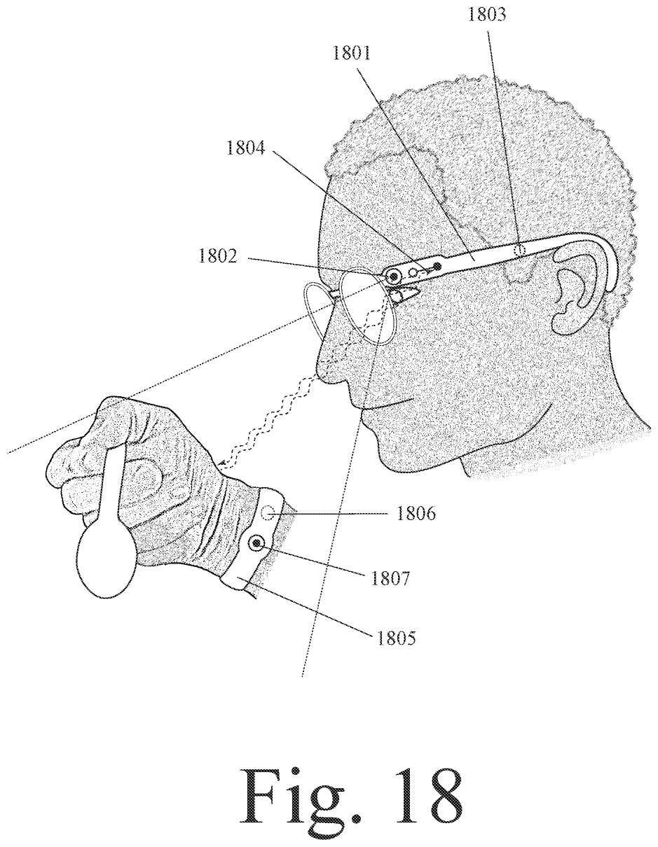

FIG. 18 shows an example of a wearable system for measuring food consumption comprising: an eyewear frame 1801 worn by a person; a chewing sensor 1803 on the eyewear frame which detects when the person eats; a proximity sensor 1804 on the eyewear frame which uses infrared light to detect when the person is eating by detecting when an object (such as the person's hand) is near the person's mouth; a smart watch (or wrist band) 1805 worn by the person; a motion sensor 1806 (e.g. accelerometer and/or gyroscope) on the smart watch (or wrist band); a first camera 1802 on the eyewear frame which records food images when activated, wherein the first camera is activated to record food images when data from the chewing sensor, data from the proximity sensor, and/or data from the motion sensor indicate that the person is eating; and a second camera 1807 on the smart watch (or wrist band) which records food images when activated, wherein the second camera is activated to record food images when data from the chewing sensor, data from the proximity sensor, and/or data from the motion sensor indicate that the person is eating. In an example, joint analysis of data from the chewing sensor, the proximity sensor, and the motion sensor can provide more accurate detection of eating than data from any of the three sensors alone or separate analysis of data from the three sensors. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

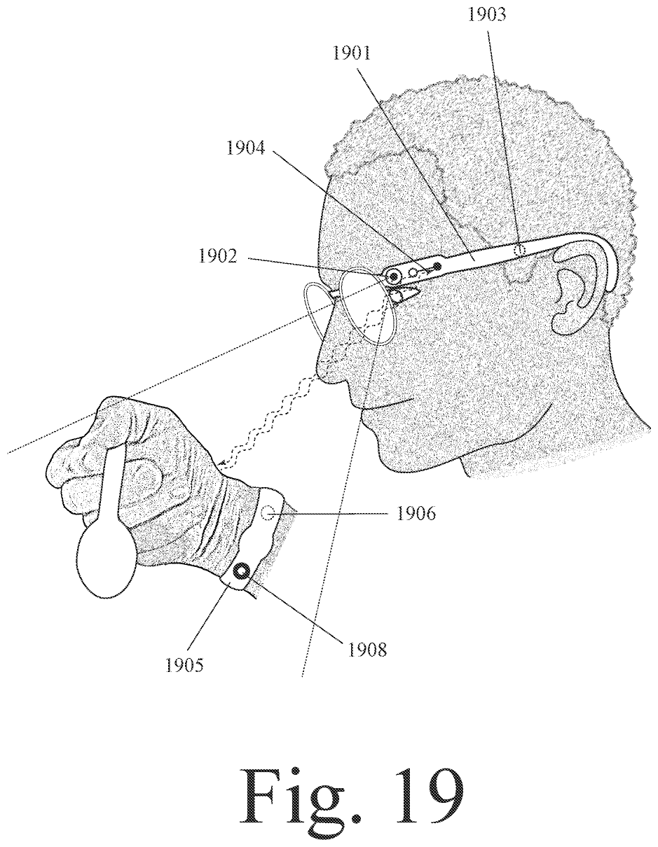

FIG. 19 shows an example of a wearable system for measuring food consumption comprising: an eyewear frame 1901 worn by a person; a chewing sensor 1903 on the eyewear frame which detects when the person eats; a proximity sensor 1904 on the eyewear frame which uses infrared light to detect when the person eats by detecting when an object (such as the person's hand) is near the person's mouth; a smart watch (or wrist band) 1905 worn by the person; a motion sensor 1906 (e.g. accelerometer and/or gyroscope) on the smart watch (or wrist band); a camera 1902 on the eyewear frame which records food images when activated, wherein the camera is activated to record food images when data from the chewing sensor, data from the proximity sensor, and/or data from the motion sensor indicate that the person is eating; and a spectroscopic sensor 1908 on the smart watch (or wrist band) which analyzes the molecular and/or nutritional composition of food. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, joint analysis of data from the chewing sensor, data from the proximity sensor, and data from the motion sensor can provide more accurate detection of eating than data from any of the three sensors alone or separate analysis of data from the three sensors. In an example, the spectroscopic sensor can be activated automatically when data from the other sensor(s) indicates that the person is eating. In an example, a person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light.

In an example, the camera can be an integral part of a sidepiece (e.g. “temple”) of smart eyewear. In an example, a camera can be attached to a sidepiece (e.g. “temple”) of a traditional eyewear. In an example, a camera can be part of (or attached to) a front section of an eyewear frame. In an example, a camera can be just under (e.g. located with 1″ of the bottom of) a person's ear. In an example, the focal direction of a camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, a camera can be directed forward toward a space 1′ to 4′ in front of the person to capture frontal hand-to-food interactions and nearby food portions, but with privacy filtering to avoid and/or blur images of people. In an example, there can be two cameras, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of a camera can be changed automatically to track a person's hands. In an example, an indicator light can be on when the camera is activated. In an example, a shutter or flap can automatically cover the camera when the camera is not activated.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

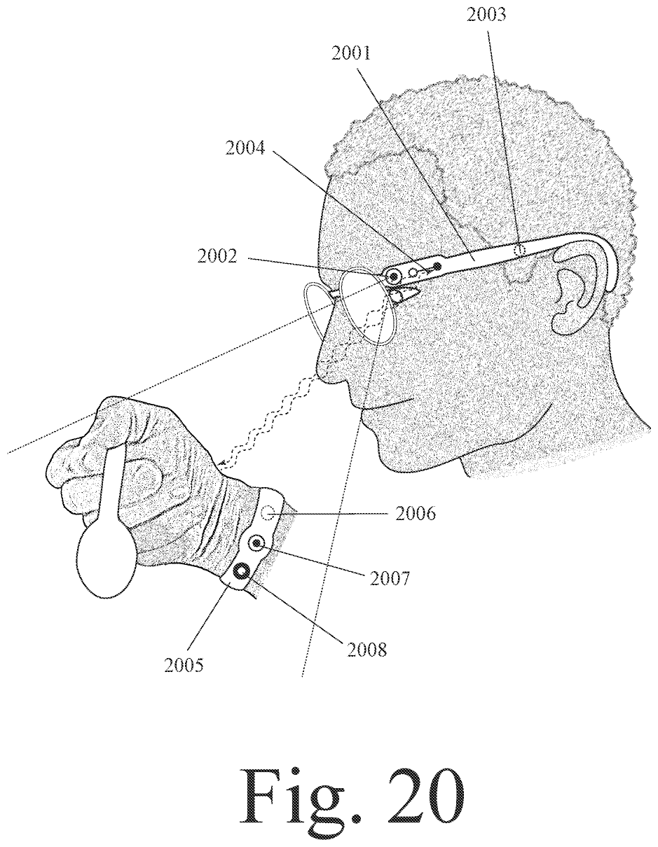

FIG. 20 shows an example of a wearable system for measuring food consumption comprising: an eyewear frame 2001 worn by a person; a chewing sensor 2003 on the eyewear frame which detects when the person eats; a proximity sensor 2004 on the eyewear frame which uses infrared light to detect when the person eats by detecting when an object (such as the person's hand) is near the person's mouth; a smart watch (or wrist band) 2005 worn by the person; a motion sensor 2006 (e.g. accelerometer and/or gyroscope) on the smart watch (or wrist band); a first camera 2002 on the eyewear frame which records food images when activated, wherein the first camera is activated to record food images when data from the chewing sensor, data from the proximity sensor, and/or data from the motion sensor indicate that the person is eating; a second camera 2007 on the smart watch (or wrist band) which records food images when activated, wherein the second camera is activated to record food images when data from the chewing sensor, data from the proximity sensor, and/or data from the motion sensor indicate that the person is eating; and a spectroscopic sensor 2008 on the smart watch (or wrist band) which analyzes the molecular and/or nutritional composition of food. In an example, eyewear can be a pair of eyeglasses. In an example, this example can comprise a finger ring instead of a smart watch or wrist band. In an example, this device or system can further comprise an electromagnetic signal emitter on smart eyeglasses, on a smart watch (or wrist band), or on both which is used to detect proximity between the smart eyeglasses and the smart watch (or wrist band).

In an example, joint analysis of data from the chewing sensor, data from the proximity sensor, and data from the motion sensor can provide more accurate detection of eating than data from any of the three sensors alone or separate analysis of data from the three sensors. In an example, the spectroscopic sensor can be activated automatically when data from the other sensor(s) indicates that the person is eating. In an example, a person can be prompted to use a spectroscopic sensor when data from the other sensor(s) indicate that the person is eating. In an example, a person can take a spectroscopic scan of food by waving their hand over food. In an example, a spectroscopic sensor can emit light away from the outer surface of a smart watch (or wrist band) and toward food. In an example, a spectroscopic sensor can emit and receive near-infrared light.

In an example, the first camera can be an integral part of a sidepiece (e.g. “temple”) of smart eyewear. In an example, the first camera can be attached to a sidepiece (e.g. “temple”) of a traditional eyewear. In an example, the first camera can be part of (or attached to) a front section of an eyewear frame. In an example, a camera can be just under (e.g. located with 1″ of the bottom of) a person's ear. In an example, the first camera can be directed forward and downward (at an angle within the range of 30 to 90 degrees relative to a longitudinal axis of an eyewear sidepiece) toward space directly in front (e.g. within 12″) of a person's mouth. In an example, the focal direction of a camera can be tilted inward (toward the center of a person's face) to capture hand-to-mouth interactions. Alternatively, the first camera can be directed forward toward a space 1′ to 4′ in front of the person to capture frontal hand-to-food interactions and nearby food portions, but with privacy filtering to avoid and/or blur images of people. In an example, there can be two cameras on the eyewear, one on each side (right and left) of eyewear, to record stereoscopic (3D) images of food. In an example, there can be two cameras on a single side of eyewear, one directed forward and downward (toward a person's mouth) and one directed straight forward (toward the person's hands). In an example, the focal direction of the first camera can be changed automatically to track a person's hands. In an example, an indicator light can be on when the camera is activated. In an example, a shutter or flap can automatically cover the camera when the camera is not activated.

In an example, the second camera can be located on the anterior side of the person's wrist (opposite the traditional location of a watch face). Alternatively, the second camera can be located on a side of the watch face housing. In an example, there can be two cameras on a smart watch, wrist band, or watch band to record images of nearby food, hand-to-food interactions, and hand-to-mouth interactions. In an example, one wrist-worn camera can be on one lateral side of a person's wrist and the other wrist-worn camera can be on the other lateral side of the person's wrist, so that one camera tends to record images of nearby food and the other camera tends to record images of the person's mouth as the person eats.

In an example, the chewing sensor can be a microphone or other sonic energy sensor which detects chewing and/or swallowing sounds during eating. In an example, a chewing sensor can be an EMG sensor or other neuromuscular activity sensor which detects muscle movement during eating. In an example, an EMG sensor can monitor activity of the lateral pterygoid muscle, the masseter muscle, the medial pterygoid muscle, and/or the temporalis muscle. In an example, a chewing sensor can be a motion and/or vibration sensor. In an example, a chewing sensor can be a (high-frequency) accelerometer. In an example, a chewing sensor can be a (piezoelectric) strain sensor. In an example, a chewing sensor can be part of (or attached to) a sidepiece of the eyewear. In an example, a chewing sensor can be posterior to (e.g. to the rear of) a camera on an eyewear frame. In an example, a chewing sensor can be located behind an ear. In an example, a chewing sensor can be located between an ear and the frontpiece of an eyewear frame. In an example, a camera can protrude outward (away from a person's body) from an eyewear sidepiece and a chewing sensor can protrude inward (toward the person's body) from the sidepiece.

In an example, a chewing sensor can be made from a non-conductive elastomeric (e.g. silicone-based) polymer (such as PDMS) which has been coated, doped, or impregnated with conductive metal. In an example, a chewing sensor can be held in close contact with a person's head by a spring mechanism, compressible foam, or inflatable chamber. In an example, a chewing sensor can protrude inward (e.g. between ⅛″ and 1″) toward a person's body from the sidepiece (e.g. “temple”) of an eyewear frame. In an example, a portion of the sidepiece of an eyewear frame can curve inward toward a person's head to bring a chewing sensor into close contact with the person's body. In an example, a chewing sensor can be behind (e.g. located within 1″ of the back of) a person's ear or under (e.g. located with 1″ of the bottom of) a person's ear.

In an example, a camera can be activated within a selected time period after eating begins and can be deactivated within a selected time period after eating stops. In an example, a camera can also be deactivated if analysis of images does not confirm eating. In another example, a swallowing sensor can be used instead of (or in addition to) a chewing sensor to detect eating and activate a camera to record food images. In an example, an intraoral sensor can be used instead of (or in addition to) an external chewing or swallowing sensor.

In an example, the proximity sensor can direct a beam of infrared light toward space in front of the person's mouth. This beam is reflected back toward the proximity sensor when an object (such as the person's hand or a food utensil) is in front of the person's mouth. In an example, the camera can be activated by the proximity sensor to confirm that the person's hand is bringing food up to their mouth, not to brush their teeth, cough, or some other hand-near-mouth gesture.

The example shown in this figure shows how the output of one type of sensor can be used to trigger operation of another type of sensor. For example, a relatively less-intrusive sensor (such as a motion sensor) can be used to continually monitor and this less-intrusive sensor may trigger operation of a more-intrusive sensor (such as an imaging sensor) only when probable food consumption is detected by the less-intrusive sensor. For example, a relatively less-intrusive sensor (such as a chewing sensor) can be used to continually monitor and this less-intrusive sensor may trigger operation of a more-intrusive sensor (such as an imaging sensor) only when probable food consumption is detected by the less-intrusive sensor.

The following device and system variations can be applied, where relevant, to examples shown in this disclosure. In an example, a wearable food consumption monitoring system can comprise: eyeglasses worn by a person; a camera on the eyeglasses; a spectroscopic sensor; and a blood pressure sensor, wherein the camera is triggered to record images and the spectroscopic sensor is activated to make spectroscopic scans when analysis of data from the blood pressure sensor indicates that the person is consuming food. In another example, a wearable food consumption monitoring system can comprise: eyeglasses worn by a person; a camera on the eyeglasses; a spectroscopic sensor; and a piezoelectric sensor, wherein the camera is triggered to record images and the spectroscopic sensor is activated to make spectroscopic scans when analysis of data from the piezoelectric sensor indicates that the person is consuming food. In another example, a wearable food consumption monitoring system can comprise: eyeglasses worn by a person; a camera on the eyeglasses; a spectroscopic sensor; and a swallowing sensor, wherein the camera is triggered to record images and the spectroscopic sensor is activated to make spectroscopic scans when analysis of data from the swallowing sensor indicates that the person is consuming food.

In an example, a wearable food consumption monitoring system can comprise: eyeglasses worn by a person; a camera on the eyeglasses; a spectroscopic sensor; and an optical sensor, wherein the camera is triggered to record images and the spectroscopic sensor is activated to make spectroscopic scans when analysis of data from the optical sensor indicates that the person is consuming food. In another embodiment, a wearable food consumption monitoring system can comprise: eyeglasses worn by a person, wherein the eyeglasses further comprise a camera; a spectroscopic sensor; and a wrist-worn or finger-worn EMG sensor, wherein the camera is triggered to record images and the spectroscopic sensor is activated to make spectroscopic scans when analysis of data from the EMG sensor indicates that the person is consuming food. Alternatively, a wearable food consumption monitoring system can comprise: eyeglasses worn by a person, wherein the eyeglasses further comprise a camera; a spectroscopic sensor; and a wrist-worn or finger-worn optical sensor, wherein the camera is triggered to record images and the spectroscopic sensor is activated to make spectroscopic scans when analysis of data from the optical sensor indicates that the person is consuming food.

In another embodiment, a wearable food consumption monitoring system can comprise: eyeglasses worn by a person, wherein the eyeglasses further comprise a camera; a spectroscopic sensor; and a wrist-worn or finger-worn strain gauge, wherein the camera is triggered to record images and the spectroscopic sensor is activated to make spectroscopic scans when analysis of data from the strain gauge indicates that the person is consuming food. In an example, a wearable food consumption monitoring device can comprise: eyeglasses worn by a person; an infrared sensor on a sidepiece (e.g. a temple) of the eyeglasses, wherein the infrared sensor points toward the person's mouth; at least one EMG sensor on the eyeglasses; and a camera on a sidepiece (e.g. a temple) of the eyeglasses, wherein the camera points toward the person's mouth, and wherein the camera is activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; and a camera on a frontpiece and/or nose bridge of the eyeglasses, wherein the camera points toward the person's mouth, and wherein the camera is activated to record food images when analysis of data from the infrared sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a frontpiece and/or nose bridge of the eyeglasses, wherein the first camera points toward the person's mouth; and second camera on a frontpiece and/or nose bridge of the eyeglasses, wherein the second camera points toward the person's mouth, and wherein the first and second cameras are activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on the eyeglasses, wherein the camera points toward the person's mouth, and wherein the camera is activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses; and a camera on a frontpiece and/or nose bridge of the eyeglasses, wherein the camera points toward the person's mouth, and wherein the camera is activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one inertial motion sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one vibration sensor indicates that the person is probably eating.

In an example, a wearable food consumption monitoring system can comprise: eyeglasses worn by a person, wherein the eyeglasses further comprise a camera; and a finger-worn motion sensor (e.g. in a smart ring), wherein the camera is triggered to record images along an imaging vector which points toward the person's mouth when analysis of data from the finger-worn motion sensor indicates that the person is consuming food. In another embodiment, a wearable food consumption monitoring system can comprise: eyeglasses worn by a person, wherein the eyeglasses further comprise a camera; and a wrist-worn motion sensor (e.g. in a smart watch), wherein the camera is triggered to record images along an imaging vector which points toward the person's mouth when analysis of data from the wrist-worn motion sensor indicates that the person is consuming food.

In an example, a wearable food consumption monitoring device can comprise: eyeglasses worn by a person, wherein the eyeglasses further comprise a camera; and wherein the eyeglasses further comprise a blood pressure sensor, wherein the camera is triggered to record images along an imaging vector which points toward the person's mouth when analysis of data from the blood pressure sensor indicates that the person is consuming food. In another example, a wearable food consumption monitoring device can comprise: eyeglasses worn by a person, wherein the eyeglasses further comprise a camera; and wherein the eyeglasses further comprise a chewing sensor, wherein a first camera is triggered to record images along an imaging vector which points toward the person's mouth and a second camera is triggered to record images along an imaging vector which points toward a reachable food source when analysis of data from the chewing sensor indicates that the person is consuming food.

In an example, a wearable food consumption monitoring device can comprise: eyeglasses worn by a person, wherein the eyeglasses further comprise a camera; and wherein the eyeglasses further comprise a GPS sensor, wherein a first camera is triggered to record images along an imaging vector which points toward the person's mouth and a second camera is triggered to record images along an imaging vector which points toward a reachable food source when analysis of data from the GPS sensor indicates that the person is consuming food. In an example, a wearable food consumption monitoring device can comprise: eyeglasses worn by a person, wherein the eyeglasses further comprise a camera; and wherein the eyeglasses further comprise a location sensor, wherein the camera is triggered to record images along an imaging vector which points toward the person's mouth when analysis of data from the location sensor indicates that the person is consuming food. In another embodiment, a wearable food consumption monitoring device can comprise: eyeglasses worn by a person, wherein the eyeglasses further comprise a camera; and wherein the eyeglasses further comprise a motion sensor, wherein the camera is triggered to record images along an imaging vector which points toward the person's mouth when analysis of data from the motion sensor indicates that the person is consuming food. In another example, a wearable food consumption monitoring device can comprise: eyeglasses worn by a person, wherein the eyeglasses further comprise a camera; and wherein the eyeglasses further comprise a piezoelectric sensor, wherein a first camera is triggered to record images along an imaging vector which points toward the person's mouth and a second camera is triggered to record images along an imaging vector which points toward a reachable food source when analysis of data from the piezoelectric sensor indicates that the person is consuming food.

In an example, a wearable food consumption monitoring device can comprise: eyeglasses worn by a person, wherein the eyeglasses further comprise a camera; and wherein the eyeglasses further comprise a proximity sensor, wherein a first camera is triggered to record images along an imaging vector which points toward the person's mouth and a second camera is triggered to record images along an imaging vector which points toward a reachable food source when analysis of data from the proximity sensor indicates that the person is consuming food. In another embodiment, a wearable food consumption monitoring device can comprise: eyeglasses worn by a person, wherein the eyeglasses further comprise a camera; and wherein the eyeglasses further comprise a smell sensor, wherein a first camera is triggered to record images along an imaging vector which points toward the person's mouth and a second camera is triggered to record images along an imaging vector which points toward a reachable food source when analysis of data from the smell sensor indicates that the person is consuming food. In another example, a wearable food consumption monitoring device can comprise: eyeglasses worn by a person, wherein the eyeglasses further comprise a camera; and wherein the eyeglasses further comprise a strain gauge, wherein a first camera is triggered to record images along an imaging vector which points toward the person's mouth and a second camera is triggered to record images along an imaging vector which points toward a reachable food source when analysis of data from the strain gauge indicates that the person is consuming food.

In an example, a wearable food consumption monitoring device can comprise: eyeglasses worn by a person, wherein the eyeglasses further comprise a camera; and wherein the eyeglasses further comprise a swallowing sensor, wherein a first camera is triggered to record images along an imaging vector which points toward the person's mouth and a second camera is triggered to record images along an imaging vector which points toward a reachable food source when analysis of data from the swallowing sensor indicates that the person is consuming food. In another embodiment, a wearable food consumption monitoring device can comprise: eyeglasses worn by a person, wherein the eyeglasses further comprise a camera; and wherein the eyeglasses further comprise an EEG sensor, wherein a first camera is triggered to record images along an imaging vector which points toward the person's mouth and a second camera is triggered to record images along an imaging vector which points toward a reachable food source when analysis of data from the EEG sensor indicates that the person is consuming food. Alternatively, a wearable food consumption monitoring device can comprise: eyeglasses worn by a person, wherein the eyeglasses further comprise a camera; and wherein the eyeglasses further comprise an electrochemical sensor, wherein the camera is triggered to record images along an imaging vector which points toward the person's mouth when analysis of data from the electrochemical sensor indicates that the person is consuming food.

In an example, a wearable food consumption monitoring device can comprise: eyeglasses worn by a person, wherein the eyeglasses further comprise a camera; and wherein the eyeglasses further comprise an EMG sensor, wherein the camera is triggered to record images along an imaging vector which points toward the person's mouth when analysis of data from the EMG sensor indicates that the person is consuming food. In another example, a wearable food consumption monitoring device can comprise: eyeglasses worn by a person, wherein the eyeglasses further comprise a camera; and wherein the eyeglasses further comprise an infrared sensor, wherein a first camera is triggered to record images along an imaging vector which points toward the person's mouth and a second camera is triggered to record images along an imaging vector which points toward a reachable food source when analysis of data from the infrared sensor indicates that the person is consuming food. In another embodiment, a wearable food consumption monitoring system can comprise: eyeglasses worn by a person, wherein the eyeglasses further comprise at least two cameras; and wrist-worn motion sensor, wherein a first camera is triggered to record images along an imaging vector which points toward the person's mouth and a second camera is triggered to record images of a reachable food source when analysis of data from the wrist-worn motion sensor indicates that the person is consuming food.

In an example, a wearable food consumption monitoring device can comprise: eyeglasses worn by a person; at least one EMG sensor on a portion of the eyeglasses which curves around the rear of the person's ear; and a camera on a frontpiece and/or nose bridge of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one EMG sensor indicates that the person is probably eating. In another embodiment, a wearable food consumption monitoring device can comprise: eyeglasses worn by a person; at least one EMG sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one EMG sensor indicates that the person is probably eating.

In an example, a wearable food consumption monitoring device can comprise: eyeglasses worn by a person; at least one EMG sensor on the eyeglasses; and a camera on a sidepiece (e.g. a temple) of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one inertial motion sensor indicates that the person is probably eating.

In another embodiment, a wearable food consumption monitoring device can comprise: eyeglasses worn by a person; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one vibration sensor indicates that the person is probably eating. In an example, a wearable food consumption monitoring device can comprise: eyeglasses worn by a person; at least one vibration sensor on the eyeglasses; and a camera on the eyeglasses, wherein the camera points toward the person's mouth, and wherein the camera is activated to record food images when analysis of data from the at least one vibration sensor indicates that the person is probably eating.

In another embodiment, a wearable food consumption monitoring system can comprise: eyeglasses worn by a person; at least one wrist-worn or finger-worn inertial motion sensor (e.g. gyroscope and/or accelerometer on a smart watch or smart ring); and a camera on a frontpiece and/or nose bridge of the eyeglasses, wherein the camera points toward the person's mouth, and wherein the camera is activated to record food images when analysis of data from the at least one wrist-worn or finger-worn inertial motion sensor indicates that the person is probably eating. In an example, a wearable food consumption monitoring device can comprise: eyeglasses worn by a person, wherein the eyeglasses further comprise a camera; wherein the eyeglasses further comprise a motion sensor; and wherein the eyeglasses further comprise an infrared sensor which tracks the location of the person's hands, wherein the camera is triggered to record images along an imaging vector which points toward the person's mouth when joint analysis of data from the motion sensor and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise a chewing sensor, wherein a first camera is triggered to record images along an imaging vector which points toward the person's mouth and a second camera is triggered to record images of a reachable food source when analysis of data from the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone) and an EEG sensor, wherein a first camera is triggered to record images along an imaging vector which points toward the person's mouth and a second camera is triggered to record images of a reachable food source when joint analysis of data from the sound sensor (e.g. microphone) and the EEG sensor indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone), an EEG sensor, and an infrared sensor, wherein a first camera is triggered to record images along an imaging vector which points toward the person's mouth and a second camera is triggered to record images of a reachable food source when joint analysis of data from the sound sensor (e.g. microphone), the EEG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), a chewing sensor, and an infrared sensor, wherein a first camera is triggered to record images along an imaging vector which points toward the person's mouth and a second camera is triggered to record images of a reachable food source when joint analysis of data from the sound sensor (e.g. microphone), the chewing sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone) and a motion sensor, wherein a first camera is triggered to record images along an imaging vector which points toward the person's mouth and a second camera is triggered to record images of a reachable food source when joint analysis of data from the sound sensor (e.g. microphone) and the motion sensor indicates that the person is consuming food. In an example, a wearable food consumption monitoring device can comprise: eyeglasses worn by a person; wherein the eyeglasses further comprise at least two cameras; and wherein the eyeglasses further comprise a swallow sensor and an EMG sensor, wherein a first camera is triggered to record images along an imaging vector which points toward the person's mouth and a second camera is triggered to record images of a reachable food source when joint analysis of data from the swallow sensor and the EMG sensor indicates that the person is consuming food.

In another embodiment, a wearable food consumption monitoring device can comprise: eyeglasses worn by a person; wherein the eyeglasses further comprise at least two cameras; and wherein the eyeglasses further comprise a swallow sensor, a motion sensor, and an infrared sensor, wherein a first camera is triggered to record images along an imaging vector which points toward the person's mouth and a second camera is triggered to record images of a reachable food source when joint analysis of data from the swallow sensor, the motion sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor, wherein a first camera is triggered to record images along an imaging vector which points toward the person's mouth and a second camera is triggered to record images of a reachable food source when analysis of data from the swallow sensor indicates that the person is consuming food. Alternatively, a wearable food consumption monitoring device can comprise: eyeglasses worn by a person; wherein the eyeglasses further comprise at least two cameras; and wherein the eyeglasses further comprise a swallowing sensor and a chewing sensor, wherein a first camera is triggered to record images along an imaging vector which points toward the person's mouth and a second camera is triggered to record images of a reachable food source when joint analysis of data from the swallowing sensor and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor, an EMG sensor, and an infrared sensor, wherein a first camera is triggered to record images along an imaging vector which points toward the person's mouth and a second camera is triggered to record images of a reachable food source when joint analysis of data from the swallowing sensor, the EMG sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an accelerometer and a chewing sensor, wherein a first camera is triggered to record images along an imaging vector which points toward the person's mouth and a second camera is triggered to record images of a reachable food source when joint analysis of data from the accelerometer and the chewing sensor indicates that the person is consuming food. In an example, a wearable food consumption monitoring device can comprise: eyeglasses worn by a person; wherein the eyeglasses further comprise at least two cameras; and wherein the eyeglasses further comprise an EEG sensor and an accelerometer, wherein a first camera is triggered to record images along an imaging vector which points toward the person's mouth and a second camera is triggered to record images of a reachable food source when joint analysis of data from the EEG sensor and the accelerometer indicates that the person is consuming food.

In another embodiment, a wearable food consumption monitoring device can comprise: eyeglasses worn by a person; wherein the eyeglasses further comprise at least two cameras; and wherein the eyeglasses further comprise an EEG sensor and an accelerometer, wherein a first camera is triggered to record images along an imaging vector which points toward the person's mouth and a second camera is triggered to record images of a reachable food source when joint analysis of data from the EEG sensor and the accelerometer indicates that the person is consuming food. In an example, a wearable food consumption monitoring device can comprise: eyeglasses worn by a person; wherein the eyeglasses further comprise at least two cameras; and wherein the eyeglasses further comprise an EEG sensor, wherein a first camera is triggered to record images along an imaging vector which points toward the person's mouth and a second camera is triggered to record images of a reachable food source when analysis of data from the EEG sensor indicates that the person is consuming food. In another example, a wearable food consumption monitoring device can comprise: eyeglasses worn by a person; wherein the eyeglasses further comprise at least two cameras; and wherein the eyeglasses further comprise an EMG sensor, a sound sensor (e.g. microphone), and an infrared sensor, wherein a first camera is triggered to record images along an imaging vector which points toward the person's mouth and a second camera is triggered to record images of a reachable food source when joint analysis of data from the EMG sensor, the sound sensor (e.g. microphone), and the infrared sensor indicates that the person is consuming food.

In another embodiment, a wearable food consumption monitoring device can comprise: eyeglasses worn by a person; wherein the eyeglasses further comprise at least two cameras; and wherein the eyeglasses further comprise an EMG sensor and a sound sensor (e.g. microphone), wherein a first camera is triggered to record images along an imaging vector which points toward the person's mouth and a second camera is triggered to record images of a reachable food source when joint analysis of data from the EMG sensor and the sound sensor (e.g. microphone) indicates that the person is consuming food. In an example, a wearable food consumption monitoring device can comprise: eyeglasses worn by a person; wherein the eyeglasses further comprise at least two cameras; and wherein the eyeglasses further comprise an motion sensor and an infrared sensor, wherein a first camera is triggered to record images along an imaging vector which points toward the person's mouth and a second camera is triggered to record images of a reachable food source when joint analysis of data from the motion sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a chewing sensor and an infrared sensor, wherein at least one camera is triggered to record images along an imaging vector which points toward the person's mouth when joint analysis of data from the chewing sensor and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a sound sensor (e.g. microphone) and an EEG sensor, wherein at least one camera is triggered to record images along an imaging vector which points toward the person's mouth when joint analysis of data from the sound sensor (e.g. microphone) and the EEG sensor indicates that the person is consuming food. Alternatively, a wearable food consumption monitoring device can comprise: eyeglasses worn by a person; wherein the eyeglasses further comprise one or more cameras; and wherein the eyeglasses further comprise a sound sensor (e.g. microphone), an EEG sensor, and an infrared sensor, wherein at least one camera is triggered to record images along an imaging vector which points toward the person's mouth when joint analysis of data from the sound sensor (e.g. microphone), the EEG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and an EEG sensor, wherein at least one camera is triggered to record images along an imaging vector which points toward the person's mouth when joint analysis of data from the sound sensor (e.g. microphone) and the EEG sensor indicates that the person is consuming food.

In an example, a wearable food consumption monitoring device can comprise: eyeglasses worn by a person; wherein the eyeglasses further comprise one or more cameras; and wherein the eyeglasses further comprise a sound sensor (e.g. microphone), a motion sensor, and an infrared sensor, wherein at least one camera is triggered to record images along an imaging vector which points toward the person's mouth when joint analysis of data from the sound sensor (e.g. microphone), the motion sensor, and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise a swallow sensor and an EMG sensor, wherein at least one camera is triggered to record images along an imaging vector which points toward the person's mouth when joint analysis of data from the swallow sensor and the EMG sensor indicates that the person is consuming food. In an example, a wearable food consumption monitoring device can comprise: eyeglasses worn by a person; wherein the eyeglasses further comprise one or more cameras; and wherein the eyeglasses further comprise a swallow sensor, a motion sensor, and an infrared sensor, wherein at least one camera is triggered to record images along an imaging vector which points toward the person's mouth when joint analysis of data from the swallow sensor, the motion sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor, wherein at least one camera is triggered to record images along an imaging vector which points toward the person's mouth when analysis of data from the swallow sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor and a chewing sensor, wherein at least one camera is triggered to record images along an imaging vector which points toward the person's mouth when joint analysis of data from the swallowing sensor and the chewing sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor, an accelerometer, and an infrared sensor, wherein at least one camera is triggered to record images along an imaging vector which points toward the person's mouth when joint analysis of data from the swallowing sensor, the accelerometer, and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise an accelerometer and a chewing sensor, wherein at least one camera is triggered to record images along an imaging vector which points toward the person's mouth when joint analysis of data from the accelerometer and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise an EEG sensor and an accelerometer, wherein at least one camera is triggered to record images along an imaging vector which points toward the person's mouth when joint analysis of data from the EEG sensor and the accelerometer indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor and an accelerometer, wherein at least one camera is triggered to record images along an imaging vector which points toward the person's mouth when joint analysis of data from the EEG sensor and the accelerometer indicates that the person is consuming food. In another embodiment, a wearable food consumption monitoring device can comprise: eyeglasses worn by a person; wherein the eyeglasses further comprise one or more cameras; and wherein the eyeglasses further comprise an EEG sensor, wherein at least one camera is triggered to record images along an imaging vector which points toward the person's mouth when analysis of data from the EEG sensor indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor and a sound sensor (e.g. microphone), wherein at least one camera is triggered to record images along an imaging vector which points toward the person's mouth when joint analysis of data from the EMG sensor and the sound sensor (e.g. microphone) indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, a motion sensor, and an infrared sensor, wherein at least one camera is triggered to record images along an imaging vector which points toward the person's mouth when joint analysis of data from the EMG sensor, the motion sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor, wherein at least one camera is triggered to record images along an imaging vector which points toward the person's mouth when analysis of data from the EMG sensor indicates that the person is consuming food. In an example, a wearable food consumption monitoring device can comprise: eyeglasses worn by a person; wherein the eyeglasses further comprise one or more cameras; and wherein the eyeglasses further comprise an motion sensor, a chewing sensor, and an infrared sensor, wherein at least one camera is triggered to record images along an imaging vector which points toward the person's mouth when joint analysis of data from the motion sensor, the chewing sensor, and the infrared sensor indicates that the person is consuming food. In another embodiment, a wearable food consumption monitoring system can comprise: eyeglasses worn by a person; a camera on the eyeglasses; a spectroscopic sensor; and a chewing sensor, wherein the camera is triggered to record images and the spectroscopic sensor is activated to make spectroscopic scans when analysis of data from the chewing sensor indicates that the person is consuming food.

In an example, a wearable food consumption monitoring system can comprise: eyeglasses worn by a person; a camera on the eyeglasses; a spectroscopic sensor; and a pressure sensor, wherein the camera is triggered to record images and the spectroscopic sensor is activated to make spectroscopic scans when analysis of data from the pressure sensor indicates that the person is consuming food. In another example, a wearable food consumption monitoring system can comprise: eyeglasses worn by a person; a camera on the eyeglasses; a spectroscopic sensor; and an EEG sensor, wherein the camera is triggered to record images and the spectroscopic sensor is activated to make spectroscopic scans when analysis of data from the EEG sensor indicates that the person is consuming food.

In another embodiment, a wearable food consumption monitoring system can comprise: eyeglasses worn by a person, wherein the eyeglasses further comprise a camera; a spectroscopic sensor; and a wrist-worn or finger-worn blood pressure sensor, wherein the camera is triggered to record images and the spectroscopic sensor is activated to make spectroscopic scans when analysis of data from the blood pressure sensor indicates that the person is consuming food. In another example, a wearable food consumption monitoring system can comprise: eyeglasses worn by a person, wherein the eyeglasses further comprise a camera; a spectroscopic sensor; and a wrist-worn or finger-worn piezoelectric sensor, wherein the camera is triggered to record images and the spectroscopic sensor is activated to make spectroscopic scans when analysis of data from the piezoelectric sensor indicates that the person is consuming food.

In an example, a wearable food consumption monitoring device can comprise: eyeglasses worn by a person; an infrared sensor on a sidepiece (e.g. a temple) of the eyeglasses, wherein the infrared sensor points toward the person's mouth; at least one inertial motion sensor (e.g. gyroscope and/or accelerometer) on the eyeglasses; and a camera on a sidepiece (e.g. a temple) of the eyeglasses, wherein the camera points toward the person's mouth, and wherein the camera is activated to record food images when analysis of data from the infrared sensor and the at least one inertial motion sensor indicates that the person is probably eating. In an example, a wearable food consumption monitoring device can comprise: eyeglasses worn by a person; an infrared sensor on the eyeglasses, wherein the infrared sensor points toward the person's mouth; and a camera on the eyeglasses, wherein the camera points toward the person's mouth, and wherein the camera is activated to record food images when analysis of data from the infrared sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one EMG sensor indicates that the person is probably eating. In another embodiment, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses; and a camera on the eyeglasses, wherein the camera points toward the person's mouth, and wherein the camera is activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; and a camera on a frontpiece and/or nose bridge of the eyeglasses, wherein the camera points toward the person's mouth, and wherein the camera is activated to record food images when analysis of data from the infrared sensor and the at least one inertial motion sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one vibration sensor indicates that the person is probably eating. Alternatively, a wearable food consumption monitoring system can comprise: eyeglasses worn by a person, wherein the eyeglasses further comprise at least two cameras; and a finger-worn motion sensor, wherein a first camera is triggered to record images along an imaging vector which points toward the person's mouth and a second camera is triggered to record images of a reachable food source when analysis of data from the wrist-worn motion sensor indicates that the person is consuming food.

In another embodiment, a wearable food consumption monitoring system can comprise: eyeglasses worn by a person, wherein the eyeglasses further comprise a camera; and a wrist-worn motion sensor, wherein the camera is triggered to record food images when analysis of data from the wrist-worn motion sensor indicates that the person is consuming food. In another example, a wearable food consumption monitoring device can comprise: eyeglasses worn by a person, wherein the eyeglasses further comprise a camera; and wherein the eyeglasses further comprise a blood pressure sensor, wherein a first camera is triggered to record images along an imaging vector which points toward the person's mouth and a second camera is triggered to record images along an imaging vector which points toward a reachable food source when analysis of data from the blood pressure sensor indicates that the person is consuming food.

In an example, a wearable food consumption monitoring device can comprise: eyeglasses worn by a person, wherein the eyeglasses further comprise a camera; and wherein the eyeglasses further comprise a chewing sensor, wherein the camera is triggered to record images along an imaging vector which points toward the person's mouth when analysis of data from the chewing sensor indicates that the person is consuming food. In another embodiment, a wearable food consumption monitoring device can comprise: eyeglasses worn by a person, wherein the eyeglasses further comprise a camera; and wherein the eyeglasses further comprise a GPS sensor, wherein the camera is triggered to record images along an imaging vector which points toward the person's mouth when analysis of data from the GPS sensor indicates that the person is consuming food. In another example, a wearable food consumption monitoring device can comprise: eyeglasses worn by a person, wherein the eyeglasses further comprise a camera; and wherein the eyeglasses further comprise a microphone, wherein the camera is triggered to record images of the interaction between food and the person's mouth when analysis of data from sensor indicates that the person is consuming food.

In an example, a wearable food consumption monitoring device can comprise: eyeglasses worn by a person, wherein the eyeglasses further comprise a camera; and wherein the eyeglasses further comprise a motion sensor, wherein a first camera is triggered to record images along an imaging vector which points toward the person's mouth and a second camera is triggered to record images along an imaging vector which points toward a reachable food source when analysis of data from the motion sensor indicates that the person is consuming food. In an example, a wearable food consumption monitoring device can comprise: eyeglasses worn by a person, wherein the eyeglasses further comprise a camera; and wherein the eyeglasses further comprise a piezoelectric sensor, wherein the camera is triggered to record images along an imaging vector which points toward the person's mouth when analysis of data from the piezoelectric sensor indicates that the person is consuming food.

In an example, a wearable food consumption monitoring device can comprise: eyeglasses worn by a person, wherein the eyeglasses further comprise a camera; and wherein the eyeglasses further comprise a proximity sensor, wherein the camera is triggered to record images along an imaging vector which points toward the person's mouth when analysis of data from the proximity sensor indicates that the person is consuming food. In an example, a wearable food consumption monitoring device can comprise: eyeglasses worn by a person, wherein the eyeglasses further comprise a camera; and wherein the eyeglasses further comprise a smell sensor, wherein the camera is triggered to record images along an imaging vector which points toward the person's mouth when analysis of data from the smell sensor indicates that the person is consuming food. In an example, a wearable food consumption monitoring device can comprise: eyeglasses worn by a person, wherein the eyeglasses further comprise a camera; and wherein the eyeglasses further comprise a strain gauge, wherein the camera is triggered to record images along an imaging vector which points toward the person's mouth when analysis of data from the strain gauge indicates that the person is consuming food. In an example, a wearable food consumption monitoring device can comprise: eyeglasses worn by a person, wherein the eyeglasses further comprise a camera; and wherein the eyeglasses further comprise a swallowing sensor, wherein the camera is triggered to record images along an imaging vector which points toward the person's mouth when analysis of data from the swallowing sensor indicates that the person is consuming food.

In another embodiment, a wearable food consumption monitoring device can comprise: eyeglasses worn by a person, wherein the eyeglasses further comprise a camera; and wherein the eyeglasses further comprise an EEG sensor, wherein the camera is triggered to record images along an imaging vector which points toward the person's mouth when analysis of data from the EEG sensor indicates that the person is consuming food. In an example, a wearable food consumption monitoring device can comprise: eyeglasses worn by a person, wherein the eyeglasses further comprise a camera; and wherein the eyeglasses further comprise an EMG sensor, wherein a first camera is triggered to record images along an imaging vector which points toward the person's mouth and a second camera is triggered to record images along an imaging vector which points toward a reachable food source when analysis of data from the EMG sensor indicates that the person is consuming food. In another example, a wearable food consumption monitoring device can comprise: eyeglasses worn by a person, wherein the eyeglasses further comprise a camera; and wherein the eyeglasses further comprise an EMG sensor, wherein the camera is triggered to record images of the interaction between food and the person's mouth when analysis of data from sensor indicates that the person is consuming food. Alternatively, a wearable food consumption monitoring device can comprise: eyeglasses worn by a person, wherein the eyeglasses further comprise a camera; and wherein the eyeglasses further comprise an infrared sensor, wherein the camera is triggered to record images along an imaging vector which points toward the person's mouth when analysis of data from the infrared sensor indicates that the person is consuming food.

In an example, a wearable food consumption monitoring device can comprise: eyeglasses worn by a person; at least one EMG sensor on a portion of the eyeglasses which curves around the rear of the person's ear; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one EMG sensor indicates that the person is probably eating. In another example, a wearable food consumption monitoring device can comprise: eyeglasses worn by a person; at least one EMG sensor on a portion of the eyeglasses which curves around the rear of the person's ear; and a camera on a sidepiece (e.g. a temple) of the eyeglasses, wherein the camera points toward the person's mouth, and wherein the camera is activated to record food images when analysis of data from the at least one EMG sensor indicates that the person is probably eating.

In an example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one EMG sensor indicates that the person is probably eating. In an example, a wearable food consumption monitoring device can comprise: eyeglasses worn by a person; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one EMG sensor indicates that the person is probably eating.

In another embodiment, a wearable food consumption monitoring device can comprise: eyeglasses worn by a person; at least one EMG sensor on the eyeglasses; and a camera on the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; and a camera on a frontpiece and/or nose bridge of the eyeglasses, wherein the camera points toward the person's mouth, and wherein the camera is activated to record food images when analysis of data from the at least one inertial motion sensor indicates that the person is probably eating. In an example, a wearable food consumption monitoring device can comprise: eyeglasses worn by a person; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one vibration sensor indicates that the person is probably eating.

In another embodiment, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one wrist-worn or finger-worn inertial motion sensor indicates that the person is probably eating. In an example, a wearable food consumption monitoring system can comprise: eyeglasses worn by a person; at least one wrist-worn or finger-worn inertial motion sensor (e.g. gyroscope and/or accelerometer on a smart watch or smart ring); and a camera on a sidepiece (e.g. a temple) of the eyeglasses, wherein the camera points toward the person's mouth, and wherein the camera is activated to record food images when analysis of data from the at least one wrist-worn or finger-worn inertial motion sensor indicates that the person is probably eating.

In another embodiment, a wearable food consumption monitoring device can comprise: eyeglasses worn by a person, wherein the eyeglasses further comprise a camera; wherein the eyeglasses further comprise an EMG sensor; and wherein the eyeglasses further comprise an infrared sensor which tracks the location of the person's hands, wherein the camera is triggered to record images along an imaging vector which points toward the person's mouth when joint analysis of data from the EMG sensor and the infrared sensor indicates that the person is consuming food. Alternatively, a wearable food consumption monitoring device can comprise: eyeglasses worn by a person; wherein the eyeglasses further comprise at least two cameras; and wherein the eyeglasses further comprise a motion sensor, wherein a first camera is triggered to record images along an imaging vector which points toward the person's mouth and a second camera is triggered to record images of a reachable food source when analysis of data from the motion sensor indicates that the person is consuming food.

In another embodiment, a wearable food consumption monitoring device can comprise: eyeglasses worn by a person; wherein the eyeglasses further comprise at least two cameras; and wherein the eyeglasses further comprise a sound sensor (e.g. microphone) and a motion sensor, wherein a first camera is triggered to record images along an imaging vector which points toward the person's mouth and a second camera is triggered to record images of a reachable food source when joint analysis of data from the sound sensor (e.g. microphone) and the motion sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone), a motion sensor, and an infrared sensor, wherein a first camera is triggered to record images along an imaging vector which points toward the person's mouth and a second camera is triggered to record images of a reachable food source when joint analysis of data from the sound sensor (e.g. microphone), the motion sensor, and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise a sound sensor (e.g. microphone) and an infrared sensor, wherein a first camera is triggered to record images along an imaging vector which points toward the person's mouth and a second camera is triggered to record images of a reachable food source when joint analysis of data from the sound sensor (e.g. microphone) and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), a motion sensor, and an infrared sensor, wherein a first camera is triggered to record images along an imaging vector which points toward the person's mouth and a second camera is triggered to record images of a reachable food source when joint analysis of data from the sound sensor (e.g. microphone), the motion sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor and a sound sensor (e.g. microphone), wherein a first camera is triggered to record images along an imaging vector which points toward the person's mouth and a second camera is triggered to record images of a reachable food source when joint analysis of data from the swallow sensor and the sound sensor (e.g. microphone) indicates that the person is consuming food. Alternatively, a wearable food consumption monitoring device can comprise: eyeglasses worn by a person; wherein the eyeglasses further comprise at least two cameras; and wherein the eyeglasses further comprise a swallow sensor, a sound sensor (e.g. microphone), and an infrared sensor, wherein a first camera is triggered to record images along an imaging vector which points toward the person's mouth and a second camera is triggered to record images of a reachable food source when joint analysis of data from the swallow sensor, the sound sensor (e.g. microphone), and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor and an infrared sensor, wherein a first camera is triggered to record images along an imaging vector which points toward the person's mouth and a second camera is triggered to record images of a reachable food source when joint analysis of data from the swallowing sensor and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallowing sensor and an accelerometer, wherein a first camera is triggered to record images along an imaging vector which points toward the person's mouth and a second camera is triggered to record images of a reachable food source when joint analysis of data from the swallowing sensor and the accelerometer indicates that the person is consuming food. In an example, a wearable food consumption monitoring device can comprise: eyeglasses worn by a person; wherein the eyeglasses further comprise at least two cameras; and wherein the eyeglasses further comprise a swallowing sensor, an accelerometer, and an infrared sensor, wherein a first camera is triggered to record images along an imaging vector which points toward the person's mouth and a second camera is triggered to record images of a reachable food source when joint analysis of data from the swallowing sensor, the accelerometer, and the infrared sensor indicates that the person is consuming food.

In another embodiment, a wearable food consumption monitoring device can comprise: eyeglasses worn by a person; wherein the eyeglasses further comprise at least two cameras; and wherein the eyeglasses further comprise an accelerometer and an infrared sensor, wherein a first camera is triggered to record images along an imaging vector which points toward the person's mouth and a second camera is triggered to record images of a reachable food source when joint analysis of data from the accelerometer and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an EEG sensor and a chewing sensor, wherein a first camera is triggered to record images along an imaging vector which points toward the person's mouth and a second camera is triggered to record images of a reachable food source when joint analysis of data from the EEG sensor and the chewing sensor indicates that the person is consuming food. Alternatively, a wearable food consumption monitoring device can comprise: eyeglasses worn by a person; wherein the eyeglasses further comprise at least two cameras; and wherein the eyeglasses further comprise an EEG sensor, a chewing sensor, and an infrared sensor, wherein a first camera is triggered to record images along an imaging vector which points toward the person's mouth and a second camera is triggered to record images of a reachable food source when joint analysis of data from the EEG sensor, the chewing sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor and an accelerometer, wherein a first camera is triggered to record images along an imaging vector which points toward the person's mouth and a second camera is triggered to record images of a reachable food source when joint analysis of data from the EMG sensor and the accelerometer indicates that the person is consuming food. In another example, a wearable food consumption monitoring device can comprise: eyeglasses worn by a person; wherein the eyeglasses further comprise at least two cameras; and wherein the eyeglasses further comprise an EMG sensor and an infrared sensor, wherein a first camera is triggered to record images along an imaging vector which points toward the person's mouth and a second camera is triggered to record images of a reachable food source when joint analysis of data from the EMG sensor and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise an EMG sensor, an EEG sensor, and an infrared sensor, wherein a first camera is triggered to record images along an imaging vector which points toward the person's mouth and a second camera is triggered to record images of a reachable food source when joint analysis of data from the EMG sensor, the EEG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an motion sensor and a chewing sensor, wherein a first camera is triggered to record images along an imaging vector which points toward the person's mouth and a second camera is triggered to record images of a reachable food source when joint analysis of data from the motion sensor and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise a chewing sensor, wherein at least one camera is triggered to record images along an imaging vector which points toward the person's mouth when analysis of data from the chewing sensor indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone) and a motion sensor, wherein at least one camera is triggered to record images along an imaging vector which points toward the person's mouth when joint analysis of data from the sound sensor (e.g. microphone) and the motion sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone), a motion sensor, and an infrared sensor, wherein at least one camera is triggered to record images along an imaging vector which points toward the person's mouth when joint analysis of data from the sound sensor (e.g. microphone), the motion sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone) and a chewing sensor, wherein at least one camera is triggered to record images along an imaging vector which points toward the person's mouth when joint analysis of data from the sound sensor (e.g. microphone) and the chewing sensor indicates that the person is consuming food. In another embodiment, a wearable food consumption monitoring device can comprise: eyeglasses worn by a person; wherein the eyeglasses further comprise one or more cameras; and wherein the eyeglasses further comprise a sound sensor (e.g. microphone) and an infrared sensor, wherein at least one camera is triggered to record images along an imaging vector which points toward the person's mouth when joint analysis of data from the sound sensor (e.g. microphone) and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor and a sound sensor (e.g. microphone), wherein at least one camera is triggered to record images along an imaging vector which points toward the person's mouth when joint analysis of data from the swallow sensor and the sound sensor (e.g. microphone) indicates that the person is consuming food. In another example, a wearable food consumption monitoring device can comprise: eyeglasses worn by a person; wherein the eyeglasses further comprise one or more cameras; and wherein the eyeglasses further comprise a swallow sensor, a sound sensor (e.g. microphone), and an infrared sensor, wherein at least one camera is triggered to record images along an imaging vector which points toward the person's mouth when joint analysis of data from the swallow sensor, the sound sensor (e.g. microphone), and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise a swallowing sensor and an infrared sensor, wherein at least one camera is triggered to record images along an imaging vector which points toward the person's mouth when joint analysis of data from the swallowing sensor and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor and an accelerometer, wherein at least one camera is triggered to record images along an imaging vector which points toward the person's mouth when joint analysis of data from the swallowing sensor and the accelerometer indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor, a sound sensor (e.g. microphone), and an infrared sensor, wherein at least one camera is triggered to record images along an imaging vector which points toward the person's mouth when joint analysis of data from the swallowing sensor, the sound sensor (e.g. microphone), and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise an accelerometer and an infrared sensor, wherein at least one camera is triggered to record images along an imaging vector which points toward the person's mouth when joint analysis of data from the accelerometer and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor and a chewing sensor, wherein at least one camera is triggered to record images along an imaging vector which points toward the person's mouth when joint analysis of data from the EEG sensor and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise an EEG sensor, a chewing sensor, and an infrared sensor, wherein at least one camera is triggered to record images along an imaging vector which points toward the person's mouth when joint analysis of data from the EEG sensor, the chewing sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an EMG sensor and an accelerometer, wherein at least one camera is triggered to record images along an imaging vector which points toward the person's mouth when joint analysis of data from the EMG sensor and the accelerometer indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor and an infrared sensor, wherein at least one camera is triggered to record images along an imaging vector which points toward the person's mouth when joint analysis of data from the EMG sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, a sound sensor (e.g. microphone), and an infrared sensor, wherein at least one camera is triggered to record images along an imaging vector which points toward the person's mouth when joint analysis of data from the EMG sensor, the sound sensor (e.g. microphone), and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an motion sensor and a chewing sensor, wherein at least one camera is triggered to record images along an imaging vector which points toward the person's mouth when joint analysis of data from the motion sensor and the chewing sensor indicates that the person is consuming food.

In another embodiment, a wearable food consumption monitoring device can comprise: eyeglasses worn by a person; wherein the eyeglasses further comprise one or more cameras; and wherein the eyeglasses further comprise an motion sensor, wherein at least one camera is triggered to record images along an imaging vector which points toward the person's mouth when analysis of data from the motion sensor indicates that the person is consuming food. In another example, a wearable food consumption monitoring system can comprise: eyeglasses worn by a person; a camera on the eyeglasses; a spectroscopic sensor; and a GPS sensor, wherein the camera is triggered to record images and the spectroscopic sensor is activated to make spectroscopic scans when analysis of data from the GPS sensor indicates that the person is consuming food. In an example, a wearable food consumption monitoring system can comprise: eyeglasses worn by a person; a camera on the eyeglasses; a spectroscopic sensor; and a proximity sensor, wherein the camera is triggered to record images and the spectroscopic sensor is activated to make spectroscopic scans when analysis of data from the proximity sensor indicates that the person is consuming food. Alternatively, a wearable food consumption monitoring system can comprise: eyeglasses worn by a person; a camera on the eyeglasses; a spectroscopic sensor; and an electrochemical sensor, wherein the camera is triggered to record images and the spectroscopic sensor is activated to make spectroscopic scans when analysis of data from the electrochemical sensor indicates that the person is consuming food.

In another embodiment, a wearable food consumption monitoring system can comprise: eyeglasses worn by a person, wherein the eyeglasses further comprise a camera; a spectroscopic sensor; and a wrist-worn or finger-worn chewing sensor, wherein the camera is triggered to record images and the spectroscopic sensor is activated to make spectroscopic scans when analysis of data from the chewing sensor indicates that the person is consuming food. In another example, a wearable food consumption monitoring system can comprise: eyeglasses worn by a person, wherein the eyeglasses further comprise a camera; a spectroscopic sensor; and a wrist-worn or finger-worn infrared sensor, wherein the camera is triggered to record images and the spectroscopic sensor is activated to make spectroscopic scans when analysis of data from the infrared sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn pressure sensor, wherein the camera is triggered to record images and the spectroscopic sensor is activated to make spectroscopic scans when analysis of data from the pressure sensor indicates that the person is consuming food. In another embodiment, a wearable food consumption monitoring device can comprise: eyeglasses worn by a person; an infrared sensor on a sidepiece (e.g. a temple) of the eyeglasses, wherein the infrared sensor points toward the person's mouth; and a camera on a sidepiece (e.g. a temple) of the eyeglasses, wherein the camera points toward the person's mouth, and wherein the camera is activated to record food images when analysis of data from the infrared sensor indicates that the person is probably eating. In another example, a wearable food consumption monitoring device can comprise: eyeglasses worn by a person; an infrared sensor on a sidepiece (e.g. a temple) of the eyeglasses, wherein the infrared sensor points toward the person's mouth; at least one vibration sensor on the eyeglasses; and a camera on a sidepiece (e.g. a temple) of the eyeglasses, wherein the camera points toward the person's mouth, and wherein the camera is activated to record food images when analysis of data from the infrared sensor and the at least one vibration sensor indicates that the person is probably eating.

In an example, a wearable food consumption monitoring device can comprise: eyeglasses worn by a person; an infrared sensor on the eyeglasses, wherein the infrared sensor points toward the person's mouth; and a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one inertial motion sensor indicates that the person is probably eating. In another example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; and a camera on the eyeglasses, wherein the camera points toward the person's mouth, and wherein the camera is activated to record food images when analysis of data from the infrared sensor and the at least one inertial motion sensor indicates that the person is probably eating. In an example, a wearable food consumption monitoring device can comprise: eyeglasses worn by a person; an infrared sensor on the eyeglasses, wherein the infrared sensor points toward the person's mouth; at least one vibration sensor on the eyeglasses; and a camera on a frontpiece and/or nose bridge of the eyeglasses, wherein the camera points toward the person's mouth, and wherein the camera is activated to record food images when analysis of data from the infrared sensor and the at least one vibration sensor indicates that the person is probably eating.

In another embodiment, a wearable food consumption monitoring system can comprise: eyeglasses worn by a person, wherein the eyeglasses further comprise a camera; and a finger-worn motion sensor, wherein the camera is triggered to record food images when analysis of data from the wrist-worn motion sensor indicates that the person is consuming food. In another example, a wearable food consumption monitoring system can comprise: eyeglasses worn by a person, wherein the eyeglasses further comprise a camera; and a wrist-worn motion sensor, wherein the camera is triggered to record images along an imaging vector which points toward the person's mouth when analysis of data from the wrist-worn motion sensor indicates that the person is consuming food. In an example, a wearable food consumption monitoring device can comprise: eyeglasses worn by a person, wherein the eyeglasses further comprise a camera; and wherein the eyeglasses further comprise a chewing sensor, wherein a first camera is triggered to record images along an imaging vector which points toward the person's mouth and a second camera is triggered to record images along an imaging vector which points toward a reachable food source when analysis of data from the chewing sensor indicates that the person is consuming food.

In another embodiment, a wearable food consumption monitoring device can comprise: eyeglasses worn by a person, wherein the eyeglasses further comprise a camera; and wherein the eyeglasses further comprise a chewing sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise a location sensor, wherein a first camera is triggered to record images along an imaging vector which points toward the person's mouth and a second camera is triggered to record images along an imaging vector which points toward a reachable food source when analysis of data from the location sensor indicates that the person is consuming food. Alternatively, a wearable food consumption monitoring device can comprise: eyeglasses worn by a person, wherein the eyeglasses further comprise a camera; and wherein the eyeglasses further comprise a motion sensor, wherein a first camera is triggered to record images along an imaging vector which points toward the person's mouth and a second camera is triggered to record images along an imaging vector which points toward a reachable food source when analysis of data from the motion sensor indicates that the person is consuming food.

In an example, a wearable food consumption monitoring device can comprise: eyeglasses worn by a person, wherein the eyeglasses further comprise a camera; and wherein the eyeglasses further comprise a optical sensor, wherein a first camera is triggered to record images along an imaging vector which points toward the person's mouth and a second camera is triggered to record images along an imaging vector which points toward a reachable food source when analysis of data from the optical sensor indicates that the person is consuming food. In another example, a wearable food consumption monitoring device can comprise: eyeglasses worn by a person, wherein the eyeglasses further comprise a camera; and wherein the eyeglasses further comprise a pressure sensor, wherein a first camera is triggered to record images along an imaging vector which points toward the person's mouth and a second camera is triggered to record images along an imaging vector which points toward a reachable food source when analysis of data from the pressure sensor indicates that the person is consuming food. In an example, a wearable food consumption monitoring device can comprise: eyeglasses worn by a person, wherein the eyeglasses further comprise a camera; and wherein the eyeglasses further comprise a proximity sensor, wherein a first camera is triggered to record images along an imaging vector which points toward the person's mouth and a second camera is triggered to record images along an imaging vector which points toward a reachable food source when analysis of data from the proximity sensor indicates that the person is consuming food.

In another embodiment, a wearable food consumption monitoring device can comprise: eyeglasses worn by a person, wherein the eyeglasses further comprise a camera; and wherein the eyeglasses further comprise a spectroscopic sensor, wherein the camera is triggered to record images of the interaction between food and the person's mouth when analysis of data from sensor indicates that the person is consuming food. Alternatively, a wearable food consumption monitoring device can comprise: eyeglasses worn by a person, wherein the eyeglasses further comprise a camera; and wherein the eyeglasses further comprise a swallow sensor, wherein a first camera is triggered to record images along an imaging vector which points toward the person's mouth and a second camera is triggered to record images along an imaging vector which points toward a reachable food source when analysis of data from the swallow sensor indicates that the person is consuming food. In another embodiment, a wearable food consumption monitoring device can comprise: eyeglasses worn by a person, wherein the eyeglasses further comprise a camera; and wherein the eyeglasses further comprise a swallowing sensor, wherein the camera is triggered to record images of the interaction between food and the person's mouth when analysis of data from sensor indicates that the person is consuming food.

In an example, a wearable food consumption monitoring device can comprise: eyeglasses worn by a person, wherein the eyeglasses further comprise a camera; and wherein the eyeglasses further comprise an electrochemical sensor, wherein a first camera is triggered to record images along an imaging vector which points toward the person's mouth and a second camera is triggered to record images along an imaging vector which points toward a reachable food source when analysis of data from the electrochemical sensor indicates that the person is consuming food. In another example, a wearable food consumption monitoring device can comprise: eyeglasses worn by a person, wherein the eyeglasses further comprise a camera; and wherein the eyeglasses further comprise an EMG sensor, wherein the camera is triggered to record images along an imaging vector which points toward the person's mouth when analysis of data from the EMG sensor indicates that the person is consuming food. In an example, a wearable food consumption monitoring device can comprise: eyeglasses worn by a person, wherein the eyeglasses further comprise a camera; and wherein the eyeglasses further comprise an infrared sensor which tracks the location of the person's hands, wherein the camera is triggered to record images along an imaging vector which points toward the person's mouth when analysis of data from the infrared sensor indicates that the person is consuming food.

In an example, a wearable food consumption monitoring device can comprise: eyeglasses worn by a person, wherein the eyeglasses further comprise a camera; and wherein the eyeglasses further comprise an infrared sensor, wherein a first camera is triggered to record images along an imaging vector which points toward the person's mouth and a second camera is triggered to record images along an imaging vector which points toward a reachable food source when analysis of data from the infrared sensor indicates that the person is consuming food. In another example, a wearable food consumption monitoring device can comprise: eyeglasses worn by a person; at least one EMG sensor on a portion of the eyeglasses which curves around the rear of the person's ear; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one EMG sensor indicates that the person is probably eating. In another embodiment, a wearable food consumption monitoring device can comprise: eyeglasses worn by a person; at least one EMG sensor on a portion of the eyeglasses which curves around the rear of the person's ear; and a camera on the eyeglasses, wherein the camera points toward the person's mouth, and wherein the camera is activated to record food images when analysis of data from the at least one EMG sensor indicates that the person is probably eating.

In an example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a frontpiece and/or nose bridge of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one EMG sensor indicates that the person is probably eating. In another embodiment, a wearable food consumption monitoring device can comprise: eyeglasses worn by a person; at least one inertial motion sensor (e.g. gyroscope and/or accelerometer) on the eyeglasses; and a camera on a sidepiece (e.g. a temple) of the eyeglasses, wherein the camera points toward the person's mouth, and wherein the camera is activated to record food images when analysis of data from the at least one inertial motion sensor indicates that the person is probably eating.

In an example, a wearable food consumption monitoring device can comprise: eyeglasses worn by a person; at least one inertial motion sensor (e.g. gyroscope and/or accelerometer) on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one inertial motion sensor indicates that the person is probably eating. Alternatively, a wearable food consumption monitoring device can comprise: eyeglasses worn by a person; at least one vibration sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one vibration sensor indicates that the person is probably eating. In an example, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one wrist-worn or finger-worn inertial motion sensor indicates that the person is probably eating.

In an example, a wearable food consumption monitoring system can comprise: eyeglasses worn by a person; at least one wrist-worn or finger-worn inertial motion sensor (e.g. gyroscope and/or accelerometer on a smart watch or smart ring); and a camera on the eyeglasses, wherein the camera points toward the person's mouth, and wherein the camera is activated to record food images when analysis of data from the at least one wrist-worn or finger-worn inertial motion sensor indicates that the person is probably eating. In another example, a wearable food consumption monitoring device can comprise: eyeglasses worn by a person, wherein the eyeglasses further comprise a camera; wherein the eyeglasses further comprise an EMG sensor; and wherein the eyeglasses further comprise an infrared sensor which tracks the location of the person's hands, wherein the camera is triggered to record images when joint analysis of data from the EMG sensor and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone), wherein a first camera is triggered to record images along an imaging vector which points toward the person's mouth and a second camera is triggered to record images of a reachable food source when analysis of data from the sound sensor (e.g. microphone) indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone) and an infrared sensor, wherein a first camera is triggered to record images along an imaging vector which points toward the person's mouth and a second camera is triggered to record images of a reachable food source when joint analysis of data from the sound sensor (e.g. microphone) and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone), wherein a first camera is triggered to record images along an imaging vector which points toward the person's mouth and a second camera is triggered to record images of a reachable food source when analysis of data from the sound sensor (e.g. microphone) indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone) and an EEG sensor, wherein a first camera is triggered to record images along an imaging vector which points toward the person's mouth and a second camera is triggered to record images of a reachable food source when joint analysis of data from the sound sensor (e.g. microphone) and the EEG sensor indicates that the person is consuming food.

In an example, a wearable food consumption monitoring device can comprise: eyeglasses worn by a person; wherein the eyeglasses further comprise at least two cameras; and wherein the eyeglasses further comprise a swallow sensor and an accelerometer, wherein a first camera is triggered to record images along an imaging vector which points toward the person's mouth and a second camera is triggered to record images of a reachable food source when joint analysis of data from the swallow sensor and the accelerometer indicates that the person is consuming food. In another example, a wearable food consumption monitoring device can comprise: eyeglasses worn by a person; wherein the eyeglasses further comprise at least two cameras; and wherein the eyeglasses further comprise a swallow sensor and a motion sensor, wherein a first camera is triggered to record images along an imaging vector which points toward the person's mouth and a second camera is triggered to record images of a reachable food source when joint analysis of data from the swallow sensor and the motion sensor indicates that the person is consuming food. In an example, a wearable food consumption monitoring device can comprise: eyeglasses worn by a person; wherein the eyeglasses further comprise at least two cameras; and wherein the eyeglasses further comprise a swallow sensor, an accelerometer, and an infrared sensor, wherein a first camera is triggered to record images along an imaging vector which points toward the person's mouth and a second camera is triggered to record images of a reachable food source when joint analysis of data from the swallow sensor, the accelerometer, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor and a sound sensor (e.g. microphone), wherein a first camera is triggered to record images along an imaging vector which points toward the person's mouth and a second camera is triggered to record images of a reachable food source when joint analysis of data from the swallowing sensor and the sound sensor (e.g. microphone) indicates that the person is consuming food. In another example, a wearable food consumption monitoring device can comprise: eyeglasses worn by a person; wherein the eyeglasses further comprise at least two cameras; and wherein the eyeglasses further comprise a swallowing sensor and a motion sensor, wherein a first camera is triggered to record images along an imaging vector which points toward the person's mouth and a second camera is triggered to record images of a reachable food source when joint analysis of data from the swallowing sensor and the motion sensor indicates that the person is consuming food. In an example, a wearable food consumption monitoring device can comprise: eyeglasses worn by a person; wherein the eyeglasses further comprise at least two cameras; and wherein the eyeglasses further comprise a swallowing sensor, a sound sensor (e.g. microphone), and an infrared sensor, wherein a first camera is triggered to record images along an imaging vector which points toward the person's mouth and a second camera is triggered to record images of a reachable food source when joint analysis of data from the swallowing sensor, the sound sensor (e.g. microphone), and the infrared sensor indicates that the person is consuming food.

In another embodiment, a wearable food consumption monitoring device can comprise: eyeglasses worn by a person; wherein the eyeglasses further comprise at least two cameras; and wherein the eyeglasses further comprise an accelerometer and a chewing sensor, wherein a first camera is triggered to record images along an imaging vector which points toward the person's mouth and a second camera is triggered to record images of a reachable food source when joint analysis of data from the accelerometer and the chewing sensor indicates that the person is consuming food. In an example, a wearable food consumption monitoring device can comprise: eyeglasses worn by a person; wherein the eyeglasses further comprise at least two cameras; and wherein the eyeglasses further comprise an EEG sensor and an EMG sensor, wherein a first camera is triggered to record images along an imaging vector which points toward the person's mouth and a second camera is triggered to record images of a reachable food source when joint analysis of data from the EEG sensor and the EMG sensor indicates that the person is consuming food. In another embodiment, a wearable food consumption monitoring device can comprise: eyeglasses worn by a person; wherein the eyeglasses further comprise at least two cameras; and wherein the eyeglasses further comprise an EEG sensor, an EMG sensor, and an infrared sensor, wherein a first camera is triggered to record images along an imaging vector which points toward the person's mouth and a second camera is triggered to record images of a reachable food source when joint analysis of data from the EEG sensor, the EMG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor and a chewing sensor, wherein a first camera is triggered to record images along an imaging vector which points toward the person's mouth and a second camera is triggered to record images of a reachable food source when joint analysis of data from the EMG sensor and the chewing sensor indicates that the person is consuming food. In another example, a wearable food consumption monitoring device can comprise: eyeglasses worn by a person; wherein the eyeglasses further comprise at least two cameras; and wherein the eyeglasses further comprise an EMG sensor and an accelerometer, wherein a first camera is triggered to record images along an imaging vector which points toward the person's mouth and a second camera is triggered to record images of a reachable food source when joint analysis of data from the EMG sensor and the accelerometer indicates that the person is consuming food. In an example, a wearable food consumption monitoring device can comprise: eyeglasses worn by a person; wherein the eyeglasses further comprise at least two cameras; and wherein the eyeglasses further comprise an EMG sensor, an accelerometer, and an infrared sensor, wherein a first camera is triggered to record images along an imaging vector which points toward the person's mouth and a second camera is triggered to record images of a reachable food source when joint analysis of data from the EMG sensor, the accelerometer, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an motion sensor and an infrared sensor, wherein a first camera is triggered to record images along an imaging vector which points toward the person's mouth and a second camera is triggered to record images of a reachable food source when joint analysis of data from the motion sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone), wherein at least one camera is triggered to record images along an imaging vector which points toward the person's mouth when analysis of data from the sound sensor (e.g. microphone) indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone) and an infrared sensor, wherein at least one camera is triggered to record images along an imaging vector which points toward the person's mouth when joint analysis of data from the sound sensor (e.g. microphone) and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone), an accelerometer, and an infrared sensor, wherein at least one camera is triggered to record images along an imaging vector which points toward the person's mouth when joint analysis of data from the sound sensor (e.g. microphone), the accelerometer, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and an accelerometer, wherein at least one camera is triggered to record images along an imaging vector which points toward the person's mouth when joint analysis of data from the sound sensor (e.g. microphone) and the accelerometer indicates that the person is consuming food. Alternatively, a wearable food consumption monitoring device can comprise: eyeglasses worn by a person; wherein the eyeglasses further comprise one or more cameras; and wherein the eyeglasses further comprise a swallow sensor and an accelerometer, wherein at least one camera is triggered to record images along an imaging vector which points toward the person's mouth when joint analysis of data from the swallow sensor and the accelerometer indicates that the person is consuming food.

In another embodiment, a wearable food consumption monitoring device can comprise: eyeglasses worn by a person; wherein the eyeglasses further comprise one or more cameras; and wherein the eyeglasses further comprise a swallow sensor and a motion sensor, wherein at least one camera is triggered to record images along an imaging vector which points toward the person's mouth when joint analysis of data from the swallow sensor and the motion sensor indicates that the person is consuming food. In another example, a wearable food consumption monitoring device can comprise: eyeglasses worn by a person; wherein the eyeglasses further comprise one or more cameras; and wherein the eyeglasses further comprise a swallow sensor, an accelerometer, and an infrared sensor, wherein at least one camera is triggered to record images along an imaging vector which points toward the person's mouth when joint analysis of data from the swallow sensor, the accelerometer, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise a swallowing sensor and a sound sensor (e.g. microphone), wherein at least one camera is triggered to record images along an imaging vector which points toward the person's mouth when joint analysis of data from the swallowing sensor and the sound sensor (e.g. microphone) indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor and a motion sensor, wherein at least one camera is triggered to record images along an imaging vector which points toward the person's mouth when joint analysis of data from the swallowing sensor and the motion sensor indicates that the person is consuming food. Alternatively, a wearable food consumption monitoring device can comprise: eyeglasses worn by a person; wherein the eyeglasses further comprise one or more cameras; and wherein the eyeglasses further comprise a swallowing sensor, a chewing sensor, and an infrared sensor, wherein at least one camera is triggered to record images along an imaging vector which points toward the person's mouth when joint analysis of data from the swallowing sensor, the chewing sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an accelerometer and a chewing sensor, wherein at least one camera is triggered to record images along an imaging vector which points toward the person's mouth when joint analysis of data from the accelerometer and the chewing sensor indicates that the person is consuming food. In another embodiment, a wearable food consumption monitoring device can comprise: eyeglasses worn by a person; wherein the eyeglasses further comprise one or more cameras; and wherein the eyeglasses further comprise an EEG sensor and an EMG sensor, wherein at least one camera is triggered to record images along an imaging vector which points toward the person's mouth when joint analysis of data from the EEG sensor and the EMG sensor indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor, an EMG sensor, and an infrared sensor, wherein at least one camera is triggered to record images along an imaging vector which points toward the person's mouth when joint analysis of data from the EEG sensor, the EMG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor and a chewing sensor, wherein at least one camera is triggered to record images along an imaging vector which points toward the person's mouth when joint analysis of data from the EMG sensor and the chewing sensor indicates that the person is consuming food. In another embodiment, a wearable food consumption monitoring device can comprise: eyeglasses worn by a person; wherein the eyeglasses further comprise one or more cameras; and wherein the eyeglasses further comprise an EMG sensor and an accelerometer, wherein at least one camera is triggered to record images along an imaging vector which points toward the person's mouth when joint analysis of data from the EMG sensor and the accelerometer indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, an EEG sensor, and an infrared sensor, wherein at least one camera is triggered to record images along an imaging vector which points toward the person's mouth when joint analysis of data from the EMG sensor, the EEG sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an motion sensor and an infrared sensor, wherein at least one camera is triggered to record images along an imaging vector which points toward the person's mouth when joint analysis of data from the motion sensor and the infrared sensor indicates that the person is consuming food. In another embodiment, a wearable food consumption monitoring system can comprise: eyeglasses worn by a person; a camera on the eyeglasses; a spectroscopic sensor; and a location sensor, wherein the camera is triggered to record images and the spectroscopic sensor is activated to make spectroscopic scans when analysis of data from the location sensor indicates that the person is consuming food. In another example, a wearable food consumption monitoring system can comprise: eyeglasses wom by a person; a camera on the eyeglasses; a spectroscopic sensor; and a smell sensor, wherein the camera is triggered to record images and the spectroscopic sensor is activated to make spectroscopic scans when analysis of data from the smell sensor indicates that the person is consuming food. In an example, a wearable food consumption monitoring system can comprise: eyeglasses worn by a person; a camera on the eyeglasses; a spectroscopic sensor; and an EMG sensor, wherein the camera is triggered to record images and the spectroscopic sensor is activated to make spectroscopic scans when analysis of data from the EMG sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn location sensor, wherein the camera is triggered to record images and the spectroscopic sensor is activated to make spectroscopic scans when analysis of data from the location sensor indicates that the person is consuming food. In another example, a wearable food consumption monitoring system can comprise: eyeglasses worn by a person, wherein the eyeglasses further comprise a camera; a spectroscopic sensor; and a wrist-worn or finger-worn proximity sensor, wherein the camera is triggered to record images and the spectroscopic sensor is activated to make spectroscopic scans when analysis of data from the proximity sensor indicates that the person is consuming food. In an example, a wearable food consumption monitoring device can comprise: eyeglasses worn by a person; an infrared sensor on a sidepiece (e.g. a temple) of the eyeglasses, wherein the infrared sensor points toward the person's mouth; at least one EMG sensor on a portion of the eyeglasses which curves around the rear of the person's ear; and a camera on a sidepiece (e.g. a temple) of the eyeglasses, wherein the camera points toward the person's mouth, and wherein the camera is activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor indicates that the person is probably eating.

In another embodiment, a wearable food consumption monitoring device can comprise: eyeglasses worn by a person; an infrared sensor on the eyeglasses, wherein the infrared sensor points toward the person's mouth; and a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor indicates that the person is probably eating. In another example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; and a camera on a frontpiece and/or nose bridge of the eyeglasses, wherein the camera points toward the person's mouth, and wherein the camera is activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one EMG sensor indicates that the person is probably eating. In another embodiment, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one inertial motion sensor indicates that the person is probably eating. In another example, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one vibration sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; and a camera on the eyeglasses, wherein the camera points toward the person's mouth, and wherein the camera is activated to record food images when analysis of data from the infrared sensor and the at least one vibration sensor indicates that the person is probably eating. In an example, a wearable food consumption monitoring system can comprise: eyeglasses worn by a person, wherein the eyeglasses further comprise a camera; and a finger-worn motion sensor, wherein the camera is triggered to record images along an imaging vector which points toward the person's mouth when analysis of data from the wrist-worn motion sensor indicates that the person is consuming food.

In an example, a wearable food consumption monitoring system can comprise: eyeglasses worn by a person, wherein the eyeglasses further comprise a camera; and a wrist-worn motion sensor, wherein the camera is triggered to record food images when analysis of data from the wrist-worn motion sensor indicates that the person is consuming food. In another example, a wearable food consumption monitoring device can comprise: eyeglasses worn by a person, wherein the eyeglasses further comprise a camera; and wherein the eyeglasses further comprise a chewing sensor, wherein the camera is triggered to record images along an imaging vector which points toward the person's mouth when analysis of data from the chewing sensor indicates that the person is consuming food. In an example, a wearable food consumption monitoring device can comprise: eyeglasses worn by a person, wherein the eyeglasses further comprise a camera; and wherein the eyeglasses further comprise a GPS sensor, wherein a first camera is triggered to record images along an imaging vector which points toward the person's mouth and a second camera is triggered to record images along an imaging vector which points toward a reachable food source when analysis of data from the GPS sensor indicates that the person is consuming food.

In an example, a wearable food consumption monitoring device can comprise: eyeglasses worn by a person, wherein the eyeglasses further comprise a camera; and wherein the eyeglasses further comprise a location sensor, wherein the camera is triggered to record images along an imaging vector which points toward the person's mouth when analysis of data from the location sensor indicates that the person is consuming food. In another example, a wearable food consumption monitoring device can comprise: eyeglasses worn by a person, wherein the eyeglasses further comprise a camera; and wherein the eyeglasses further comprise a motion sensor, wherein the camera is triggered to record images along an imaging vector which points toward the person's mouth when analysis of data from the motion sensor indicates that the person is consuming food. In another embodiment, a wearable food consumption monitoring device can comprise: eyeglasses worn by a person, wherein the eyeglasses further comprise a camera; and wherein the eyeglasses further comprise a piezoelectric sensor, wherein a first camera is triggered to record images along an imaging vector which points toward the person's mouth and a second camera is triggered to record images along an imaging vector which points toward a reachable food source when analysis of data from the piezoelectric sensor indicates that the person is consuming food.

In an example, a wearable food consumption monitoring device can comprise: eyeglasses worn by a person, wherein the eyeglasses further comprise a camera; and wherein the eyeglasses further comprise a pressure sensor, wherein the camera is triggered to record images along an imaging vector which points toward the person's mouth when analysis of data from the pressure sensor indicates that the person is consuming food. In another example, a wearable food consumption monitoring device can comprise: eyeglasses worn by a person, wherein the eyeglasses further comprise a camera; and wherein the eyeglasses further comprise a smell sensor, wherein a first camera is triggered to record images along an imaging vector which points toward the person's mouth and a second camera is triggered to record images along an imaging vector which points toward a reachable food source when analysis of data from the smell sensor indicates that the person is consuming food. In another embodiment, a wearable food consumption monitoring device can comprise: eyeglasses worn by a person, wherein the eyeglasses further comprise a camera; and wherein the eyeglasses further comprise a strain gauge, wherein a first camera is triggered to record images along an imaging vector which points toward the person's mouth and a second camera is triggered to record images along an imaging vector which points toward a reachable food source when analysis of data from the strain gauge indicates that the person is consuming food.

In an example, a wearable food consumption monitoring device can comprise: eyeglasses worn by a person, wherein the eyeglasses further comprise a camera; and wherein the eyeglasses further comprise a swallowing sensor, wherein the camera is triggered to record images along an imaging vector which points toward the person's mouth when analysis of data from the swallowing sensor indicates that the person is consuming food. In another embodiment, a wearable food consumption monitoring device can comprise: eyeglasses worn by a person, wherein the eyeglasses further comprise a camera; and wherein the eyeglasses further comprise an EEG sensor, wherein a first camera is triggered to record images along an imaging vector which points toward the person's mouth and a second camera is triggered to record images along an imaging vector which points toward a reachable food source when analysis of data from the EEG sensor indicates that the person is consuming food. In an example, a wearable food consumption monitoring device can comprise: eyeglasses worn by a person, wherein the eyeglasses further comprise a camera; and wherein the eyeglasses further comprise an electrochemical sensor, wherein the camera is triggered to record images along an imaging vector which points toward the person's mouth when analysis of data from the electrochemical sensor indicates that the person is consuming food.

In an example, a wearable food consumption monitoring device can comprise: eyeglasses worn by a person, wherein the eyeglasses further comprise a camera; and wherein the eyeglasses further comprise an EMG sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise an infrared sensor which tracks the location of the person's hands, wherein the camera is triggered to record images when analysis of data from the infrared sensor indicates that the person is consuming food. Alternatively, a wearable food consumption monitoring device can comprise: eyeglasses worn by a person, wherein the eyeglasses further comprise a camera; and wherein the eyeglasses further comprise an optical sensor, wherein the camera is triggered to record images along an imaging vector which points toward the person's mouth when analysis of data from the optical sensor indicates that the person is consuming food.

In an example, a wearable food consumption monitoring device can comprise: eyeglasses worn by a person; at least one EMG sensor on a portion of the eyeglasses which curves around the rear of the person's ear; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one EMG sensor indicates that the person is probably eating. In another example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one EMG sensor indicates that the person is probably eating. In another embodiment, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a sidepiece (e.g. a temple) of the eyeglasses, wherein the camera points toward the person's mouth, and wherein the camera is activated to record food images when analysis of data from the at least one EMG sensor indicates that the person is probably eating.

In an example, a wearable food consumption monitoring device can comprise: eyeglasses worn by a person; at least one EMG sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one inertial motion sensor indicates that the person is probably eating. In another embodiment, a wearable food consumption monitoring device can comprise: eyeglasses worn by a person; at least one inertial motion sensor (e.g. gyroscope and/or accelerometer) on the eyeglasses; and a camera on the eyeglasses, wherein the camera points toward the person's mouth, and wherein the camera is activated to record food images when analysis of data from the at least one inertial motion sensor indicates that the person is probably eating.

In an example, a wearable food consumption monitoring device can comprise: eyeglasses worn by a person; at least one vibration sensor on the eyeglasses; and a camera on a frontpiece and/or nose bridge of the eyeglasses, wherein the camera points toward the person's mouth, and wherein the camera is activated to record food images when analysis of data from the at least one vibration sensor indicates that the person is probably eating. In another example, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one wrist-worn or finger-worn inertial motion sensor indicates that the person is probably eating. In another embodiment, a wearable food consumption monitoring device can comprise: eyeglasses worn by a person, wherein the eyeglasses further comprise a camera; wherein the eyeglasses further comprise a motion sensor; and wherein the eyeglasses further comprise an infrared sensor which tracks the location of the person's hands, wherein the camera is triggered to record images along an imaging vector which points toward the person's mouth when joint analysis of data from the motion sensor and the infrared sensor indicates that the person is consuming food.

In an example, a wearable food consumption monitoring device can comprise: eyeglasses worn by a person, wherein the eyeglasses further comprise a camera; wherein the eyeglasses further comprise an EMG sensor; and wherein the eyeglasses further comprise an infrared sensor which tracks the location of the person's hands, wherein the camera is triggered to record images along an imaging vector which points toward the person's mouth when joint analysis of data from the EMG sensor and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise a sound sensor (e.g. microphone) and an accelerometer, wherein a first camera is triggered to record images along an imaging vector which points toward the person's mouth and a second camera is triggered to record images of a reachable food source when joint analysis of data from the sound sensor (e.g. microphone) and the accelerometer indicates that the person is consuming food. Alternatively, a wearable food consumption monitoring device can comprise: eyeglasses worn by a person; wherein the eyeglasses further comprise at least two cameras; and wherein the eyeglasses further comprise a sound sensor (e.g. microphone), an accelerometer, and an infrared sensor, wherein a first camera is triggered to record images along an imaging vector which points toward the person's mouth and a second camera is triggered to record images of a reachable food source when joint analysis of data from the sound sensor (e.g. microphone), the accelerometer, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), an accelerometer, and an infrared sensor, wherein a first camera is triggered to record images along an imaging vector which points toward the person's mouth and a second camera is triggered to record images of a reachable food source when joint analysis of data from the sound sensor (e.g. microphone), the accelerometer, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone) and a chewing sensor, wherein a first camera is triggered to record images along an imaging vector which points toward the person's mouth and a second camera is triggered to record images of a reachable food source when joint analysis of data from the sound sensor (e.g. microphone) and the chewing sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallow sensor and a chewing sensor, wherein a first camera is triggered to record images along an imaging vector which points toward the person's mouth and a second camera is triggered to record images of a reachable food source when joint analysis of data from the swallow sensor and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a swallow sensor and an infrared sensor, wherein a first camera is triggered to record images along an imaging vector which points toward the person's mouth and a second camera is triggered to record images of a reachable food source when joint analysis of data from the swallow sensor and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor, an EEG sensor, and an infrared sensor, wherein a first camera is triggered to record images along an imaging vector which points toward the person's mouth and a second camera is triggered to record images of a reachable food source when joint analysis of data from the swallow sensor, the EEG sensor, and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallowing sensor and an EEG sensor, wherein a first camera is triggered to record images along an imaging vector which points toward the person's mouth and a second camera is triggered to record images of a reachable food source when joint analysis of data from the swallowing sensor and the EEG sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor, wherein a first camera is triggered to record images along an imaging vector which points toward the person's mouth and a second camera is triggered to record images of a reachable food source when analysis of data from the swallowing sensor indicates that the person is consuming food. In another example, a wearable food consumption monitoring device can comprise: eyeglasses worn by a person; wherein the eyeglasses further comprise at least two cameras; and wherein the eyeglasses further comprise a swallowing sensor, a motion sensor, and an infrared sensor, wherein a first camera is triggered to record images along an imaging vector which points toward the person's mouth and a second camera is triggered to record images of a reachable food source when joint analysis of data from the swallowing sensor, the motion sensor, and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise an accelerometer, a chewing sensor, and an infrared sensor, wherein a first camera is triggered to record images along an imaging vector which points toward the person's mouth and a second camera is triggered to record images of a reachable food source when joint analysis of data from the accelerometer, the chewing sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise an EEG sensor and a motion sensor, wherein a first camera is triggered to record images along an imaging vector which points toward the person's mouth and a second camera is triggered to record images of a reachable food source when joint analysis of data from the EEG sensor and the motion sensor indicates that the person is consuming food. In another example, a wearable food consumption monitoring device can comprise: eyeglasses worn by a person; wherein the eyeglasses further comprise at least two cameras; and wherein the eyeglasses further comprise an EEG sensor, a motion sensor, and an infrared sensor, wherein a first camera is triggered to record images along an imaging vector which points toward the person's mouth and a second camera is triggered to record images of a reachable food source when joint analysis of data from the EEG sensor, the motion sensor, and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise an EMG sensor and an EEG sensor, wherein a first camera is triggered to record images along an imaging vector which points toward the person's mouth and a second camera is triggered to record images of a reachable food source when joint analysis of data from the EMG sensor and the EEG sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor, a chewing sensor, and an infrared sensor, wherein a first camera is triggered to record images along an imaging vector which points toward the person's mouth and a second camera is triggered to record images of a reachable food source when joint analysis of data from the EMG sensor, the chewing sensor, and the infrared sensor indicates that the person is consuming food. Alternatively, a wearable food consumption monitoring device can comprise: eyeglasses worn by a person; wherein the eyeglasses further comprise at least two cameras; and wherein the eyeglasses further comprise an EMG sensor, wherein a first camera is triggered to record images along an imaging vector which points toward the person's mouth and a second camera is triggered to record images of a reachable food source when analysis of data from the EMG sensor indicates that the person is consuming food. In another example, a wearable food consumption monitoring device can comprise: eyeglasses worn by a person; wherein the eyeglasses further comprise at least two cameras; and wherein the eyeglasses further comprise an motion sensor, a chewing sensor, and an infrared sensor, wherein a first camera is triggered to record images along an imaging vector which points toward the person's mouth and a second camera is triggered to record images of a reachable food source when joint analysis of data from the motion sensor, the chewing sensor, and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone) and an accelerometer, wherein at least one camera is triggered to record images along an imaging vector which points toward the person's mouth when joint analysis of data from the sound sensor (e.g. microphone) and the accelerometer indicates that the person is consuming food. In an example, a wearable food consumption monitoring device can comprise: eyeglasses worn by a person; wherein the eyeglasses further comprise one or more cameras; and wherein the eyeglasses further comprise a sound sensor (e.g. microphone), an accelerometer, and an infrared sensor, wherein at least one camera is triggered to record images along an imaging vector which points toward the person's mouth when joint analysis of data from the sound sensor (e.g. microphone), the accelerometer, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone), an EEG sensor, and an infrared sensor, wherein at least one camera is triggered to record images along an imaging vector which points toward the person's mouth when joint analysis of data from the sound sensor (e.g. microphone), the EEG sensor, and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a sound sensor (e.g. microphone), wherein at least one camera is triggered to record images along an imaging vector which points toward the person's mouth when analysis of data from the sound sensor (e.g. microphone) indicates that the person is consuming food. In an example, a wearable food consumption monitoring device can comprise: eyeglasses worn by a person; wherein the eyeglasses further comprise one or more cameras; and wherein the eyeglasses further comprise a swallow sensor and a chewing sensor, wherein at least one camera is triggered to record images along an imaging vector which points toward the person's mouth when joint analysis of data from the swallow sensor and the chewing sensor indicates that the person is consuming food. In another example, a wearable food consumption monitoring device can comprise: eyeglasses worn by a person; wherein the eyeglasses further comprise one or more cameras; and wherein the eyeglasses further comprise a swallow sensor and an infrared sensor, wherein at least one camera is triggered to record images along an imaging vector which points toward the person's mouth when joint analysis of data from the swallow sensor and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise a swallow sensor, an EEG sensor, and an infrared sensor, wherein at least one camera is triggered to record images along an imaging vector which points toward the person's mouth when joint analysis of data from the swallow sensor, the EEG sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise a swallowing sensor and an EEG sensor, wherein at least one camera is triggered to record images along an imaging vector which points toward the person's mouth when joint analysis of data from the swallowing sensor and the EEG sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor, an EEG sensor, and an infrared sensor, wherein at least one camera is triggered to record images along an imaging vector which points toward the person's mouth when joint analysis of data from the swallowing sensor, the EEG sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor, wherein at least one camera is triggered to record images along an imaging vector which points toward the person's mouth when analysis of data from the swallowing sensor indicates that the person is consuming food. In an example, a wearable food consumption monitoring device can comprise: eyeglasses worn by a person; wherein the eyeglasses further comprise one or more cameras; and wherein the eyeglasses further comprise an accelerometer, a chewing sensor, and an infrared sensor, wherein at least one camera is triggered to record images along an imaging vector which points toward the person's mouth when joint analysis of data from the accelerometer, the chewing sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an EEG sensor and a motion sensor, wherein at least one camera is triggered to record images along an imaging vector which points toward the person's mouth when joint analysis of data from the EEG sensor and the motion sensor indicates that the person is consuming food.

In another embodiment, a wearable food consumption monitoring device can comprise: eyeglasses worn by a person; wherein the eyeglasses further comprise one or more cameras; and wherein the eyeglasses further comprise an EEG sensor, a motion sensor, and an infrared sensor, wherein at least one camera is triggered to record images along an imaging vector which points toward the person's mouth when joint analysis of data from the EEG sensor, the motion sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor and an EEG sensor, wherein at least one camera is triggered to record images along an imaging vector which points toward the person's mouth when joint analysis of data from the EMG sensor and the EEG sensor indicates that the person is consuming food. Alternatively, a wearable food consumption monitoring device can comprise: eyeglasses worn by a person; wherein the eyeglasses further comprise one or more cameras; and wherein the eyeglasses further comprise an EMG sensor and an infrared sensor, wherein at least one camera is triggered to record images along an imaging vector which points toward the person's mouth when joint analysis of data from the EMG sensor and the infrared sensor indicates that the person is consuming food.

In another embodiment, a wearable food consumption monitoring device can comprise: eyeglasses worn by a person; wherein the eyeglasses further comprise one or more cameras; and wherein the eyeglasses further comprise an EMG sensor, an accelerometer, and an infrared sensor, wherein at least one camera is triggered to record images along an imaging vector which points toward the person's mouth when joint analysis of data from the EMG sensor, the accelerometer, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an motion sensor and a chewing sensor, wherein at least one camera is triggered to record images along an imaging vector which points toward the person's mouth when joint analysis of data from the motion sensor and the chewing sensor indicates that the person is consuming food. In an example, a wearable food consumption monitoring system can comprise: eyeglasses worn by a person; a camera on the eyeglasses; a spectroscopic sensor; and a motion sensor, wherein the camera is triggered to record images and the spectroscopic sensor is activated to make spectroscopic scans when analysis of data from the motion sensor indicates that the person is consuming food.

In another embodiment, a wearable food consumption monitoring system can comprise: eyeglasses worn by a person; a camera on the eyeglasses; a spectroscopic sensor; and a strain gauge, wherein the camera is triggered to record images and the spectroscopic sensor is activated to make spectroscopic scans when analysis of data from the strain gauge indicates that the person is consuming food. In another example, a wearable food consumption monitoring system can comprise: eyeglasses worn by a person; a camera on the eyeglasses; a spectroscopic sensor; and an infrared sensor, wherein the camera is triggered to record images and the spectroscopic sensor is activated to make spectroscopic scans when analysis of data from the infrared sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn electrochemical sensor, wherein the camera is triggered to record images and the spectroscopic sensor is activated to make spectroscopic scans when analysis of data from the electrochemical sensor indicates that the person is consuming food. Alternatively, a wearable food consumption monitoring system can comprise: eyeglasses worn by a person, wherein the eyeglasses further comprise a camera; a spectroscopic sensor; and a wrist-worn or finger-worn motion sensor, wherein the camera is triggered to record images and the spectroscopic sensor is activated to make spectroscopic scans when analysis of data from the motion sensor indicates that the person is consuming food.

In an example, a wearable food consumption monitoring system can comprise: eyeglasses worn by a person, wherein the eyeglasses further comprise a camera; a spectroscopic sensor; and a wrist-worn or finger-worn smell sensor, wherein the camera is triggered to record images and the spectroscopic sensor is activated to make spectroscopic scans when analysis of data from the smell sensor indicates that the person is consuming food. In another example, a wearable food consumption monitoring device can comprise: eyeglasses worn by a person; an infrared sensor on a sidepiece (e.g. a temple) of the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a sidepiece (e.g. a temple) of the eyeglasses, wherein the camera points toward the person's mouth, and wherein the camera is activated to record food images when analysis of data from the infrared sensor and the at least one EMG sensor indicates that the person is probably eating.

In an example, a wearable food consumption monitoring device can comprise: eyeglasses worn by a person; an infrared sensor on the eyeglasses, wherein the infrared sensor points toward the person's mouth; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor indicates that the person is probably eating. In another embodiment, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on a portion of the eyeglasses which curves around the rear of the person's ear; and a camera on the eyeglasses, wherein the camera points toward the person's mouth, and wherein the camera is activated to record food images when analysis of data from the infrared sensor and the at least one EMG sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on a frontpiece and/or nose bridge of the eyeglasses, wherein the camera points toward the person's mouth, and wherein the camera is activated to record food images when analysis of data from the infrared sensor and the at least one EMG sensor indicates that the person is probably eating. In an example, a wearable food consumption monitoring device can comprise: eyeglasses worn by a person; an infrared sensor on the eyeglasses, wherein the infrared sensor points toward the person's mouth; at least one EMG sensor on the eyeglasses; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the infrared sensor and the at least one EMG sensor indicates that the person is probably eating. In another example, a wearable food consumption monitoring device can comprise: eyeglasses worn by a person; an infrared sensor on the eyeglasses, wherein the infrared sensor points toward the person's mouth; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the infrared sensor and the at least one inertial motion sensor indicates that the person is probably eating.

In another embodiment, a wearable food consumption monitoring device can comprise: eyeglasses worn by a person; an infrared sensor on the eyeglasses, wherein the infrared sensor points toward the person's mouth; at least one vibration sensor on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the infrared sensor and the at least one vibration sensor indicates that the person is probably eating. In an example, a wearable food consumption monitoring system can comprise: eyeglasses worn by a person, wherein the eyeglasses further comprise at least two cameras; and a finger-worn motion sensor (e.g. in a smart ring), wherein a first camera is triggered to record images along an imaging vector which points toward the person's mouth and a second camera is triggered to record images of a reachable food source when analysis of data from the finger-worn motion sensor indicates that the person is consuming food. In another example, a wearable food consumption monitoring device can comprise: eyeglasses worn by a person, wherein the eyeglasses further comprise a camera; and wherein the eyeglasses further comprise a blood pressure sensor, wherein a first camera is triggered to record images along an imaging vector which points toward the person's mouth and a second camera is triggered to record images along an imaging vector which points toward a reachable food source when analysis of data from the blood pressure sensor indicates that the person is consuming food.

In an example, a wearable food consumption monitoring system can comprise: eyeglasses worn by a person, wherein the eyeglasses further comprise a camera; and a finger-worn motion sensor, wherein the camera is triggered to record food images when analysis of data from the wrist-worn motion sensor indicates that the person is consuming food. In an example, a wearable food consumption monitoring device can comprise: eyeglasses worn by a person, wherein the eyeglasses further comprise a camera; and wherein the eyeglasses further comprise a chewing sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise a GPS sensor, wherein the camera is triggered to record images along an imaging vector which points toward the person's mouth when analysis of data from the GPS sensor indicates that the person is consuming food.

In an example, a wearable food consumption monitoring device can comprise: eyeglasses worn by a person, wherein the eyeglasses further comprise a camera; and wherein the eyeglasses further comprise a location sensor, wherein a first camera is triggered to record images along an imaging vector which points toward the person's mouth and a second camera is triggered to record images along an imaging vector which points toward a reachable food source when analysis of data from the location sensor indicates that the person is consuming food. In another embodiment, a wearable food consumption monitoring device can comprise: eyeglasses worn by a person, wherein the eyeglasses further comprise a camera; and wherein the eyeglasses further comprise a motion sensor, wherein the camera is triggered to record images of the interaction between food and the person's mouth when analysis of data from sensor indicates that the person is consuming food.

In an example, a wearable food consumption monitoring device can comprise: eyeglasses worn by a person, wherein the eyeglasses further comprise a camera; and wherein the eyeglasses further comprise a piezoelectric sensor, wherein the camera is triggered to record images along an imaging vector which points toward the person's mouth when analysis of data from the piezoelectric sensor indicates that the person is consuming food. In another example, a wearable food consumption monitoring device can comprise: eyeglasses worn by a person, wherein the eyeglasses further comprise a camera; and wherein the eyeglasses further comprise a pressure sensor, wherein a first camera is triggered to record images along an imaging vector which points toward the person's mouth and a second camera is triggered to record images along an imaging vector which points toward a reachable food source when analysis of data from the pressure sensor indicates that the person is consuming food. In another embodiment, a wearable food consumption monitoring device can comprise: eyeglasses worn by a person, wherein the eyeglasses further comprise a camera; and wherein the eyeglasses further comprise a smell sensor, wherein the camera is triggered to record images along an imaging vector which points toward the person's mouth when analysis of data from the smell sensor indicates that the person is consuming food.

In an example, a wearable food consumption monitoring device can comprise: eyeglasses worn by a person, wherein the eyeglasses further comprise a camera; and wherein the eyeglasses further comprise a strain gauge, wherein the camera is triggered to record images along an imaging vector which points toward the person's mouth when analysis of data from the strain gauge indicates that the person is consuming food. In another embodiment, a wearable food consumption monitoring device can comprise: eyeglasses worn by a person, wherein the eyeglasses further comprise a camera; and wherein the eyeglasses further comprise a swallowing sensor, wherein the camera is triggered to record images of the interaction between food and the person's mouth when analysis of data from sensor indicates that the person is consuming food. In another example, a wearable food consumption monitoring device can comprise: eyeglasses worn by a person, wherein the eyeglasses further comprise a camera; and wherein the eyeglasses further comprise an EEG sensor, wherein the camera is triggered to record images along an imaging vector which points toward the person's mouth when analysis of data from the EEG sensor indicates that the person is consuming food.

In an example, a wearable food consumption monitoring device can comprise: eyeglasses worn by a person, wherein the eyeglasses further comprise a camera; and wherein the eyeglasses further comprise an electrochemical sensor, wherein a first camera is triggered to record images along an imaging vector which points toward the person's mouth and a second camera is triggered to record images along an imaging vector which points toward a reachable food source when analysis of data from the electrochemical sensor indicates that the person is consuming food. Alternatively, a wearable food consumption monitoring device can comprise: eyeglasses worn by a person, wherein the eyeglasses further comprise a camera; and wherein the eyeglasses further comprise an EMG sensor, wherein a first camera is triggered to record images along an imaging vector which points toward the person's mouth and a second camera is triggered to record images along an imaging vector which points toward a reachable food source when analysis of data from the EMG sensor indicates that the person is consuming food. In another example, a wearable food consumption monitoring device can comprise: eyeglasses worn by a person, wherein the eyeglasses further comprise a camera; and wherein the eyeglasses further comprise an infrared sensor which tracks the location of the person's hands, wherein the camera is triggered to record images along an imaging vector which points toward the person's mouth when analysis of data from the infrared sensor indicates that the person is consuming food.

In an example, a wearable food consumption monitoring system can comprise: eyeglasses worn by a person, wherein the eyeglasses further comprise at least two cameras; and wrist-worn motion sensor (e.g. in a smart watch), wherein a first camera is triggered to record images along an imaging vector which points toward the person's mouth and a second camera is triggered to record images of a reachable food source when analysis of data from the wrist-worn motion sensor indicates that the person is consuming food. In another example, a wearable food consumption monitoring device can comprise: eyeglasses worn by a person; at least one EMG sensor on a portion of the eyeglasses which curves around the rear of the person's ear; a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one EMG sensor indicates that the person is probably eating. In an example, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); a first camera on a frontpiece and/or nose bridge of the eyeglasses, wherein the first camera points toward the person's mouth; and a second camera on a frontpiece and/or nose bridge of the eyeglasses, wherein the second camera points toward the person's mouth, and wherein the first and second cameras are activated to record food images when analysis of data from the at least one EMG sensor indicates that the person is probably eating.

In another embodiment, a wearable food consumption monitoring device can comprise: eyeglasses worn by a person; at least one EMG sensor on the eyeglasses, wherein the EMG sensor is made from a generally non-conductive elastomeric polymer (e.g. PDMS) which has been doped, impregnated, or coated with conductive particles (e.g. silver, aluminum, or carbon nanotubes); and a camera on the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one EMG sensor on the eyeglasses; and a camera on a frontpiece and/or nose bridge of the eyeglasses, wherein the camera points toward the person's mouth, and wherein the camera is activated to record food images when analysis of data from the at least one EMG sensor indicates that the person is probably eating. In another example, a wearable food consumption monitoring device can comprise: eyeglasses worn by a person; at least one inertial motion sensor (e.g. gyroscope and/or accelerometer) on the eyeglasses; a first camera on a frontpiece and/or nose bridge of the eyeglasses, wherein the first camera points toward the person's mouth; and a second camera on a frontpiece and/or nose bridge of the eyeglasses, wherein the second camera points toward the person's hand and/or in front of the person, and wherein the first and second cameras are activated to record food images when analysis of data from the at least one inertial motion sensor indicates that the person is probably eating.

In an example, a wearable food consumption monitoring device can comprise: eyeglasses worn by a person; at least one vibration sensor on the eyeglasses; a first camera on a first sidepiece (e.g. a first temple) of the eyeglasses, wherein the first camera points toward the person's mouth; and a second camera on a second sidepiece (e.g. a second temple) of the eyeglasses, wherein the second camera points toward the person's hand and/or in front of the person, and wherein the first and second cameras are activated to record food images when analysis of data from the at least one vibration sensor indicates that the person is probably eating. In another example, a wearable food consumption monitoring device can comprise: eyeglasses worn by a person; at least one vibration sensor on the eyeglasses; and a camera on a sidepiece (e.g. a temple) of the eyeglasses, wherein the camera points toward the person's mouth, and wherein the camera is activated to record food images when analysis of data from the at least one vibration sensor indicates that the person is probably eating. In another embodiment, a wearable food consumption monitoring system can comprise: eyeglasses worn by a person; at least one wrist-worn or finger-worn inertial motion sensor (e.g. gyroscope and/or accelerometer on a smart watch or smart ring); a first camera on a right sidepiece (e.g. a right temple) of the eyeglasses, wherein the first camera points toward the person's mouth; and a second camera on a left sidepiece (e.g. a left temple) of the eyeglasses, wherein the second camera points toward the person's mouth, and wherein the first and second cameras are activated to record food images when analysis of data from the at least one wrist-worn or finger-worn inertial motion sensor indicates that the person is probably eating.

In an example, a wearable food consumption monitoring device can comprise: eyeglasses worn by a person, wherein the eyeglasses further comprise a camera; wherein the eyeglasses further comprise a motion sensor; and wherein the eyeglasses further comprise an infrared sensor which tracks the location of the person's hands, wherein the camera is triggered to record images when joint analysis of data from the motion sensor and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a chewing sensor and an infrared sensor, wherein a first camera is triggered to record images along an imaging vector which points toward the person's mouth and a second camera is triggered to record images of a reachable food source when joint analysis of data from the chewing sensor and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a sound sensor (e.g. microphone) and a chewing sensor, wherein a first camera is triggered to record images along an imaging vector which points toward the person's mouth and a second camera is triggered to record images of a reachable food source when joint analysis of data from the sound sensor (e.g. microphone) and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise a sound sensor (e.g. microphone), a chewing sensor, and an infrared sensor, wherein a first camera is triggered to record images along an imaging vector which points toward the person's mouth and a second camera is triggered to record images of a reachable food source when joint analysis of data from the sound sensor (e.g. microphone), the chewing sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise a sound sensor (e.g. microphone), an EEG sensor, and an infrared sensor, wherein a first camera is triggered to record images along an imaging vector which points toward the person's mouth and a second camera is triggered to record images of a reachable food source when joint analysis of data from the sound sensor (e.g. microphone), the EEG sensor, and the infrared sensor indicates that the person is consuming food. Alternatively, a wearable food consumption monitoring device can comprise: eyeglasses worn by a person; wherein the eyeglasses further comprise at least two cameras; and wherein the eyeglasses further comprise a sound sensor (e.g. microphone) and an accelerometer, wherein a first camera is triggered to record images along an imaging vector which points toward the person's mouth and a second camera is triggered to record images of a reachable food source when joint analysis of data from the sound sensor (e.g. microphone) and the accelerometer indicates that the person is consuming food.

In an example, a wearable food consumption monitoring device can comprise: eyeglasses worn by a person; wherein the eyeglasses further comprise at least two cameras; and wherein the eyeglasses further comprise a swallow sensor and an EEG sensor, wherein a first camera is triggered to record images along an imaging vector which points toward the person's mouth and a second camera is triggered to record images of a reachable food source when joint analysis of data from the swallow sensor and the EEG sensor indicates that the person is consuming food. In another example, a wearable food consumption monitoring device can comprise: eyeglasses worn by a person; wherein the eyeglasses further comprise at least two cameras; and wherein the eyeglasses further comprise a swallow sensor, a chewing sensor, and an infrared sensor, wherein a first camera is triggered to record images along an imaging vector which points toward the person's mouth and a second camera is triggered to record images of a reachable food source when joint analysis of data from the swallow sensor, the chewing sensor, and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallow sensor, an EMG sensor, and an infrared sensor, wherein a first camera is triggered to record images along an imaging vector which points toward the person's mouth and a second camera is triggered to record images of a reachable food source when joint analysis of data from the swallow sensor, the EMG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor and an EMG sensor, wherein a first camera is triggered to record images along an imaging vector which points toward the person's mouth and a second camera is triggered to record images of a reachable food source when joint analysis of data from the swallowing sensor and the EMG sensor indicates that the person is consuming food. In another embodiment, a wearable food consumption monitoring device can comprise: eyeglasses worn by a person; wherein the eyeglasses further comprise at least two cameras; and wherein the eyeglasses further comprise a swallowing sensor, an EEG sensor, and an infrared sensor, wherein a first camera is triggered to record images along an imaging vector which points toward the person's mouth and a second camera is triggered to record images of a reachable food source when joint analysis of data from the swallowing sensor, the EEG sensor, and the infrared sensor indicates that the person is consuming food.

In an example, a wearable food consumption monitoring device can comprise: eyeglasses worn by a person; wherein the eyeglasses further comprise at least two cameras; and wherein the eyeglasses further comprise a swallowing sensor, a chewing sensor, and an infrared sensor, wherein a first camera is triggered to record images along an imaging vector which points toward the person's mouth and a second camera is triggered to record images of a reachable food source when joint analysis of data from the swallowing sensor, the chewing sensor, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an accelerometer, wherein a first camera is triggered to record images along an imaging vector which points toward the person's mouth and a second camera is triggered to record images of a reachable food source when analysis of data from the accelerometer indicates that the person is consuming food.

In an example, a wearable food consumption monitoring device can comprise: eyeglasses worn by a person; wherein the eyeglasses further comprise at least two cameras; and wherein the eyeglasses further comprise an EEG sensor and an infrared sensor, wherein a first camera is triggered to record images along an imaging vector which points toward the person's mouth and a second camera is triggered to record images of a reachable food source when joint analysis of data from the EEG sensor and the infrared sensor indicates that the person is consuming food. In another embodiment, a wearable food consumption monitoring device can comprise: eyeglasses worn by a person; wherein the eyeglasses further comprise at least two cameras; and wherein the eyeglasses further comprise an EEG sensor, an accelerometer, and an infrared sensor, wherein a first camera is triggered to record images along an imaging vector which points toward the person's mouth and a second camera is triggered to record images of a reachable food source when joint analysis of data from the EEG sensor, the accelerometer, and the infrared sensor indicates that the person is consuming food. In another example, a wearable food consumption monitoring device can comprise: eyeglasses worn by a person; wherein the eyeglasses further comprise at least two cameras; and wherein the eyeglasses further comprise an EMG sensor and a motion sensor, wherein a first camera is triggered to record images along an imaging vector which points toward the person's mouth and a second camera is triggered to record images of a reachable food source when joint analysis of data from the EMG sensor and the motion sensor indicates that the person is consuming food.

In an example, a wearable food consumption monitoring device can comprise: eyeglasses worn by a person; wherein the eyeglasses further comprise at least two cameras; and wherein the eyeglasses further comprise an EMG sensor, a motion sensor, and an infrared sensor, wherein a first camera is triggered to record images along an imaging vector which points toward the person's mouth and a second camera is triggered to record images of a reachable food source when joint analysis of data from the EMG sensor, the motion sensor, and the infrared sensor indicates that the person is consuming food. In an example, a wearable food consumption monitoring device can comprise: eyeglasses worn by a person; wherein the eyeglasses further comprise at least two cameras; and wherein the eyeglasses further comprise an motion sensor and a chewing sensor, wherein a first camera is triggered to record images along an imaging vector which points toward the person's mouth and a second camera is triggered to record images of a reachable food source when joint analysis of data from the motion sensor and the chewing sensor indicates that the person is consuming food.

In an example, a wearable food consumption monitoring device can comprise: eyeglasses worn by a person; wherein the eyeglasses further comprise at least two cameras; and wherein the eyeglasses further comprise an motion sensor, wherein a first camera is triggered to record images along an imaging vector which points toward the person's mouth and a second camera is triggered to record images of a reachable food source when analysis of data from the motion sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and a chewing sensor, wherein at least one camera is triggered to record images along an imaging vector which points toward the person's mouth when joint analysis of data from the sound sensor (e.g. microphone) and the chewing sensor indicates that the person is consuming food.

In an example, a wearable food consumption monitoring device can comprise: eyeglasses worn by a person; wherein the eyeglasses further comprise one or more cameras; and wherein the eyeglasses further comprise a sound sensor (e.g. microphone), a chewing sensor, and an infrared sensor, wherein at least one camera is triggered to record images along an imaging vector which points toward the person's mouth when joint analysis of data from the sound sensor (e.g. microphone), the chewing sensor, and the infrared sensor indicates that the person is consuming food. In another embodiment, a wearable food consumption monitoring device can comprise: eyeglasses worn by a person; wherein the eyeglasses further comprise one or more cameras; and wherein the eyeglasses further comprise a sound sensor (e.g. microphone), a chewing sensor, and an infrared sensor, wherein at least one camera is triggered to record images along an imaging vector which points toward the person's mouth when joint analysis of data from the sound sensor (e.g. microphone), the chewing sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a sound sensor (e.g. microphone) and a motion sensor, wherein at least one camera is triggered to record images along an imaging vector which points toward the person's mouth when joint analysis of data from the sound sensor (e.g. microphone) and the motion sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor and an EEG sensor, wherein at least one camera is triggered to record images along an imaging vector which points toward the person's mouth when joint analysis of data from the swallow sensor and the EEG sensor indicates that the person is consuming food. In another embodiment, a wearable food consumption monitoring device can comprise: eyeglasses worn by a person; wherein the eyeglasses further comprise one or more cameras; and wherein the eyeglasses further comprise a swallow sensor, a chewing sensor, and an infrared sensor, wherein at least one camera is triggered to record images along an imaging vector which points toward the person's mouth when joint analysis of data from the swallow sensor, the chewing sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallow sensor, an EMG sensor, and an infrared sensor, wherein at least one camera is triggered to record images along an imaging vector which points toward the person's mouth when joint analysis of data from the swallow sensor, the EMG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor and an EMG sensor, wherein at least one camera is triggered to record images along an imaging vector which points toward the person's mouth when joint analysis of data from the swallowing sensor and the EMG sensor indicates that the person is consuming food. In another example, a wearable food consumption monitoring device can comprise: eyeglasses worn by a person; wherein the eyeglasses further comprise one or more cameras; and wherein the eyeglasses further comprise a swallowing sensor, an EMG sensor, and an infrared sensor, wherein at least one camera is triggered to record images along an imaging vector which points toward the person's mouth when joint analysis of data from the swallowing sensor, the EMG sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise a swallowing sensor, a motion sensor, and an infrared sensor, wherein at least one camera is triggered to record images along an imaging vector which points toward the person's mouth when joint analysis of data from the swallowing sensor, the motion sensor, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an accelerometer, wherein at least one camera is triggered to record images along an imaging vector which points toward the person's mouth when analysis of data from the accelerometer indicates that the person is consuming food.

In an example, a wearable food consumption monitoring device can comprise: eyeglasses worn by a person; wherein the eyeglasses further comprise one or more cameras; and wherein the eyeglasses further comprise an EEG sensor and an infrared sensor, wherein at least one camera is triggered to record images along an imaging vector which points toward the person's mouth when joint analysis of data from the EEG sensor and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an EEG sensor, an accelerometer, and an infrared sensor, wherein at least one camera is triggered to record images along an imaging vector which points toward the person's mouth when joint analysis of data from the EEG sensor, the accelerometer, and the infrared sensor indicates that the person is consuming food. In an example, a wearable food consumption monitoring device can comprise: eyeglasses worn by a person; wherein the eyeglasses further comprise one or more cameras; and wherein the eyeglasses further comprise an EMG sensor and a motion sensor, wherein at least one camera is triggered to record images along an imaging vector which points toward the person's mouth when joint analysis of data from the EMG sensor and the motion sensor indicates that the person is consuming food. In another example, a wearable food consumption monitoring device can comprise: eyeglasses worn by a person; wherein the eyeglasses further comprise one or more cameras; and wherein the eyeglasses further comprise an EMG sensor, a chewing sensor, and an infrared sensor, wherein at least one camera is triggered to record images along an imaging vector which points toward the person's mouth when joint analysis of data from the EMG sensor, the chewing sensor, and the infrared sensor indicates that the person is consuming food.

In an example, a wearable food consumption monitoring device can comprise: eyeglasses worn by a person; wherein the eyeglasses further comprise one or more cameras; and wherein the eyeglasses further comprise an EMG sensor, an EEG sensor, and an infrared sensor, wherein at least one camera is triggered to record images along an imaging vector which points toward the person's mouth when joint analysis of data from the EMG sensor, the EEG sensor, and the infrared sensor indicates that the person is consuming food. In another example, a wearable food consumption monitoring device can comprise: eyeglasses worn by a person; wherein the eyeglasses further comprise one or more cameras; and wherein the eyeglasses further comprise an motion sensor and an infrared sensor, wherein at least one camera is triggered to record images along an imaging vector which points toward the person's mouth when joint analysis of data from the motion sensor and the infrared sensor indicates that the person is consuming food.

==============C===============

In an example, a wrist-worn device for tracking food intake can comprise: a camera which is held on a person's wrist by a wrist-worn band (e.g. the band of a smart watch), wherein the camera records food images, wherein the food images are analyzed to identify food types and quantities, wherein there is also a primary display (e.g. a watch face) which is held on the person's wrist by the wrist-worn band, wherein the primary display is centered on a first location on the circumference of the band, wherein the camera is centered on a second location on the circumference of the band; and an eating detector which collects data which is analyzed to detect when the person is eating, wherein the eating detector further comprises one or more components selected from the group consisting of: an accelerometer, a gyroscope, a magnetometer, a microphone, a vibration sensor, and an EMG sensor.

In an example, a wrist-worn device for tracking food intake can comprise: a camera which is held on a person's wrist by a wrist-worn band (e.g. the band of a smart watch), wherein the camera records food images, wherein the food images are analyzed to identify food types and quantities, wherein there is also a primary display (e.g. a watch face) which is held on the person's wrist by the wrist-worn band, wherein the primary display is centered on a first location on the circumference of the band, wherein the camera is centered on a second location on the circumference of the band; a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, a vibration sensor, and an EMG sensor.

In an example, a wrist-worn device for tracking food intake can comprise: a camera which is held on a person's wrist by a wrist-worn band (e.g. the band of a smart watch), wherein the camera records food images, wherein the food images are analyzed to identify food types and quantities, wherein there is also a primary display (e.g. a watch face) which is held on the person's wrist by the wrist-worn band, wherein the primary display is centered on a first location on the circumference of the band, wherein the camera is centered on a second location on the circumference of the band; a camera viewfinder which held on the person's wrist by the wrist-worn band, wherein the camera viewfinder displays the food images recorded by the camera, wherein the camera viewfinder is centered on a third location on the circumference of the band; a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, a vibration sensor, and an EMG sensor.

In an example, a wrist-worn device for tracking food intake can comprise: a camera which is held on a person's wrist by a wrist-worn band (e.g. the band of a smart watch), wherein the camera records food images, wherein the food images are analyzed to identify food types and quantities, wherein there is also a primary display (e.g. a watch face) which is held on the person's wrist by the wrist-worn band, wherein the primary display is centered on a first location on the circumference of the band, wherein the camera is centered on a second location on the circumference of the band, and wherein the second location is at least 45 degrees around the band circumference away from the first location in a first (e.g. clockwise) direction; a camera viewfinder which held on the person's wrist by the wrist-worn band, wherein the camera viewfinder displays the food images recorded by the camera, wherein the camera viewfinder is centered on a third location on the circumference of the band, and wherein the third location is at least 45 degrees around the band circumference away from the first location in a second (e.g. counter-clockwise) direction; a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, a vibration sensor, and an EMG sensor.

In an example, a wrist-worn device for tracking food intake can comprise: a camera which is held on a person's wrist by a wrist-worn band (e.g. the band of a smart watch), wherein the camera records food images, wherein the food images are analyzed to identify food types and quantities, wherein there is also a primary display (e.g. a watch face) which is held on the person's wrist by the wrist-worn band, wherein the primary display is centered on a first location on the circumference of the band, wherein the camera is centered on a second location on the circumference of the band, and wherein the second location is between 60 to 110 degrees around the band circumference away from the first location in a first (e.g. clockwise) direction; a camera viewfinder which held on the person's wrist by the wrist-worn band, wherein the camera viewfinder displays the food images recorded by the camera, wherein the camera viewfinder is centered on a third location on the circumference of the band, and wherein the third location is between 70 and 110 degrees around the band circumference away from the first location in a second (e.g. counter-clockwise) direction which is opposite the first direction; a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, a vibration sensor, and an EMG sensor.

The term “food” as used herein is broadly defined to include liquid nourishment, such as beverages, in addition to solid food. The phrase “reachable food source” is defined as a source of food that a person can access and from which they can bring a piece (or portion) to their mouth by moving their arm and hand. Arm and hand movement can include movement of the person's shoulder, elbow, wrist, and finger joints. In an example, a reachable food source can be selected from the group consisting of: food on a plate, food in a bowl, food in a glass, food in a cup, food in a bottle, food in a can, food in a package, food in a container, food in a wrapper, food in a bag, food in a box, food on a table, food on a counter, food on a shelf, and food in a refrigerator.

In an example, a camera for recording food images can be located on the narrow side of a person's wrist, between the dorsal and ventral sides of the person's wrist. In an example, a camera for recording food images can be located on the narrow side of a person's wrist which faces away from the person's body. In an example, the location of a camera for recording food images can be approximately 90 degrees (around the circumference of the wrist) away from the location of a watch face on a smart watch. In an example, a camera can be removably-attached to a watch band, at a location which is approximately 90 degrees (around the circumference of the wrist) away from the location of a watch face on a smart watch. In an example, a camera can be integrated into a smart watch band at a location which is approximately 90 degrees (around the circumference of the wrist) away from the location of a watch face on a smart watch. In an example, a camera can be removably-attached to a watch band, at a location which is between 70 and 110 degrees (around the circumference of the wrist) away from the location of a watch face on a smart watch. In an example, a camera can be integrated into a smart watch band at a location which is between 70 and 110 degrees (around the circumference of the wrist) away from the location of a watch face on a smart watch.

In another example, a camera for recording food images can be located on the ventral side of a person's wrist. In another example, the location of a camera for recording food images can be approximately 180 degrees (around the circumference of the wrist) away from the location of a watch face on a smart watch. In another example, a camera can be removably-attached to a watch band, at a location which is approximately 180 degrees (around the circumference of the wrist) away from the location of a watch face on a smart watch. In another example, a camera can be integrated into a smart watch band at a location which is approximately 180 degrees (around the circumference of the wrist) away from the location of a watch face on a smart watch. In another example, the location of a camera for recording food images can be on the opposite side of a person's wrist from the location of a watch face on a smart watch. In an example, a camera can be incorporated into the clasp or buckle of a smart watch band which is generally on the opposite side of a wrist from a primary watch housing (e.g. watch face)

In an example, an attachment mechanism which attaches a camera for recording food images to a smart watch band can enable a person to manually adjust (e.g. slide) the location of the camera along the circumference of the band. For example, the attachment can loop around the band with a mechanism which can be tightened to fix the camera at a given location or loosened to move the camera to a different location. The same can be true for a spectroscopic sensor. In an example, both a camera and a spectroscopic sensor can be in the same housing, wherein this housing can be manually slid along the circumference of the band and thereby attached to different locations along the circumference of the band.

In another example, a camera for recording food images can be on a flip-up component which flips, tilts, pivots, rotates, and/or pops up from the housing of a smart watch. Having a camera on a flip-up component can reduce the extent to which the camera's field-of-vision is obscured by the person's wrist.

In an example, a person can manually activate a camera to record food images before, during, and/or after eating. In an example, a device can automatically prompt a person to activate a camera to record food images when an eating detector detects that the person has started to eat, is eating, or has stopped eating. In an example, a device can automatically activate a camera to record food images when data from an eating detector indicates that a person has started to eat, is eating, or has stopped eating.

In an example, a person can manually direct the focal vector of a camera on a wrist-worn device toward food by moving their wrist. In an example, a camera on a wrist-worn device can have an automated mechanism which: scans nearby space for food items; recognizes food items based on image analysis; and adjusts the focal vector of the camera to maintain focal direction toward the food items once they are identified. In an example, such an automated mechanism can maintain a focal direction toward food items as a person waives their hand over food, thereby automatically capturing images of the food items from different angles. This can be useful for creating three-dimensional images of food for estimating the volume of food items.

In an example, the focal direction of a camera can be outward and generally perpendicular to the circumference of a device. In an example, the focal direction of a camera from a location can be changed by a person via a mechanism selected from the group consisting of: a touch screen (e.g. touching and/or swiping a touch-screen on the display); a motion sensor (e.g. a motion sensor which recognizes the orientation of the device and/or changes in this orientation); a gesture recognition sensor (e.g. an optical, electromagnetic radiation, or motion sensor which recognizes hand gestures); a rotating bezel or ring (e.g. a bezel which is rotated around a circular display); a rotating knob (e.g. a rotating knob which is perpendicular to the display surface); and voice command recognition (e.g. recognition of vocal commands recorded by a microphone). In an example, the focal direction of a camera can be automatically adjusted to maintain focus on a food item. In an example, images from a camera can be analyzed by pattern recognition to recognize a food item in the camera's field of view and to maintain a stabilized focal direction toward that food item. In an example, a stabilized direct line of sight to a food item in the camera's field of view can be maintained by adjusting the focal direction of the camera to compensate for motion of a wrist-worn device.

In an example, a wrist-worn device can automatically adjust the focal vector of a camera in real time based on data from a motion sensor (e.g. an accelerometer and gyroscope) in order to stabilize a food image. In an example, the focal direction of a camera can be automatically adjusted to maintain focal direction toward a food item. In an example, images from a camera can be analyzed by pattern recognition to recognize food items in a camera's field of view and to maintain a stabilized focal direction toward those items. In an example, a stabilized direct line of sight to a food item can be maintained by adjusting the focal direction of the camera to compensate for motion of a wrist-worn device as detected by an inertial motion sensor.

In an example, a camera on a wearable device (e.g. a smart watch or augmented reality eyewear) can start recording images only when data from an eating detector indicates that a person is eating. This can reduce privacy concerns as compared to a camera that records images all the time. In an example, a camera can automatically begin recording images when data from sensors on a wearable device indicate that a person is probably eating. In an example, food images can be recorded from at least two different perspectives in order to create virtual three-dimensional models of food.

Pattern recognition software can identify types of food at a reachable food source by: analyzing the shapes, colors, textures, and volumes of food items in an image; or by analyzing food packaging in an image. In an example, one or more methods to analyze food images in order to estimate the types and quantities of food present and/or consumed can be selected from the group consisting of: pattern recognition; food recognition; word recognition; logo recognition; bar code recognition; gesture recognition; and human motion recognition. In an example, food images can be analyzed with one or more methods selected from the group consisting of: pattern recognition or identification; human motion recognition or identification; gesture recognition or identification; food recognition or identification; word recognition or identification; logo recognition or identification; bar code recognition or identification; and 3D modeling.

In an example, food consumed by a person can be tracked by analyzing one or more factors selected from the group consisting of: number and type of reachable food sources; changes (e.g. before vs. after a meal) in the volume of food observed at a reachable food source; number and size of chewing movements; number and size of swallowing movements; number of times that pieces (or portions) of food travel along the food consumption pathway; and size of pieces (or portions) of food traveling along a food consumption pathway. In an example, one or more of these factors can be used to analyze images to estimate the types and quantities of food consumed by a person.

In an example, the types and quantities of food consumed by a person can be estimated based on: pattern recognition of food at a reachable food source; changes in food at that source; analysis of images of food traveling along a food consumption pathway from a food source to the person's mouth; and/or the number of cycles of food moving along the food consumption pathway. In an example, food can be identified by pattern recognition of food itself, by recognition of words on food packaging or containers, by recognition of food brand images and logos, or by recognition of product identification codes (such as “bar codes”). In an example, analysis of food images can occur in real time, as a person is eating. In an example, analysis of images can happen after a person has consumed food.

In an example, a spectroscopic sensor can scan food items to obtain information about food composition. In an example, a spectroscopic sensor can further comprise a light emitter which emits light rays and a light receiver which receives light rays. In an example, a spectroscopic sensor can further comprise a light emitter which emits light rays toward food and a light receiver which receives light rays reflected from the food. In an example, spectroscopic analysis of light rays reflected by the food can provide information concerning food composition which is not available from analysis of food images.

In an example, a person can manually activate a spectroscopic sensor to scan food before, during, and/or after eating. In an example, a device can automatically prompt a person to activate a spectroscopic sensor to scam food when an eating detector detects that the person has started to eat, is eating, or has stopped eating. In an example, a device can automatically activate a spectroscopic sensor to scan food when an eating detector detects that a person has started to eat, is eating, or has stopped eating.

In an example, a spectroscopic sensor can comprise one or more light emitters. In an example, a light emitter can be a LED (Light Emitting Diode). In an example, a light emitter can be a laser diode. In an example, a light emitter can emit infrared or near-infrared light. In an example, a light emitter can emit visible light. In an example, a spectroscopic sensor can further comprise a circular array of light emitters. In an example, a spectroscopic sensor can further comprise a polygonal (e.g. square) array of light emitters. In an example, a spectroscopic sensor can further comprise an array of light emitters around a central light receiver. In an example, a spectroscopic sensor can further comprise a circular array of light receivers. In an example, a spectroscopic sensor can further comprise a polygonal (e.g. square) array of light receivers. In an example, a spectroscopic sensor can further comprise an array of light receivers around a central light emitter. In an example, a spectroscopic sensor can comprise a spectrometer.

In an example, a spectroscopic sense can further comprise a plurality of light emitters which each emit light at a different wavelength. In an example, a spectroscopic sense can further comprise one or more light emitters which emit light at different wavelengths at different times. In an example, a spectroscopic sense can further comprise one or more light emitters which emit light at time-varying wavelengths. In an example, a spectroscopic sense can further comprise a plurality of light emitters which each emit light with a different intensity level. In an example, a spectroscopic sense can further comprise one or more light emitters which emit light at different intensity levels at different times. In an example, a spectroscopic sense can further comprise one or more light emitters which emit light with time-varying intensity levels. In an example, a spectroscopic sense can further comprise a plurality of light emitters which each emit light at a different angle relative to a wrist-band. In an example, a spectroscopic sense can further comprise one or more light emitters which emit light along different vectors at different times. In an example, a spectroscopic sense can further comprise one or more light emitters which emit light along time-varying vectors.

In an example, a spectroscopic sensor can scan food and analyze reflected light, first with the light emitters off and then with the light emitters on, in order to control for baseline reflected ambient light. In an example, spectral values of reflected light received when the light emitters are off can be subtracted from spectral values of reflected light received when the light emitters are on. In an alternative example, a spectroscopic sensor can comprise light receivers only and only measure reflected ambient light. In the case of a spectroscopic sensor which uses ambient light only, differences in reflected light spectra from food items (experimental data) compared to non-food items (control data) can be used to isolate and analyze the composition of food items.

In an example, a spectroscopic sensor can further comprise a transparent cover which protects light emitters and light receivers from direct physical contact with food or other optical contaminants. In an example, a spectroscopic sense can further comprise an opaque light shield which at least partially shields the optical path of light from the sensor to food (and vice versa). In an example, a light shield can protect this optical path from contamination by ambient light. In an example, a light shield can reduce the amount of light traveling directly from a light emitter to a light receiver without first being reflected from food. In an example, this light shield can be convex, with a light emitter, a light receiver, or both inside the convexity. In an example, a spectroscopic sensor can have one or more light shields around one or more light emitters only. In an example, a spectroscopic sensor can have one or more light shields around one or more light emitters only. In an example, a spectroscopic sensor can have a light shield between each pair-wise path between a light emitter and light receiver. In an example, a light shield can be compressible, soft, elastomeric, and/or low-durometer.

In an example, a spectroscopic sensor can further comprise a light concentrator. In an example, a spectroscopic sensor can further comprise an optical diffuser. In an example, a spectroscopic sensor can further comprise one or more optical filters. In an example, a spectroscopic sensor can further comprise one or more lenses. In an example, a spectroscopic sensor can further comprise a curved reflector. In an example, a spectroscopic sensor can further comprise a beam splitter.

In an example, a wrist-worn device with a camera and spectroscopic sensor can project a visible beam of (coherent) light which enables a person to see where the camera and/or the spectroscopic sensor are directed. In an example, this beam can from a light emitter in a spectroscopic sensor. In an example, this projected beam can be from a light emitter which is separate from the spectroscopic sensor. In an example, this projected visible beam of light can be used to direct the camera and/or the spectroscopic sensor sequentially toward individual food items in a meal. In an example, food images recorded by a camera can be matched with spectroscopic scans recorded by the spectroscopic sensor in order to better analyze the composition of individual food items in a meal.

In an example, this visible beam of light can project a single point of light. In an example, a single point of light can identify the center of a food image or spectroscopic scan. In an example, this visible beam of light can project a polygonal or circular array of light points. In an example, a polygonal or circular array of light points can identify the perimeter of a food image or spectroscopic scan. In an example, this visible beam of light can project a polygonal or circular shaped light projection. In an example, a polygonal or circular light projection can identify the perimeter of a food image or spectroscopic scan. In an example, a projected pattern of light can be a grid. In an example, the size and shape of a light pattern projected onto food or near food can be analyze the viewing distance and angle from the wrist-worn device to food. In an example, distortion of a projected light circle, polygon, or grid can be analyzed to evaluate the viewing angle between the wrist-worn device and the food.

In an example, a visible beam of (coherent) light emitted from a wrist-worn device can project text-based or graphic-based information onto or near food. In an example, a visible beam of light can project information about food quantity, nutritional composition, or health effects onto a surface near food or onto food itself. In an example, a wrist-worn device can further comprise a microprojector. In an example, the device can project a preliminarily determination of food type (based on its analysis of camera and spectroscopic data) and then project this information near the food for the person to confirm, modify, or reject the preliminary determination.

In an example, a wrist-worn device for tracking food intake can include a visible light emitter which illuminates food when there is insufficient ambient light otherwise to record good food images. In an example, such a light emitter can be co-located in a housing with a camera and/or a spectroscopic sensor. In another example, a wrist-worn device can have a light emitter which emits light in a selected spectral range (e.g. infrared light or near-infrared light) to capture images of food under different spectral conditions. In another example, a device can have a filter which captures images of light from food in a selected spectral range (e.g. infrared imaging).

In an example, an eating detector can be a motion sensor. In an example, a motion-based eating detector can comprise one or more sensors selected from the group consisting of: accelerometer, gyroscope, magnetometer, inclinometer, and GPS component. In an example, a motion-based eating detector can detect when a person eats by identification of a pattern of arm (and hand or finger) motions and/or gestures selected from the group consisting of: cutting food (e.g. cutting food with a fork and knife), scooping or piercing food, grasping a beverage container (e.g. a glass, can, or cup), scooping food with chop sticks, bringing food up to their mouth, tilting a beverage container, inserting food into their mouth (e.g. inserting a fork or spoon), drinking from a raising beverage container, and lowering their arm after inserting food into their mouth.

In an example, a motion-based eating detector can be worn on a person's non-dominant arm. In an example, a motion sensor in a conventional smart watch worn on a person's non-dominant arm can serve as a motion-based eating detector. In an example, a motion-based eating detector can be removably attached to the band of a conventional smart watch. In an example, a motion-based eating detector can be integrated into a smart band which can be worn with a conventional smart watch. Alternatively, a motion-based eating detector can be worn on a person's dominant arm and in wireless communication with a smart watch worn on the person's dominant arm. In another example, a wearable system for tracking food intake can include wrist-worn devices on both arms, one of which includes a display, camera, and spectroscopic sensor and the other includes a motion sensor to detect eating.

In an example, an eating detector can comprise one or more EMG (electromyographic) sensors which are worn on a person's arm, wrist, and/or hand. In an example, an EMG-based eating detector can detect when a person eats by identification of a pattern of arm (and hand or finger) muscle motions and/or gestures selected from the group consisting of: cutting food (e.g. cutting food with a fork and knife), scooping or piercing food, grasping a beverage container (e.g. a glass, can, or cup), scooping food with chop sticks, bringing food up to their mouth, tilting a beverage container, inserting food into their mouth (e.g. inserting a fork or spoon), drinking from a raising beverage container and lowering their arm after inserting food into their mouth.

In an example, a EMG-based eating detector can be worn on a person's non-dominant arm. In an example, a EMG sensor in a conventional smart watch worn on a person's non-dominant arm can serve as a EMG-based eating detector. In an example, a EMG-based eating detector can be removably attached to the band of a conventional smart watch. In an example, a EMG-based eating detector can be integrated into a smart band which can be worn with a conventional smart watch. Alternatively, a EMG-based eating detector can be worn on a person's dominant arm and in wireless communication with a smart watch worn on the person's dominant arm. In another example, a wearable system for tracking food intake can include wrist-worn devices on both arms, one of which includes a display, camera, and spectroscopic sensor and the other includes an EMG sensor to detect eating.

In an example, an eating detector can comprise one or more EMG (electromyographic) sensors which are worn on a person's head or neck. In an example, an EMG-based eating detector can detect when a person eats by identifying electromagnetic neuromuscular activity associated chewing and/or swallowing. In an example, an EMG sensor for eating detection can be attached to a person's ear. In an example, an EMG sensor for eating detection can be attached to a person's temple area. In an example, an EMG sensor for eating detection can be attached to a person's jaw. In an example, an EMG sensor for eating detection can be attached to a person's neck. In an example, an EMG electrode for eating detection can be adhered to a person's ear. In an example, an EMG electrode for eating detection can be adhered to a person's temple area. In an example, an EMG electrode for eating detection can be adhered to a person's jaw. In an example, an EMG electrode for eating detection can be adhered to a person's neck. In an example, an EMG-based eating detector can be removably attached to eyewear. In an example, an EMG-based eating detector can be integrated into smart eyewear.

In an example, an eating detector can comprise one or more sound and/or vibration sensors which are worn on a person's head or neck. In an example, a sound-or-vibration-based eating detector can detect when a person eats by identifying sounds and/or vibrations associated chewing and/or swallowing. In an example, a sound and/or vibration sensor for eating detection can be attached to a person's ear. In an example, a sound and/or vibration sensor for eating detection can be attached to a person's temple area. In an example, a sound and/or vibration sensor for eating detection can be attached to a person's jaw. In an example, a sound and/or vibration sensor for eating detection can be attached to a person's neck.

In an example, a sound electrode for eating detection can be adhered to a person's ear. In an example, a sound electrode for eating detection can be adhered to a person's temple area. In an example, a sound electrode for eating detection can be adhered to a person's jaw. In an example, a sound electrode for eating detection can be adhered to a person's neck. In an example, a sound-or-vibration-based eating detector can be removably attached to eyewear. In an example, a sound-or-vibration-based eating detector can be integrated into smart eyewear. In an example, a sound-or-vibration-based eating detector can be integrated into a smart necklace.

In an example, an eating detector can comprise a plurality of sensors selected from the group consisting of: electromagnetic impedance or capacitance sensor, EMG (electromyographic) or other neuromuscular sensor, glucose sensor, heart rate sensor, inclinometer, inertial motion sensor (e.g. accelerometer and/or gyroscope), magnetometer, microphone, oxygenation sensor, pressure sensor, sweat sensor, temperature sensor, and vibration sensor. In an example, eating can be detected by multivariate analysis of data from a plurality of sensors selected from the group consisting of: electromagnetic impedance or capacitance sensor, EMG (electromyographic) or other neuromuscular sensor, glucose sensor, heart rate sensor, inclinometer, inertial motion sensor (e.g. accelerometer and/or gyroscope), magnetometer, microphone, oxygenation sensor, pressure sensor, sweat sensor, temperature sensor, and vibration sensor. In an example, eating can be detected by multivariate analysis of data from a plurality of sensors housed in different components of a wearable system, wherein the components are selected from the group consisting of: smart watch or other wrist-worn device, smart eyeglasses, ear bud or other ear-worn device, adhesive patch with embedded sensors, and dental implant with embedded sensors.

In an example, one or more sensors that detect eating can be selected from the group consisting of: accelerometer, inclinometer, motion sensor, sound sensor, smell sensor, blood pressure sensor, heart rate sensor, EEG sensor, ECG sensor, EMG sensor, electrochemical sensor, gastric activity sensor, GPS sensor, location sensor, image sensor, optical sensor, piezoelectric sensor, respiration sensor, strain gauge, electrogoniometer, chewing sensor, swallow sensor, temperature sensor, and pressure sensor. In an example, indications that a person is eating can be selected from the group consisting of: acceleration, inclination, twisting, or rolling of the person's hand, wrist, or arm; acceleration or inclination of the person's lower arm or upper arm; bending of the person's shoulder, elbow, wrist, or finger joints; movement of the person's jaw, such as bending of the jaw joint; smells suggesting food that are detected by an artificial olfactory sensor (e.g. possibly branded as “Eater Odors”); detection of chewing, swallowing, or other eating sounds by one or more microphones; electromagnetic waves from the person's stomach, heart, brain, or other organs; GPS or other location-based indications that a person is in an eating establishment (such as a restaurant) or food source location (such as a kitchen).

In an example, a wrist-worn device for tracking food intake can prompt a person to activate a camera and/or a spectroscopic sensor when eating is detected. In an example, a wrist-worn device for tracking food intake can prompt a person to activate a camera and/or a spectroscopic sensor via a vibrating component on a watch band or housing. In an example, a wrist-worn device for tracking food intake can prompt a person to activate a camera and/or a spectroscopic sensor via a sound tone or pattern. In an example, a wrist-worn device for tracking food intake can prompt a person to activate a camera and/or a spectroscopic sensor via an indicator light and/or light pattern. In an example, a wrist-worn device for tracking food intake can prompt a person to activate a camera and/or a spectroscopic sensor via a message displayed on a screen.

In an example, a wrist-worn device for tracking food intake can comprise a secondary display (apart from a primary display which serves as a watch face). In an example, a secondary display can be a touch screen. In an example, the secondary display can serve as a viewfinder for a camera which records food images. This secondary display is useful for viewing images when a person has to rotate and/or tilt their wrist in order to orient a wrist-worn camera toward nearby food to record food images. When a person rotates and/or tilts their wrist, a primary display can be tilted away from the person's line of sight so that images on the primary display are not clearly visible. However, images on a secondary display at a different circumferential location can be clearly visible when the person rotates and/or tilts their wrist. In an example, a device can automatically determine whether it is better to display camera images on a primary display, a secondary display, or both—based on data from motion sensors which indicates the orientation of the person's wrist relative to the person's line of sight.

In an example, a secondary display (which serves as a camera viewfinder) can be located at least 45 degrees around the wrist circumference away from a primary display. In an example, a secondary display (which services as a camera viewfinder) can be located at least 45 degrees around the wrist circumference in a counter-clockwise direction away from a primary display. In an example, a secondary display (which serves as a camera viewfinder) can be at a location which is between 70 and 110 degrees around the wrist circumference away from a primary display. In an example, a secondary display (which serves as a camera viewfinder) can be located 180 degrees around the wrist circumference away from a camera for which it acts as a viewfinder. In an example, a secondary display (which serves as a camera viewfinder) can be at a location which is between 160 and 200 degrees around the wrist circumference away from a camera for which it acts as a viewfinder. In an example, a secondary display (which serves as a camera viewfinder) can be located on the opposite side of a person's wrist from a camera for which it acts as a viewfinder.

In an example, a display on a wrist-worn device for tracking food intake can display food images recorded by a camera on the device. In an example, a display on a wrist-worn device for tracking food intake can display food images recorded by a camera on the device in real time. In an example, a display on a wrist-worn device for tracking food intake can serve as a viewfinder for a camera on the device. In an example, a display on a wrist-worn device for tracking food intake can display information about nearby food based on analysis of food images and/or spectroscopic scans of the food. In an example, a display on a wrist-worn device for tracking food intake can display nutritional information about nearby food based on analysis of food images and/or spectroscopic scans of the food. In an example, a display on a wrist-worn device for tracking food intake can display eating recommendations about nearby food based on analysis of food images and/or spectroscopic scans of the food. In an example, a display on a wrist-worn device for tracking food intake can display recommendations concerning which nearby foods to eat or not based on analysis of food images and/or spectroscopic scans of the food. In an example, a display on a wrist-worn device for tracking food intake can display recommendations concerning the quantities of nearby foods to eat based on analysis of food images and/or spectroscopic scans of the food.

In an example, a wrist-worn device for tracking food intake can have a flip-up component which includes a display. In an example, a wrist-worn device for tracking food intake can have a flip-up display. A flip-up display can serve as a viewfinder for a wrist-worn camera, especially if the person needs to rotate their wrist to direct the camera toward nearby food. A flip-up display can be useful to enable a person to see a display image even when the person rotates their wrist to orient a camera toward nearby food. The surface of a flip-up display can be more orthogonal to a person's line of sight than a surface which is tangential to a wrist band when the person rotates their wrist to direct a camera toward nearby food.

In an example, a flip-up display can have a first configuration in which it is generally flat along the wrist band and a second configuration in which it flips, pivots, rotates, tilts, or pops up and out from the wrist band. In an example, a flip-up display can have a first configuration in which it is generally recessed into a housing on the wrist band and a second configuration in which it flips, pivots, rotates, tilts, or pops up from the housing. In an example, a flip-up display can have a first configuration wherein the virtual plane which best fits the display is generally tangential to the arcuate perimeter of the wrist band and a second configuration in which this virtual plan intersects the tangent line of the wrist band perimeter at an angle between 30 and 90 degrees.

In an example, a wrist-worn device for tracking food intake can have a flip-up component. In an example, a flip-up component can have a first configuration in which it is generally flat along a wrist band and a second configuration in which it flips, pivots, rotates, tilts, or pops up from the wrist band. In an example, a flip-up component can have a first configuration in which it is generally recessed into a housing on the wrist band and a second configuration in which it flips, pivots, rotates, tilts, or pops up and out from the housing. In an example, a flip-up component can have a first configuration wherein the virtual plane which best fits the component is generally tangential to the arcuate perimeter of the wrist band and a second configuration in which this virtual plan intersects the tangent line of the wrist band perimeter at an angle between 30 and 90 degrees. In an example, there can be a display on one side of the flip-up component and a camera on the opposite side of the flip-up component. In an example, the display can serve as a viewfinder for the camera on the opposite side of the flip-up component.

In an example, a wrist-worn device for tracking food intake can have a flip-up component with a first configuration in which it is recessed (into a housing) and a second configuration in which it extends out (from the housing). In an example, a wrist-worn device can have a flip-up component with a first configuration in which it is recessed (along a band or sleeve) and a second configuration in which it extends out (from the band or sleeve). In an example, a flip-up component can be substantially flush with the surface of a housing in its first configuration and extend outward from the housing in its second configuration. In an example, a flip-up component can be substantially parallel with a housing in its first configuration and substantially perpendicular to the housing in its second configuration. In an example, a flip-up component can be substantially parallel with a housing in its first configuration and intersect the housing at an adjustable angle between 10 and 90 degrees in its second configuration. In an example, a flip-up component can be substantially parallel with a housing in its first configuration and intersect the housing at an adjustable and lockable angle between 10 and 90 degrees in its second configuration.

In an example, a flip-up component can have a locking mechanism which temporarily locks the component in a flipped-up or popped-up configuration. In an example, a flip-up component can have a locking mechanism which enables the component to be selectively locked at different flip-up angles (e.g. not just a 90-degree angle) relative to the housing. In an example, a flip-up component can have a locking mechanism which enables the component to be selectively locked at either a 45 or 90 degree angle relative to the housing. In an example, a flip-up component can have a locking mechanism which enables the component to be selectively locked at a 45, 60, 75, or 90 degree angle relative to the housing. In an example, a flip-up component can have a locking mechanism which temporarily locks the component in a flipped-up or popped-up configuration and also a pressure-release mechanism which releases the lock if a selected level of force is applied to the component to reduce the chance of breakage if the component is snagged on something while in a flipped-up or popped-up configuration.

In an example, a flip-up component can be connected to a housing on a wrist-worn device by: a hinge; a joint; a flexible band or strap; or a cord, cable, or wire. In an example, a flip-up component can be in electromagnetic communication with a data processing unit in a housing by a cord, cable, or wire. In an example, a flip-up component can pivot and/or rotate around an end of the component which is movably connected to a housing by a hinge, joint, or strap. In an example, one side or end of a flip-up component can be connected to a housing by a hinge, joint, or strap and the other side or end can pivot and/or rotate relative to the housing.

In an example, a flip-up component with a camera can be flipped-up (or popped-up) from of a housing by a mechanism and/or user action selected from the group consisting of: making a hand motion or gesture (recognized by the device via motion and/or EMG sensors); pinching or squeezing a flip-up component or housing; pressing a bevel or perimeter of a flip-up component or housing; pressing a button on a flip-up component or housing; pressing down on a display, flip-up component, or housing; pulling (or inserting) a pin or other connective protrusion; pulling or pushing a clip, latch, or clasp on a flip-up component or housing; rotating a bevel or perimeter of a flip-up component or housing; rotating or twisting a display, flip-up component, or housing; sliding a flip-up component along a track or slot on a housing; touching or swiping a touch display; and turning a knob on a flip-up component or housing.

In an example, one or more housings for a camera and a spectroscopic sensor can be removably-attached to a conventional watch band. In an example, one or more housings for a camera, a spectroscopic sensor, and/or a secondary display which acts as a camera viewfinder can be reversibly-attached to a conventional watch band using one or more mechanisms selected from the group consisting of: belt, clamp, clasp, clip, hook, hook and loop fabric, latch, magnet, plug, prongs, and snap. In an example, one or more housings for a camera, a spectroscopic sensor, and/or a secondary display can be in wireless communication with the primary display housing (e.g. watch face) of a smart watch.

In an example, a camera and a spectroscopic sensor can both be in the same housing and general location on a portion of a wrist-band. In an example, a camera and a spectroscopic sensor can be in different housings and/or locations. In another example, a camera and a spectroscopic sensor can be integrated into a specialized watch band which is an (interchangeable) option for use with a conventional smart watch. In another example, a primary display, a camera, and a spectroscopic sensor can all be integrated into a single specialized wrist-band device (e.g. a specialized food tracking band and/or customized food-tracking smart watch).

In an example, a wrist-worn device for tracking food intake can enable a person to activate a camera, activate a spectroscopic scanner, and/or flip-up a flip-up component by one or more actions selected from the group consisting of: making a selected hand motion or gesture as long as it is polite; rotating a bezel or ring on a display and/or housing; rotating a knob or “crown” on a display and/or housing; touching and/or swiping a display in a selected manner or location; touching and/or swiping a selected icon on a display screen; and voice command. In an example, a wrist-worn device for tracking food intake can enable a person to activate a camera to record food images via one or more components selected from the group consisting of: a touch screen (e.g. touching and/or swiping a touch-screen on the display); a motion sensor (e.g. a motion sensor which recognizes the orientation of the device and/or changes in this orientation); a gesture recognition sensor (e.g. an optical, electromagnetic radiation, or motion sensor which recognizes hand gestures); a rotating bezel or ring (e.g. a bezel which is rotated around a circular display); a rotating knob (e.g. a rotating knob which is perpendicular to the display surface); and voice command recognition (e.g. recognition of vocal commands recorded by a microphone).

In an example, a wrist-worn device for tracking food intake can enable a person to change the focal direction and/or distance of a camera via one or more components selected from the group consisting of: a touch screen (e.g. touching and/or swiping a touch-screen on the display); a motion sensor (e.g. a motion sensor which recognizes the orientation of the device and/or changes in this orientation); a gesture recognition sensor (e.g. an optical, electromagnetic radiation, or motion sensor which recognizes hand gestures); a rotating bezel or ring (e.g. a bezel which is rotated around a circular display); a rotating knob (e.g. a rotating knob which is perpendicular to the display surface); and voice command recognition (e.g. recognition of vocal commands recorded by a microphone).

In an example, a wrist-worn device for tracking food intake can enable a person to activate a spectroscopic sensor to scan nearby food via one or more components selected from the group consisting of: a touch screen (e.g. touching and/or swiping a touch-screen on the display); a motion sensor (e.g. a motion sensor which recognizes the orientation of the device and/or changes in this orientation); a gesture recognition sensor (e.g. an optical, electromagnetic radiation, or motion sensor which recognizes hand gestures); a rotating bezel or ring (e.g. a bezel which is rotated around a circular display); a rotating knob (e.g. a rotating knob which is perpendicular to the display surface); and voice command recognition (e.g. recognition of vocal commands recorded by a microphone).

In an example, a wrist-worn device for tracking food intake can enable a person to flip-up a flip-up display via one or more components selected from the group consisting of: a touch screen (e.g. touching and/or swiping a touch-screen on the display); a motion sensor (e.g. a motion sensor which recognizes the orientation of the device and/or changes in this orientation); a gesture recognition sensor (e.g. an optical, electromagnetic radiation, or motion sensor which recognizes hand gestures); a rotating bezel or ring (e.g. a bezel which is rotated around a circular display); a rotating knob (e.g. a rotating knob which is perpendicular to the display surface); and voice command recognition (e.g. recognition of vocal commands recorded by a microphone).

In an example, a wrist-worn device for tracking food intake can provide vibratory, tactile, audible, and/or visual feedback to a person when a satisfactory (e.g. clear, focused, and proper angle) food image is obtained. This feedback can guide the person concerning how to move their wrist relative to food in order to record satisfactory food images. For example, if a person's wrist is too far from a food item, then a device can emit a low pitched sound; if the person's wrist is too close to the food item, then the device can emit a high pitched sound. However, when the device is the proper distance to record a satisfactory image, then the device emits a selected audio signal (or no sound at all). Alternatively, if one is dining out and wishes to avoid nasty glares from other diners, then if a person's wrist is too far from a food item, then a device can vibrate at a low frequency; if the person's wrist is too close to a food item, then the device can vibrate at a high frequency. However, when the device is the proper distance to record a satisfactory image, then the device does not vibrate at all. In an example, a person can move their wrist (and thereby move a wrist-worn device) over different food items to capture sequential images of different food items in a meal, wherein the device provides vibratory, tactile, audible, and/or visual feedback each time a satisfactory image of an individual food item is obtained.

In an example, a wrist-worn device for tracking food intake can provide vibratory, tactile, audible, and/or visual feedback to a person when a satisfactory spectroscopic food scan is obtained. This feedback can guide the person concerning how to move their wrist relative to food in order to record a satisfactory spectroscopic food scan. For example, if a person's wrist is too far from a food item, then a device can emit a low pitched sound; if the person's wrist is too close to the food item, then the device can emit a high pitched sound. However, when the device is the proper distance to make a good spectroscopic scan, then the device emits a selected audio signal (or no sound at all). Alternatively, if a person's wrist is too far from a food item, then a device can vibrate at a low frequency; if the person's wrist is too close to a food item, then the device can vibrate at a high frequency. However, when the device is the proper distance to make a good spectroscopic scan, then the device does not vibrate at all. In an example, a person can move their wrist (and thereby move a wrist-worn device) over different food items to capture spectroscopic scans of different food items in a meal, wherein the device provides vibratory, tactile, audible, and/or visual feedback each time a satisfactory scan of an individual food item is obtained.

In an example, a wrist-worn device can be part of a system for tracking food intake which includes one or more other wearable components. In an example, these different wearable components can be in wireless communication with each other. In an example, different wearable components of a system for tracking food intake can serve different functions. For example, a first component can be the primary mechanism for detecting when the person is eating, a second component can be the primary mechanism for recording food images, and a third components can be the primary mechanism for performing spectroscopic scans of food. In an example, a wearable device or system for tracking food consumption can be embodied in one or more components selected from the group consisting of: adhesive patch, arm band, bracelet, brooch, ear bud, ear ring, eyewear, finger ring, fitness band, head-band, necklace, pendant, smart button, smart shirt, smart watch, smart watch band, and wrist band.

In an example, a system of wearable components for tracking food intake can comprise a smart wrist-worn device and smart eyewear which are in wireless communication with each other. In an example, sensors on the smart wrist-worn device can be primary mechanism for detecting eating and spectroscopic scans, while the smart eyewear has a camera which is the primary mechanism for recording food images. In an example, sensors on the smart wrist-worn device can be primary mechanism for spectroscopic scans, while the smart eyewear has a camera which is the primary mechanism for recording food images and a chewing sensor (e.g. EMG sensor, vibration sensor, stretch sensor, and/or microphone) which is the primary mechanism for detecting eating.

In an example, a wrist-worn device can be integrated with augmented reality eyewear into a wearable system for tracking food intake. For example, virtual images displayed in a person's field of view by augmented reality eyewear can guide a person concerning how to move their wrist over food items in order to record satisfactory food images and/or spectroscopic scans. In another example, information on the quantity and/or nutritional composition of food items identified by the system can be virtually displayed in a person's field of view by augmented reality eyewear. In an example, an eyewear-based camera can track the location of a moving wrist relative to individual food items in order to match those food items with the results of sequential spectroscopic scans.

In an example, a system for tracking food intake can include two wrist-worn devices, wherein one device is worn on each wrist. This can be especially useful if a person generally wears a watch (or other wrist bands) on their non-dominant arm and primarily eats with their dominant arm. In this case, tracking motions of the dominant arm can detect eating more accurately than tracking motions of the non-dominant arm, so having two wrist-worn devices which are in wireless communication with each other can more accurately detect eating than having just one wrist-worn device on the non-dominant arm. In an example, a first wrist-worn device which a person wears on their first (e.g. left) wrist can record food images, take spectroscopic scans of food, and display food images; a second wrist-worn device which a person wears on their second (e.g. right) wrist can detect eating; and the first and second wrist-worn devices can be wireless communication with each other. In an example, when motion sensors on the second wrist-worn device indicate that a person is eating, then the camera and spectroscopic sensor on the first wrist-worn device can be automatically activated to record food images and spectroscopically scan food.

In an example, a system for tracking food intake can comprise a wrist-worn device with an eating detector which is worn on a person's dominant arm and smart eyewear, wherein the wrist-worn device and eyewear are in wireless communication with each other. In an example, a system for tracking food intake can comprise a wrist-worn device with one or more motion sensors (e.g. accelerometer, gyroscope, magnetometer, and/or inclinometer) which is worn on a person's dominant arm and smart eyewear, wherein a camera in the eyewear is automatically activated to record food images when analysis of data from the motion sensors indicates that the person wearing the system is eating. In an example, a system for tracking food intake can comprise a wrist-worn device with one or more EMG sensors which is worn on a person's dominant arm and smart eyewear, wherein a camera in the eyewear is automatically activated to record food images when analysis of data from the EMG sensors indicates that the person wearing the system is eating.

In an example, a system for tracking food intake can comprise a wrist-worn device and a cellphone. In an example, the wrist-worn device can serve the eating detection function and the cellphone can serve the imaging function. In an example, when a motion sensor on the wrist-worn device indicates that a person is eating, the system can prompt the person to record food images. Since most smart watches already have motion sensors and most cellphones already have cameras, such a system for tracking food intake could be created with existing hardware via new software and wireless communication between the wrist-worn device and the cellphone. Adding spectroscopic scanning functionality to such a system would require additional hardware to current smart watch and cellphone devices, but if spectroscopic capability becomes standard in either in future years, then spectroscopic scanning could also be added to such a system.

In an example, a wrist-worn device for tracking food intake can further comprise one or more components selected from the group consisting of: a battery, a data processor, a data transmitter and/or receiver, a GPS component, a microphone, a microprojector, an infrared distance finder and/or range sensor, a push button, a rotatable crown, a sweat sensor, a temperature sensor, a vibrating protrusions, an ambient light sensor, and an electromagnetic impedance or capacitance sensor.

In an example, a method for tracking food intake can comprise: receiving data from a motion sensor on a smart watch or other wrist-worn device worn on a person's wrist; analyzing the data to detect when the person is eating; and prompting the person (e.g. with a vibrational, auditory, or visual stimulus) to record food images with a cellphone when the person is eating. In an example, a method for tracking food intake can comprise: receiving data from a motion sensor on a smart watch or other wrist-worn device worn on a person's wrist; analyzing the data to detect when the person is eating; and prompting the person (e.g. with a vibrational, auditory, or visual stimulus) to record food images with a camera on the smart watch or other wrist-worn device when the person is eating.

In an example, a method for tracking food intake can comprise: receiving data from a motion sensor on a smart watch or other wrist-worn device worn on a person's wrist; analyzing the data to detect when the person is eating; and prompting the person (e.g. with a vibrational, auditory, or visual stimulus) to record food images and a spectroscopic scan of the food with a cellphone when the person is eating. In an example, a method for tracking food intake can comprise: receiving data from a motion sensor on a smart watch or other wrist-worn device worn on a person's wrist; analyzing the data to detect when the person is eating; and prompting the person (e.g. with a vibrational, auditory, or visual stimulus) to record food images with a camera on the smart watch or other wrist-worn device and record a spectroscopic scan of the food with a spectroscopic sensor on the smart watch or other wrist-worn device when the person is eating.

In an example, tracking a person's food consumption can be partially automatic and partially refined by human evaluation or interaction. In an example, initial estimates of the types and quantities of food consumed by a person can be subsequently refined by human evaluation and/or interaction. In an example, this human evaluation and/or interaction can involve the person whose food consumption is being tracked. Alternatively, this human evaluation and/or interaction can involve other people (e.g. via remote image analysis by experts or crowd-source evaluators). In an example, a wearable device can prompt a person with clarifying questions concerning the types and quantities of food that the person is consuming or has consumed. These questions can be asked in real time, as a person eats, at a subsequent time, or periodically. In an example, a device can prompt a person with queries to refine initial automatically-generated estimates of the types and quantities of food consumed.

In an example, analysis of food images and estimation of food consumed can be entirely automatic or can be a mixture of automated estimates plus human refinement. Even a partially-automated method for calorie monitoring and estimation can be superior to relying completely on manual calorie counting and/or manual entry of food items consumed. In an example, images can be automatically, or semi-automatically, analyzed to estimate the types of quantities of food that a person consumes. These estimates are, in turn, used to estimate the person's caloric intake. In an example, the caloric intake estimation provided can become the energy-input measuring component of an overall system for energy balance and weight management.

In an example, a wearable device for tracking food consumption can be incorporated into an overall device, system, and method for human energy balance and weight management. In an example, estimates of the types and quantities of food consumed can be used to estimate human caloric intake. These estimates of human caloric intake can then be used in combination with estimates of human caloric expenditure as part of an overall system for human energy balance and weight management. In an example, estimates of the types and quantities of food consumed can be used to estimate human caloric intake, wherein these estimates of human caloric intake are used in combination with estimates of human caloric expenditure as part of an overall system for human energy balance and human weight management. This overall device, system, and method can be used to help a person to lose weight or to maintain a desirable weight. In an example, such a device and method can be used as part of a system with a human-energy input measuring component and a human-energy output measuring component.

Information from a wearable device that measures a person's consumption of at least one selected type of food, ingredient, and/or nutrient can be combined with information from a separate caloric expenditure monitoring device that measures a person's caloric expenditure to comprise an overall system for energy balance, fitness, weight management, and health improvement. In an example, a wearable device to track food intake can be in wireless communication with a separate fitness monitoring device. In an example, capability for monitoring food consumption can be combined with capability for monitoring caloric expenditure within a single device. In an example, a single device can be used to measure the types and amounts of food, ingredients, and/or nutrients that a person consumes as well as the types and durations of the calorie-expending activities in which the person engages.

Information from a wearable device that measures a person's consumption of at least one selected type of food, ingredient, and/or nutrient can also be combined with a computer-to-human interface that provides feedback to encourage the person to eat healthy foods and to limit excess consumption of unhealthy foods. In an example, a wearable device to track food intake can be in wireless communication with a separate feedback device that modifies the person's eating behavior. In an example, capability for monitoring food consumption can be combined with capability for providing behavior-modifying feedback within a single device. In an example, a single device can be used to measure the selected types and amounts of foods, ingredients, and/or nutrients that a person consumes and to provide visual, auditory, tactile, or other feedback to encourage the person to eat in a healthier manner.

A combined device and system for measuring and modifying caloric intake and caloric expenditure can be a useful part of an overall approach for good nutrition, energy balance, fitness, weight management, and good health. As part of such an overall system, a device that measures a person's consumption of at least one selected type of food, ingredient, and/or nutrient can play a key role in helping that person to achieve their goals with respect to proper nutrition, food consumption modification, energy balance, weight management, and good health outcomes.

In order to be really useful for achieving good nutrition and health goals, a device should be able to differentiate between a person's consumption of healthy foods vs. unhealthy foods. This requires the ability to identify consumption of selected types of foods, ingredients, and/or nutrients, as well as estimating the amounts of such consumption. It also requires selection of certain types and/or amounts of food, ingredients, and/or nutrients as healthy vs. unhealthy.

Generally, the technical challenges of identifying consumption of selected types of foods, ingredients, and/or nutrients are greater than the challenges of identifying which types are healthy or unhealthy. Accordingly, while this disclosure covers both food identification and classification, it focuses in greatest depth on identification of consumption of selected types of foods, ingredients, and nutrients. In this disclosure, food consumption is broadly defined to include consumption of liquid beverages and gelatinous food as well as solid food.

In an example, a device can identify consumption of at least one selected type of food. In such an example, selected types of ingredients or nutrients can be estimated indirectly using a database that links common types and amounts of food with common types and amounts of ingredients or nutrients. In another example, a device can directly identify consumption of at least one selected type of ingredient or nutrient. The latter does not rely on estimates from a database, but does require more complex ingredient-specific or nutrient-specific sensors. Since the concepts of food identification, ingredient identification, and nutrient identification are closely related, we consider them together for many portions of this disclosure, although we consider them separately in some sections for greater methodological detail. Various embodiments of the device and method disclosed herein can identify specific nutrients indirectly (through food identification and use of a database) or directly (through the use of nutrient-specific sensors).

Many people consume highly-processed foods whose primary ingredients include multiple types of sugar. The total amount of sugar is often obscured or hidden, even from those who read ingredients on labels. Sometimes sugar is disguised as “evaporated cane syrup.” Sometimes different types of sugar are labeled as different ingredients (such as “plain sugar,” “brown sugar,” “maltose”, “dextrose,” and “evaporated cane syrup”) in a single food item. In such cases, “sugar” does not appear as the main ingredient. However, when one adds up all the different types of sugar in different priority places on the ingredient list, then sugar really is the main ingredient. These highly-processed conglomerations of sugar (often including corn syrup, fats, and/or caffeine) often have colorful labels with cheery terms like “100% natural” or “high-energy.” However, they are unhealthy when eaten in the quantities to which many Americans have become accustomed. It is no wonder that there is an obesity epidemic. The device and method disclosed herein is not be fooled by deceptive labeling of ingredients.

In various examples, a wearable device for tracking food intake can measure one or more types selected from the group consisting of: a selected type of carbohydrate, a class of carbohydrates, or all carbohydrates; a selected type of sugar, a class of sugars, or all sugars; a selected type of fat, a class of fats, or all fats; a selected type of cholesterol, a class of cholesterols, or all cholesterols; a selected type of protein, a class of proteins, or all proteins; a selected type of fiber, a class of fiber, or all fibers; a specific sodium compound, a class of sodium compounds, or all sodium compounds; high-carbohydrate food, high-sugar food, high-fat food, fried food, high-cholesterol food, high-protein food, high-fiber food, and/or high-sodium food.

In various examples, a wearable device for tracking food intake can measure one or more types selected from the group consisting of: simple carbohydrates, simple sugars, saturated fat, trans fat, Low Density Lipoprotein (LDL), and salt. In an example, a wearable device for tracking food intake can measure a person's consumption of simple carbohydrates. In an example, a wearable device for tracking food intake can measure a person's consumption of simple sugars. In an example, a wearable device for tracking food intake can measure a person's consumption of saturated fats. In an example, a wearable device for tracking food intake can measure a person's consumption of trans fats. In an example, a wearable device for tracking food intake can measure a person's consumption of Low Density Lipoprotein (LDL). In an example, a wearable device for tracking food intake can measure a person's consumption of sodium.

In various examples, a food-identifying sensor can detect one or more nutrients selected from the group consisting of: amino acid or protein (a selected type or general class), carbohydrate (a selected type or general class, such as single carbohydrates or complex carbohydrates), cholesterol (a selected type or class, such as HDL or LDL), dairy products (a selected type or general class), fat (a selected type or general class, such as unsaturated fat, saturated fat, or trans fat), fiber (a selected type or class, such as insoluble fiber or soluble fiber), mineral (a selected type), vitamin (a selected type), nuts (a selected type or general class, such as peanuts), sodium compounds (a selected type or general class), sugar (a selected type or general class, such as glucose), and water. In an example, food can be classified into general categories such as fruits, vegetables, or meat.

In an example, a wearable device for tracking food intake can measure a person's consumption of food that is high in simple carbohydrates. In an example, a wearable device for tracking food intake can measure a person's consumption of food that is high in simple sugars. In an example, a wearable device for tracking food intake can measure a person's consumption of food that is high in saturated fats. In an example, a wearable device for tracking food intake can measure a person's consumption of food that is high in trans fats. In an example, a wearable device for tracking food intake can measure a person's consumption of food that is high in Low Density Lipoprotein (LDL). In an example, a wearable device for tracking food intake can measure a person's consumption of food that is high in sodium.

In an example, a wearable device for tracking food intake can measure a person's consumption of food wherein a high proportion of its calories comes from simple carbohydrates. In an example, a wearable device for tracking food intake can measure a person's consumption of food wherein a high proportion of its calories comes from simple sugars. In an example, a wearable device for tracking food intake can measure a person's consumption of food wherein a high proportion of its calories comes from saturated fats. In an example, a wearable device for tracking food intake can measure a person's consumption of food wherein a high proportion of its calories comes from trans fats. In an example, a wearable device for tracking food intake can measure a person's consumption of food wherein a high proportion of its calories comes from Low Density Lipoprotein (LDL). In an example, a wearable device for tracking food intake can measure a person's consumption of food wherein a high proportion of its weight or volume is comprised of sodium compounds.

In an example, a wearable device for tracking food intake can track the quantities of selected chemicals that a person consumes via food consumption. In various examples, these consumed chemicals can be selected from the group consisting of carbon, hydrogen, nitrogen, oxygen, phosphorus, and sulfur. In an example, a wearable device for tracking food intake can selectively detect consumption of one or more types of unhealthy food, wherein unhealthy food is selected from the group consisting of: food that is high in simple carbohydrates; food that is high in simple sugars; food that is high in saturated or trans fat; fried food; food that is high in Low Density Lipoprotein (LDL); and food that is high in sodium.

In a broad range of examples, a food-identifying sensor can measure one or more types selected from the group consisting of: a selected food, ingredient, or nutrient that has been designated as unhealthy by a health care professional organization or by a specific health care provider for a specific person; a selected substance that has been identified as an allergen for a specific person; peanuts, shellfish, or dairy products; a selected substance that has been identified as being addictive for a specific person; alcohol; a vitamin or mineral; vitamin A, vitamin B1, thiamin, vitamin B12, cyanocobalamin, vitamin B2, riboflavin, vitamin C, ascorbic acid, vitamin D, vitamin E, calcium, copper, iodine, iron, magnesium, manganese, niacin, pantothenic acid, phosphorus, potassium, riboflavin, thiamin, and zinc; a selected type of carbohydrate, class of carbohydrates, or all carbohydrates; a selected type of sugar, class of sugars, or all sugars; simple carbohydrates, complex carbohydrates; simple sugars, complex sugars, monosaccharides, glucose, fructose, oligosaccharides, polysaccharides, starch, glycogen, disaccharides, sucrose, lactose, starch, sugar, dextrose, disaccharide, fructose, galactose, glucose, lactose, maltose, monosaccharide, processed sugars, raw sugars, and sucrose; a selected type of fat, class of fats, or all fats; fatty acids, monounsaturated fat, polyunsaturated fat, saturated fat, trans fat, and unsaturated fat; a selected type of cholesterol, a class of cholesterols, or all cholesterols; Low Density Lipoprotein (LDL), High Density Lipoprotein (HDL), Very Low Density Lipoprotein (VLDL), and triglycerides; a selected type of protein, a class of proteins, or all proteins; dairy protein, egg protein, fish protein, fruit protein, grain protein, legume protein, lipoprotein, meat protein, nut protein, poultry protein, tofu protein, vegetable protein, complete protein, incomplete protein, or other amino acids; a selected type of fiber, a class of fiber, or all fiber; dietary fiber, insoluble fiber, soluble fiber, and cellulose; a specific sodium compound, a class of sodium compounds, and all sodium compounds; salt; a selected type of meat, a class of meats, and all meats; a selected type of vegetable, a class of vegetables, and all vegetables; a selected type of fruit, a class of fruits, and all fruits; a selected type of grain, a class of grains, and all grains; high-carbohydrate food, high-sugar food, high-fat food, fried food, high-cholesterol food, high-protein food, high-fiber food, and high-sodium food.

In an example, a wearable device for tracking food intake that can analyze food composition can also identify one or more potential food allergens, toxins, or other substances selected from the group consisting of: ground nuts, tree nuts, dairy products, shell fish, eggs, gluten, pesticides, animal hormones, and antibiotics. In an example, a device can analyze food composition to identify one or more types of food whose consumption is prohibited or discouraged for religious, moral, and/or cultural reasons, such as pork or meat products of any kind.

Having discussed different ways to classify types of foods, ingredients, and nutrients, we now turn to different metrics for measuring the amounts of foods, ingredients, and nutrients consumed. Overall, amounts or quantities of food, ingredients, and nutrients consumed can be measured in terms of volume, mass, or weight. Volume measures how much space the food occupies. Mass measures how much matter the food contains. Weight measures the pull of gravity on the food. The concepts of mass and weight are related, but not identical. Food, ingredient, or nutrient density can also be measured, sometimes as a step toward measuring food mass.

Volume can be expressed in metric units (such as cubic millimeters, cubic centimeters, or liters) or U.S. (historically English) units (such as cubic inches, teaspoons, tablespoons, cups, pints, quarts, gallons, or fluid ounces). Mass (and often weight in colloquial use) can be expressed in metric units (such as milligrams, grams, and kilograms) or U.S. (historically English) units (ounces or pounds). In an example, cone heads can consume mass quantities of food. The density of specific ingredients or nutrients within food is sometimes measured in terms of the volume of specific ingredients or nutrients per total food volume or measured in terms of the mass of specific ingredients or nutrients per total food mass.

In an example, the amount of a specific ingredient or nutrient within (a portion of) food can be measured directly by a sensing mechanism. In an example, the amount of a specific ingredient or nutrient within (a portion of) food can be estimated indirectly by measuring the amount of food and then linking this amount of food to amounts of ingredients or nutrients using a database that links specific foods with standard amounts of ingredients or nutrients.

In an example, an amount of a selected type of food, ingredient, or nutrient consumed can be expressed as an absolute amount. In an example, an amount of a selected type of food, ingredient, or nutrient consumed can be expressed as a percentage of a standard amount. In an example, an amount of a selected type of food, ingredient, or nutrient consumed can be displayed as a portion of a standard amount such as in a bar chart, pie chart, thermometer graphic, or battery graphic.

In an example, a standard amount can be selected from the group consisting of: daily recommended minimum amount; daily recommended maximum amount or allowance; weekly recommended minimum amount; weekly recommended maximum amount or allowance; target amount to achieve a health goal; and maximum amount or allowance per meal. In an example, a standard amount can be a Reference Daily Intake (RDI) value or a Daily Reference Value.

In an example, the volume of food consumed can be estimated by analyzing one or more pictures of that food. In an example, volume estimation can include the use of a physical or virtual fiduciary marker or object of known size for estimating the size of a portion of food. In an example, a physical fiduciary marker can be placed in the field of view of an imaging system for use as a point of reference or a measure. In an example, this fiduciary marker can be a plate, utensil, or other physical place setting member of known size. In an example, this fiduciary marker can be created virtually by the projection of coherent light beams. In an example, a device can project (laser) light points onto food and, in conjunction with infrared reflection or focal adjustment, use those points to create a virtual fiduciary marker. A fiduciary marker may be used in conjunction with a distance-finding mechanism (such as infrared range finder) that determines the distance from the camera and the food.

In an example, volume estimation can include obtaining video images of food or multiple still pictures of food in order to obtain pictures of food from multiple perspectives. In an example, pictures of food from multiple perspectives can be used to create three-dimensional or volumetric models of that food in order to estimate food volume. In an example, such methods can be used prior to food consumption and again after food consumption, in order to estimate the volume of food consumed based on differences in food volume measured. In an example, food volume estimation can be done by analyzing one or more pictures of food before (and after) consumption. In an example, multiple pictures of food from different angles can enable three-dimensional modeling of food volume. In an example, multiple pictures of food at different times (such as before and after consumption) can enable estimation of the amount of proximal food that is actually consumed vs. just being served in proximity to the person.

In a non-imaging example of food volume estimation, a utensil or other apportioning device can be used to divide food into mouthfuls. Then, the number of times that the utensil is used to bring food up to the person's mouth can be tracked. Then, the number of utensil motions is multiplied times the estimated volume of food per mouthful in order to estimate the cumulative volume of food consumed. In an example, the number of hand motions or mouth motions can be used to estimate the quantity of food consumed. In an example, a motion sensor worn on a person's wrist or incorporated into a utensil can measure the number of hand-to-mouth motions. In an example, a motion sensor, sound sensor, or electromagnetic sensor in communication with a person's mouth can measure the number of chewing motions which, in turn, can be used to estimate food volume.

In an example, a wearable device for tracking food intake can measure the weight or mass of food that the person consumes. In an example, a wearable device for tracking food intake can include a food scale that measures the weight of food. In an example a food scale can measure the weight of food prior to consumption and the weight of unconsumed food remaining after consumption in order to estimate the weight of food consumed based on the difference in pre vs. post consumption measurements. In an example, a food scale can be a stand-alone device. In an example, a food scale can be incorporated into a plate, glass, cup, glass coaster, place mat, or other place setting. In an example a plate can include different sections which separately measure the weights of different foods on the plate. In an example, a food scale embedded into a place setting or smart utensil can automatically transmit data concerning food weight to a computer.

In an example, a food scale can be incorporated into a smart utensil. In an example, a food scale can be incorporated into a utensil rest on which a utensil is placed for each bite or mouthful. In an example, a food scale can be incorporated into a smart utensil which tracks the cumulative weight of cumulative mouthfuls of food during an eating event. In an example, a smart utensil can approximate the weight of mouthfuls of food by measuring the effect of food carried by the utensil on an accelerometer or other inertial sensor. In an example, a smart utensil can incorporate a spring between the food-carrying portion and the hand-held portion of a utensil and food weight can be estimated by measuring distension of the spring as food is brought up to a person's mouth.

In an example, a smart utensil can use an inertial sensor, accelerometer, or strain gauge to estimate the weight of the food-carrying end of utensil at a first time (during an upswing motion as the utensil carries a mouthful of food up to the person's mouth), can use this sensor to estimate the weight of the food-carrying end of the utensil at a second time (during a downswing motion as the person lowers the utensil from their mouth), and can estimate the weight of the mouthful of food by calculating the difference in weight between the first and second times.

In an example, a device or system can measure nutrient density or concentration as part of an automatic food, ingredient, or nutrient identification method. In an example, such nutrient density can be expressed as the average amount of a specific ingredient or nutrient per unit of food weight. In an example, such nutrient density can be expressed as the average amount of a specific ingredient or nutrient per unit of food volume. In an example, food density can be estimated by interacting food with light, sound, or electromagnetic energy and measuring the results of this interaction. Such interaction can include energy absorption or reflection.

In an example, nutrient density can be determined by reading a label on packaging associated with food consumed. In an example, nutrient density can be determined by receipt of wirelessly transmitted information from a grocery store display, electronically-functional restaurant menu, or vending machine. In an example, food density can be estimated by ultrasonic scanning of food. In an example, food density and food volume can be jointly analyzed to estimate food weight or mass.

In an example, for some foods with standardized sizes (such as foods that are manufactured in standard sizes at high volume), food weight can be estimated as part of food identification. In an example, information concerning the weight of food consumed can be linked to nutrient quantities in a computer database in order to estimate cumulative consumption of selected types of nutrients.

In an example, a method for measuring a person's consumption of at least one selected type of food, ingredient, or nutrient can comprise monitoring changes in the volume or weight of food at a reachable location near the person. In an example, pictures of food can be taken at multiple times before, during, and after food consumption in order to better estimate the amount of food that the person actually consumes, which can differ from the amount of food served to the person or the amount of food left over after the person eats. In an example, estimates of the amount of food that the person actually consumes can be made by digital image subtraction and/or 3D modeling. In an example, changes in the volume or weight of nearby food can be correlated with hand motions in order to estimate the amount of food that a person actually eats. In an example, a device can track the cumulative number of hand-to-mouth motions, number of chewing motions, or number of swallowing motions. In an example, estimation of food consumed can also involve asking the person whether they ate all the food that was served to them.

In an example, a wearable device for tracking food intake can collect data that enables tracking the cumulative amount of a type of food, ingredient, or nutrient which the person consumes during a period of time (such as an hour, day, week, or month) or during a particular eating event. In an example, the time boundaries of a particular eating event can be defined by a maximum time between chews or mouthfuls during a meal and/or a minimum time between chews or mouthfuls between meals. In an example, the time boundaries of a particular eating event can be defined by Fourier Transformation analysis of the variable frequencies of chewing, swallowing, or biting during meals vs. between meals.

In an example, a wearable device for tracking food intake can track the cumulative amount of that food, ingredient, or nutrient consumed by the person and provide feedback to the person based on the person's cumulative consumption relative to a target amount. In an example, a device can provide negative feedback when a person exceeds a target amount of cumulative consumption. In an example, a device and system can sound an alarm or provide other real-time feedback to a person when the cumulative consumed amount of a selected type of food, ingredient, or nutrient exceeds an allowable amount (in total, per meal, or per unit of time).

In various examples, a target amount of consumption can be based on one or more factors selected from the group consisting of: the selected type of selected food, ingredient, or nutrient; amount of this type recommended by a health care professional or governmental agency; specificity or breadth of the selected nutrient type; the person's age, gender, and/or weight; the person's diagnosed health conditions; the person's exercise patterns and/or caloric expenditure; the person's physical location; the person's health goals and progress thus far toward achieving them; one or more general health status indicators; magnitude and/or certainty of the effects of past consumption of the selected nutrient on the person's health; the amount and/or duration of the person's consumption of healthy food or nutrients; changes in the person's weight; time of day; day of the week; occurrence of a holiday or other occasion involving special meals; dietary plan created for the person by a health care provider; input from a social network and/or behavioral support group; input from a virtual health coach; health insurance copay and/or health insurance premium; financial payments, constraints, and/or incentives; cost of food; speed or pace of nutrient consumption; and accuracy of the sensor in detecting the selected nutrient.

A wearable device for tracking food intake can include: a general food-consumption monitor that detects when a person is probably eating, but does not identify the selected types of foods, ingredients, or nutrients that the person is eating; and a food-identifying sensor that identifies the person's consumption of at least one selected type of food, ingredient, or nutrient.

In an example, operation of a food-identifying sensor can be triggered by the results of a general food-consumption monitor. In an example, a general food-consumption monitor with low privacy intrusion (but low food identification accuracy) can operate continually and trigger the operation of a food-identifying sensor with high privacy intrusion (but high food identification accuracy) when the person is eating. In an example, a general food-consumption monitor with low privacy intrusion (but low power or resource requirements) can operate continually and trigger the operation of a food-identifying sensor with high privacy intrusion (but high power or resource requirements) when the person is eating. In an example, the combination of a general food-consumption monitor and a food-identifying sensor can achieve relatively-high food identification accuracy with relatively-low privacy intrusion or resource requirements.

In an example, a food-consumption monitor or food-identifying sensor can measure food weight, mass, volume, or density. In an example, such a sensor can be a scale, strain gauge, or inertial sensor. In an example, such a sensor can measure the weight or mass of an entire meal, a portion of one type of food within that meal, or a mouthful of a type of food that is being conveyed to a person's mouth. In general, a weight, mass, or volume sensor is more useful for general detection of food consumption and food amount than it is for identification of consumption of selected types of foods, ingredients, and nutrients. However, it can be very useful when used in combination with a specific food-identifying sensor.

In an example, a food-consumption monitor can be a motion sensor. In various examples, a motion sensor can be selected from the group consisting of: bubble accelerometer, dual-axial accelerometer, electrogoniometer, gyroscope, inclinometer, inertial sensor, multi-axis accelerometer, piezoelectric sensor, piezo-mechanical sensor, pressure sensor, proximity detector, single-axis accelerometer, strain gauge, stretch sensor, and tri-axial accelerometer. In an example, a motion sensor can collect data concerning the movement of a person's wrist, hand, fingers, arm, head, mouth, jaw, or neck. In an example, analysis of this motion data can be used to identify when the person is probably eating. In general, a motion sensor is more useful for general detection of food consumption and food amount than it is for identification of consumption of selected types of foods, ingredients, and nutrients. However, it can be very useful when used in combination with a specific food-identifying sensor.

In an example, there can be an identifiable pattern of movement that is highly-associated with food consumption and a motion sensor can monitor a person's movements to identify times when the person is probably eating. In an example, this movement can include repeated movement of a person's hand up to their mouth. In an example, this movement can include a combination of three-dimensional roll, pitch, and yaw by a person's wrist and/or hand. In an example, this movement can include repeated bending of a person's elbow. In an example, this movement can include repeated movement of a person's jaws. In an example, this movement can include peristaltic motion of the person's esophagus that is detectable from contact with a person's neck.

In an example, a motion sensor can be used to estimate the quantity of food consumed based on the number of motion cycles. In an example, a motion sensor can be used to estimate the speed of food consumption based on the speed or frequency of motion cycles. In an example, a proximity sensor can detect when a person's hand gets close to their mouth. In an example, a proximity sensor can detect when a wrist (or hand or finger) is in proximity to a person's mouth. However, a proximity detector can be less useful than a motion detector because it does not identify complex three-dimensional motions that can differentiate eating from other hand-to-mouth motions such as coughing, yawning, smoking, and tooth brushing.

In various examples, a wearable device for tracking food intake can include a motion sensor that collects data concerning movement of the person's body. In an example, this data can be used to detect when a person is consuming food. In an example, this data can be used to aid in the identification of what types and amounts of food the person is consuming. In an example, analysis of this data can be used to trigger additional data collection to resolve uncertainty concerning the types and amounts of food that the person is consuming.

In an example, a motion sensor can include one or more accelerometers, inclinometers, electrogoniometers, and/or strain gauges. In an example, movement of a person's body that can be monitored and analyzed can be selected from the group consisting of: finger movements, hand movements, wrist movements, arm movements, elbow movements, eye movements, and head movements; tilting movements, lifting movements; hand-to-mouth movements; angles of rotation in three dimensions around the center of mass known as roll, pitch and yaw; and Fourier Transformation analysis of repeated body member movements. In an example, each hand-to-mouth movement that matches a certain pattern can be used to estimate bite or mouthful of food. In an example, the speed of hand-to-mouth movements that match a certain pattern can be used to estimate eating speed. In an example, this pattern can include upward and tilting hand movement, followed by a pause, following by a downward and tilting hand movement.

In an example, a motion sensor that is used to detect food consumption can be worn on a person's wrist, hand, arm, or finger. In an example, a motion sensor can be incorporated into a smart watch, fitness watch, or watch phone. In an example, a fitness watch that already uses an accelerometer to measure motion for estimating caloric expenditure can also use an accelerometer to detect (and estimate the quantity of) food consumption.

Motion-sensing devices that are worn on a person's wrist, hand, arm, or finger can continuously monitor a person's movements to detect food consumption with high compliance and minimal privacy intrusion. They do not require that a person carry a particular piece of electronic equipment everywhere they go and consistently bring that piece of electronic equipment out for activation each time that they eat a meal or snack. However, a motion-detecting device that is worn constantly on a person's wrist, hand, arm, or finger can be subject to false alarms due to motions (such as coughing, yawning, smoking, and tooth brushing) that can be similar to eating motions. To the extent that there is a distinctive pattern of hand and/or arm movement associated with bringing food up to one's mouth, such a device can detect when food consumption is occurring.

In an example, a motion-sensing device that is worn on a person's wrist, hand, arm, or finger can measure how rapidly or often the person brings their hand up to their mouth. A common use of such information is to encourage a person to eat at a slower pace. The idea that a person will eat less if they eat at a slower pace is based on the lag between food consumption and the feeling of satiety from internal gastric organs. If a person eats slower, then they will tend to not overeat past the point of internal identification of satiety.

In an example, a smart watch, fitness watch, watch phone, smart ring, or smart bracelet can measure the speed, pace, or rate at which a person brings food up to their mouth while eating and provide feedback to the person to encourage them to eat slower if the speed, pace, or rate is high. In an example, feedback can be sound-based, such as an alarm, buzzer, or computer-generated voice. In an example, feedback can be tactile, such as vibration or pressure. In an example, such feedback can be visual, such as a light, image, or display screen. In an alternative example, eating speed can be inferred indirectly by a plate, dish, bowl, glass or other place setting member that measures changes in the weight of food on the member. Negative feedback can be provided to the person if the weight of food on the plate, dish, bowl, or glass decreases in a manner that indicates that food consumption is too fast.

In an example, a motion sensor that is used to detect food consumption can be incorporated into, or attached to, a food utensil such as a fork or spoon. A food utensil with a motion sensor can be less prone to false alarms than a motion sensor worn on a person's wrist, hand, arm, or finger because the utensil is only used when the person eats food. Since the utensil is only used for food consumption, analysis of complex motion and differentiation of food consumption actions vs. other hand gestures is less important with a utensil than it is with a device that is worn on the person's body. In an example, a motion sensor can be incorporated into a smart utensil. In an example, a smart utensil can estimate the amount of food consumed by the number of hand-to-mouth motions (combined with information concerning how much food is conveyed by the utensil with each movement). In an example, a smart utensil can encourage a person to eat slower. The idea is that if the person eats more slowly, then they will tend to not overeat past the point of internal identification of satiety.

In an example, a food-consumption monitor or food-identifying sensor can be a light-based sensor that records the interaction between light and food. In an example, a light-based sensor can be a camera, mobile phone, or other conventional imaging device that takes plain-light pictures of food. In an example, a light-based food consumption or identification sensor can comprise a camera that takes video pictures or still pictures of food. In an example, such a camera can take pictures of the interaction between a person and food, including food apportionment, hand-to-mouth movements, and chewing movements.

In an example, a wearable device for tracking food intake can include a camera, or other picture-taking device, that takes pictures of food. In the following section, we discuss different examples of how a camera or other imaging-device can be used to take pictures of food and how those pictures can be analyzed to identify the types and amounts of food consumed. After that section, we discuss some other light-based approaches to food identification (such as spectroscopy) that do not rely on conventional imaging devices and plain-light food pictures.

A food-consumption monitor or food-identifying sensor can be a camera or other imaging device that is carried and held by a person. In an example, a camera that is used for food identification can be part of a mobile phone, cell phone, electronic tablet, or smart food utensil. In an example, a food-consumption monitor or food-identifying sensor can be a camera or other imaging device that is worn on a person's body or clothing. In an example, a camera can be incorporated into a smart watch, smart bracelet, smart button, or smart necklace.

In an example, a camera that is used for monitoring food consumption and/or identifying consumption of at least one selected type of food, ingredient, or nutrient can be a dedicated device that is specifically designed for this purpose. In an example, a camera that is used for monitoring food consumption and/or identifying consumption of specific foods can be a part of a general purpose device (such as a mobile phone, cell phone, electronic tablet, or digital camera) and in wireless communication with a dedicated device for monitoring food consumption and identifying specific food types.

In an example, use of a hand-held camera, mobile phone, or other imaging device to identify food depends on a person's manually aiming and triggering the device for each eating event. In an example, the person must bring the imaging device with them to each meal or snack, orient it toward the food to be consumed, and activate taking a picture of the food by touch or voice command. In an example, a camera, smart watch, smart necklace or other imaging device that is worn on a person's body or clothing can move passively as the person moves. In an example, the field of vision of an imaging device that is worn on a person's wrist, hand, arm, or finger can move as the person brings food up to their mouth when eating. In an example, such an imaging device can passively capture images of a reachable food source and interaction between food and a person's mouth.

In another example, the imaging vector and/or focal range of an imaging device worn on a person's body or clothing can be actively and deliberately adjusted to better track the person's hands and mouth to better monitor for possible food consumption. In an example, a device can optically scan the space surrounding the person for reachable food sources, hand-to-food interaction, and food-to-mouth interaction. In an example, in the interest of privacy, an imaging device that is worn on a person's body or clothing can only take pictures when some other sensor or information indicates that the person is probably eating.

In an example, a camera that is used for identifying food consumption can have a variable focal length. In an example, the imaging vector and/or focal distance of a camera can be actively and automatically adjusted to focus on: the person's hands, space surrounding the person's hands, a reachable food source, a food package, a menu, the person's mouth, and the person's face. In an example, in the interest of privacy, the focal length of a camera can be automatically adjusted in order to focus on food and not other people.

In an example, a device for measuring a person's consumption of at least one selected type of food, ingredient, or nutrient can include an imaging component that the person must manually aim toward food and manually activate for taking food pictures (such as through touch or voice commands). In an example, the taking of food pictures in this manner requires at least one specific voluntary human action associated with each food consumption event, apart from the actual act of eating, in order to take pictures of food during that food consumption event. In an example, such specific voluntary human actions can be selected from the group consisting of: transporting a mobile imaging device to a meal; aiming an imaging device at food; clicking a button to activate picture taking; touching a screen to activate picture taking; and speaking a voice command to activate picture taking.

In an example, a device for measuring a person's consumption of at least one selected type of food, ingredient, or nutrient can prompt a person to take pictures of food when a non-imaging sensor or other source of information indicates that the person is probably eating. In an alternative example, a device for measuring a person's consumption of at least one selected type of food, ingredient, or nutrient can automatically take pictures of food consumed without the need for specific action by the person in association with a specific eating event apart from the act of eating.

In an example, a device and method for measuring food consumption can include taking multiple pictures of food. In an example, such a device and method can include taking pictures of food from at least two different angles in order to better segment a meal into different types of foods, estimate the three-dimensional volume of each type of food, and control for lighting and shading differences. In an example, a camera or other imaging device can take pictures of food from multiple perspectives to create a virtual three-dimensional model of food in order to determine food volume. In an example, an imaging device can estimate the quantities of specific foods from pictures or images of those foods by volumetric analysis of food from multiple perspectives and/or by three-dimensional modeling of food from multiple perspectives.

In an example, a camera can use an object of known size within its field of view as a fiduciary marker in order to measure the size or scale of food. In an example, a camera can use projected laser beams to create a virtual or optical fiduciary marker in order to measure food size or scale. In an example, pictures of food can be taken at different times. In an example, a camera can be used to take pictures of food before and after consumption. The amount of food that a person actually consumes (not just the amount ordered or served) can be estimated by the difference in observed food volume from the pictures before and after consumption.

In an example, images of food can be automatically analyzed in order to identify the types and quantities of food consumed. In an example, pictures of food taken by a camera or other picture-taking device can be automatically analyzed to estimate the types and amounts of specific foods, ingredients, or nutrients that a person is consumes. In an example, an initial stage of an image analysis system can comprise adjusting, normalizing, or standardizing image elements for better food segmentation, identification, and volume estimation. These elements can include: color, texture, shape, size, context, geographic location, adjacent food, place setting context, and temperature (infrared). In an example, a device can identify specific foods from pictures or images by image segmentation, color analysis, texture analysis, and pattern recognition.

In various examples, automatic identification of food types and quantities can be based on: color and texture analysis; image segmentation; image pattern recognition; volumetric analysis based on a fiduciary marker or other object of known size; and/or three-dimensional modeling based on pictures from multiple perspectives. In an example, a device can collect food images that are used to extract a vector of food parameters (such as color, texture, shape, and size) that are automatically associated with vectors of food parameters in a database of such parameters for food identification.

In an example, a device can collect food images that are automatically associated with images of food in a food image database for food identification. In an example, specific ingredients or nutrients that are associated with these selected types of food can be estimated based on a database linking foods to ingredients and nutrients. In another example, specific ingredients or nutrients can be measured directly. In various examples, a device for measuring consumption of food, ingredient, or nutrients can directly (or indirectly) measure consumption at least one selected type of food, ingredient, or nutrient.

In an example, food image information can be transmitted from a wearable or hand-held device to a remote location where automatic food identification occurs and the results can be transmitted back to the wearable or hand-held device. In an example, identification of the types and quantities of foods, ingredients, or nutrients that a person consumes from pictures of food can be a combination of, or interaction between, automated identification food methods and human-based food identification methods.

We now transition to discussion of light-based methods for measuring food consumption that do not rely of conventional imaging devices and plain-light images. Probably the simplest such method involves identifying food by scanning a barcode or other machine-readable code on the food's packaging (such as a Universal Product Code or European Article Number), on a menu, on a store display sign, or otherwise in proximity to food at the point of food selection, sale, or consumption. In an example, the type of food (and/or specific ingredients or nutrients within the food) can be identified by machine-recognition of a food label, nutritional label, or logo on food packaging, menu, or display sign. However, there are many types of food and food consumption situations in which food is not accompanied by such identifying packaging. Accordingly, a robust imaged-based device and method for measuring food consumption should not rely on bar codes or other identifying material on food packaging.

In an example, selected types of foods, ingredients, and/or nutrients can be identified by the patterns of light that are reflected from, or absorbed by, the food at different wavelengths. In an example, a light-based sensor can detect food consumption or can identify consumption of a specific food, ingredient, or nutrient based on the reflection of light from food or the absorption of light by food at different wavelengths. In an example, an optical sensor can detect fluorescence. In an example, an optical sensor can detect whether food reflects light at a different wavelength than the wavelength of light shone on food. In an example, an optical sensor can be a fluorescence polarization immunoassay sensor, chemiluminescence sensor, thermoluminescence sensor, or piezoluminescence sensor.

In an example, a light-based food-identifying sensor can collect information concerning the wavelength spectra of light reflected from, or absorbed by, food. In an example, an optical sensor can be a chromatographic sensor, spectrographic sensor, analytical chromatographic sensor, liquid chromatographic sensor, gas chromatographic sensor, optoelectronic sensor, photochemical sensor, and photocell. In an example, an optical sensor can analyze modulation of light wave parameters by the interaction of that light with a portion of food. In an example, an optical sensor can detect modulation of light reflected from, or absorbed by, a receptor when the receptor is exposed to food. In an example, an optical sensor can emit and/or detect white light, infrared light, or ultraviolet light.

In an example, a light-based food-identifying sensor can identify consumption of a selected type of food, ingredient, or nutrient with a spectral analysis sensor. In various examples, a food-identifying sensor can identify a selected type of food, ingredient, or nutrient with a sensor that detects light reflection spectra, light absorption spectra, or light emission spectra. In an example, a spectral measurement sensor can be a spectroscopy sensor or a spectrometry sensor. In an example, a spectral measurement sensor can be a white light spectroscopy sensor, an infrared spectroscopy sensor, a near-infrared spectroscopy sensor, an ultraviolet spectroscopy sensor, an ion mobility spectroscopic sensor, a mass spectrometry sensor, a backscattering spectrometry sensor, or a spectrophotometer. In an example, light at different wavelengths can be absorbed by, or reflected off, food and the results can be analyzed in spectral analysis.

In an example, a food-consumption monitor or food-identifying sensor can be a microphone or other type of sound sensor. In an example, a sensor to detect food consumption and/or identify consumption of a selected type of food, ingredient, or nutrient can be a sound sensor. In an example, a sound sensor can be an air conduction microphone or bone conduction microphone. In an example, a microphone or other sound sensor can monitor for sounds associated with chewing or swallowing food. In an example, data collected by a sound sensor can be analyzed to differentiate sounds from chewing or swallowing food from other types of sounds such as speaking, singing, coughing, and sneezing.

In an example, a sound sensor can include speech recognition or voice recognition to receive verbal input from a person concerning food that the person consumes. In an example, a sound sensor can include speech recognition or voice recognition to extract food selecting, ordering, purchasing, or consumption information from other sounds in the environment.

In an example, a sound sensor can be worn or held by a person. In an example, a sound sensor can be part of a general purpose device, such as a cell phone or mobile phone, which has multiple applications. In an example, a sound sensor can measure the interaction of sound waves (such as ultrasonic sound waves) and food in order to identify the type and quantity of food that a person is eating.

In an example, a food-consumption monitor or food-identifying sensor can be a chemical sensor. In an example, a chemical sensor can include a receptor to which at least one specific nutrient-related analyte binds and this binding action creates a detectable signal. In an example, a chemical sensor can include measurement of changes in energy wave parameters that are caused by the interaction of that energy with food. In an example, a chemical sensor can be incorporated into a smart utensil to identify selected types of foods, ingredients, or nutrients. In an example, a chemical sensor can be incorporated into a portable food probe to identify selected types of foods, ingredients, or nutrients. In an example, a sensor can analyze the chemical composition of a person's saliva. In an example, a chemical sensor can be incorporated into an intraoral device that analyzes micro-samples of a person's saliva. In an example, such an intraoral device can be adhered to a person's upper palate.

In various examples, a food-consumption monitor or food-identifying sensor can be selected from the group consisting of: receptor-based sensor, enzyme-based sensor, reagent based sensor, antibody-based receptor, biochemical sensor, membrane sensor, pH level sensor, osmolality sensor, nucleic acid-based sensor, or DNA/RNA-based sensor; a biomimetic sensor (such as an artificial taste bud or an artificial olfactory sensor), a chemiresistor, a chemoreceptor sensor, a electrochemical sensor, an electroosmotic sensor, an electrophoresis sensor, or an electroporation sensor; a specific nutrient sensor (such as a glucose sensor, a cholesterol sensor, a fat sensor, a protein-based sensor, or an amino acid sensor); a color sensor, a colorimetric sensor, a photochemical sensor, a chemiluminescence sensor, a fluorescence sensor, a chromatography sensor (such as an analytical chromatography sensor, a liquid chromatography sensor, or a gas chromatography sensor), a spectrometry sensor (such as a mass spectrometry sensor), a spectrophotometer sensor, a spectral analysis sensor, or a spectroscopy sensor (such as a near-infrared spectroscopy sensor); and a laboratory-on-a-chip or microcantilever sensor.

In an example, a food-consumption monitor or food-identifying sensor can be an electromagnetic sensor. In an example, a device for measuring food consumption or identifying specific nutrients can emit and measure electromagnetic energy. In an example, a device can expose food to electromagnetic energy and collect data concerning the effects of this interaction which are used for food identification. In various examples, the results of this interaction can include measuring absorption or reflection of electromagnetic energy by food. In an example, an electromagnetic sensor can detect the modulation of electromagnetic energy that is interacted with food.

In an example, an electromagnetic sensor that detects food or nutrient consumption can detect electromagnetic signals from the body in response to the consumption or digestion of food. In an example, analysis of this electromagnetic energy can help to identify the types of food that a person consumes. In an example, a device can measure electromagnetic signals emitted by a person's stomach, esophagus, mouth, tongue, afferent nervous system, or brain in response to general food consumption. In an example, a device can measure electromagnetic signals emitted by a person's stomach, esophagus, mouth, tongue, afferent nervous system, or brain in response to consumption of selected types of foods, ingredients, or nutrients.

In various examples, a sensor to detect food consumption or identify consumption of a selected type of nutrient can be selected from the group consisting of: neuroelectrical sensor, action potential sensor, ECG sensor, EKG sensor, EEG sensor, EGG sensor, capacitance sensor, conductivity sensor, impedance sensor, galvanic skin response sensor, variable impedance sensor, variable resistance sensor, interferometer, magnetometer, RF sensor, electrophoretic sensor, optoelectronic sensor, piezoelectric sensor, and piezocapacitive sensor.

In an example, a sensor to monitor, detect, or sense food consumption or to identify a selected type of food, ingredient, or nutrient consumed can be pressure sensor or touch sensor. In an example, a pressure or touch sensor can sense pressure or tactile information from contact with food that will be consumed. In an example, a pressure or touch sensor can be incorporated into a smart food utensil or food probe. In an example, a pressure or touch based sensor can be incorporated into a pad on which a food utensil is placed between mouthfuls or when not in use. In an example, a pressure or touch sensor can sense pressure or tactile information from contact with a body member whose internal pressure or external shape is affected by food consumption. In various examples, a pressure or touch sensor can be selected from the group consisting of: food viscosity sensor, blood pressure monitor, muscle pressure sensor, button or switch on a food utensil, jaw motion pressure sensor, and hand-to-mouth contact sensor.

In an example, a food-consumption monitor or food-identifying sensor can be a thermal energy sensor. In an example, a thermal sensor can detect or measure the temperature of food. In an example, a thermal sensor can detect or measure the temperature of a portion of a person's body wherein food consumption changes the temperature of this member. In various examples, a food-consumption monitor can be selected from the group consisting of: a thermometer, a thermistor, a thermocouple, and an infrared energy detector.

In an example, a food-consumption monitor or food-identifying sensor can be a location sensor. In an example, such a sensor can be geographic location sensor or an intra-building location sensor. A device for detecting food consumption and/or indentifying a selected type of food, ingredient, or nutrient consumed can use information concerning a person's location as part of the means for food consumption detection and/or food identification. In an example, a device can identify when a person in a geographic location that is associated with probable food consumption. In an example, a device can use information concerning the person's geographic location as measured by a global positioning system or other geographic location identification system. In an example, if a person is located at a restaurant with a known menu or at a store with a known food inventory, then information concerning this menu or food inventory can be used to narrow down the likely types of food being consumed. In an example, if a person is located at a restaurant, then the sensitivity of automated detection of food consumption can be adjusted. In an example, if a person is located at a restaurant or grocery store, then visual, auditory, or other information collected by a sensor can be interpreted within the context of that location.

In an example, a device can identify when a person is in a location within a building that is associated with probable food consumption. In an example, if a person is in a kitchen or in a dining room within a building, then the sensitivity of automated detection of food consumption can be adjusted. In an example, a food-consumption monitoring system can increase the continuity or level of automatic data collection when a person is in a restaurant, in a grocery store, in a kitchen, or in a dining room. In an example, a person's location can be inferred from analysis of visual signals or auditory signals instead of via a global positioning system. In an example, a person's location can be inferred from interaction between a device and local RF beacons or local wireless networks.

In an example, a food-consumption monitor or food-identifying sensor can have a biological component. In an example, a food-identifying sensor can use biological or biomimetic components to identify specific foods, ingredients, or nutrients. In various examples, a food-identifying sensor can use one or more biological or biomimetic components selected from the group consisting of: biochemical sensor, antibodies or antibody-based chemical receptor, enzymes or enzyme-based chemical receptor, protein or protein-based chemical receptor, biomarker for a specific dietary nutrient, biomembrane or biomembrane-based sensor, porous polymer or filter paper containing a chemical reagent, nucleic acid-based sensor, polynucleotide-based sensor, artificial taste buds or biomimetic artificial tongue, and taste bud cells in communication with an electromagnetic sensor.

In an example, a food-consumption monitor or food-identifying sensor can be a taste or smell sensor. In an example, a sensor can be an artificial taste bud that emulates the function of a natural taste bud. In an example, a sensor can be an artificial olfactory receptor that emulates the function of a natural olfactory receptor. In an example, a sensor can comprise biological taste buds or olfactory receptors that are configured to be in electrochemical communication with an electronic device. In an example, a sensor can be an electronic tongue. In an example, a sensor can be an electronic nose.

In an example, a food-consumption monitor or food-identifying sensor can be a high-energy sensor. In an example, a high-energy sensor can identify a selected type of food, ingredient, or nutrient based on the interaction of microwaves or x-rays with a portion of food. In various examples a high-energy sensor to detect food consumption or identify consumption of a selected type of nutrient can be selected from the group consisting of: a microwave sensor, a microwave spectrometer, and an x-ray detector.

In an example, a person's consumption of food or the identification of a selected type of food, ingredient, or nutrient can be done by a sensor array. A sensor array can comprise multiple sensors of different types. In an example, multiple sensors in a sensor array can operate simultaneously in order to jointly identify food consumption or to jointly identify a selected type of food, ingredient, or nutrient. In an example, a sensor array can comprise multiple cross-reactive sensors. In an example, different sensors in a sensor array can operate independently to identify different types of foods, ingredients, or nutrients. In an example, a single sensor can detect different types of foods, ingredients, or nutrients.

In various examples, a food-consumption monitor or food-identifying sensor can be selected from the group consisting of: chemical sensor, biochemical sensor, amino acid sensor, chemiresistor, chemoreceptor, photochemical sensor, optical sensor, chromatography sensor, fiber optic sensor, infrared sensor, optoelectronic sensor, spectral analysis sensor, spectrophotometer, olfactory sensor, electronic nose, metal oxide semiconductor sensor, conducting polymer sensor, quartz crystal microbalance sensor, electromagnetic sensor, variable impedance sensor, variable resistance sensor, conductance sensor, neural impulse sensor, EEG sensor, EGG sensor, EMG sensor, interferometer, galvanic skin response sensor, cholesterol sensor, HDL sensor, LDL sensor, electrode, neuroelectrical sensor, neural action potential sensor, Micro Electrical Mechanical System (MEMS) sensor, laboratory-on-a-chip, or medichip, micronutrient sensor, osmolality sensor, protein-based sensor or reagent-based sensor, saturated fat sensor or trans fat sensor, action potential sensor, biological sensor, enzyme-based sensor, protein-based sensor, reagent-based sensor, camera, video camera, fixed focal-length camera, variable focal-length camera, pattern recognition sensor, microfluidic sensor, motion sensor, accelerometer, flow sensor, strain gauge, electrogoniometer, inclinometer, peristalsis sensor, multiple-analyte sensor array, an array of cross-reactive sensors, pH level sensor, sodium sensor, sonic energy sensor, microphone, sound-based chewing sensor, sound-based swallow sensor, ultrasonic sensor, sugar sensor, glucose sensor, temperature sensor, thermometer, and thermistor.

In an example, a sensor to monitor, detect, or sense food consumption or to identify consumption of a selected type of food, ingredient, or nutrient can be a wearable sensor that is worn by the person whose food consumption is monitored, detected, or sensed. In an example, a wearable food-consumption monitor or food-identifying sensor can be worn directly on a person's body. In an example a wearable food-consumption monitor or food-identifying sensor can be worn on, or incorporated into, a person's clothing.

In various examples, a wearable sensor can be worn on a person in a location selected from the group consisting of: wrist, neck, finger, hand, head, ear, eyes, nose, teeth, mouth, torso, chest, waist, and leg. In various examples, a wearable sensor can be attached to a person or to a person's clothing by a means selected from the group consisting of: strap, clip, clamp, snap, pin, hook and eye fastener, magnet, and adhesive.

In various examples, a wearable sensor can be worn on a person in a manner like a clothing accessory or piece of jewelry selected from the group consisting of: wristwatch, wristphone, wristband, bracelet, cufflink, armband, armlet, and finger ring; necklace, neck chain, pendant, dog tags, locket, amulet, necklace phone, and medallion; eyewear, eyeglasses, spectacles, sunglasses, contact lens, goggles, monocle, and visor; clip, tie clip, pin, brooch, clothing button, and pin-type button; headband, hair pin, headphones, ear phones, hearing aid, earring; and dental appliance, palatal vault attachment, and nose ring.

In an example, a sensor to monitor, detect, or sense food consumption or to identify consumption of a selected type of food, ingredient or nutrient can be a utensil-based sensor such as a spoon or fork. In an example, a utensil-based food-consumption monitor or food-identifying sensor can be attached to a generic food utensil. In an example, a utensil-based sensor can be incorporated into specialized “smart utensil.” In an example, a sensor can be attached to, or incorporated into a smart fork or smart spoon. In an example, a sensor can be attached to, or incorporated into, a beverage holding member such as a glass, cup, mug, or can. In an example, a food-identifying sensor can be incorporated into a portable food probe.

In an example, a wearable device for tracking food intake can comprise one or more sensors that are integrated into a place setting. In various examples, sensors can be integrated into one or more of the following place setting members: plate, glass, cup, bowl, serving dish, place mat, fork, spoon, knife, and smart utensil. In various examples, a place setting member can incorporate a sensor selected from the group consisting of: scale, camera, chemical receptor, spectroscopy sensor, infrared sensor, electromagnetic sensor. In an example, a place setting member with an integrated food sensor can collect data concerning food with which the place setting member is in contact at different times. In an example, changes in measurements concerning food at different times can be used to estimate the amount of food that a person is served, the amount of food that a person actually eats, and the amount of left-over food that a person does not eat.

In an example, a sensor to detect food consumption or to identify consumption of a selected type of food, ingredient, or nutrient can be incorporated into a multi-purpose mobile electronic device such as a cell phone, mobile phone, smart phone, smart watch, electronic tablet device, electronic book reader, electronically-functional jewelry, or other portable consumer electronics device. In an example, a smart phone application can turn the camera function of a smart phone into a means of food identification. In an example, such a smart phone application can be in wireless communication with a wearable device that is worn by the person whose food consumption is being measured.

In an example, a wearable device can prompt a person to collect information concerning food consumption using a smart phone application. In an example, a wearable device can automatically activate a smart phone or other portable electronic device to collect information concerning food consumption. In an example, a wearable device can automatically trigger a smart phone or other portable electronic device to start recording audio information using the smart phone's microphone when the wearable device detects that the person is probably eating. In an example, a wearable device can automatically trigger a smart phone or other portable electronic device to start recording visual information using the smart phone's camera when the wearable device detects that the person is probably eating.

In an example, a food-consumption monitor or specific food-identifying sensor can monitor, detect, and/or analyze chewing or swallowing actions by a person. In particular, such a monitor or sensor can differentiate between chewing and swallowing actions that are probably associated with eating vs. other activities. In various examples, chewing or swallowing can be monitored, detected, sensed, or analyzed based on sonic energy (differentiated from speaking, talking, singing, coughing, or other non-eating sounds), motion (differentiated from speaking or other mouth motions), imaging (differentiated from other mouth-related activities) or electromagnetic energy (such as electromagnetic signals from mouth muscles). There are differences in food consumed per chew or per swallow between people, and even for the same person over time, based on the type of food, the person's level of hunger, and other variables. This can make it difficult to estimate the amount of food consumed based only on the number of chews or swallows.

In an example, a food-consumption monitor or food-identifying sensor can monitor a particular body member. In various examples, such a monitor or sensor can be selected from the group consisting of: a blood monitor (for example using a blood pressure monitor, a blood flow monitor, or a blood glucose monitor); a brain monitor (such as an electroencephalographic monitor); a heart monitor (such as electrocardiographic monitor, a heartbeat monitor, or a pulse rate monitor); a mouth function monitor (such as a chewing sensor, a biting sensor, a jaw motion sensor, a swallowing sensor, or a saliva composition sensor); a muscle function monitor (such as an electromyographic monitor or a muscle pressure sensor); a nerve monitor or neural monitor (such as a neural action potential monitor, a neural impulse monitor, or a neuroelectrical sensor); a respiration monitor (such as a breathing monitor, an oxygen consumption monitor, an oxygen saturation monitor, a tidal volume sensor, or a spirometry monitor); a skin sensor (such as a galvanic skin response monitor, a skin conductance sensor, or a skin impedance sensor); and a stomach monitor (such as an electrogastrographic monitor or a stomach motion monitor). In various examples, a sensor can monitor sonic energy or electromagnetic energy from selected portions of a person's gastrointestinal tract (ranging from the mouth to the intestines) or from nerves which innervate those portions. In an example, a monitor or sensor can monitor peristaltic motion or other movement of selected portions of a person's gastrointestinal tract.

In an example, a monitor or sensor to detect food consumption or to identify a selected type of food, ingredient, or nutrient can be a micro-sampling sensor. In an example, a micro-sampling sensor can automatically extract and analyze micro-samples of food, intra-oral fluid, saliva, intra-nasal air, chyme, or blood. In an example, a micro-sampling sensor can collect and analyze micro-samples periodically. In an example, a micro-sampling sensor can collect and analyze micro-samples randomly. In an example, a micro-sampling sensor can collect and analyze micro-samples when a different sensor indicates that a person is probably consuming food. In an example, a micro-sampling sensor can be selected from the group consisting of: microfluidic sampling system, microfluidic sensor array, and micropump.

In an example, a sensor to detect food consumption and/or identify consumption of a selected type of food, ingredient, or nutrient can incorporate microscale or nanoscale technology. In various examples, a sensor to detect food consumption or identify a specific food, ingredient, or nutrient can be selected from the group consisting of: micro-cantilever sensor, microchip sensor, microfluidic sensor, nano-cantilever sensor, nanotechnology sensor, Micro Electrical Mechanical System (MEMS) sensor, laboratory-on-a-chip, and medichip.

In an example, a food-consumption monitor or food-identifying sensor can be incorporated into a smart watch or other device that is worn on a person's wrist. In an example, a food-consumption monitor or food-identifying sensor can be worn on, or attached to, other members of a person's body or to a person's clothing. In an example, a device for measuring a person's consumption of at least one selected type of food, ingredient, or nutrient can be worn on, or attached to, a person's body or clothing. In an example, a device can be worn on, or attached to, a part of a person's body that is selected from the group consisting of: wrist (one or both), hand (one or both), or finger; neck or throat; eyes (directly such as via contact lens or indirectly such as via eyewear); mouth, jaw, lips, tongue, teeth, or upper palate; arm (one or both); waist, abdomen, or torso; nose; ear; head or hair; and ankle or leg.

In an example, a device for measuring a person's consumption of at least one selected type of food, ingredient, or nutrient can be worn in a manner similar to a piece of jewelry or accessory. In various examples, a food consumption measuring device can be worn in a manner similar to a piece of jewelry or accessory selected from the group consisting of: smart watch, wrist band, wrist phone, wrist watch, fitness watch, or other wrist-worn device; finger ring or artificial finger nail; arm band, arm bracelet, charm bracelet, or smart bracelet; smart necklace, neck chain, neck band, or neck-worn pendant; smart eyewear, smart glasses, electronically-functional eyewear, virtual reality eyewear, or electronically-functional contact lens; cap, hat, visor, helmet, or goggles; smart button, brooch, ornamental pin, clip, smart beads; pin-type, clip-on, or magnetic button; shirt, blouse, jacket, coat, or dress button; head phones, ear phones, hearing aid, ear plug, or ear-worn bluetooth device; dental appliance, dental insert, upper palate attachment or implant; tongue ring, ear ring, or nose ring; electronically-functional skin patch and/or adhesive patch; undergarment with electronic sensors; head band, hair band, or hair clip; ankle strap or bracelet; belt or belt buckle; and key chain or key ring.

In an example, a device for measuring a person's consumption of at least one selected type of food, ingredient, or nutrient can be incorporated or integrated into an article of clothing or a clothing-related accessory. In various examples, a device for measuring food consumption can be incorporated or integrated into one of the following articles of clothing or clothing-related accessories: belt or belt buckle; neck tie; shirt or blouse; shoes or boots; underwear, underpants, briefs, undershirt, or bra; cap, hat, or hood; coat, jacket, or suit; dress or skirt; pants, jeans, or shorts; purse; socks; and sweat suit.

In an example, a device for measuring a person's consumption of at least one selected type of food, ingredient, or nutrient can be attached to a person's body or clothing. In an example, a device to measure food consumption can be attached to a person's body or clothing using an attachment means selected from the group consisting of: band, strap, chain, hook and eye fabric, ring, adhesive, bracelet, buckle, button, clamp, clip, elastic band, eyewear, magnet, necklace, piercing, pin, string, suture, tensile member, wrist band, and zipper. In an example, a device can be incorporated into the creation of a specific article of clothing. In an example, a device to measure food consumption can be integrated into a specific article of clothing by a means selected from the group consisting of: adhesive, band, buckle, button, clip, elastic band, hook and eye fabric, magnet, pin, pocket, pouch, sewing, strap, tensile member, and zipper.

In an example, a wearable device for measuring a person's consumption of at least one selected type of food, ingredient, or nutrient can comprise one or more sensors selected from the group consisting of: motion sensor, accelerometer (single multiple axis), electrogoniometer, or strain gauge; optical sensor, miniature still picture camera, miniature video camera, miniature spectroscopy sensor; sound sensor, miniature microphone, speech recognition software, pulse sensor, ultrasound sensor; electromagnetic sensor, skin galvanic response (Galvanic Skin Response) sensor, EMG sensor, chewing sensor, swallowing sensor; temperature sensor, thermometer, infrared sensor; and chemical sensor, chemical sensor array, miniature spectroscopy sensor, glucose sensor, cholesterol sensor, or sodium sensor.

In an example, a device and system for measuring a person's consumption of at least one selected type of food, ingredient, or nutrient can be entirely wearable or include a wearable component. In an example, a wearable device or component can be worn directly on a person's body, can be worn on a person's clothing, or can be integrated into a specific article of clothing. In an example, a wearable device for measuring food consumption can be in wireless communication with an external device. In various examples, a wearable device for measuring food consumption can be in wireless communication with an external device selected from the group consisting of: a cell phone, an electronic tablet, electronically-functional eyewear, a home electronics portal, an internet portal, a laptop computer, a mobile phone, a remote computer, a remote control unit, a smart phone, a smart utensil, a television set, and a virtual menu system.

In an example, a wearable device for measuring a person's consumption of at least one selected type of food, ingredient, or nutrient can comprise multiple components selected from the group consisting of: Central Processing Unit (CPU) or microprocessor; food-consumption monitoring component (motion sensor, electromagnetic sensor, optical sensor, and/or chemical sensor); graphic display component (display screen and/or coherent light projection); human-to-computer communication component (speech recognition, touch screen, keypad or buttons, and/or gesture recognition); memory component (flash, RAM, or ROM); power source and/or power-transducing component; time keeping and display component; wireless data transmission and reception component; and strap or band.

In an example, a device, method, and system for measuring consumption of selected types of foods, ingredients, or nutrients can include a hand-held component in addition to a wearable component. In an example, a hand-held component can be linked or combined with a wearable component to form an integrated system for measuring a person's consumption of at least one selected type of food, ingredient, or nutrient. In an example, the combination and integration of a wearable member and a hand-held member can provide advantages that are not possible with either a wearable member alone or a hand-held member alone. In an example, a wearable member of such a system can be a food-consumption monitor. In an example, a hand-held member of such a system can be a food-identifying sensor.

In an example, a wearable member can continually monitor to detect when the person is consuming food, wherein this continual monitoring does not significantly intrude on the person's privacy. In an example, a hand-held member may be potentially more intrusive with respect to privacy when it operates, but is only activated to operate when food consumption is detected by the wearable member. In an example, wearable and hand-held components of such a system can be linked by wireless communication. In an example, wearable and held-held components of such a system can be physically linked by a flexible wire. In an example, a hand-held component can be removably attached to the wearable member and detached for use in identifying at least one selected type of food, ingredient, or nutrient.

In an example, a hand-held component of a device or system for measuring a person's consumption of at least one selected type of food, ingredient, or nutrient can be a hand-held smart food utensil or food probe. In an example, a hand-held component of a device or system for measuring a person's consumption of at least one selected type of food, ingredient, or nutrient can be a hand-held mobile phone or other general consumer electronics device that performs multiple functions. In an example, a mobile phone application can link or integrate the operation of the mobile phone with the operation of a wearable component of a system for measuring a person's consumption of at least one selected type of food, ingredient, or nutrient.

In various examples, a hand-held component can be selected from the group consisting of: smart utensil, smart spoon, smart fork, smart food probe, smart bowl, smart chop stick, smart dish, smart glass, smart plate, electronically-functional utensil, electronically-functional spoon, electronically-functional fork, electronically-functional food probe, electronically-functional bowl, electronically-functional chop stick, electronically-functional dish, electronically-functional glass, electronically-functional plate, smart phone, mobile phone, cell phone, electronic tablet, and digital camera.

In various examples, a food-consumption monitoring and nutrient identifying system can comprise a combination of a wearable component and a hand-held component that is selected from the group consisting of: smart watch and smart food utensil; smart watch and food probe; smart watch and mobile phone; smart watch and electronic tablet; smart watch and digital camera; smart bracelet and smart food utensil; smart bracelet and food probe; smart bracelet and mobile phone; smart bracelet and electronic tablet; smart bracelet and digital camera; smart necklace and smart food utensil; smart necklace and food probe; smart necklace and mobile phone; smart necklace and electronic tablet; and smart necklace and digital camera.

In an example, a wearable food-consumption monitor (such as may be embodied in a smart watch, smart bracelet, or smart necklace) and a hand-held food-identifying sensor (such as may be embodied in a smart utensil, food probe, or smart phone) can be linked or combined together into an integrated system for measuring a person's consumption of at least one selected type of food, ingredient, or nutrient. In an example, wearable and held-held components such a system can be separate components that are linked by wireless communication. In an example, wearable and held-held components of such a system can be physically connected by a flexible element. In an example, wearable and hand-held components can be physically attached or detached for use. In an example, a hand-held component can be a removable part of a wearable component for easier portability and increased user compliance for all eating events. In an example, a smart utensil or food probe can be removed from a wearable component to identify food prior to, or during consumption. This can increase ease of use and user compliance with food identification for all eating events.

A smart food utensil can be a food utensil that is specifically designed to help measure a person's consumption of at least one selected type of food, ingredient, or nutrient. In an example, a smart utensil can be a food utensil that is equipped with electronic and/or sensory functionality. In an example, a smart food utensil can be designed to function like a regular food utensil, but is also enhanced with sensors in order to detect food consumption and/or identify consumption of selected types of foods, ingredients, or nutrients.

A regular food utensil can be narrowly defined as a tool that is commonly used to convey a single mouthful of food up to a person's mouth. In this narrow definition, a food utensil can be selected from the group consisting of: fork, spoon, spork, and chopstick. In an example, a food utensil can be more broadly defined as a tool that is used to apportion food into mouthfuls during food consumption or to convey a single mouthful of food up to a person's mouth during food consumption. This broader definition includes cutlery and knives used at the time of food consumption in addition to forks, spoons, sporks, and chopsticks.

In an example, a food utensil may be more broadly defined to also include tools and members that are used to convey amounts of food that are larger than a single mouthful and to apportion food into servings prior to food consumption by an individual. Broadly defined in such a manner, a food utensil can be selected from the group consisting of: fork, spoon, spork, knife, chopstick, glass, cup, mug, straw, can, tablespoon, teaspoon, ladle, scoop, spatula, tongs, dish, bowl, and plate. In an example, a smart utensil is an electronically-functional utensil. In an example, a smart utensil can be a utensil with one or built-in functions selected from the group consisting of: detecting use to convey food; detecting food consumption; measuring the speed, rate, or pace of food consumption; measuring the amount of food consumed; identifying the type of food consumed; and communicating information concerning food consumption to other devices or system components.

In an example, a food-consumption monitor or food-identifying sensor can be incorporated into, or attached to, a food utensil. In an example, such a sensor can be an integral part of a specialized smart utensil that is specifically designed to measure food consumption or detect consumption of at least one selected type of food, ingredient, or nutrient. In an example, such a sensor can be designed to be removably attached to a generic food utensil so that any generic utensil can be used. In an example, a sensor can be attached to a generic utensil by tension, a clip, an elastic band, magnetism, or adhesive.

In an example, such a sensor, or a smart utensil of which this sensor is a part, can be in wireless communication with a smart watch or other member that is worn on a person's wrist, hand, or arm. In this manner, a system or device can tell if a person is using the smart utensil when they eat based on the relative movements and/or proximity of a smart utensil to a smart watch. In an example, a smart utensil can be a component of a multi-component system to measure of person's consumption of at least one selected type of food, ingredient, or nutrient.

In an example, a smart food utensil or food probe can identify the types and amounts of consumed foods, ingredients, or nutrients by being in optical communication with food. In an example, a smart food utensil can identify the types and amounts of food consumed by taking pictures of food. In an example, a smart food utensil can take pictures of food that is within a reachable distance of a person. In an example, a smart food utensil can take pictures of food on a plate. In an example, a smart food utensil can take pictures of a portion of food as that food is conveyed to a person's mouth via the utensil.

In an example, a smart food utensil can identify the type of food by optically analyzing food being consumed. In an example, a smart food utensil can identify the types and amounts of food consumed by recording the effects light that is interacted with food. In an example, a smart food utensil can identify the types and amounts of food consumed via spectroscopy. In an example, a smart food utensil can perform spectroscopic analysis of a portion of food as that food is conveyed to a person's mouth via the utensil. In an example, a smart food utensil can measure the amount of food consumed using a photo-detector.

In an example, a smart food utensil or food probe can identify the types and amounts of consumed foods, ingredients, or nutrients by performing chemical analysis of food. In an example, a smart food utensil can identify the types and amounts of food consumed by performing chemical analysis of the chemical composition of food. In an example, a smart food utensil can collect data that is used to analyze the chemical composition of food by direct contact with food. In an example, a smart food utensil can identify the type of food, ingredient, or nutrient being consumed by being in fluid or gaseous communication with food. In an example, a smart food utensil can include an array of chemical sensors with which a sample of food interacts.

In an example, a smart food utensil can collect data that is used to analyze the chemical composition of food by measuring the absorption of light, sound, or electromagnetic energy by food that is in proximity to the person whose consumption is being monitored. In an example, a smart food utensil can collect data that is used to analyze the chemical composition of food by measuring the reflection of light, sound, or electromagnetic energy by food that is in proximity to the person whose consumption is being monitored. In an example, a smart food utensil can collect data that is used to analyze the chemical composition of food by measuring the reflection of different wavelengths of light, sound, or electromagnetic energy by food that is in proximity to the person whose consumption is being monitored.

In an example, a smart food utensil can identify the types and amounts of food consumed by measuring the effects of interacting food with electromagnetic energy. In an example, a smart food utensil can estimate the amount of food that a person consumes by tracking utensil motions with an accelerometer. In various examples, one or more sensors that are part of, or attached to, a smart food utensil can be selected from the group consisting of: motion sensor, accelerometer, strain gauge, inertial sensor, scale, weight sensor, or pressure sensor; miniature camera, video camera, optical sensor, optoelectronic sensor, spectrometer, spectroscopy sensor, or infrared sensor; chemical sensor, chemical receptor array, or spectroscopy sensor; microphone, sound sensor, or ultrasonic sensor; and electromagnetic sensor, capacitive sensor, inductance sensor, or piezoelectric sensor.

In an example, a wearable member (such as a smart watch) can continually monitor for possible food consumption, but a smart utensil is only used when the person is eating. In an example, a device or system for measuring food consumption can compare the motion of a smart utensil with the motion of a wearable member (such as a smart watch) in order to detect whether the smart utensil is being properly used whenever the person is eating food. In an example, a device or system for measuring food consumption can track the movement of a smart utensil that a person should use consistently to eat food, track the movement of a wearable motion sensor (such as a smart watch) that a person wears continuously, and compare the movements to determine whether the person always uses the smart utensil to eat. In an example, this device or system can prompt the person to use the smart utensil when comparison of the motion of the smart utensil with the motion of a wearable motion sensor (such as a smart watch) indicates that the person is not using the smart utensil when they are eating.

In an example, a device or system for measuring food consumption can monitor the proximity of a smart utensil to a wearable member (such as a smart watch) in order to detect whether the smart utensil is being properly used whenever the person is eating food. In an example, this device or system can prompt the person to use the smart utensil when lack of proximity between the smart utensil and a wearable member (such as a smart watch) indicates that the person is not using the smart utensil when they are eating. In an example, a device or system for measuring food consumption can detect if a smart utensil is attached to, or near to, a smart watch. In an example, a device or system for measuring food consumption can prompt a person to use a smart utensil if the smart utensil is not attached to, or near to, a smart watch when the person is eating.

In an example, a food-consumption monitoring and nutrient identifying system can include a hand-held component that is selected from the group consisting of: smart phone, mobile phone, cell phone, holophone, or application of such a phone; electronic tablet, other flat-surface mobile electronic device, Personal Digital Assistant (PDA), or laptop; digital camera; and smart eyewear, electronically-functional eyewear, or augmented reality eyewear. In an example, such a hand-held component can be in wireless communication with a wearable component of such a system. In an example, a device, method, or system for detecting food consumption or measuring consumption of a selected type of food, ingredient, or nutrient can include integration with a general-purpose mobile device that is used to collects data concerning food consumption. In an example, the hand-held component of such a system can be a general purpose device, of which collecting data for food identification is only one among many functions that it performs. In an example, a system for measuring a person's consumption of at least one selected type of food, ingredient, or nutrient can comprise: a wearable member that continually monitors for possible food consumption; a hand-held smart phone that is used to take pictures of food that will be consumed; wireless communication between the wearable member and the smart phone; and software that integrates the operation of the wearable member and the smart phone.

In an example, the hand-held component of a food-consumption monitoring and nutrient identifying system can be a general purpose smart phone which collects information concerning food by taking pictures of food. In an example, this smart phone can be in wireless communication with a wearable component of the system, such as a smart watch. In an example, the hand-held component of such a system must be brought into physical proximity with food that will be consumed in order to measure the results of interaction between food and light, sound, or electromagnetic energy.

In an example, a hand-held component of such a system requires voluntary action by a person in order to collect data for food identification in association with each eating event apart from the actual act of eating. In an example, a mobile phone must be pointed toward food by a person and triggered to take pictures of that food. In an example, a hand-held component of such a system must be brought into fluid or gaseous communication with food in order to chemically analyze the composition of food. In an example, a wearable member (such as a smart watch) can continually monitor for possible food consumption, but a smart phone is only used for food identification when the person is eating. In an example, this device or system can prompt the person to use a smart phone for food identification when the person is eating.

In an example, a smart phone can identify the types and amounts of consumed foods, ingredients, or nutrients by being in optical communication with food. In an example, a smart phone can collect information for identifying the types and amounts of food consumed by taking pictures of food. In an example, a smart phone can take pictures of food that is within a reachable distance of a person. In an example, a smart phone can take pictures of food on a plate.

In an example, a smart phone can collect data that is used to analyze the chemical composition of food by measuring the absorption of light, sound, or electromagnetic energy by food that is in proximity to the person whose consumption is being monitored. In an example, a smart phone can collect data that is used to analyze the chemical composition of food by measuring the reflection of different wavelengths of light, sound, or electromagnetic energy by food that is in proximity to the person whose consumption is being monitored. In various examples, one or more sensors that are part of, or attached to, a smart phone can be selected from the group consisting of: miniature camera, video camera, optical sensor, optoelectronic sensor, spectrometer, spectroscopy sensor, and infrared sensor.

In an example, a device for measuring a person's consumption of at least one selected type of food, ingredient, or nutrient can include a human-to-computer interface for communication from a human to a computer. In various examples, a device for measuring a person's consumption of at least one selected type of food, ingredient, or nutrient can include a human-to-computer interface selected from the group consisting of: speech recognition or voice recognition interface; touch screen or touch pad; physical keypad/keyboard, virtual keypad or keyboard, control buttons, or knobs; gesture recognition interface or holographic interface; motion recognition clothing; eye movement detector, smart eyewear, and/or electronically-functional eyewear; head movement tracker; conventional flat-surface mouse, 3D blob mouse, track ball, or electronic stylus; graphical user interface, drop down menu, pop-up menu, or search box; and neural interface or EMG sensor.

In an example, such a human-to-computer interface can enable a user to directly enter information concerning food consumption. In an example, such direct communication of information can occur prior to food consumption, during food consumption, and/or after food consumption. In an example, such a human-to-computer interface can enable a user to indirectly collect information concerning food consumption. In an example, such indirect collection of information can occur prior to food consumption, during food consumption, and/or after food consumption.

In an example, a device for measuring a person's consumption of at least one selected type of food, ingredient, or nutrient can include a computer-to-human interface for communication from a computer to a human. In an example, a device and method for monitoring and measuring a person's food consumption can provide feedback to the person. In an example, a computer-to-human interface can communicate information about the types and amounts of food that a person has consumed, should consume, or should not consume. In an example, a computer-to-human interface can provide feedback to a person concerning their eating habits and the effects of those eating habits. In an example, this feedback can prompt the person to collect more information concerning the types and amounts of food that the person is consuming. In an example, a computer-to-human interface can be used to not just provide information concerning eating behavior, but also to change eating behavior.

In various examples, a device for measuring a person's consumption of at least one selected type of food, ingredient, or nutrient can provide feedback to the person that is selected from the group consisting of: auditory feedback (such as a voice message, alarm, buzzer, ring tone, or song); feedback via computer-generated speech; mild external electric charge or neural stimulation; periodic feedback at a selected time of the day or week; phantom taste or smell; phone call; pre-recorded audio or video message by the person from an earlier time; television-based messages; and tactile, vibratory, or pressure-based feedback.

In various examples, a device for measuring a person's consumption of at least one selected type of food, ingredient, or nutrient can provide feedback to the person that is selected from the group consisting of: feedback concerning food consumption (such as types and amounts of foods, ingredients, and nutrients consumed, calories consumed, calories expended, and net energy balance during a period of time); information about good or bad ingredients in nearby food; information concerning financial incentives or penalties associated with acts of food consumption and achievement of health-related goals; information concerning progress toward meeting a weight, energy-balance, and/or other health-related goal; information concerning the calories or nutritional components of specific food items; and number of calories consumed per eating event or time period.

In various examples, a device for measuring a person's consumption of at least one selected type of food, ingredient, or nutrient can provide feedback to the person that is selected from the group consisting of: augmented reality feedback (such as virtual visual elements superimposed on foods within a person's field of vision); changes in a picture or image of a person reflecting the likely effects of a continued pattern of food consumption; display of a person's progress toward achieving energy balance, weight management, dietary, or other health-related goals; graphical display of foods, ingredients, or nutrients consumed relative to standard amounts (such as embodied in pie charts, bar charts, percentages, color spectrums, icons, emoticons, animations, and morphed images); graphical representations of food items; graphical representations of the effects of eating particular foods; holographic display; information on a computer display screen (such as a graphical user interface); lights, pictures, images, or other optical feedback; touch screen display; and visual feedback through electronically-functional eyewear.

In various examples, a device for measuring a person's consumption of at least one selected type of food, ingredient, or nutrient can provide feedback to the person that is selected from the group consisting of: advice concerning consumption of specific foods or suggested food alternatives (such as advice from a dietician, nutritionist, nurse, physician, health coach, other health care professional, virtual agent, or health plan); electronic verbal or written feedback (such as phone calls, electronic verbal messages, or electronic text messages); live communication from a health care professional; questions to the person that are directed toward better measurement or modification of food consumption; real-time advice concerning whether to eat specific foods and suggestions for alternatives if foods are not healthy; social feedback (such as encouragement or admonitions from friends and/or a social network); suggestions for meal planning and food consumption for an upcoming day; and suggestions for physical activity and caloric expenditure to achieve desired energy balance outcomes.

In an example, a wearable and/or hand-held member of a device for measuring a person's consumption of at least one selected type of food, ingredient, or nutrient can comprise multiple components selected from the group consisting of: a food-consumption monitor or food-identifying sensor; a central processing unit (CPU) such as a microprocessor; a database of different types of food and food attributes; a memory to store, record, and retrieve data such as the cumulative amount consumed for at least one selected type of food, ingredient, or nutrient; a communications member to transmit data to from external sources and to receive data from external sources; a power source such as a battery or power transducer; a human-to-computer interface such as a touch screen, keypad, or voice recognition interface; and a computer-to-human interface such as a display screen or voice-producing interface.

In an example, the power source for a wearable and/or hand-held member of a device for measuring a person's consumption of at least one selected type of food, ingredient, or nutrient can be selected from the group consisting of: power from a power source that is internal to the device during regular operation (such as an internal battery, capacitor, energy-storing microchip, or wound coil or spring); power that is obtained, harvested, or transduced from a power source other than the person's body that is external to the device (such as a rechargeable battery, electromagnetic inductance from external source, solar energy, indoor lighting energy, wired connection to an external power source, ambient or localized radiofrequency energy, or ambient thermal energy); and power that is obtained, harvested, or transduced from the person's body (such as kinetic or mechanical energy from body motion, electromagnetic energy from the person's body, blood flow or other internal fluid flow, glucose metabolism, or thermal energy from the person's body.

In an example, a device or system for measuring a person's consumption of at least one selected type of food, ingredient, or nutrient can include one or more communications components for wireless transmission and reception of data. In an example, multiple communications components can enable wireless communication (including data exchange) between separate components of such a device and system. In an example, a communications component can enable wireless communication with an external device or system. In various examples, the means of this wireless communication can be selected from the group consisting of: radio transmission, Bluetooth transmission, Wi-Fi, and infrared energy.

In various examples, a device and system for measuring food consumption can be in wireless communication with an external device or system selected from the group consisting of: internet portal; smart phone, mobile phone, cell phone, holophone, or application of such a phone; electronic tablet, other flat-surface mobile electronic device, Personal Digital Assistant (PDA), remote control unit, or laptop; smart eyewear, electronically-functional eyewear, or augmented reality eyewear; electronic store display, electronic restaurant menu, or vending machine; and desktop computer, television, or mainframe computer. In various examples, a device can receive food-identifying information from a source selected from the group consisting of: electromagnetic transmissions from a food display or RFID food tag in a grocery store, electromagnetic transmissions from a physical menu or virtual user interface at a restaurant, and electromagnetic transmissions from a vending machine.

In an example, data concerning food consumption that is collected by a wearable or hand-held device can be analyzed by a data processing unit within the device in order to identify the types and amounts of foods, ingredients, or nutrients that a person consumes. In an example, data concerning food consumption that is collected by a smart watch can be analyzed within the housing of the watch. In an example, data concerning food consumption that is collected by a smart food utensil can be analyzed within the housing of the utensil.

In another example, data concerning food consumption that is collected by a wearable or hand-held device can be transmitted to an external device or system for analysis at a remote location. In an example, pictures of food can be transmitted to an external device or system for food identification at a remote location. In an example, chemical analysis results can be transmitted to an external device or system for food identification at a remote location. In an example, the results of analysis at a remote location can be transmitted back to a wearable or hand-held device.

In an example, a device for measuring a person's consumption of at least one selected type of food, ingredient, or nutrient can identify and track the selected types and amounts of foods, ingredients, or nutrients that the person consumes in an entirely automatic manner. In an example, such identification can occur in a partially automatic manner in which there is interaction between automated and human identification methods.

In an example, a device for measuring a person's consumption of at least one selected type of food, ingredient, or nutrient can identify and track food consumption at the point of selection or point of sale. In an example, a device for monitoring food consumption or consumption of selected types of foods, ingredients, or nutrients can approximate such measurements by tracking a person's food selections and purchases at a grocery store, at a restaurant, or via a vending machine. Tracking purchases can be relatively easy to do, since financial transactions are already well-supported by existing information technology. In an example, such tracking can be done with specific methods of payment, such as a credit card or bank account. In an example, such tracking can be done with electronically-functional food identification means such as bar codes, RFID tags, or electronically-functional restaurant menus. Electronic communication for food identification can also occur between a wearable device to track food intake and a vending machine.

In an example, a device for measuring a person's consumption of at least one selected type of food, ingredient, or nutrient can identify food using information from a food's packaging or container. In an example, this information can be detected optically by means of a picture or optical scanner. In an example, food can be identified directly by automated optical recognition of information on food packaging, such as a logo, label, or barcode. In various examples, optical information on a food's packaging or container that is used to identify the type and/or amount of food can be selected from the group consisting of: bar code, food logo, food trademark design, nutritional label, optical text recognition, and UPC code. With respect to meals ordered at restaurants, some restaurants (especially fast-food restaurants) have standardized menu items with standardized food ingredients. In such cases, identification of types and amounts of food, ingredients, or nutrients can be conveyed at the point of ordering (via an electronically-functional menu) or purchase (via purchase transaction). In an example, food can be identified directly by wireless information received from a food display, RFID tag, electronically-functional restaurant menu, or vending machine. In an example, food or its nutritional composition can be identified directly by wireless transmission of information from a food display, menu, food vending machine, food dispenser, or other point of food selection or sale and a device that is worn, held, or otherwise transported with a person.

However, there are limitations to estimating food consumption based on food selections or purchases in a store or restaurant. First, a person might not eat everything that they purchase through venues that are tracked by the system. The person might purchase food that is eaten by their family or other people and might throw out some of the food that they purchase. Second, a person might eat food that they do not purchase through venues that are tracked by the system. The person might purchase some food with cash or in venues that are otherwise not tracked. The person might eat food that someone else bought, as when eating as a guest or family member. Third, timing differences between when a person buys food and when they eat it, especially for non-perishable foods, can confound efforts to associate caloric intake with caloric expenditure to manage energy balance during a defined period of time. For these reasons, a robust device for measuring food consumption should (also) be able to identify food at the point of consumption.

In an example, a device, method, or system for measuring a person's consumption of at least one selected type of food, ingredient, or nutrient can identify and track a person's food consumption at the point of consumption. In an example, such a device, method, or system can include a database of different types of food. In an example, such a device, method, or system can be in wireless communication with an externally-located database of different types of food. In an example, such a database of different types of food and their associated attributes can be used to help identify selected types of foods, ingredients, or nutrients. In an example, a database of attributes for different types of food can be used to associate types and amounts of specific ingredients, nutrients, and/or calories with selected types and amounts of food.

In an example, such a database of different types of foods can include one or more elements selected from the group consisting of: food color, food name, food packaging bar code or nutritional label, food packaging or logo pattern, food picture (individually or in combinations with other foods), food shape, food texture, food type, common geographic or intra-building locations for serving or consumption, common or standardized ingredients (per serving, per volume, or per weight), common or standardized nutrients (per serving, per volume, or per weight), common or standardized size (per serving), common or standardized number of calories (per serving, per volume, or per weight), common times or special events for serving or consumption, and commonly associated or jointly-served foods.

In an example, a picture of a meal as a whole can be automatically segmented into portions of different types of food for comparison with different types of food in a food database. In an example, the boundaries between different types of food in a picture of a meal can be automatically determined to segment the meal into different food types before comparison with pictures in a food database. In an example, a picture of a meal with multiple types of food can be compared as a whole with pictures of meals with multiple types of food in a food database. In an example, a picture of a food or a meal comprising multiple types of food can be compared directly with pictures of food in a food database.

In an example, a picture of food or a meal comprising multiple types of food can be adjusted, normalized, or standardized before it is compared with pictures of food in a food database. In an example, food color can be adjusted, normalized, or standardized before comparison with pictures in a food database. In an example, food size or scale can be adjusted, normalized, or standardized before comparison with pictures in a food database. In an example, food texture can be adjusted, normalized, or standardized before comparison with pictures in a food database. In an example, food lighting or shading can be adjusted, normalized, or standardized before comparison with pictures in a food database.

In an example, a food database can be used to identify the amount of calories that are associated with an indentified type and amount of food. In an example, a food database can be used to identify the type and amount of at least one selected type of food that a person consumes. In an example, a food database can be used to identify the type and amount of at least one selected type of ingredient that is associated with an identified type and amount of food. In an example, a food database can be used to identify the type and amount of at least one selected type of nutrient that is associated with an identified type and amount of food. In an example, an ingredient or nutrient can be associated with a type of food on a per-portion, per-volume, or per-weight basis.

In an example, a vector of food characteristics can be extracted from a picture of food and compared with a database of such vectors for common foods. In an example, analysis of data concerning food consumption can include comparison of food consumption parameters between a specific person and a reference population. In an example, data analysis can include analysis of a person's food consumption patterns over time. In an example, such analysis can track the cumulative amount of at least one selected type of food, ingredient, or nutrient that a person consumes during a selected period of time.

In various examples, data concerning food consumption can be analyzed to identify and track consumption of selected types and amounts of foods, ingredients, or nutrient consumed using one or more methods selected from the group consisting of: linear regression and/or multivariate linear regression, logistic regression and/or probit analysis, Fourier transformation and/or fast Fourier transform (FFT), linear discriminant analysis, non-linear programming, analysis of variance, chi-squared analysis, cluster analysis, energy balance tracking, factor analysis, principal components analysis, survival analysis, time series analysis, volumetric modeling, neural network and machine learning.

In an example, a device for measuring a person's consumption of at least one selected type of food, ingredient, or nutrient can identify the types and amounts of food consumed in an automated manner based on images of that food. In various examples, food pictures can be analyzed for automated food identification using methods selected from the group consisting of: image attribute adjustment or normalization; inter-food boundary determination and food portion segmentation; image pattern recognition and comparison with images in a food database to identify food type; comparison of a vector of food characteristics with a database of such characteristics for different types of food; scale determination based on a fiduciary marker and/or three-dimensional modeling to estimate food quantity; and association of selected types and amounts of ingredients or nutrients with selected types and amounts of food portions based on a food database that links common types and amounts of foods with common types and amounts of ingredients or nutrients. In an example, automated identification of selected types of food based on images and/or automated association of selected types of ingredients or nutrients with that food can occur within a wearable or hand-held device. In an example, data collected by a wearable or hand-held device can be transmitted to an external device where automated identification occurs and the results can then be transmitted back to the wearable or hand-held device.

In an example, a device and system for measuring a person's consumption of at least one selected type of food, ingredient, or nutrient can take pictures of food using a digital camera. In an example, a device and system for measuring a person's consumption of at least one selected type of food, ingredient, or nutrient can take pictures of food using an imaging device selected from the group consisting of: smart watch, smart bracelet, fitness watch, fitness bracelet, watch phone, bracelet phone, wrist band, or other wrist-worn device; arm bracelet; and smart ring or finger ring. In an example, a device and system for measuring a person's consumption of at least one selected type of food, ingredient, or nutrient can take pictures of food using an imaging device selected from the group consisting of: smart phone, mobile phone, cell phone, holophone, and electronic tablet.

In an example, a device and system for measuring a person's consumption of at least one selected type of food, ingredient, or nutrient can take pictures of food using an imaging device selected from the group consisting of: smart glasses, visor, or other eyewear; electronically-functional glasses, visor, or other eyewear; augmented reality glasses, visor, or other eyewear; virtual reality glasses, visor, or other eyewear; and electronically-functional contact lens. In an example, a device and system for measuring a person's consumption of at least one selected type of food, ingredient, or nutrient can take pictures of food using an imaging device selected from the group consisting of: smart utensil, fork, spoon, food probe, plate, dish, or glass; and electronically-functional utensil, fork, spoon, food probe, plate, dish, or glass. In an example, a device and system for measuring a person's consumption of at least one selected type of food, ingredient, or nutrient can take pictures of food using an imaging device selected from the group consisting of: smart necklace, smart beads, smart button, neck chain, and neck pendant.

In an example, an imaging device can take multiple still pictures or moving video pictures of food. In an example, an imaging device can take multiple pictures of food from different angles in order to perform three-dimensional analysis or modeling of the food to better determine the volume of food. In an example, an imaging device can take multiple pictures of food from different angles in order to better control for differences in lighting and portions of food that are obscured from some perspectives. In an example, an imaging device can take multiple pictures of food from different angles in order to perform three-dimensional modeling or volumetric analysis to determine the three-dimensional volume of food in the picture. In an example, an imaging device can take multiple pictures of food at different times, such as before and after an eating event, in order to better determine how much food the person actually ate (as compared to the amount of food served). In an example, changes in the volume of food in sequential pictures before and after consumption can be compared to the cumulative volume of food conveyed to a person's mouth by a smart utensil to determine a more accurate estimate of food volume consumed. In various examples, a person can be prompted by a device to take pictures of food from different angles or at different times.

In an example, a device that indentifies a person's food consumption based on images of food can receive food images from an imaging component or other imaging device that the person holds in their hand to operate. In an example, a device that indentifies a person's food consumption based on images of food can receive food images from an imaging component or other imaging device that the person wears on their body or clothing. In an example, a wearable imaging device can be worn in a relatively fixed position on a person's neck or torso so that it always views the space in front of a person. In an example, a wearable imaging device can be worn on a person's wrist, arm, or finger so that the field of vision of the device moves as the person moves their arm, wrist, and/or fingers. In an example, a device with a moving field of vision can monitor both hand-to-food interaction and hand-to-mouth interaction as the person moves their arm, wrist, and/or hand. In an example, a wearable imaging device can comprise a smart watch with a miniature camera that monitors the space near a person's hands for possible hand-to-food interaction and monitors the near a person's mouth for hand-to-mouth interaction.

In an example, selected attributes or parameters of a food image can be adjusted, standardized, or normalized before the food image is compared to images in a database of food images or otherwise analyzed for identifying the type of food. In various examples, these image attributes or parameters can be selected from the group consisting of: food color, food texture, scale, image resolution, image brightness, and light angle.

In an example, a device and system for identifying types and amounts of food consumed based on food images can include the step of automatically segmenting regions of a food image into different types or portions of food. In an example, a device and system for identifying types and amounts of food consumed based on food images can include the step of automatically identifying boundaries between different types of food in an image that contains multiple types or portions of food. In an example, the creation of boundaries between different types of food and/or segmentation of a meal into different food types can include edge detection, shading analysis, texture analysis, and three-dimensional modeling. In an example, this process can also be informed by common patterns of jointly-served foods and common boundary characteristics of such jointly-served foods.

In an example, estimation of specific ingredients or nutrients consumed from information concerning food consumed can be done using a database that links specific foods (and quantities thereof) with specific ingredients or nutrients (and quantities thereof). In an example, food in a picture can be classified and identified based on comparison with pictures of known foods in a food image database. In an example, such food identification can be assisted by pattern recognition software. In an example, types and quantities of specific ingredients or nutrients can be estimated from the types and quantities of food consumed.

In an example, attributes of food in an image can be represented by a multi-dimensional food attribute vector. In an example, this food attribute vector can be statistically compared to the attribute vector of known foods in order to automate food identification. In an example, multivariate analysis can be done to identify the most likely identification category for a particular portion of food in an image. In various examples, a multi-dimensional food attribute vector can include attributes selected from the group consisting of: food color; food texture; food shape; food size or scale; geographic location of selection, purchase, or consumption; timing of day, week, or special event; common food combinations or pairings; image brightness, resolution, or lighting direction; infrared light reflection; spectroscopic analysis; and person-specific historical eating patterns.

In an example, a method for measuring a person's consumption of at least one selected type of food, ingredient, or nutrient can comprise collecting primary data concerning food consumption and collecting secondary data concerning food consumption. In an example, a device and system for measuring a person's consumption of at least one selected type of food, ingredient, or nutrient can comprise a primary data collection component and a secondary data collection component. In an example, primary data and secondary data can be jointly analyzed to identify the types and amounts of foods, ingredients, or nutrients that a person consumes.

In an example, primary data collection can occur automatically, without the need for any specific action by a person in association with a specific eating event, apart from the actual act of eating. In an example, a primary data component can operate automatically, without the need for any specific action by the person in association with a specific eating event apart from the actual act of eating. In an example, primary data is collected continuously, but secondary data is only collected when primary data indicates that a person is probably eating food. In an example, a primary data collection component operates continuously, but a secondary data collection component only operates when primary data indicates that a person is probably eating food.

In an example, primary data is collected automatically, but secondary data is only collected when triggered, activated, or operated by a person via a specific action in association with a specific eating event other than the act of eating. In an example, a primary data collection component operates automatically, but a secondary data collection component only operates when it is triggered, activated, or operated by a person via a specific action in association with a specific eating event other than the act of eating.

In an example, collection of secondary data can require a specific triggering or activating action by a person, apart from the act of eating, for each specific eating event. In an example, a device to measure food consumption can prompt a person to trigger, activate, or operate secondary data collection in association with a specific eating event when analysis of primary data indicates that this person is probably eating. In an example, a device to measure food consumption can prompt a person to trigger, activate, or operate a secondary data collection component in association with a specific eating event when analysis of primary data indicates that this person is probably eating. In an example, a component of this device that automatically collects primary data to detect when a person is probably eating can prompt the person to collect secondary data to identify food consumed when the person is probably eating. In an example, a device can prompt a person to collect secondary data in association with a specific eating event when analysis of primary data indicates that the person is probably eating and the person has not yet collected secondary data.

In an example, primary data can be collected by a wearable member and secondary data can be collected by a hand-held member. In an example, a person can be prompted to use a hand-held member to collect secondary data when primary data indicates that this person is probably eating. In an example, the wearable member can detect when a person is eating something, but is not very good at identifying what selected types of food the person is eating. In an example, the hand-held member is better at identifying what selected types of food the person is eating, but only when the hand-held member is used, which requires specific action by the person for each eating event.

In an example, a device and system can prompt a person to use a hand-held member (such as a mobile phone or smart utensil) to take pictures of food when a wearable member (such as a smart watch or smart bracelet) indicates that the person is probably eating. In an example, a person can be prompted to use a digital camera to take pictures of food when a wearable food-consumption monitor detects that the person is consuming food.

In an example, a person can be prompted to use a smart utensil to take pictures of food when a wearable food-consumption monitor detects that the person is consuming food. In an example, a device and system can prompt a person to use a hand-held member (such as a smart utensil or food probe) to analyze the chemical composition of food when a wearable member (such as a smart watch or smart bracelet) indicates that the person is probably eating. In an example, a person can be prompted to use a smart utensil for chemical analysis of food when a wearable food-consumption monitor detects that the person is consuming food.

In an example, a device for measuring food consumption can prompt a person to collect secondary data in real time, while a person is eating, when food consumption is indicated by primary data. In an example, a device for measuring food consumption can prompt a person to collect secondary data after food consumption, after food consumption has been indicated by primary data. In various examples, a device can prompt a person to take one or more actions to collect secondary data that are selected from the group consisting of: use a specific smart utensil for food consumption; use a specific set of smart place setting components (dish, plate, utensils, glass, etc) to record information about types and quantities of food; use a special food scale; touch food with a food probe or smart utensil; take a still picture or multiple still pictures of food from different angles; record a video of food from different angles; and expose food to light, electromagnetic, microwave, sonic, or other energy and record the results of interaction between food and this energy.

In an example, the process of collecting primary data can be less intrusive than the process of collecting secondary data with respect to a person's privacy. In an example, secondary data can enable more accurate food identification than primary data with respect to measuring a person's consumption of at least one selected type of food, ingredient, or nutrient. In an example, a coordinated system of primary and secondary data collection can achieve a greater level of measurement accuracy for a selected level of privacy intrusion than either primary data collection or secondary data collection alone. In an example, a coordinated system of primary and secondary data collection can achieve a lower level of privacy intrusion for a selected level of measurement accuracy than either primary data collection or secondary data collection alone.

In an example, primary data can be collected by a device or device component that a person wears on their body or clothing. In an example, primary data can be collected by a smart watch, smart bracelet, or other wrist-worn member. In an example, primary data can be collected by a smart necklace or other neck-worn member. In an example, primary data can be collected by smart glasses or other electronically-functional eyewear. In an example, primary data can be data concerning a person's movements that is collected using a motion detector. In an example, a primary data collection component can monitor a person's movements for movements that indicate that the person is probably eating food. In an example, primary data can be data concerning electromagnetic signals from a person's body. In an example, a primary data collection component can monitor electromagnetic signals from the person's body for signals that indicate that the person is probably eating food.

In an example, secondary data can be collected by a device or device component that a person holds in their hand. In an example, secondary data can be collected by a smart phone, mobile phone, smart utensil, or smart food probe. In an example, secondary data can be images of food. In an example, collection of secondary data can require that the person aim a camera at food and take one or more pictures of food. In an example, a camera-based food-identifying sensor automatically starts taking pictures when data collected by the monitor indicates that a person is probably consuming food, but the person is prompted to manually aim the camera toward food being consumed when data collected by the monitor indicates that a person is probably consuming food.

In an example, secondary data can be the results of chemical analysis of food. In an example, collection of secondary data can require that the person bring a nutrient-identifying utensil or sensor into physical contact with food. In an example, collection of secondary data can require that the person speak into a voice-recognizing device and verbally identify the food that they are eating. In an example, collection of secondary data can require that the person use a computerized menu-interface to identify the food that they are eating.

In an example, a device for measuring a person's consumption of at least one selected type of food, ingredient, or nutrient can collect primary data concerning food consumption without the need for a specific action by the person in association with an eating event apart from the act of eating. In an example, a device for measuring a person's consumption of at least one selected type of food, ingredient, or nutrient can collect primary data automatically. In an example, a device for measuring a person's consumption of at least one selected type of food, ingredient, or nutrient can collect primary data continually.

In an example, a device for measuring a person's consumption of at least one selected type of food, ingredient, or nutrient automatically collects secondary data concerning food consumption during a specific eating event, but only when analysis of primary data indicates that the person is eating. In an example, a device for measuring a person's consumption of at least one selected type of food, ingredient, or nutrient only collects secondary data concerning food consumption during a specific eating when it is triggered, activated, or operated by the person for that eating event by an action apart from the act of eating. In an example, a device can prompt the person to trigger, activate, or operate secondary data collection when primary data indicates that the person is eating.

In an example, a device for measuring a person's food consumption can automatically start collecting secondary data when primary data detects: reachable food sources; hand-to-food interaction; physical location in a restaurant, kitchen, dining room, or other location associated with probable food consumption; hand or arm motions associated with bringing food up to the person's mouth; physiologic responses by the person's body that are associated with probable food consumption; smells or sounds that are associated with probable food consumption; and/or speech patterns that are associated with probable food consumption.

In an example, a device for measuring a person's food consumption can prompt a person to collect secondary data when primary data detects: reachable food sources; hand-to-food interaction; physical location in a restaurant, kitchen, dining room, or other location associated with probable food consumption; hand or arm motions associated with bringing food up to the person's mouth; physiologic responses by the person's body that are associated with probable food consumption; smells or sounds that are associated with probable food consumption; and/or speech patterns that are associated with probable food consumption.

In an example, a device for measuring a person's consumption of at least one selected type of food, ingredient, or nutrient can include a combination of food identification methods or steps that are performed automatically by a computer and food identification methods or steps that are performed by a human. In an example, a device and method for detecting food consumption and identifying consumption of specific ingredients or nutrients can comprise multiple types of data collection and analysis involving interaction between automated analysis and human entry of information. In an example, a person can play a role in segmenting an image of a multi-food meal into different types of food by creating a virtual boundary between foods, such as by moving their finger across a touch-screen image of the meal. In an example, the person may review images of food consumed after an eating event and manually enter food identification information. In an example, a person can select one or more food types and/or quantities from a menu provided in response to a picture or other recorded evidence of an eating event.

In an example, redundant food identification can be performed by both a computer and a human during a calibration period, after which food identification is performed only by a computer. In an example, a device and system can automatically calibrate sensors and responses based on known quantities and outcomes. In an example, a person can eat food with known amounts of specific ingredients or nutrients. In an example, measured amounts can be compared to known amounts in order to calibrate device or system sensors. In an example, a device and system can track actual changes in a person's weight or Body Mass Index (BMI) and use these actual changes to calibrate device or system sensors. In an example, a device or system for measuring a person's consumption of at least one specific food, ingredient, or nutrient can be capable of adaptive machine learning. In an example, such a device or system can include a neural network. In an example, such a device and system can iteratively adjust the weights given to human responses based on feedback and health outcomes

In an example, initial estimates of the types and amounts of food consumed can be made by a computer in an automated manner and then refined by human review as needed. In an example, if automated methods for identification of the types and amounts of food consumed do not produce results with a required level of certainty, then a device and system can prompt a person to collect and/or otherwise provide supplemental information concerning the types of food that the person is consuming. In an example, a device and system can track the accuracy of food consumption information provided by an automated process vs. that provided by a human by comparing predicted to actual changes in a person's weight. In an example, the relative weight which a device and system places on information from automated processes vs. information from human input can be adjusted based on their relatively accuracy in predicting weight changes. Greater weight can be given to the information source which is more accurate based on empirical validation.

In an example, a device can ask a person clarifying questions concerning food consumed. In an example, a device can prompt the person with queries to refine initial automatically-generated estimates of the types and quantities of food consumed. In an example, these questions can be asked in real time, as a person is eating, or in a delayed manner, after a person has finished eating or at a particular time of the day. In an example, the results of preliminary automated food identification can be presented to a human via a graphical user interface and the human can then refine the results using a touch screen. In an example, the results of automated food identification can be presented to a human via verbal message and the human can refine the results using a speech recognition interface. In an example, data can be transmitted (such as by the internet) to a review center where food is identified by a dietician or other specialist. In various examples, a human-to-computer interface for entering information concerning food consumption can comprise one or more interface elements selected the group consisting of: microphone, speech recognition, and/or voice recognition interface; touch screen, touch pad, keypad, keyboard, buttons, or other touch-based interface; camera, motion recognition, gesture recognition, eye motion tracking, or other motion detection interface; interactive food-identification menu with food pictures and names; and interactive food-identification search box.

In an example, a device and method for measuring consumption of a selected type of food, ingredient, or nutrient can comprise: a wearable motion sensor that is worn by a person that automatically collects data concerning the person's body motion, wherein this body motion data is used to determine when this person is consuming food; and a user interface that prompts the person to provide additional information concerning the selected types of foods, ingredients, or nutrients that the person is eating when the body motion data indicates that the person is consuming food.

In an example, a device and method for measuring consumption of a selected type of food, ingredient, or nutrient can comprise: a wearable sound sensor that is worn by a person that automatically collects data concerning sounds from the person's body or the environment, wherein this sound data is used to determine when this person is consuming food; and a user interface that prompts the person to provide additional information concerning the selected types of foods, ingredients, or nutrients that the person is eating when the sound data indicates that the person is consuming food.

In an example, a device and method for measuring consumption of a selected type of food, ingredient, or nutrient can comprise: a wearable imaging sensor that is worn by a person that automatically collects image data, wherein this image data is used to determine when this person is consuming food; and a user interface that prompts the person to provide additional information concerning the selected types of foods, ingredients, or nutrients that the person is eating when the imaging data indicates that the person is consuming food.

In an example, a device for measuring a person's consumption of at least one selected type of food, ingredient, or nutrient can comprise a wearable camera that continually takes pictures of the space surrounding a person. In an example, a camera can continually track the locations of a person's hands and only focus on the space near those hands to detect possible hand-and-food interaction. In an example, a device for monitoring a person's food consumption can optically monitor the space around a person for reachable food sources that may result in food consumption. In an example, a device for monitoring a person's food consumption can monitor the person's movements for hand-to-mouth gestures that may indicate food consumption.

In an example, a device can automatically recognize people within its range of vision and restrict picture focal range or content to not record pictures of people. In an example, this camera can automatically defocus images of other people for the sake of privacy. As an alternative way to address privacy issues, this camera can only be triggered to take record pictures when there are visual, sonic, olfactory, or locational indicators that the person is eating food or likely to eat food. As another way to address privacy issues, this camera can have a manual shut-off that the person can use to shut off the camera.

In an example, a wearable device and system for measuring a person's consumption of at least one selected type of food, ingredient, or nutrient can be tamper resistant. In an example, a wearable device can detect when it has been removed from the person's body by monitoring signals from the body such as pulse, motion, heat, skin electromagnetism, or proximity to an implanted device. In an example, a wearable device for measuring food consumption can detect if it has been removed from the person's body by detecting a lack of motion, lack of a pulse, and/or lack of electromagnetic response from skin. In various examples, a wearable device for measuring food consumption can continually monitor optical, electromagnetic, temperature, pressure, or motion signals that indicate that the device is properly worn by a person. In an example, a wearable device can trigger feedback if the device is removed from the person and the signals stop.

In an example, a wearable device for measuring food consumption can detect if its mode of operation becomes impaired. In an example, a wearable device for measuring food consumption that relies on taking pictures of food can detect if its line-of-sight to a person's hands or mouth is blocked. In an example, a wearable device can automatically track the location of a person's hands or mouth and can trigger feedback if this tracking is impaired. In an example, wrist-worn devices can be worn on both wrists to make monitoring food consumption more inclusive and to make it more difficult for a person to circumvent detection of food consumption by the combined devices or system. In an example, a wearable device for measuring food consumption that relies on a smart food utensil can detect if a person is consuming food without using the smart utensil. In an example, a device or system can detect when a utensil or food probe is not in functional linkage with wearable member. In an example, functional linkage can be monitored by common movement, common sound patterns, or physical proximity. In an example, a device or system can trigger feedback or behavioral modification if its function is impaired.

In an example, a person can be prompted to use a hand-held food-identifying sensor to identify the type of food being consumed when a smart watch detects that the person is consuming food and the hand-held food-identifying sensor is not already being used. In an example, a device and system for monitoring, sensing, detecting, and/or tracking a person's consumption of one or more selected types of foods, ingredients, or nutrients can comprise a wearable food-consumption monitor (such as a smart watch or smart necklace) and a hand-held food-identifying sensor (such as a smart utensil or smart phone), wherein data collected by the monitor and sensor are jointly analyzed to measure the types and amounts of specific foods, ingredients, and/or nutrients that the person consumes.

In an example, a person can be prompted to use a hand-held food-identifying sensor for chemical analysis of food when a smart watch detects that the person is consuming food. In an example, a person can be prompted to use a smart utensil for chemical analysis of food when a smart watch detects that the person is consuming food. In an example, a person can be prompted to use a food probe for chemical analysis of food when a smart watch detects that the person is consuming food.

In an example, a person can be prompted to use a hand-held food-identifying sensor to take pictures of food when a smart watch detects that the person is consuming food. In an example, a person can be prompted to use a mobile phone to take pictures of food when a smart watch detects that the person is consuming food. In an example, a person can be prompted to use a smart utensil to take pictures of food when a smart watch detects that the person is consuming food. In an example, a person can be prompted to use a digital camera to take pictures of food when a smart watch detects that the person is consuming food.

In an example, a device and method for monitoring, sensing, detecting, and/or tracking a person's consumption of one or more selected types of foods, ingredients, or nutrients can comprise a wearable device with primary and second modes, mechanisms, or levels of data collection concerning a person's food consumption. The primary mode of data collection can be continuous, not requiring action by the person in association with an eating event apart from the act of eating, and be more useful for general detection of food consumption than it is for identification of consumption of selected types of foods, ingredients, and/or nutrients by the person. The secondary mode of data collection can be non-continuous, requiring action by the person in association with an eating event apart from the act of eating, and can be very useful for identification of consumption of selected types of foods, ingredients, and/or nutrients by the person.

In an example, both primary and secondary data collection can be performed by a device that a person wears on their wrist (such as a smart watch or watch phone). In example, both primary and secondary data collection can be performed by a device that a person wears around their neck (such as a smart necklace or necklace phone). In an example, primary and secondary data can be jointly analyzed to measure the types and amounts of specific foods, ingredients, and/or nutrients that the person consumes. In an example, a person can be prompted to collect secondary data when primary data indicates that the person is probably consuming food.

In an example, data collection by a hand-held food-identifying sensor (such as a smart utensil, food probe, or smart phone) concerning a particular eating event requires action by a person in association with this eating event apart from the actual act of eating. In an example, the person can be prompted to collect data using the hand-held food-identifying sensor when: data that is automatically collected by a wearable food-consumption monitor indicates that the person is probably consuming food; and the person has not already collected data concerning this particular eating event.

In an example, data collection by a hand-held food-identifying sensor can require that a person bring a food-identifying sensor into contact with food, wherein the person is prompted to bring the food-identifying sensor into contact with food when: data that is automatically collected by a wearable food-consumption monitor indicates that the person is probably consuming food; and the person has not already brought the food-identifying sensor into contact with this food. In an example, data collection by a hand-held food-identifying sensor can require that the person aim a camera and take a picture of food, wherein the person is prompted to aim a camera and take a picture of food when: data that is automatically collected by a wearable food-consumption monitor indicates that the person is probably consuming food; and the person has not already taken a picture of this food.

In an example, data collection by a hand-held food-identifying sensor can require that a person enter information concerning food consumed into a hand-held member by touch, keyboard, speech, or gesture. The person can be prompted to enter information concerning food consumed into a hand-held member by touch, keyboard, speech, or gesture when: data that is automatically collected by a wearable food-consumption monitor indicates that the person is probably consuming food; and the person has not already entered information concerning this food.

In an example, a device for measuring a person's consumption of at least one selected type of food, ingredient, or nutrient can comprise: a wearable food-consumption monitor that detects when the person is probably consuming food; and a hand-held food-identifying sensor that detects the person's consumption of at least one selected type of food, ingredient, or nutrient. In an example, the person can be prompted to use the hand-held food-identifying sensor when the wearable consumption monitor indicates that the person is consuming food. In an example, the hand-held food-identifying sensor can be automatically activated or triggered when the food-consumption monitor indicates that the person is consuming food.

In an example, a device for measuring, monitoring, sensing, detecting, and/or tracking a person's consumption of at least one selected type of food, ingredient, or nutrient can comprise: a wearable food-consumption monitor that automatically monitors and detects when the person consumes food, wherein operation of this monitor to detect food consumption does not require any action associated with a particular eating event by the person apart from the actual act of eating; and a hand-held food-identifying sensor that identifies the selected types of foods, ingredients, and/or nutrients that the person consumes, wherein operation of this sensor to identify foods, ingredients, and/or nutrients during a particular eating event requires action by the person apart associated with that eating event apart from the actual act of eating, and wherein the person is prompted to use the hand-held food-identifying sensor when the wearable consumption monitor indicates that the person is consuming food.

In an example, a method for measuring a person's consumption of at least one selected type of food, ingredient, or nutrient can comprise: collecting primary data concerning food consumption using a wearable food-consumption monitor to detect when a person is consuming food; and collecting secondary data concerning food consumption using a hand-held food-identifying sensor when analysis of primary data indicates that the person is consuming food. In an example, collection of secondary data can be automatic when primary data indicates that the person is consuming food. In an example, collection of secondary data can require a triggering action by the person in association with a particular eating event apart from the actual act of eating. In an example, the person can be prompted to take the triggering action necessary to collect secondary data when primary data indicates that the person is consuming food.

In an example, a method for measuring, monitoring, sensing, detecting, and/or tracking a person's consumption of at least one selected type of food, ingredient, or nutrient can comprise: collecting primary data using a wearable food-consumption monitor to detect when a person is probably consuming food, wherein this detector is worn on the person, and wherein primary data collection does not require action by the person at the time of food consumption apart from the act of consuming food; and collecting secondary data using a hand-held food-identifying sensor to identify the selected types of foods, ingredients, or nutrients that the person is consuming, wherein secondary data collection by the hand-held food-identifying sensor requires action by the person at the time of food consumption apart from the act of consuming food, and wherein the person is prompted to take this action when primary data indicates that the person is consuming food and secondary data has not already been collected.

In an example, a method for measuring a person's consumption of at least one selected type of food, ingredient, or nutrient can comprise: (a) having the person wear a motion sensor that is configured to be worn on at least one body member selected from the group consisting of wrist, hand, finger, and arm; wherein this motion sensor continually monitors body motion to provide primary data that is used to detect when a person is consuming food; (b) prompting the person to collect secondary data concerning food consumption when this primary data indicates that the person is consuming food; wherein secondary data is selected from the group consisting of: data from the interaction between food and reflected, absorbed, or emitted light energy including pictures, chromatographic results, fluorescence results, absorption spectra, reflection spectra, infrared radiation, and ultraviolet radiation; data from the interaction between food and electromagnetic energy including electrical conductivity, electrical resistance, and magnetic interaction; data from the interaction between food and sonic energy including ultrasonic energy; data from the interaction between food and chemical receptors including reagents, enzymes, biological cells, and microorganisms; and data from the interaction between food and mass measuring devices including scales and inertial sensors; and (c) using both primary and secondary data to identify the types and quantities of food consumed in a manner that is at least a partially-automatic; wherein the identification of food type and quantity includes one or more methods selected from the group consisting of: motion pattern analysis and identification; image pattern analysis and identification; chromatography; electromagnetic energy pattern analysis and identification; sound pattern analysis and identification; mass, weight, and/or density; and chemical composition analysis.

In an example, a device for measuring a person's consumption of at least one selected type of food, ingredient, or nutrient can comprise: a wearable motion sensor that automatically collects data concerning body motion, wherein this body motion data is used to determine when a person is consuming food; and an imaging sensor that collects images of food, wherein these food images are used to identify the type and quantity of food, ingredients, or nutrients that a person is consuming food. In an example, an imaging sensor that requires action by the person to pictures of food during an eating event. In an example, the device can prompt the person to use the imaging sensor to take pictures of food when body motion data indicates that the person is consuming food. In an example, a device for measuring a person's consumption of at least one selected type of food, ingredient, or nutrient can comprise: a wearable motion sensor that is worn by a person, wherein this motion sensor automatically and continuously collects data concerning the person's body motion, and wherein the body motion data is used to determine when a person is consuming food; and a wearable imaging sensor that is worn by the person, wherein this imaging sensor does not continuously take pictures, but rather only collects images of eating activity when body motion data indicates that the person is consuming food.

In an example, an imaging sensor need not collect images continuously, but rather requires specific action by the person to initiate imaging at the time of food consumption apart from the actual action of eating. In an example, a person can be prompted to take pictures of food when body motion data collected by a wearable motion sensor indicates that the person is consuming food. In an example, a person can be prompted to take pictures of food when sound data collected by a wearable sound sensor indicates that the person is consuming food.

In an example, a device for measuring a person's consumption of at least one selected type of food, ingredient, or nutrient can comprise: a wearable motion sensor that automatically collects data concerning body motion, wherein this body motion data is used to determine when a person is consuming food; and a chemical composition sensor that analyzes the chemical composition of food, wherein results of this chemical analysis are used to identify the type and quantity of food, ingredients, or nutrients that a person is consuming food. In an example, a device for measuring a person's consumption of at least one selected type of food, ingredient, or nutrient can comprise: a wearable motion sensor that is worn by a person, wherein this motion sensor automatically and continuously collects data concerning the person's body motion, and wherein the body motion data is used to determine when a person is consuming food; and a chemical composition sensor, wherein this chemical composition sensor does not continuously monitor the chemical composition of material within the person's mouth or gastrointestinal tract, but rather only collects information concerning the chemical composition of material within the person's mouth or gastrointestinal tract when body motion data indicates that the person is consuming food.

In an example, a chemical composition sensor can identify the type of food, ingredient, or nutrient based on: physical contact between the sensor and food; or the effects of interaction between food and electromagnetic energy or light energy. In an example, a chemical composition sensor need not collect chemical information continuously, but rather requires specific action by the person to initiate chemical analysis at the time of food consumption apart from the actual action of consuming food. In an example, a person can be prompted to activate a sensor to perform chemical analysis of food when body motion data collected by a wearable motion sensor indicates that the person is consuming food. In an example, a person can be prompted to activate a sensor to perform chemical analysis of food when sound data collected by a wearable sound sensor indicates that the person is consuming food.

In an example, a device for measuring a person's consumption of at least one selected type of food, ingredient, or nutrient can comprise: a wearable sound sensor that automatically collects data concerning body or environmental sounds, wherein this sound data is used to determine when a person is consuming food; and an imaging sensor that collects images of food, wherein these food images are used to identify the type and quantity of food, ingredients, or nutrients that a person is consuming food. In an example, this imaging sensor can require action by the person to pictures of food during an eating event. In an example, the person can be prompted to use the imaging sensor to take pictures of food when sound data indicates that the person is consuming food. In an example, a device for measuring a person's consumption of at least one selected type of food, ingredient, or nutrient can comprise: a wearable sound sensor that is worn by a person, wherein this sound sensor automatically and continuously collects data concerning sounds from the person's body, and wherein this sound data is used to determine when a person is consuming food; and a wearable imaging sensor that is worn by the person, wherein this imaging sensor does not continuously take pictures, but rather only collects images of eating activity when sound data indicates that the person is consuming food.

In an example, a device for measuring a person's consumption of at least one selected type of food, ingredient, or nutrient can comprise: a wearable sound sensor that automatically collects data concerning body or environmental sound, wherein this sound data is used to determine when a person is consuming food; and a chemical composition sensor that analyzes the chemical composition of food, wherein results of this chemical analysis are used to identify the type and quantity of food, ingredients, or nutrients that a person is consuming food. In an example, a device for measuring a person's consumption of at least one selected type of food, ingredient, or nutrient can comprise: a wearable sound sensor that is worn by a person, wherein this motion sensor automatically and continuously collects data concerning sound from the person's body, and wherein this sound data is used to determine when a person is consuming food; and a chemical composition sensor, wherein this chemical composition sensor does not continuously monitor the chemical composition of material within the person's mouth or gastrointestinal tract, but rather only collects information concerning the chemical composition of material within the person's mouth or gastrointestinal tract when sound data indicates that the person is consuming food.

In an example, a method for measuring a person's consumption of at least one selected type of food, ingredient, or nutrient can comprise: collecting a first set of data to detect when a person is probably consuming food in an automatic and continuous manner that does not require action by the person at the time of food consumption apart from the act of consuming food; collecting a second set of data to identify what selected types of foods, ingredients, or nutrients a person is consuming when the first set of data indicates that the person is probably consuming food; and jointly analyzing both the first and second sets of data to estimate consumption of at least one specific food, ingredient, or nutrient by the person.

In an example, a device or system for measuring a person's consumption of at least one selected type of food, ingredient, or nutrient can comprise: (a) a wearable food-consumption monitor that is configured to be worn on a person's body or clothing, wherein this monitor automatically collects primary data that is used to detect when the person is consuming food; (b) a food-identifying sensor that collects secondary data that is used to measure the person's consumption of at least one selected type of food, ingredient, or nutrient, and wherein secondary data collection in association with a specific food consumption event requires a specific action by the person in association with that specific food consumption event apart from the act of consuming food; and (c) a computer-to-human prompting interface, wherein this interface prompts the person to take the specific action required for secondary data collection in association with a specific food consumption event when the primary data indicates that the person is consuming food and the person has not already taken this specific action. In an example, primary data can be body movement data or data concerning electromagnetic signals from the person's body. In an example, secondary data can be collected by a mobile phone, smart utensil, food probe, smart necklace, smart eyewear, or a smart watch.

In an example, a device or system for measuring a person's consumption of at least one selected type of food, ingredient, or nutrient can comprise: (a) a wearable food-consumption monitor that is configured to be worn on a person's body or clothing, wherein this monitor automatically collects primary data that is used to detect when the person is consuming food; (b) an imaging component that collects secondary data that is used to measure the person's consumption of at least one selected type of food, ingredient, or nutrient, wherein this secondary data comprises pictures of food, and wherein taking pictures of food in association with a specific food consumption event requires a specific action by the person in association with that specific food consumption event apart from the act of consuming food; and (c) a computer-to-human prompting interface, wherein this interface prompts the person to take pictures of food in association with a specific food consumption event when the primary data indicates that the person is consuming food and pictures of this food have not already been taken. In an example, primary data can be body movement data or data concerning electromagnetic signals from the person's body. In an example, secondary data can be collected by a mobile phone, smart utensil, food probe, smart necklace, smart eyewear, or a smart watch.

In an example, a device or system for measuring a person's consumption of at least one selected type of food, ingredient, or nutrient can comprise: (a) a wearable food-consumption monitor that is configured to be worn on a person's body or clothing, wherein this monitor automatically collects primary data that is used to detect when the person is consuming food; (b) an chemical-analyzing component that collects secondary data that is used to measure the person's consumption of at least one selected type of food, ingredient, or nutrient, wherein this secondary data comprises chemical analysis of food, and wherein performing chemical analysis of food in association with a specific food consumption event requires a specific action by the person in association with that specific food consumption event apart from the act of consuming food; and (c) a computer-to-human prompting interface, wherein this interface prompts the person to take the action required to perform chemical analysis of food in association with a specific food consumption event when the primary data indicates that the person is consuming food and chemical analysis of this food has not already been performed. In an example, primary data can be body movement data or data concerning electromagnetic signals from the person's body. In an example, secondary data can be collected by a mobile phone, smart utensil, food probe, smart necklace, smart eyewear, or a smart watch.

In an example, a device or system for measuring a person's consumption of at least one selected type of food, ingredient, or nutrient can comprise: (a) a wearable food-consumption monitor that is configured to be worn on a person's body or clothing, wherein this monitor automatically collects primary data that is used to detect when the person is consuming food; (b) a computer-to-human prompting interface which a person uses to enter secondary data concerning the person's consumption of at least one selected type of food, ingredient, or nutrient, wherein this interface selected from the group consisting of: speech or voice recognition, touch or gesture recognition, motion recognition or eye tracking, and buttons or keys, and wherein this interface prompts the person to enter secondary data in association with a specific food consumption event when the primary data indicates that the person is consuming food and the person has not already entered this data. In an example, primary data can be body movement data or data concerning electromagnetic signals from the person's body. In an example, secondary data can be collected by a mobile phone, smart utensil, food probe, smart necklace, smart eyewear, or a smart watch.

In an example, a device or system for measuring a person's consumption of at least one selected type of food, ingredient, or nutrient can comprise: (a) a wearable food-consumption monitor that is configured to be worn on a person's body or clothing, wherein this monitor automatically collects primary data that is used to detect when the person is consuming food; (b) a food-identifying sensor that automatically collects secondary data that is used to measure the person's consumption of at least one selected type of food, ingredient, or nutrient in association with a specific food consumption event when the primary data indicates that the person is consuming food. In an example, primary data can be body movement data or data concerning electromagnetic signals from the person's body. In an example, secondary data can be collected by a mobile phone, smart utensil, food probe, smart necklace, smart eyewear, or a smart watch.

In an example, a device or system for measuring a person's consumption of at least one selected type of food, ingredient, or nutrient can comprise: (a) a smart watch that is configured to be worn on a person's wrist, hand, or arm, wherein this smart watch automatically collects primary data that is used to detect when the person is consuming food; (b) a food-identifying sensor that collects secondary data that is used to measure the person's consumption of at least one selected type of food, ingredient, or nutrient, and wherein secondary data collection in association with a specific food consumption event requires a specific action by the person in association with that specific food consumption event apart from the act of consuming food; and (c) a computer-to-human prompting interface, wherein this interface prompts the person to take the specific action required for secondary data collection in association with a specific food consumption event when the primary data indicates that the person is consuming food and the person has not already taken this specific action. In an example, primary data can be body movement data or data concerning electromagnetic signals from the person's body. In an example, secondary data can be collected by a mobile phone, smart utensil, food probe, smart necklace, smart eyewear, or the smart watch.

In an example, a device or system for measuring a person's consumption of at least one selected type of food, ingredient, or nutrient can comprise: (a) a smart watch that is configured to be worn on a person's wrist, hand, or arm, wherein this smart watch automatically collects primary data that is used to detect when the person is consuming food; (b) an imaging component that collects secondary data that is used to measure the person's consumption of at least one selected type of food, ingredient, or nutrient, wherein this secondary data comprises pictures of food, and wherein taking pictures of food in association with a specific food consumption event requires a specific action by the person in association with that specific food consumption event apart from the act of consuming food; and (c) a computer-to-human prompting interface, wherein this interface prompts the person to take pictures of food in association with a specific food consumption event when the primary data indicates that the person is consuming food and pictures of this food have not already been taken. In an example, primary data can be body movement data or data concerning electromagnetic signals from the person's body. In an example, secondary data can be collected by a mobile phone, smart utensil, food probe, smart necklace, smart eyewear, or the smart watch.

In an example, a device or system for measuring a person's consumption of at least one selected type of food, ingredient, or nutrient can comprise: (a) a smart watch that is configured to be worn on a person's wrist, hand, or arm, wherein this smart watch automatically collects primary data that is used to detect when the person is consuming food; (b) an chemical-analyzing component that collects secondary data that is used to measure the person's consumption of at least one selected type of food, ingredient, or nutrient, wherein this secondary data comprises chemical analysis of food, and wherein performing chemical analysis of food in association with a specific food consumption event requires a specific action by the person in association with that specific food consumption event apart from the act of consuming food; and (c) a computer-to-human prompting interface, wherein this interface prompts the person to take the action required to perform chemical analysis of food in association with a specific food consumption event when the primary data indicates that the person is consuming food and chemical analysis of this food has not already been performed. In an example, primary data can be body movement data or data concerning electromagnetic signals from the person's body. In an example, secondary data can be collected by a mobile phone, smart utensil, food probe, smart necklace, smart eyewear, or the smart watch.

In an example, a device or system for measuring a person's consumption of at least one selected type of food, ingredient, or nutrient can comprise: (a) a smart watch that is configured to be worn on a person's wrist, hand, or arm, wherein this smart watch automatically collects primary data that is used to detect when the person is consuming food; (b) a computer-to-human prompting interface which a person uses to enter secondary data concerning the person's consumption of at least one selected type of food, ingredient, or nutrient, wherein this interface selected from the group consisting of: speech or voice recognition, touch or gesture recognition, motion recognition or eye tracking, and buttons or keys, and wherein this interface prompts the person to enter secondary data in association with a specific food consumption event when the primary data indicates that the person is consuming food and the person has not already entered this data. In an example, primary data can be body movement data or data concerning electromagnetic signals from the person's body. In an example, the interface can comprise a mobile phone, smart utensil, food probe, smart necklace, smart eyewear, or the smart watch.

In an example, a device or system for measuring a person's consumption of at least one selected type of food, ingredient, or nutrient can comprise: (a) a smart watch that is configured to be worn on a person's wrist, hand, or arm, wherein this smart watch automatically collects primary data that is used to detect when the person is consuming food; (b) a food-identifying sensor that automatically collects secondary data that is used to measure the person's consumption of at least one selected type of food, ingredient, or nutrient in association with a specific food consumption event when the primary data indicates that the person is consuming food. In an example, primary data can be body movement data or data concerning electromagnetic signals from the person's body. In an example, secondary data can be collected by a mobile phone, smart utensil, food probe, smart necklace, smart eyewear, or the smart watch.

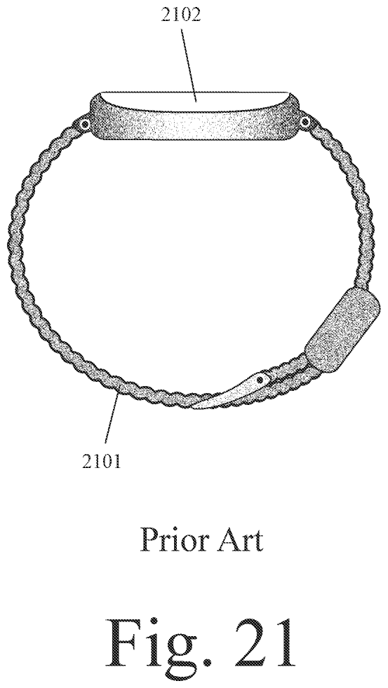

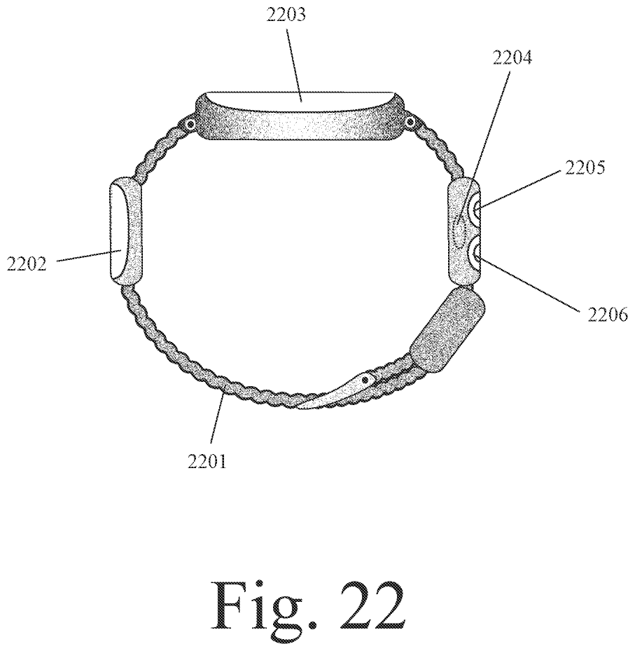

FIG. 21 shows a side view of a basic type of smart watch which is known in the prior art. This basic smart watch design includes a housing for a primary display 2102 (e.g. the watch face) and a wrist-worn band 2101 (e.g. the watch band) which holds the housing and display on a person's wrist. When in use, the smart watch is worn around a person's wrist, but a wrist is not shown here in order to provide a clearer view of the device itself. The housing for the watch face is generally worn on the dorsal side of a person's wrist, although some people may wear it on the ventral side. This is not a great location from which to record food images, especially if the housing is relatively flat against the surface of the wrist. A person generally needs to rotate and/or twist their arm in an awkward way to direct a camera on the dorsal side of the wrist to point toward nearby food. Also, when their arm is rotated and/or twisted, the primary display is tilted away from the person's line of sight, making it also a poor location to serve as a camera viewfinder for recording food images. The wrist-worn device designs disclosed herein, starting with FIG. 22, address and correct these problems.

The wrist-worn devices disclosed herein, starting with FIG. 22, address problems of the prior art by positioning a camera and/or secondary display (which serves as a camera viewfinder) at locations around the circumference of the wrist other than the location of the primary display housing. The wrist-worn devices disclosed herein also feature novel components, such as a spectroscopic sensor which enables more accurate measurement of food composition and an eating detector which can reduce selectively activate the camera to reduce camera-related privacy issues. These wrist-worn device designs generally build on the basic smart watch design by adding components which facilitate food identification and tracking food consumption.

FIG. 22 shows an example of a wrist-worn device for tracking food intake comprising: a camera which is held on a person's wrist by a wrist-worn band (e.g. the band of a smart watch), wherein the camera records food images, wherein the food images are analyzed to identify food types and quantities, wherein there is also a primary display (e.g. a watch face) which is held on the person's wrist by the wrist-worn band, wherein the primary display is centered on a first location on the circumference of the band, wherein the camera is centered on a second location on the circumference of the band, and wherein the second location is between 60 to 110 degrees around the band circumference away from the first location in a first (e.g. clockwise) direction; a camera viewfinder which held on the person's wrist by the wrist-worn band, wherein the camera viewfinder displays the food images recorded by the camera, wherein the camera viewfinder is centered on a third location on the circumference of the band, and wherein the third location is between 70 and 110 degrees around the band circumference away from the first location in a second (e.g. counter-clockwise) direction which is opposite the first direction; a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, and an EMG sensor. Again, it is assumed that the device is worn on a person's wrist, but the wrist is not shown here in order to provider a clearer view of the device itself.

With respect to specific components, FIG. 22 shows a side view of an example of wrist-worn device for tracking food intake comprising: a camera 2206 which is held on a person's wrist by a wrist-worn band 2201 (e.g. the band of a smart watch), wherein the camera records food images, wherein the food images are analyzed to identify food types and quantities, wherein there is also a primary display 2203 (e.g. a watch face) which is held on the person's wrist by the wrist-worn band, wherein the primary display is centered on a first location on the circumference of the band, wherein the camera is centered on a second location on the circumference of the band, and wherein the second location is between 70 and 110 degrees around the band circumference away from the first location in a first (e.g. clockwise) direction; a camera viewfinder 2202 which held on the person's wrist by the wrist-worn band, wherein the camera viewfinder displays the food images recorded by the camera, wherein the camera viewfinder is centered on a third location on the circumference of the band, and wherein the third location is between 60 to 110 degrees around the band circumference away from the first location in a second (e.g. counter-clockwise) direction which is opposite the first direction; a spectroscopic sensor 2205 which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector 2204 which collects data which is analyzed to detect when the person is eating, wherein the eating detector further comprises one or more components selected from the group consisting of: an accelerometer, a gyroscope, a magnetometer, a microphone, and an EMG sensor.

In an example, a camera viewfinder and a housing for a camera and spectroscopic sensor can be separate components which are (individually and removably) attached to a conventional watch band. In another example, a camera viewfinder, a camera, and a spectroscopic sensor can be integrated into a specialized watch band which is an (interchangeable) option for use with a conventional smart watch. In another example, a primary display, camera viewfinder, camera, and spectroscopic sensor can all be integrated into a single specialized wrist-worn device (e.g. a specialized food tracking band and/or customized food-tracking smart watch).

In an example, a camera and a spectroscopic sensor can both be in the same housing and general location on a portion of a wrist-band. In an example, a camera and a spectroscopic sensor can be in different housings and/or locations. In an example, an eating detector can be in the same housing as a camera and spectroscopic sensor. Alternatively, an eating detector can be in the housing for the primary display; for example, a motion sensor in a conventional smart watch can serve as an eating detector. In an example, a motion sensor can further comprise one or more components selected from the group consisting of: accelerometer, gyroscope, magnometer, and inclinometer. Example variations discussed elsewhere in this disclosure or in priority-linked disclosures can also be applied to this example where relevant.

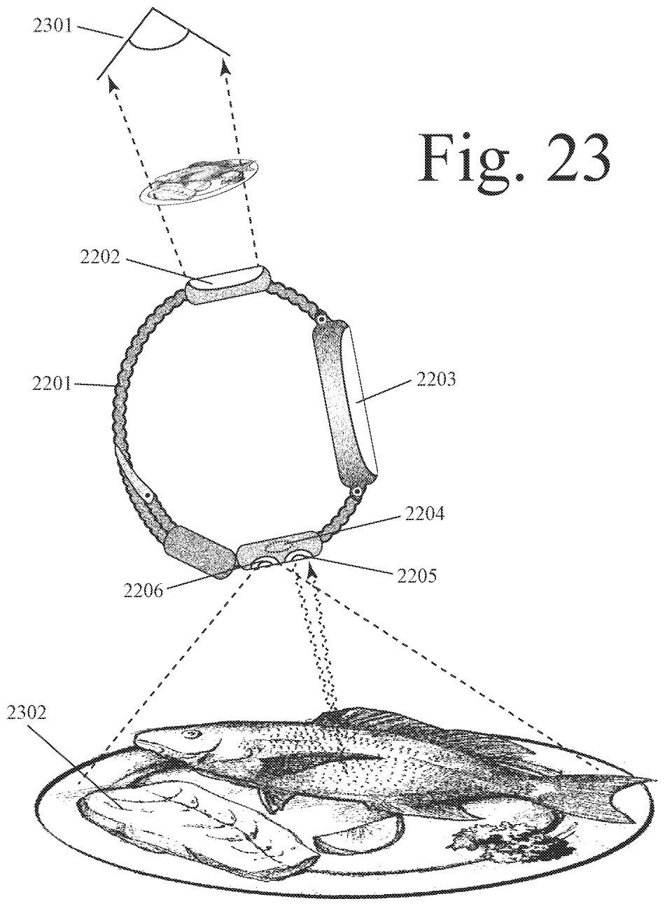

FIG. 23 shows the wrist-worn device of FIG. 22 in action. Again, the person's wrist is not shown, but it is assumed that the device is worn on a person's wrist. In FIG. 23, the person's wrist and the device have been rotated so that the camera faces toward food 2302 and the camera viewfinder faces toward the person's eye 2301. In this example, the viewfinder is on the opposite side of the wrist than the camera, providing a view as if the person were “seeing through” their wrist. This is analogous to how people take pictures with a (relatively-flat) cellphone wherein the camera is on one side of the cellphone and the display is on the opposite side of the cellphone. With this design for a wrist-worn device, a person can capture food images while holding their wrist and arm in a comfortable, natural position. In an example a person can capture food images by discreetly waiving their hand over a plate of food, perhaps adding a Jedi mind trick by saying “These are not the foods you are looking for.”

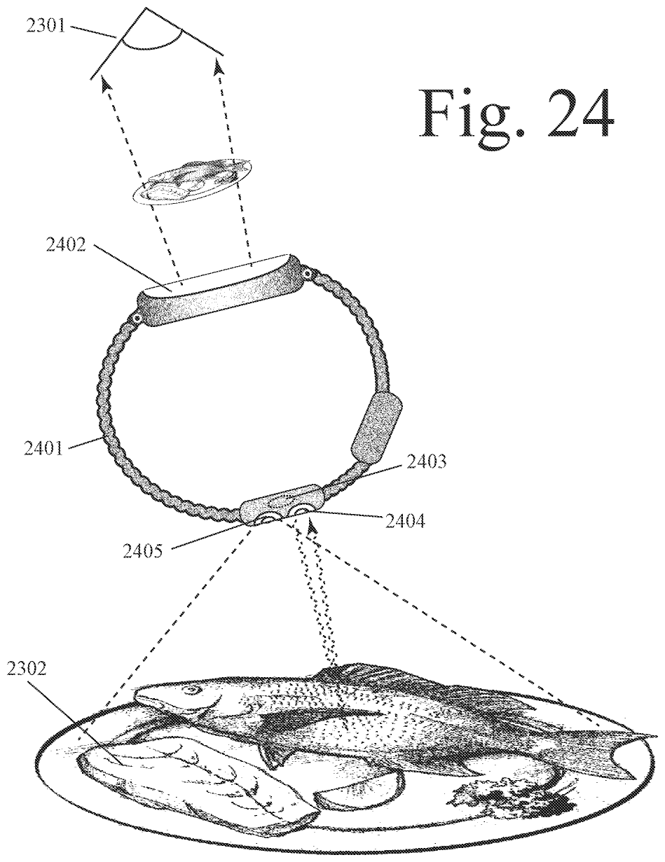

The wrist-worn device shown in FIG. 24 is similar to the one shown in FIGS. 22 and 23 except that the primary display serves as a camera viewfinder instead of having a secondary display serve as a camera viewfinder. In this example, a camera and spectroscopic sensor are located on the opposite side of the wrist from the housing for the primary display. For example, if the primary display is on the dorsal side of the wrist, then the camera is on the ventral side of the wrist. Again, it is assumed that the device is worn on a person's wrist, but the wrist is not shown here in order to provider a clearer view of the device itself.

FIG. 24 shows an example of wrist-worn device for tracking food intake comprising: a camera which is held on a person's wrist by a wrist-worn band (e.g. the band of a smart watch), wherein the camera records food images, wherein the food images are analyzed to identify food types and quantities, wherein there is also a primary display (e.g. a watch face) which is held on the person's wrist by the wrist-worn band, wherein the primary display serves as a viewfinder for the camera (e.g. displaying food images recorded by the camera), wherein the primary display is centered on a first location on the circumference of the band, wherein the camera is centered on a second location on the circumference of the band, and wherein the second location is between 160 and 200 degrees around the band circumference away from the first location (e.g. substantially on the opposite side of the wrist from the primary display); a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, and an EMG sensor.

With respect to specific components, FIG. 24 shows an example of wrist-worn device for tracking food intake comprising: a camera 2405 which is held on a person's wrist by a wrist-worn band 2401 (e.g. the band of a smart watch), wherein the camera records images of food 2302, wherein the food images are analyzed to identify food types and quantities, wherein there is also a primary display 2402 (e.g. a watch face) which is held on the person's wrist by the wrist-worn band and viewed by a person's eye(s) 2301, wherein the primary display serves as a viewfinder for the camera (e.g. displaying food images recorded by the camera), wherein the primary display is centered on a first location on the circumference of the band, wherein the camera is centered on a second location on the circumference of the band, and wherein the second location is between 160 and 200 degrees around the band circumference away from the first location (e.g. substantially on the opposite side of the wrist from the primary display); a spectroscopic sensor 2404 which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector 2403 which collects data which is analyzed to detect when the person is eating, wherein the eating detector further comprises one or more components selected from the group consisting of: an accelerometer, a gyroscope, a magnetometer, a microphone, and an EMG sensor.

In an example, one or more housings for a camera and a spectroscopic sensor can be removably-attached to a conventional watch band. In another example, a camera and a spectroscopic sensor can be integrated into a specialized watch band which is an (interchangeable) option for use with a conventional smart watch. In another example, a primary display, a camera, and a spectroscopic sensor can all be integrated into a single specialized wrist-worn device (e.g. a specialized food tracking band and/or customized food-tracking smart watch).

In an example, a camera and a spectroscopic sensor can both be in the same housing and general location on a portion of a wrist-band. In an example, a camera and a spectroscopic sensor can be in different housings and/or locations. In an example, an eating detector can be in the same housing as a camera and spectroscopic sensor. Alternatively, an eating detector can be in the housing for the primary display; for example, a motion sensor in a conventional smart watch can serve as an eating detector. In an example, a motion sensor can further comprise one or more components selected from the group consisting of: accelerometer, gyroscope, magnetometer, and inclinometer. Example variations discussed elsewhere in this disclosure or in priority-linked disclosures can also be applied to this example where relevant.

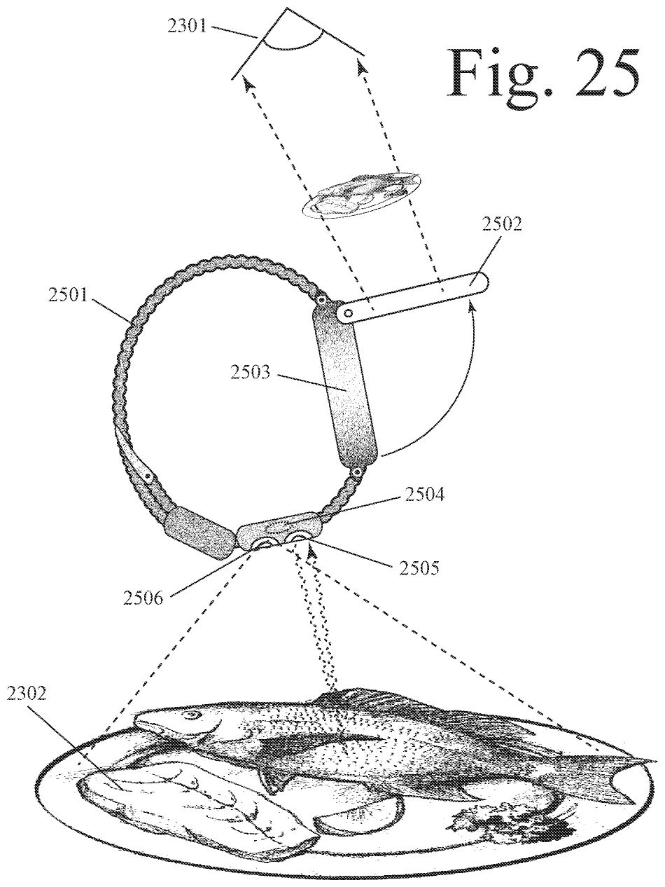

FIG. 25 shows an example of wrist-worn device for tracking food intake comprising: a primary housing which is held on a person's wrist by a wrist-worn band; a flip-up display which flips, pivots, rotates, tilts, and/or pops up from the primary housing; a camera which is held on the person's wrist by the wrist-worn band, wherein the camera records food images, wherein the food images are analyzed to identify food types and quantities, wherein the flip-up display serves as a viewfinder for the camera (e.g. displaying food images recorded by the camera), wherein the primary housing is centered on a first location on the circumference of the band, wherein the camera is centered on a second location on the circumference of the band, and wherein the second location is between 70 and 110 degrees around the band circumference away from the first location; a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, and an EMG sensor.

With respect to specific components, FIG. 25 shows an example of wrist-worn device for tracking food intake comprising: a primary housing 2503 which is held on a person's wrist by a wrist-worn band 2501; a flip-up display 2502 which flips, pivots, rotates, tilts, and/or pops up from the primary housing, wherein the flip-up display is viewed by the person's eye(s) 2301; a camera 2506 which is held on the person's wrist by the wrist-worn band, wherein the camera records images of food 2302, wherein the food images are analyzed to identify food types and quantities, wherein the flip-up display serves as a viewfinder for the camera (e.g. displaying food images recorded by the camera), wherein the primary housing is centered on a first location on the circumference of the band, wherein the camera is centered on a second location on the circumference of the band, and wherein the second location is between 70 and 110 degrees around the band circumference away from the first location; a spectroscopic sensor 2505 which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector 2504 which collects data which is analyzed to detect when the person is eating, wherein the eating detector further comprises one or more components selected from the group consisting of: an accelerometer, a gyroscope, a magnetometer, a microphone, and an EMG sensor.

In an example, one or more housings for a camera and a spectroscopic sensor can be removably-attached to a conventional watch band. In another example, a camera and a spectroscopic sensor can be integrated into a specialized watch band which is an (interchangeable) option for use with a conventional smart watch. In another example, a primary display, a camera, and a spectroscopic sensor can all be integrated into a single specialized wrist-worn device (e.g. a specialized food tracking band and/or customized food-tracking smart watch).

In an example, a camera and a spectroscopic sensor can both be in the same housing and general location on a portion of a wrist-band. In an example, a camera and a spectroscopic sensor can be in different housings and/or locations. In an example, an eating detector can be in the same housing as a camera and spectroscopic sensor. Alternatively, an eating detector can be in the housing for the primary display; for example, a motion sensor in a conventional smart watch can serve as an eating detector. In an example, a motion sensor can further comprise one or more components selected from the group consisting of: accelerometer, gyroscope, magnetometer, and inclinometer. Example variations discussed elsewhere in this disclosure or in priority-linked disclosures can also be applied to this example where relevant.

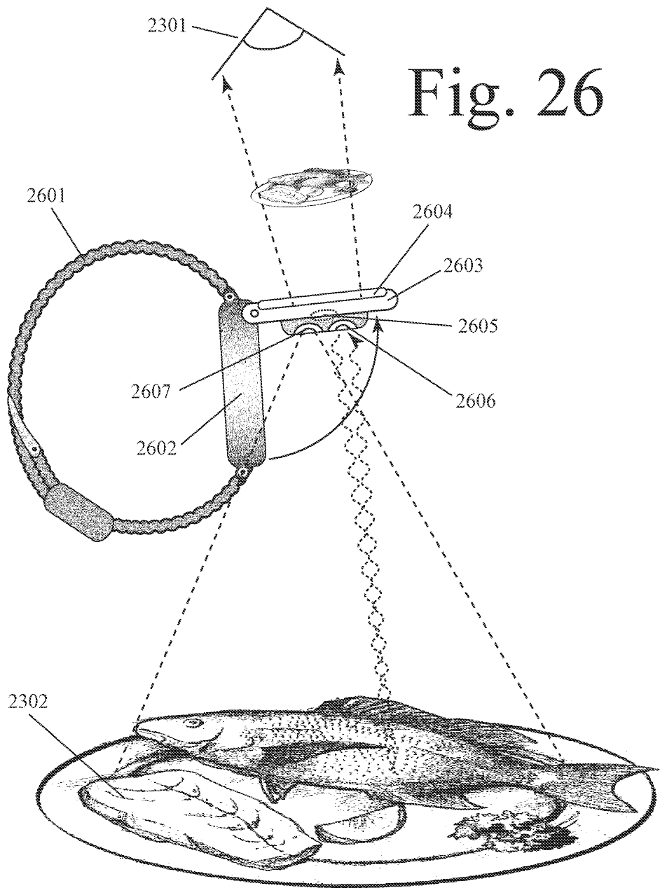

FIG. 26 shows an example of wrist-worn device for tracking food intake comprising: a housing which is held on a person's wrist by a wrist-worn band; a flip-up component which flips, pivots, rotates, tilts, and/or pops up from the housing; a display on a first side of the flip-up component; a camera on a second side of the flip-up component, wherein the camera records food images, wherein the food images are analyzed to identify food types and quantities, wherein the flip-up display serves as a viewfinder for the camera (e.g. displaying food images recorded by the camera), and wherein the second side of the flip-up component is opposite the first side of the flip-up component; a spectroscopic sensor which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector which collects data which is analyzed to detect when the person is eating, wherein the eating detector further comprises one or more components selected from the group consisting of: an accelerometer, a gyroscope, a magnetometer, a microphone, and an EMG sensor.

With respect to specific components, FIG. 26 shows an example of wrist-worn device for tracking food intake comprising: a housing 2602 which is held on a person's wrist by a wrist-worn band 2601; a flip-up component 2603 which flips, pivots, rotates, tilts, and/or pops up from the housing; a display 2604 on a first side of the flip-up component, wherein the display is viewed by the person's eye(s) 2301; a camera 2607 on a second side of the flip-up component, wherein the camera records images of food 2302, wherein the food images are analyzed to identify food types and quantities, wherein the flip-up display serves as a viewfinder for the camera (e.g. displaying food images recorded by the camera), and wherein the second side of the flip-up component is opposite the first side of the flip-up component; a spectroscopic sensor 2606 which is held on the person's wrist by the wrist-worn band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector 2605 which collects data which is analyzed to detect when the person is eating, wherein the eating detector further comprises one or more components selected from the group consisting of: an accelerometer, a gyroscope, a magnetometer, a microphone, and an EMG sensor. Example variations discussed elsewhere in this disclosure or in priority-linked disclosures can also be applied to this example where relevant.

In an example, a wearable device for tracking food intake can comprise: a band worn on a person's wrist and/or arm; a camera on the band, wherein the camera records food images, wherein the food images are analyzed to identify food types and quantities; a primary display on the band, wherein the primary display is centered on a first location on the circumference of the band, wherein the camera is centered on a second location on the circumference of the band, wherein the second location is between 60 to 110 degrees around the band circumference away from the first location in a first direction, and wherein the first direction can be clockwise; a camera viewfinder on the band, wherein the camera viewfinder displays the food images recorded by the camera, wherein the camera viewfinder is centered on a third location on the circumference of the band, and wherein the third location is between 70 and 110 degrees around the band circumference away from the first location in a second direction which is opposite the first direction, and wherein the second direction can be counter-clockwise; a spectroscopic sensor on the band, wherein the spectroscopic sensor further comprises a light emitter and a light receiver, wherein the light emitter emits light rays toward food, wherein the light receiver receives the light rays after the rays have been reflected by the food, and wherein the light rays reflected by the food are analyzed to identify food types and/or composition; and an eating detector on the band which collects data which is analyzed to detect when the person is eating, wherein the eating detector further comprises one or more components selected from the group consisting of: an accelerometer, a gyroscope, a magnetometer, a microphone, and an EMG sensor. Example variations discussed elsewhere in this disclosure or in priority-linked disclosures can also be applied to this example where relevant.

In an example, a wearable device for tracking food intake can comprise: a band worn on a person's wrist and/or arm; a camera on the band, wherein the camera records food images, wherein the food images are analyzed to identify food types and quantities; a primary display on the band, wherein the primary display displays food images recorded by the camera, wherein the primary display is centered on a first location on the circumference of the band, wherein the camera is centered on a second location on the circumference of the band, and wherein the second location is between 160 to 200 degrees around the band circumference away from the first location; a spectroscopic sensor on the band, wherein the spectroscopic sensor further comprises a light emitter and a light receiver, wherein the light emitter emits light rays toward food, wherein the light receiver receives the light rays after the rays have been reflected by the food, and wherein the light rays reflected by the food are analyzed to identify food types and/or composition; and an eating detector on the band which collects data which is analyzed to detect when the person is eating, wherein the eating detector further comprises one or more components selected from the group consisting of: an accelerometer, a gyroscope, a magnetometer, a microphone, and an EMG sensor. Example variations discussed elsewhere in this disclosure or in priority-linked disclosures can also be applied to this example where relevant.

In an example, a wearable device for tracking food intake can comprise: a band worn on a person's wrist and/or arm; a primary housing on the band; a flip-up display which flips, pivots, rotates, tilts, and/or pops up from the primary housing; a camera on the band, wherein the camera records food images, wherein the food images are analyzed to identify food types and quantities, wherein the flip-up display serves as a viewfinder for the camera, wherein the primary housing is centered on a first location on the circumference of the band, wherein the camera is centered on a second location on the circumference of the band, and wherein the second location is between 70 and 110 degrees around the band circumference away from the first location; a spectroscopic sensor on the band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and an eating detector on the band which collects data which is analyzed to detect when the person is eating, wherein the eating detector further comprises one or more components selected from the group consisting of: an accelerometer, a gyroscope, a magnetometer, a microphone, and an EMG sensor. Example variations discussed elsewhere in this disclosure or in priority-linked disclosures can also be applied to this example where relevant.

In an example, the focal direction vector of a camera on a wrist (and/or arm) band and the emission vector of light from a light emitter on the band which is part of a spectroscopic sensor can be parallel to each other. In an example, the angle between the focal direction vector of a camera on a wrist (and/or arm) band and the emission vector of light from a light emitter on the band can be between 5 and 15 degrees. In an example, the angle between the focal direction vector of a camera on a wrist (and/or arm) band and the emission vector of light from a light emitter on the band can be between 10 and 30 degrees. In an example, the focal direction vector of a camera on a wrist (and/or arm) band and the emission vector of light from a light emitter on the band can both be moved and/or scanned relative to nearby food. In an example, the focal direction vector of a camera on a wrist (and/or arm) band and the emission vector of light from a light emitter on the band can be moved together (e.g. in tandem) relative to nearby food.

In an example, a camera on a wrist (and/or arm) band can be located diametrically opposite from a primary display on the band. In an example, a camera on the band of a smart watch can be located diametrically opposite from the watch face. In an example, a spectroscopic sensor on a wrist (and/or arm) band can be located diametrically opposite from a primary display on the band. In an example, a spectroscopic sensor on the band of a smart watch can be located diametrically opposite from the watch face. In an example, a camera and a spectroscopic sensor on a wrist (and/or arm) band can both be located diametrically opposite from a primary display on the band. In an example, a camera on wrist (and/or arm) band and a spectroscopic sensor on the band can be co-located on the circumference of the band. In an example, a camera on a wrist (and/or arm) band can be 180-degrees around the circumference of the band from a camera viewfinder on the band.

In an example, a wrist (and/or arm) band can include a motion sensor (e.g. including an accelerometer and a gyroscope) and a camera, wherein the camera is activated to record food images when eating motions are detected by analysis of data from the motion sensor. In an example, a wrist (and/or arm) band on a first arm can include a motion sensor and a wrist (and/or arm) band on a second arm can include a camera, wherein the camera is activated to record food images when eating motions are detected by analysis of data from the motion sensor. In an example, a wrist (and/or arm) band can include a motion sensor and a camera, wherein the focal direction and/or distance of the camera is adjusted as the wrist (and/or arm) band is moved based on analysis of data from the motion sensor in order to keep the camera focused toward food.

In an example, the location of a camera on the circumference of a wrist (and/or arm) band can be adjusted by sliding the camera. In an example, a camera can be slid around a portion of the circumference of a wrist (and/or arm) band and then locked in place. In an example, a wrist (and/or arm) band can further comprise a track along which a camera can be slid and locked in place. In an example, the location of a spectroscopic sensor on the circumference of a wrist (and/or arm) band can be adjusted by sliding the spectroscopic sensor. In an example, a spectroscopic sensor can be slid around a portion of the circumference of a wrist (and/or arm) band and then locked in place. In an example, a wrist (and/or arm) band can further comprise a track along which a spectroscopic sensor is slid and locked in place.

In an example, a wrist (and/or arm) band can have two cameras in order to record three-dimensional food images. In an example, these two cameras can be at radial locations on a band which are separated by between 5 and 30 degrees. In an example, a wearable system for tracking food consumption can comprise a first band with a first camera on a person's first (e.g. right) arm and a second band with a second camera on the person's second (e.g. left) arm, wherein images from the first and second cameras are analyzed jointly in order to create a three-dimensional food image.

In an example, a wrist (and/or arm) band can have a flip-up component which flips, tilts, pivots, and/or rotates outward from the band. In an example, a wrist (and/or arm) band can have a flip-up component which flips, tilts, pivots, and/or rotates out from a housing on the band. In an example, the flip-up component can flip up from a recess on the housing. In an example, there can be a camera on the flip-up component. In an example, there can be a spectroscopic sensor on the flip-up component. In an example, there can be a camera viewfinder on the flip-up component. In an example, there can be a viewfinder on one side of the flip-up component and a camera on the other side of the flip-up component. In an example, a flip-up component can be detached from a wrist-worn device and moved (e.g. waved) over food for close-up imaging and/or spectroscopic analysis of the food.

In an example, the angle between a housing and light emission from a light emitter (e.g. LED) which is part of a spectroscopic sensor can be adjusted. In an example, a spectroscopic sensor can be mounted on a gimbal mechanism. In an example, light rays emitted by a light emitter can be redirected by a micromirror array (e.g. a digital micromirror array). In an example, the direction of light emitted from a light emitter on a band can be adjusted based on movement of the band in order to maintain focal direction toward food. In an example, the angle between a housing and light emission from a light emitter (e.g. LED) can be automatically adjusted.

In an example, the angle of light emission from a light emitter (e.g. LED) on a wrist (or arm) worn band can be changed in an automatic, iterative, and/or scanning manner. In an example, the angle of light emission from a light emitter (e.g. LED) on a wrist (or arm) worn band can be changed in an iterative manner in order to scan nearby food. In an example, the angle of light emission from a light emitter (e.g. LED) on a wrist (or arm) worn band can be changed automatically in order to sequentially vary the angle of light beam incidence with nearby food. In an example, the angle of light emission from a light emitter (e.g. LED) on a wrist (or arm) worn band can be changed automatically in order to sequentially vary the angle of light beam reflection from nearby food.

In an example, a spectroscopic sensor can comprise a light emitter (e.g. LED) which is between 1 mm and 5 mm away from a light receiver. In an example, a spectroscopic sensor can comprise a light emitter which is between 4 mm and 10 mm away from a light receiver. In an example, the distance between a light emitter and a light receiver which comprise a spectroscopic sensor can be between 1 mm and 5 mm. In an example, the distance between a light emitter and a light receiver which comprise a spectroscopic sensor can be between 4 mm and 10 mm. In an example, the distance between a light emitter and a light receiver which comprise a spectroscopic sensor on a wrist (and/or arm) band can be automatically adjusted based on the distance between the band and food. In an example, a light emitter can be between 8 mm and 20 mm away from a light receiver on a spectroscopic sensor.

In an example, the color of light from a light emitter (e.g. LED) in a spectroscopic sensor can be changed in an automatic, iterative, and/or scanning manner. In an example, the frequency of light from a light emitter in a spectroscopic sensor can be changed in an automatic, iterative, and/or scanning manner. In an example, the intensity and/or power of light from a light emitter in a spectroscopic sensor can be changed in an automatic, iterative, and/or scanning manner. In an example, the level of coherence of light from a light emitter in a spectroscopic sensor can be changed in an automatic, iterative, and/or scanning manner. In an example, the polarity of light from a light emitter in a spectroscopic sensor can be changed in an automatic, iterative, and/or scanning manner. In an example, the projection angle of light from a light emitter in a spectroscopic sensor can be changed in an automatic, iterative, and/or scanning manner. In an example, the spectrum of light from a light emitter in a spectroscopic sensor can be changed in an automatic, iterative, and/or scanning manner.

In an example, the frequency of light from a light emitter (e.g. LED) can be adjusted. In an example, the frequency of light from a light emitter (e.g. LED) can be automatically adjusted based on pattern recognition of food in a food image recorded by a camera. In an example, food images and spectroscopic analysis can be recorded by the same component. In an example, a spectroscopic sensor can be automatically activated with eating is detected. In an example, a camera on a wrist (and/or arm) band can be manually slid along a portion of the circumference of a wrist and/or arm band.

In an example, the results of analysis of data from a spectroscopic sensor which scans food can be superimposed on a food image which is shown on a camera viewfinder. In an example, a camera on a wrist (and/or arm) band can be 180-degrees around the circumference of the band from a watch face on the band. In an example, a wrist (and/or arm) band can include a motion sensor, wherein the focal vector of a spectroscopic sensor is adjusted as the wrist (and/or arm) band is moved to keep the camera focused toward food. In an example, a spectroscopic sensor can be slid along a quarter of the circumference of a wrist and/or arm band. In an example, a spectroscopic sensor can be slid around a portion of the circumference a band and then locked in place at a particular location on the band. In an example, a camera can be slid around a portion of the circumference a band and then locked in place at a particular location on the band.

In an example, the results of analysis of data from a spectroscopic sensor which scans food can be displayed on a watch face. In an example, a watch face can display the composition and/or types of nearby food based on the results of spectroscopic scanning of the food. In an example, the results of analysis of data from a spectroscopic sensor which scans food can be displayed on a primary display. In an example, a primary display can display the composition and/or types of nearby food based on the results of spectroscopic scanning of the food. In an example, a camera on a wrist (and/or arm) band can be diametrically-opposite a watch face on the band. In an example, the angle between the focal vector of a camera on a wrist (and/or arm) band and the emission vector of light from a light emitter on the band can be between 5 and 25 degrees. In an example, a spectroscopic sensor can be slid along a portion of the circumference of a wrist and/or arm band. In an example, the results of analysis of data from a spectroscopic sensor which scans food can be displayed on a primary display.

In an example, the results of analysis of data from a spectroscopic sensor (e.g. information on food composition, food types, nutritional composition, and/or calories) can be juxtaposed with (e.g. shown alongside) a food image shown on a wearable display (e.g. a smart watch display) or hand-held display (e.g. a cell phone display). In an example, the results of analysis of data from a spectroscopic sensor (e.g. information on food composition, food types, nutritional composition, and/or calories) can be superimposed on a food image shown on a wearable display (e.g. a smart watch display) or hand-held display (e.g. a cell phone display).

In an example, a spectroscopic sensor can slide out from a wrist-worn (or arm-worn) device to be moved closer to food. In an example, a wrist and/or arm worn device can have a recess into which a spectroscopic sensor is removably inserted. In an example, a spectroscopic sensor can be unclipped, unsnapped, or unplugged from a wrist-worn (or arm-worn) device to be moved closer to food. In an example, a wrist and/or arm worn device can have a clip, snap, or plug by which a spectroscopic sensor is removably attached to the device.

In an example, a spectroscopic sensor can be detached from a wrist (and/or arm) band and moved over (e.g. waved back and forth over) food to enable close-up spectroscopic analysis of food composition. In an example, a spectroscopic sensor can be detached from a wrist (and/or arm) band and placed on food to enable close-up spectroscopic analysis of food composition. In an example, a spectroscopic sensor can be detached from a wrist (and/or arm) band and inserted into food to enable internal spectroscopic analysis of food composition. In an example, a spectroscopic sensor can be detached from a primary housing on a band and inserted into food for internal spectroscopic analysis of the food.

In an example, a spectroscopic sensor can be located on one side of a camera on a wrist and/or arm band. In an example, a light emitter which is part of a spectroscopic sensor can be located on one side of a camera on a wrist (and/or arm) band and a light receiver which is also part of the spectroscopic sensor can be located on the opposite side of the camera. In an example, a camera on a wrist (and/or arm) band can function as both a spectroscopic sensor for analyzing food composition and as an imaging recording device to record food images.

In an example, a light emitter of a spectroscopic sensor can be on one side of a camera and a light receiver of the spectroscopic sensor can be on the opposite side of the camera. In an example, the distance between a light emitter (e.g. LED) and a light receiver can be automatically adjusted. In an example, a camera on a wrist (and/or arm) band can be diametrically-opposite a camera viewfinder on the band. In an example, the angle between a housing and light emission from a light emitter (e.g. LED) can be changed in an automatic, iterative, and/or scanning manner.

In an example, the location of a spectroscopic sensor on the circumference of a wrist (and/or arm) band can be adjusted by sliding the sensor. In an example, a spectroscopic sensor on a wrist (and/or arm) band can be diametrically-opposite a primary display on the band. In an example, a spectroscopic sensor on a wrist (and/or arm) band can be diametrically-opposite a watch face on the band. In an example, a camera on a wrist (and/or arm) band can be manually slid along one quarter of the circumference of a wrist and/or arm band. In an example, the angle of light emission from a light emitter (e.g. LED) can be automatically adjusted.

In an example, a wearable system for tracking food consumption can include two wrist-worn (and/or arm-worn) devices, one on each arm. In an example, collected data from two devices, one on each wrist (and/or arm) can provide more accurate eating detection and more accurate measurement of the types and amounts of food consumed than a single device on one wrist (and/or arm). Some people wear a watch on their non-dominant arm and primarily use their dominant arm to eat. In an example, two-device system comprising a smart watch (with a display and camera) which is worn on a person's non-dominant arm and a band with a motion sensor which is worn on the person's dominant arm can provide more accurate eating detection and more accurate measurement of the types and amounts of food consumed than a single device on one wrist (and/or arm). The person's dominant arm can be a better location for detecting eating (because it moves more during eating) and the non-dominant arm can be a better location for recording and displaying food images (because it moves less during eating).

In an example, a wearable system for tracking food consumption can comprise: a smart watch with a display and a camera which is configured to be worn on a person's first arm; and a band with a motion sensor which is configured to be worn on the person's second arm, wherein data from the motion sensor is analyzed to detect eating, and wherein the camera is activated when eating is detected. In an example, a wearable system for tracking food consumption can comprise: a smart watch with a display, camera, and spectroscopic sensor which is configured to be worn on a person's first arm; and a band with a motion sensor which is configured to be worn on the person's second arm, wherein data from the motion sensor is analyzed to detect eating, and wherein the camera and/or spectroscopic sensor are activated when eating is detected.

In an example, a wearable system for tracking food consumption can comprise: a smart watch with a display and a camera which is configured to be worn on a person's first arm; and a finger ring with a motion sensor which is configured to be worn on the person's second arm, wherein data from the motion sensor is analyzed to detect eating, and wherein the camera is activated when eating is detected. In an example, a wearable system for tracking food consumption can comprise: a smart watch with a display, camera, and spectroscopic sensor which is configured to be worn on a person's first arm; and a finger ring with a motion sensor which is configured to be worn on the person's second arm, wherein data from the motion sensor is analyzed to detect eating, and wherein the camera and/or spectroscopic sensor are activated when eating is detected.

I claim:

1. A wearable device for tracking food intake comprising:
a band worn on a person's wrist and/or arm;
a camera on the band, wherein the camera records food images, wherein the food images are analyzed to identify food types and quantities;
a primary display on the band, wherein the primary display is centered on a first location on the circumference of the band, wherein the camera is centered on a second location on the circumference of the band, wherein the second location is between 60 to 110 degrees around the band circumference away from the first location in a first direction, and wherein the first direction can be clockwise;
a camera viewfinder on the band, wherein the camera viewfinder displays the food images recorded by the camera, wherein the camera viewfinder is centered on a third location on the circumference of the band, and wherein the third location is between 70 and 110 degrees around the band circumference away from the first location in a second direction which is opposite the first direction, and wherein the second direction can be counter-clockwise;
a spectroscopic sensor on the band, wherein the spectroscopic sensor further comprises a light emitter and a light receiver, wherein the light emitter emits light rays toward food, wherein the light receiver receives the light rays after the rays have been reflected by the food, and wherein the light rays reflected by the food are analyzed to identify food types and/or composition; and
an eating detector on the band which collects data which is analyzed to detect when the person is eating, wherein the eating detector further comprises one or more components selected from the group consisting of: an accelerometer, a gyroscope, a magnetometer, a microphone, and an EMG sensor.

2. A wearable device for tracking food intake comprising:
a band worn on a person's wrist and/or arm;
a camera on the band, wherein the camera records food images, wherein the food images are analyzed to identify food types and quantities;
a primary display on the band, wherein the primary display displays food images recorded by the camera, wherein the primary display is centered on a first location on the circumference of the band, wherein the camera is centered on a second location on the circumference of the band, and wherein the second location is between 160 to 200 degrees around the band circumference away from the first location;
a spectroscopic sensor on the band, wherein the spectroscopic sensor further comprises a light emitter and a light receiver, wherein the light emitter emits light rays toward food, wherein the light receiver receives the light rays after the rays have been reflected by the food, and wherein the light rays reflected by the food are analyzed to identify food types and/or composition; and
an eating detector on the band which collects data which is analyzed to detect when the person is eating, wherein the eating detector further comprises one or more components selected from the group consisting of: an accelerometer, a gyroscope, a magnetometer, a microphone, and an EMG sensor.

3. A wearable device for tracking food intake comprising:
a band worn on a person's wrist and/or arm;
a primary housing on the band;
a flip-up display which flips, pivots, rotates, tilts, and/or pops up from the primary housing;
a camera on the band, wherein the camera records food images, wherein the food images are analyzed to identify food types and quantities, wherein the flip-up display serves as a viewfinder for the camera, wherein the primary housing is centered on a first location on the circumference of the band, wherein the camera is centered on a second location on the circumference of the band, and wherein the second location is between 70 and 110 degrees around the band circumference away from the first location;
a spectroscopic sensor on the band, wherein the spectroscopic sensor further comprises a light emitter and a light receiver, wherein the light emitter emits light rays toward the food, wherein the light receiver receives the light rays after the rays have been reflected by the food, and wherein the light rays reflected by the food are analyzed to identify food types and/or composition; and
an eating detector on the band which collects data which is analyzed to detect when the person is eating, wherein the eating detector further comprises one or more components selected from the group consisting of: an accelerometer, a gyroscope, a magnetometer, a microphone, and an EMG sensor.
